(19) United States
(12) Patent Application Publication
(10) Pub. No.: US 2024/0108766 A1
(43) Pub. Date: Apr. 4, 2024

(54) LRRC15 ANTIBODIES AND CONJUGATES THEREOF

(71) Applicants: Bayer Aktiengesellschaft, Leverkusen (DE); Bayer AS, Oslo (NO)

(72) Inventors: Mark TRAUTWEIN, Wuelfrath (DE); Urs Beat HAGEMANN, Glienicke/Nordbahn (DE); Phillipp ELLINGER, SOLINGEN (DE); Stephan MAERSCH, Paehl (DE); Joachim SCHUHMACHER, Wuppertal (DE); Antje WENGNER, Berlin (DE); Roger Malerbakken BJERKE, Oslo (NO); Helge ROIDER, Hohen Neuendorf (DE)

(21) Appl. No.: 18/262,311

(22) PCT Filed: Jan. 17, 2022
(86) PCT No.: PCT/EP2022/050831

§ 371 Date: Jul. 20, 2023

(30) Foreign Application Priority Data
Jan. 22, 2022 (EP) 21152886.4

(51) Int. Cl. A61K 51/10; A61K 51/04; A61P 35/00; C07K 16/28

(52) U.S. Cl. CPC A61K 51/1096; A61K 51/0478; A61K 51/1027; A61K 51/1045; A61P 35/00; C07K 16/28

(57) ABSTRACT

The present invention relates to conjugates comprising a chelator arranged for complexation of a radionuclide and a targeting moiety binding to LRRC15. For example, the conjugates according to the current invention can be targeted alpha therapeutics such as targeted thorium conjugates (TTCs), i.e. radioconjugates comprising an alpha emitter, such as ²²⁷Th. The present invention further relates to sequence-defined antibodies binding LRRC15 and antigen-binding fragments thereof. Also provided are conjugates comprising these antibodies or functional fragments thereof. The antibodies, functional fragments and conjugates according to the current invention can be used to treat cancer and other disorders and conditions associated with the expression of LRRC15.

### FIELD OF THE DISCLOSURE

The present invention relates to conjugates comprising a chelator arranged for complexation of a radionuclide and a targeting moiety binding to LRRC15. For example, the conjugates according to the current invention can be targeted alpha therapeutics such as targeted thorium conjugates (TTCs), i.e. radioconjugates comprising an alpha emitter, such as ²²⁷Th.

The present invention further relates to sequence-defined antibodies binding LRRC15 and antigen-binding fragments thereof. Also provided are conjugates comprising these antibodies or functional fragments thereof.

The antibodies, functional fragments and conjugates according to the current invention can be used to treat cancer and other disorders and conditions associated with the expression of LRRC15. Provided are pharmaceutical compositions and kits with instructions for use comprising the antibodies, functional fragments and conjugates according to the current invention.

The invention further provides methods and tools to generate the antibodies, functional fragments and conjugates according to the invention. For example, provided are polynucleotides encoding the foregoing antibodies or fragments, vectors containing the same, and cells for production.

### BACKGROUND

The most common methods of tumor treatment are currently surgery, chemotherapy and external beam irradiation. Targeted radionuclide therapy is, however, a promising and developing area with the potential to deliver highly cytotoxic radiation specifically to cell types associated with disease. Targeted radionuclides can be used for applications in tumor therapy, disease control or palliative care. The most common forms of radiopharmaceuticals currently authorised for use in humans employ beta-emitting and/or gamma-emitting radionuclides.

Targeted alpha therapeutics (TAT) have emerged as a promising modality in cancer therapy because of their potential for more specific cell killing. TAT take advantage of the combination of the highly potent radiobiological properties of an alpha particle emitting payload and a tumor-targeting moiety, such as a monoclonal antibody. Following systemic administration, TAT specifically accumulate and deliver high linear energy transfer (LET) a particles directly to the tumor and its microenvironment. The short-path length of 20-100 m (2-10 cell diameters) minimizes damage to the surrounding healthy tissue. High LET a particles (50-230 keV/μM) are highly cytotoxic due to the induction of difficult-to-repair clustered DNA double-strand breaks (DSBs).

Over the past two decades, a number of α-particle-emitting radionuclides, including bismuth-213 (²¹³Bi, half-life of 45.6 min), actinium-225 (²²⁵Ac, half-life of 9.9 d), astatine-211 (²¹¹At, half-life of 7.2 h), radium-223 (²²³Ra, half-life of 11.4 d), radium-224 (²²⁴Ra, half-life of 3.7 d), and thorium-227 (²²⁷Th, half-life of 18.7 d) have been investigated preclinically for TATs. Some have progressed to clinical development, while, to date only ²²³Ra has been approved by the US Food and Drug Administration and the European Medicines Agency. However, the paucity of efficient chelator systems for the conjugation of ²²³Ra to targeting moieties has prevented the development of this radionuclide as a ligand-based targeted radioimmunotherapy.

In contrast, chelators for thorium-227 (²²⁷Th) and other radionuclides have been successfully developed. ²²⁷Th can be efficiently complexed, e.g. with octadentate 3,2-hydroxypyridinone (3,2-HOPO) chelators that are conjugated to antibodies or other targeting moieties, resulting in highly stable targeted thorium conjugates (TTCs).

### Targeted Thorium Conjugates

TTCs comprise three main building blocks and represent a new promising class of TATs for cancer therapy, capable of delivering a high-energy α-particle radiation to tumors by targeting antigens specifically expressed or overexpressed in cancer tissue versus healthy tissue.

Following the β-particle decay of actinium-227 (half-life 21.8 years), α-particle-emitting radionuclide ²²⁷Th—being the first building block—is purified by ion exchange chromatography. ²²⁷Th is produced from the same supply chain as ²²³Ra, and is available in quantities that support usual drug development and commercialization programs. ²²⁷Th decays by α-particle emission with an energy of 5.9 MeV and a half-life of 18.7 d to ²²³Ra with further decay releasing four a particles with a mean energy of 6.6 MeV and two 3 particles, ending the cascade with the formation of stable ²⁰⁷Pb.

As discussed earlier, the second building block of a TTC is a chelator or chelating group. While various chelators have been developed and are suitable according to the current invention—as recognized by the skilled person—some basic structures shall be discussed in the following. One example is a siderophore-derived chelator containing HOPO groups bearing four 3-hydroxy-N-methyl-2-pyridinone moieties on a symmetrical polyamine scaffold functionalized with a carboxylic acid linker for bioconjugation. Conjugation to a targeting moiety can be achieved e.g. through the amide bond formation with the ε-amino groups of lysine residues. These octadentate 3,2-HOPO chelators can be very efficiently labeled with ²²⁷Th, with high yield, purity, and stability at ambient conditions. Compared with another chelator, tetra-azacyclododecane-1,4,7,10-tetraacetic acid (DOTA), which often requires heating, the HOPO chelators are superior due to efficient radiolabeling at ambient temperatures and high stability of formed complexes.

The third building block of a TTC is the targeting moiety. The targeting moiety has to satisfy various requirements and can be seen as a major decisive factor for the suitability of a TAT in a specific medical indication. Several different target structures have been evaluated in the past for TATs and many of them have failed. According to the current invention, stromal target LRRC15 has been evaluated as a TAT and TTC target structure and has lead to surprisingly strong anti-tumor effects both in vivo and in vitro.

The specific mode of action of TTCs and TATs leads to the fact, that a target which has been found suitable for a non radioactive antibody-drug conjugates may not be suitable for a TAT or TTC. The same holds true for a specific antibody: even where the antibody is suitable as part of an ADC, this does not necessarily predict suitability for a TAT or a TTC. Due to an inherent cross-fire effect of α-particle emitters and their ability to penetrate 2-10 cell layers in tissue, the biological activity of TTCs, in contrast to antibody-drug conjugates, does not strictly depend on antigen internalization and is broadly independent of homogeneity of antigen expression. A critical parameter for their optimal efficacy, however, is the efficient delivery, accumulation, and retention in the tumor tissue to ensure treatment efficacy and a minimal damage to the surrounding healthy tissue.

The administration setup for an alpha-emitting radioisotope has to be carefully adjusted and requires vigilant design of targeting concepts, i.e. by developing conjugates for reliable complexing of the radionuclide, careful selection of suitable biological target structures and precise targeting using optimized antibodies.

### Prior Art: Generic TTCs and 227Th Chelators

WO2011098611 and related members of the patent family disclose a tissue-targeting complex comprising a tissue targeting moiety, a 3,2-hydroxypyridinone (HOPO)-containing ligand and the ion of an alpha-emitting thorium radionuclide. In more detail, octadentate chelators are described, containing four 3,2-hydroxypyridinone groups joined to an amine-based scaffold, having a separate reactive group used for conjugation to a targeting molecule. Preferably, isothiocyanate chemistry is used as coupling chemistry. The isothiocyanate is widely used to attach labels to proteins via amine groups. The isothiocyanate group reacts with amino terminal and primary amines in proteins and has been used for the labelling of many proteins including antibodies.

WO2013167754 discloses that the use of a 4+ thorium-227 ion complexed by an octadentate hydroxypyridinone (HOPO)-type ligand comprising four HOPO moieties of which at least one is substituted with a suitable solubilising moiety can provide a dramatic improvement in solubility and corresponding properties of the complex. Furthermore, coupling of such a ligand to a CD22-binding targeting moiety can provide a conjugate having advantageous properties.

In addition WO2013167755 and WO2013167756 disclose the hydroxyalkyl/isothiocyanate conjugates applied to CD33 or CD22 targeted antibodies.

WO2013022797 and WO2015055318 both disclose PSMA-targeting peptides and linking structures for use with beta-emitting radionuclides. The applications discloses a number of specific chelators suitable for use with the peptides.

WO2014195423 and related members of the patent family disclose a method for removal of ²²³Ra from a ²²⁷Th solution.

WO2016096843 discloses 3,2-HOPO chelators radiolabeled with thorium and attached to a variety of tissue-targeted moieties. WO2016096843 furthermore discloses a method comprising: a) forming an octadentate chelator comprising four HOPO moieties, b) coupling said chelator to at least one tissue-targeting peptide or protein; and c) contacting said tissue-targeting chelator with an aqueous solution comprising an ion of at least one alpha-emitting thorium isotope.

None of these documents discloses a TTC or radioconjugate wherein the targeting moiety is directed to a stromal protein such as LRRC15. Instead, before the current invention was made, it was suspected, that the (stromal) expression pattern of LRRC15 and the internalization behavior for this target might be disadvantageous for selection of LRRC15 as a target for a TTC.

### LRRC15 and Antibodies Binding LRRC15

Membrane protein leucine-rich repeat containing 15 (LRRC15) is a TGFβ-regulated structural protein. LRRC15 is highly expressed in multiple solid tumor indications, while there is only limited expression of LRRC15 in normal tissue. Normal tissue expression of LRRC15 is limited to mesenchymal cells in restricted tissue types. These tissue types include hair follicular cells, tonsils, area of wound healing (skin), stomach (pylorus/cardia), spleen as well as pediatric bone (Purcell, James W., et al. “LRRC15 is a novel mesenchymal protein and stromal target for antibody-drug conjugates.” Cancer research 78.14 (2018): 4059-4072.).

Solid tumors with stromal fibroblasts expressing LRRC15 are e.g. lung cancer, pancreatic cancer, breast cancer and head and neck cancer. Stromal LRRC15 expression may be observed on both primary tumors as well as at metastatic sites, see example 15. For example, LRRC15 is highly expressed on cancer associated fibroblasts (CAFs) in the stromal microenvironment of breast cancer. The stroma of a cancer may, however, have an ambiguous role. For example, targeting the stroma in pancreatic ductal adenocarcinoma (PDAC) may result in undifferentiated, aggressive pancreatic cancer (Gore, Jesse, and Murray Korc. “Pancreatic cancer stroma: friend or foe?” Cancer cell 25.6 (2014): 711-712.). The success of a TTC approach targeting mainly the stroma is therefore difficult to predict and may differ from a stroma targeting ADC approach, due to the differences in the mode of action.

Expression has also been found in a subset of cancer cells of mesenchymal origin, namely in sarcoma, melanoma, and glioblastoma. Glioblastoma cancer cells directly express LRRC15. WO2005037999 relates to the treatment of cancer using antibodies binding LRRC15.

WO2005094348 discloses anti LRRC15 antibodies including murine antibody M25, e.g. for the diagnosis, prognosis and treatment of cancer, and furthermore discloses monomethyl auristatin-based LRRC15 antibody drug conjugates (ADCs).

WO2017095805 and WO2017095808 relate to auristatin-based LRRC15 ADCs, wherein the antibodies are defined by sequence. Claim 27 of WO'805 discloses anti-huLRRC15 antibodies defined by sequences, including huM25 (in the following: TPP-12942, SEQ ID No. 11-20). HuM25 is a humanized antibody of the murine precursor M25 as described in WO2005094348. WO2005037999, WO2005094348, WO2017095805 and WO2017095808 are silent with regard to thorium conjugates targeting LRRC15.

### SUMMARY

### Current Invention

A targeting antibody according to the current invention is required to bind with sufficient affinity to human LRRC15 expressed on at least some of the target cells. Cross-reactivity to monkey LRRC15, e.g. within one order of magnitude of monovalent K_{D}, is furthermore beneficial to safely reflect binding on normal tissues in the toxicology monkey model even at low surface densities under non-avidity based binding conditions. Off-target binding to structures or proteins which are expressed at healthy sides may result in accumulation of radioactivity at these sides and may damage healthy structures. Clearance behavior of the antibody and conjugate likewise influences the therapeutic suitability and may be difficult to predict.

According to the current invention there are provided several antibodies or antigen-binding fragments thereof binding human LRRC15. These antibodies were found particularly suitable as targeting moieties for radioconjugates such as targeted alpha therapeutics. Beside their suitability for radioconjugates such as TATs or TTCs, the antibodies according to the current invention can also be used for various other purposes such as imaging, antibody drug conjugates or as therapeutic agents in the absence of a payload.

In particular the antibodies
i. are high affinity binder of human LRRC15,
ii. are cross-reactive to cynomolgus LRRC15, e.g. within one order of magnitude of monovalent KD,
iii. are characterized by a favorable off-target, polyreactivity and/or polyspecificity profile, i.e. do not show substantial binding to other proteins than LRRC15,
iv. are characterized by favorable clearance rates,
v. are human or humanized and show only few germline deviations, such that they are non-immunogenic in human therapy.

All antibodies according to the current invention have an excellent affinity not only for human LRRC15 but also for cynomolgous LRRC15 (cf. Table 1, example 6). Furthermore antibodies according to the current invention show an improved temperature stability at 37° C. (example 3). TPP-17074 shows a decrease in binding only at 37° C., but not below. In this case, introduction of mutations into the CDRs resulted in an unexpected stabilization of the dissociation rate constant in a temperature gradient. Importantly, half-lives of the antibody-antigen complexes at 37° C. differ significantly (1.6 min for TPP-12942, 17.5 min for TPP-17078 and 64.2 min for TPP-17421). This feature is especially relevant for therapeutic interventions as antibody binding is required to occur at about 37° C. body temperature in a human patient. The half life of the antibody antigen complex is important not only for the anticipated time of activity at the tumor site but also to reduce unspecific distribution of radioactivity.

Furthermore, antibodies according to the current invention showed no polyreactiviy and a superior off-target profile (example 2). For example, inventive antibodies TPP-1633, TPP-14389, TPP-14392, TPP-17078 and TPP-17421 show no or strongly reduced binding to EPHB6. This off-target binding is a major issue for the prior art antibodies disclosed in WO2017095805 and WO2017095808.

Antibodies according to the current invention are characterized by a clearance rate in cynomolgus monkeys 0.5 ml kg⁻¹ h⁻¹. This makes them particularly suitable for clinical use as described elsewhere herein.

Furthermore, all antibodies according to the current invention are characterized by a low number of germline deviations to human, e.g. less than 16 deviations in the light chain and less or equal to 16 deviations in the heavy chain (example 7). A low number of germline deviations leads to an improved immunogenicity profile of the antibody for that species. In consequence, the lower number of germline deviations further contributes to an improved suitability for clinical or therapeutic use.

Finally, the inventive antibodies had an improved stability at low pH conditions during downstream processing and can thereby be provided in a more reliable and cost-effective way. Inventive antibodies TPP-14389, TPP-14392, TPP-17078 and TPP-17421 yielded a percentage of >95% intact antibody after downstream processing (example 8).

According to the current invention it has been shown for the first time that targeting LRRC15 with a TTC approach effectively reduced the tumor size for various tumor indications including pancreatic cancer, head and neck cancer, non-small cell lung cancer and syngeneic breast cancer models (example 13).

### BRIEF DESCRIPTION OF THE DRAWINGS

FIG. 1: Flow cytometry analysis of binding of TPP-12942 to HEK293 cells transfected with LRRC15/ZsGreen1, EPHB6/ZsGreen1, PIK3AP1/ZsGreen1, or ZsGreen1-only (ZS HEK). Plotted is the Median AF647 Fluorescence versus TPP-12942 concentration. The EC50 binding value of TPP-12942 to LRRC15 was determined to be 1.4+/−0.5 μg/ml. The elevated binding of TPP-12942 to EPHB6-transfected cells is evident.

FIG. 2: Flow cytometry analysis of binding of TPP-14389 to HEK293 cells transfected with LRRC15/ZsGreen1, CTSS/ZsGreen1, or ZsGreen1-only (ZS HEK). Plotted is the Median AF647 Fluorescence versus TPP-14389 concentration. The EC50 binding value of TPP-14389 to LRRC15 was determined to be 0.26+/−0.03 μg/ml. No binding of TPP-14389 to CTSS-transfected cells is evident.

FIG. 3: Flow cytometry analysis of binding of TPP-12942, TPP-17078, and TPP-17421 to HEK293 cells transfected with LRRC15/ZsGreen1 or ZsGreen1-only. Plotted is the Median AF647 Fluorescence versus antibody concentration. Background binding of each antibody to the cells (i.e. binding to ZsGreen1-only transfectants) is subtracted from the LRRC15 transfected cells at each antibody dose. The EC50 binding value of TPP-12942, TPP-17078, and TPP-17421 to LRRC15 was determined to be 0.20 μg/ml, 0.15 μg/ml, and 0.42 μg/ml, respectively. No binding of human IgG1 isotype control TPP-754 is evident.

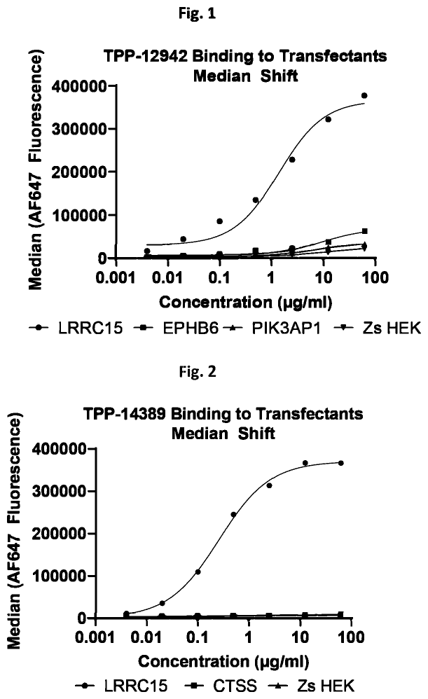

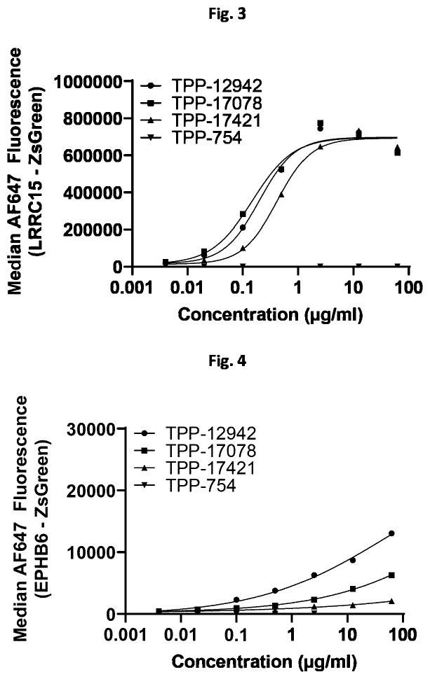

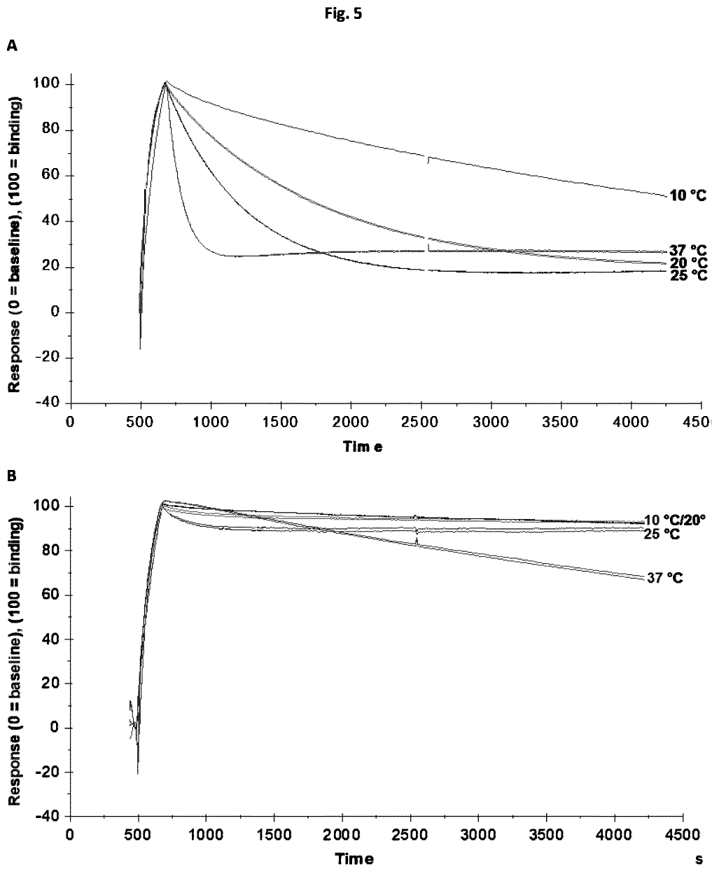

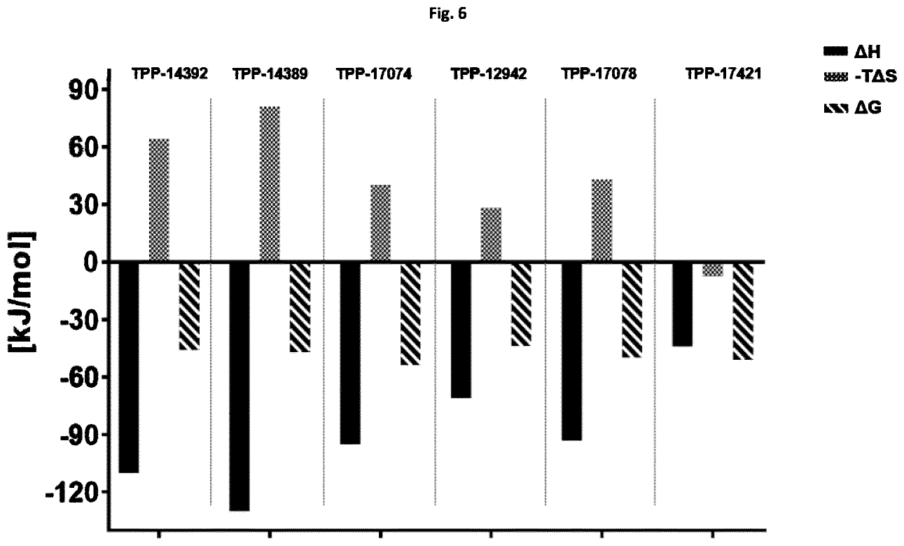

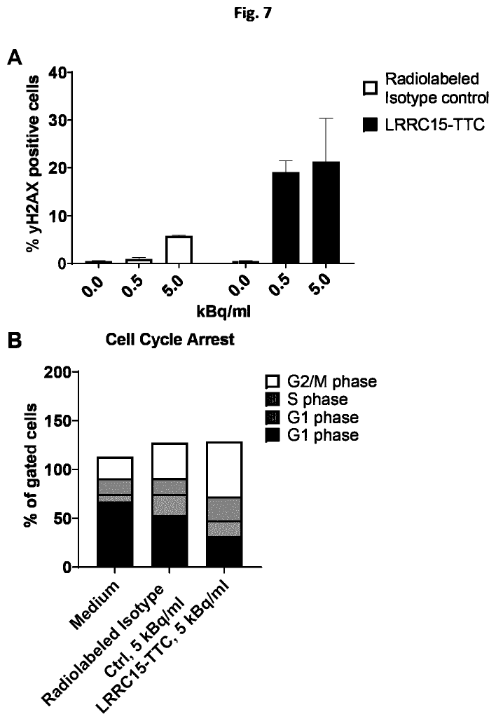

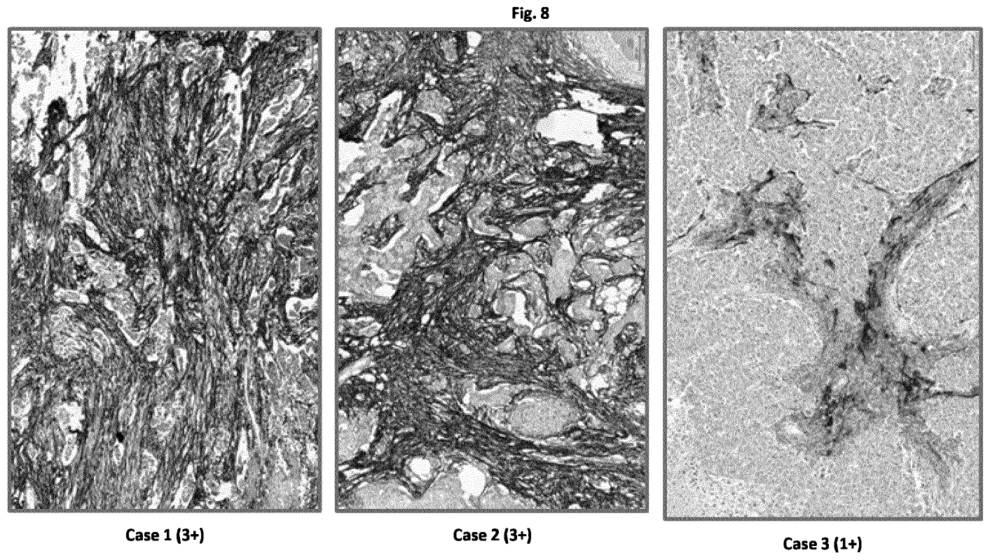

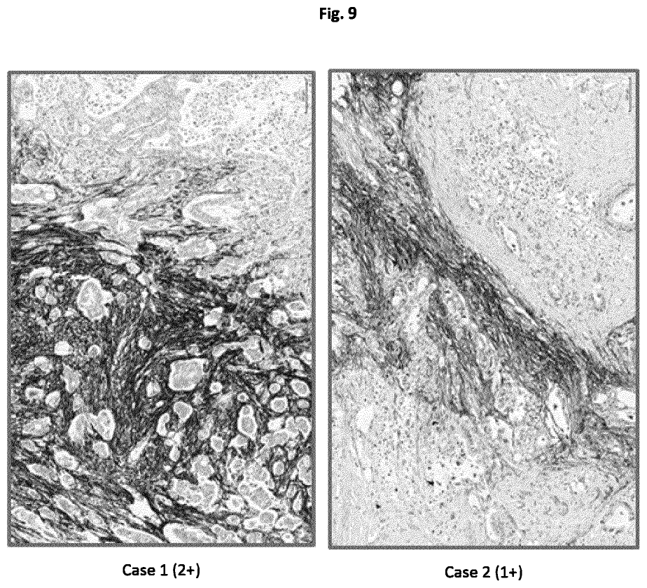

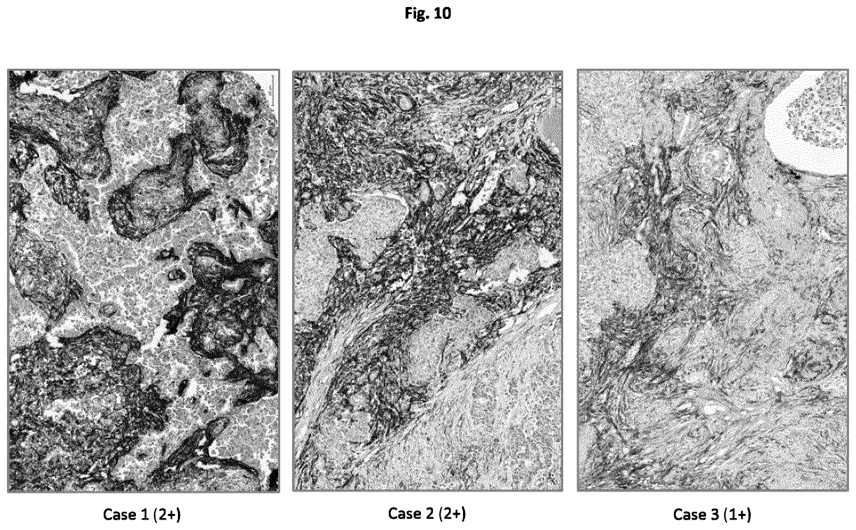

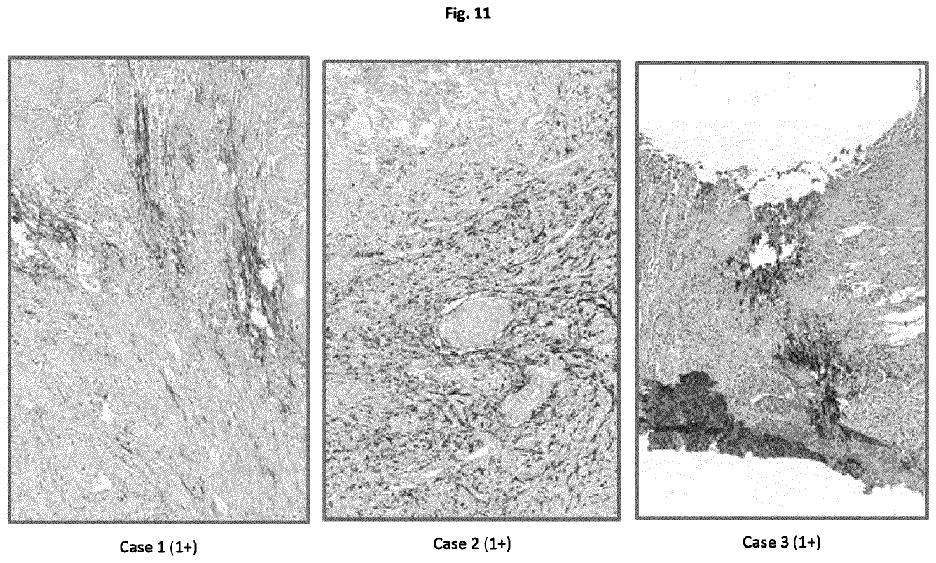

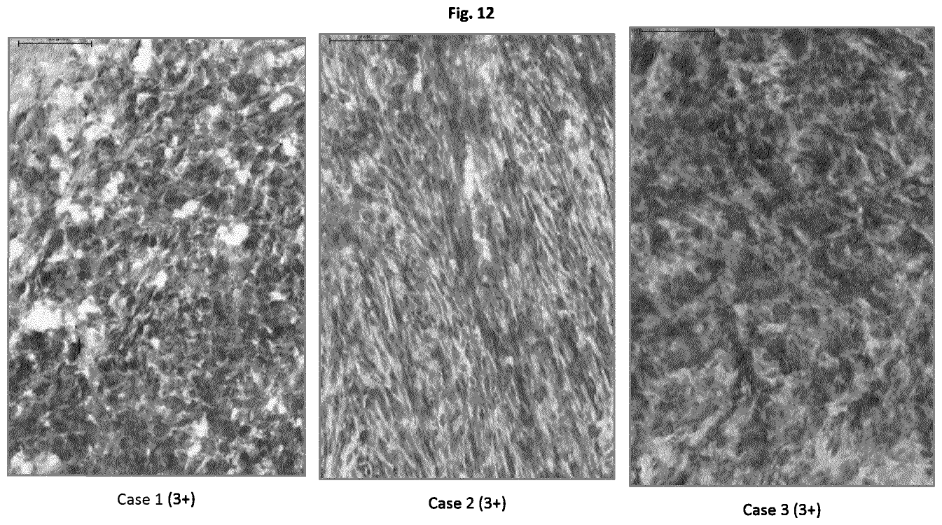

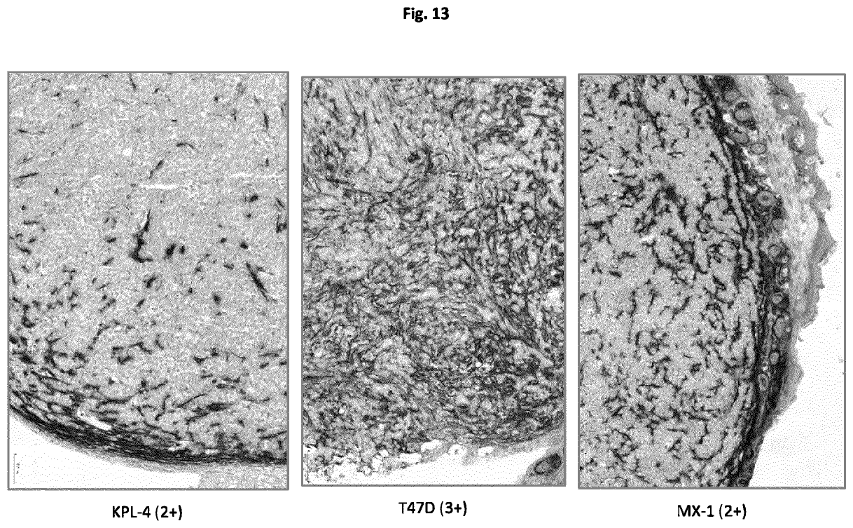

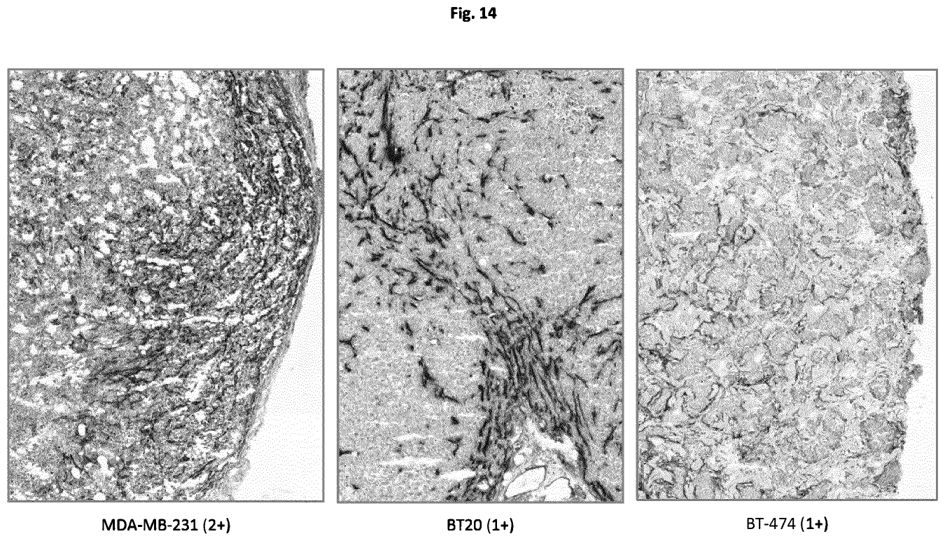

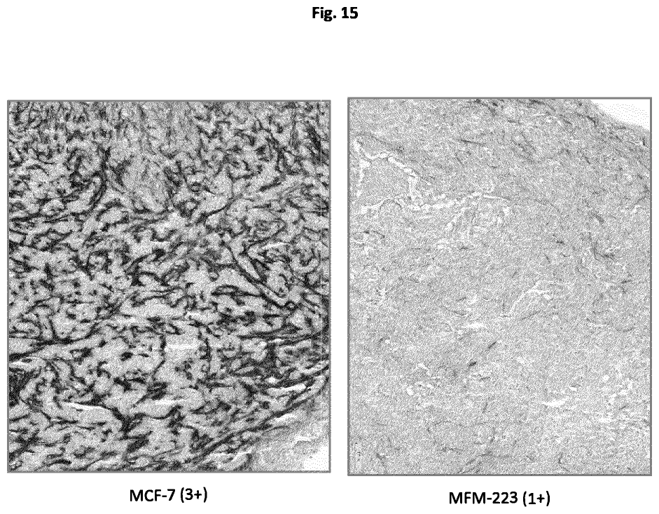

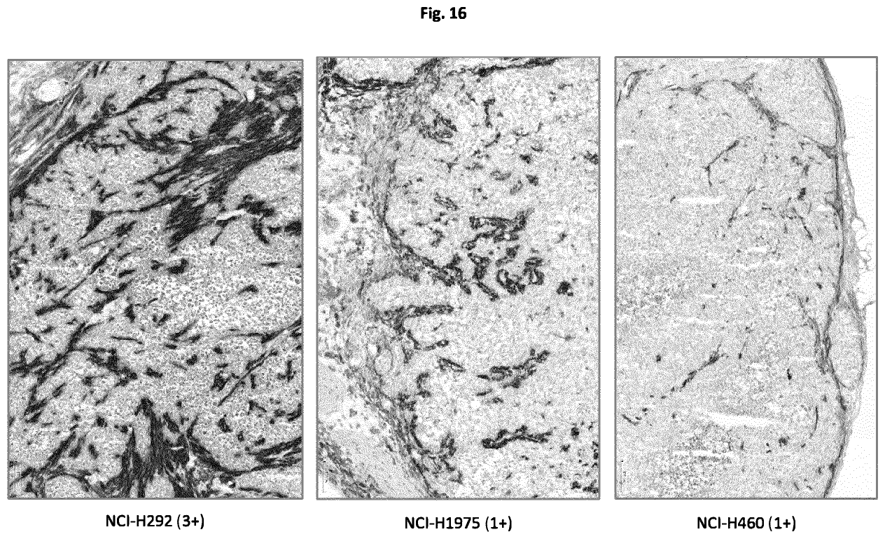

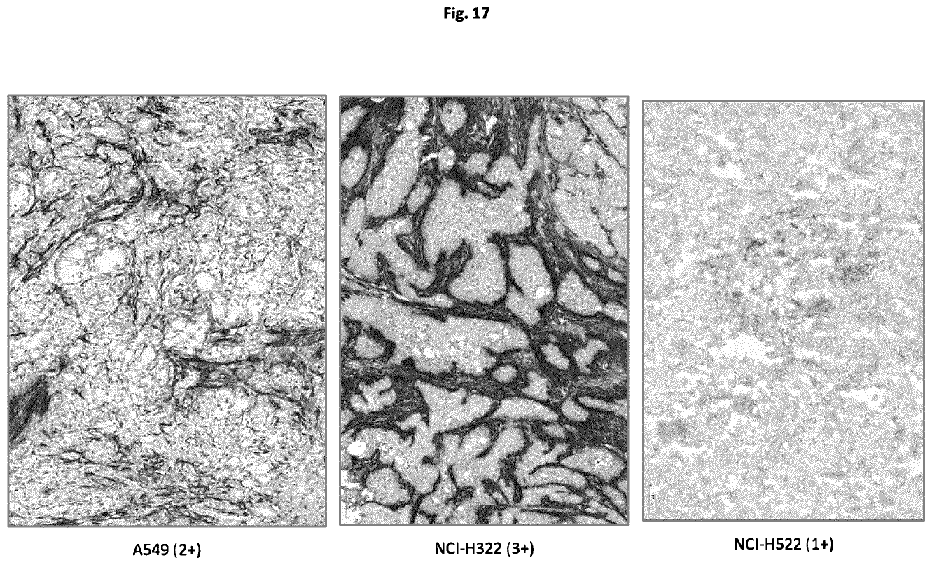

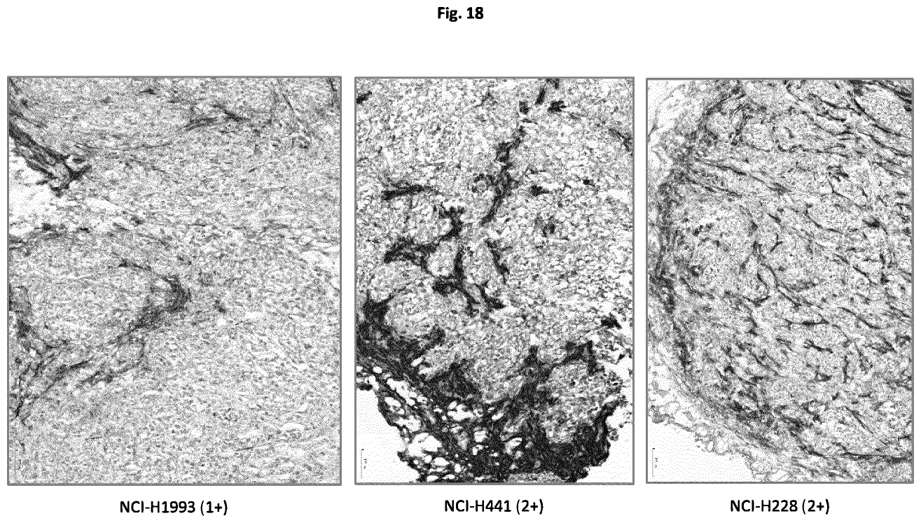

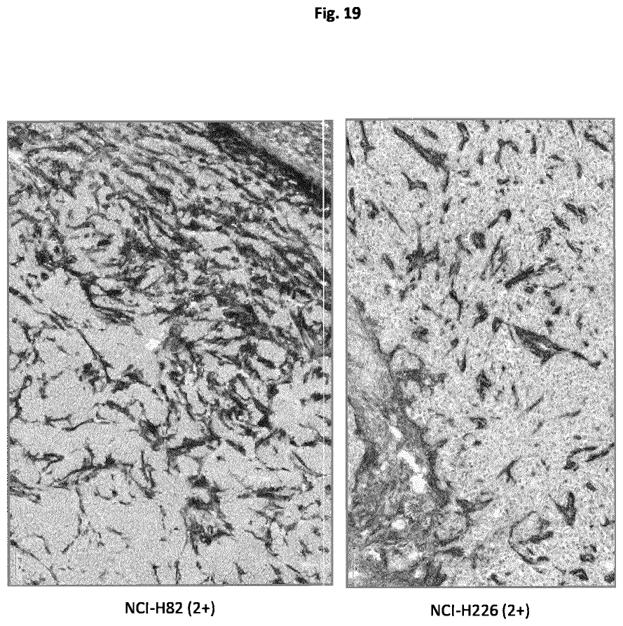

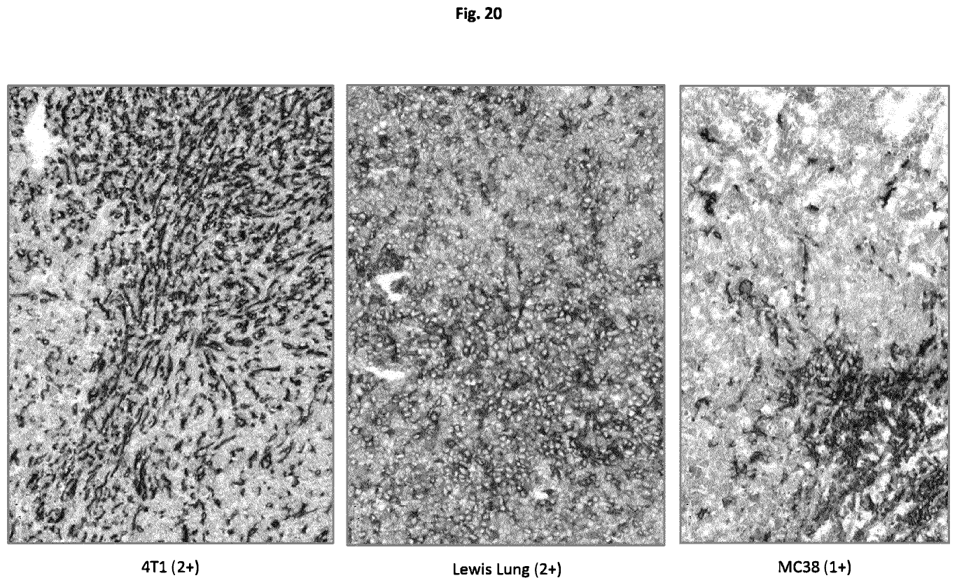

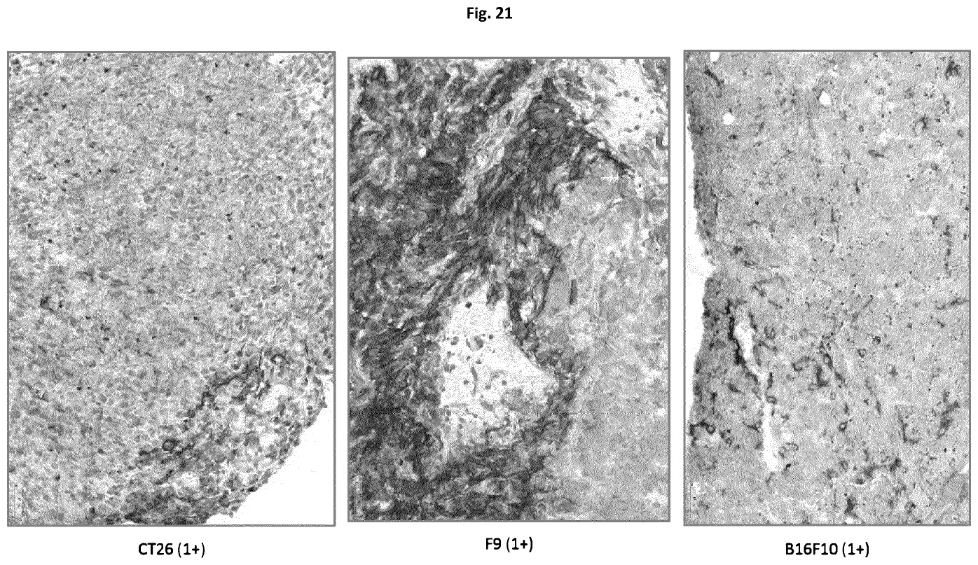

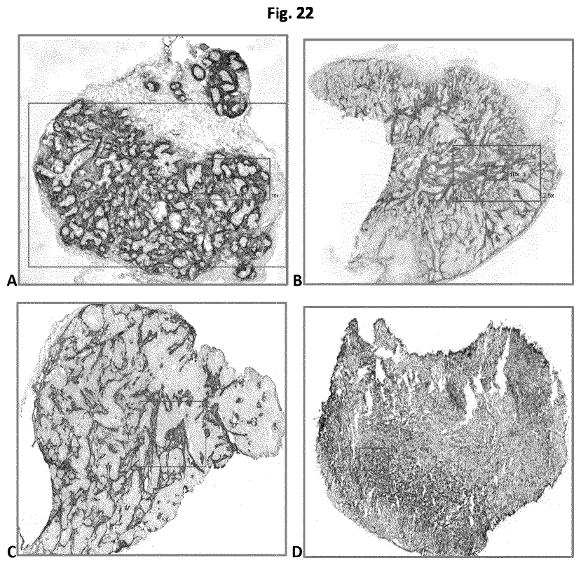

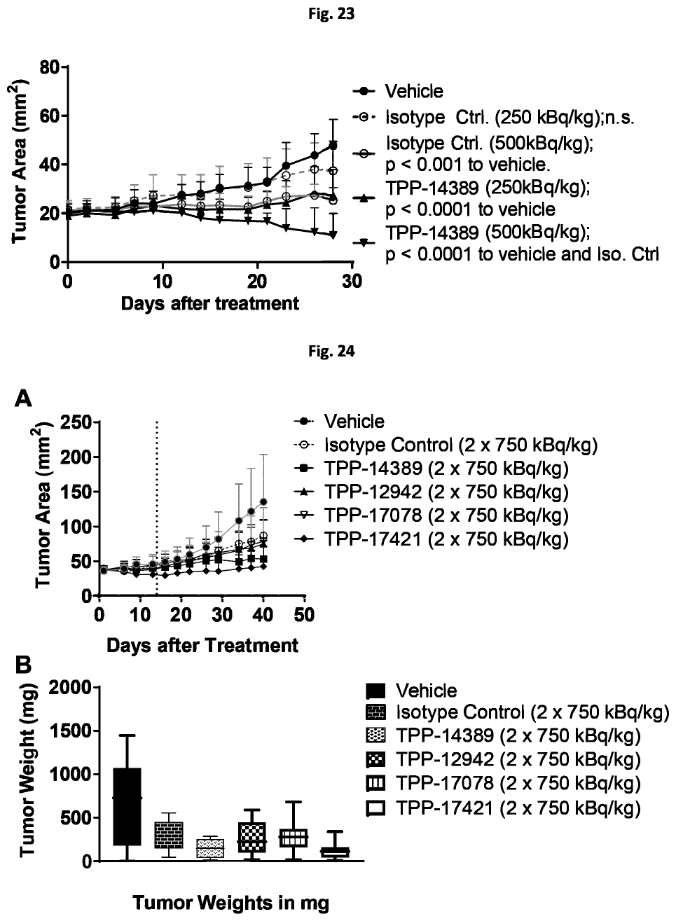

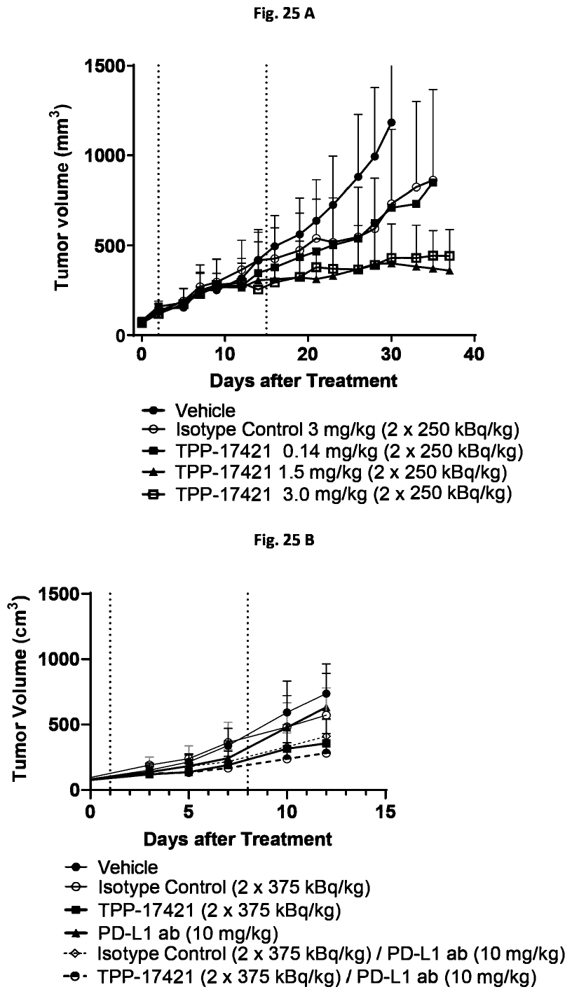

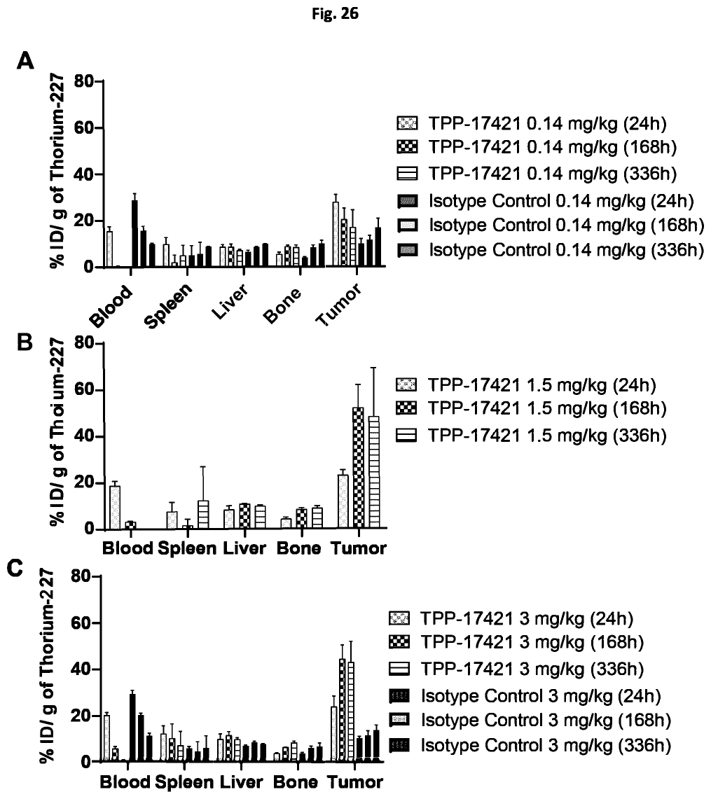

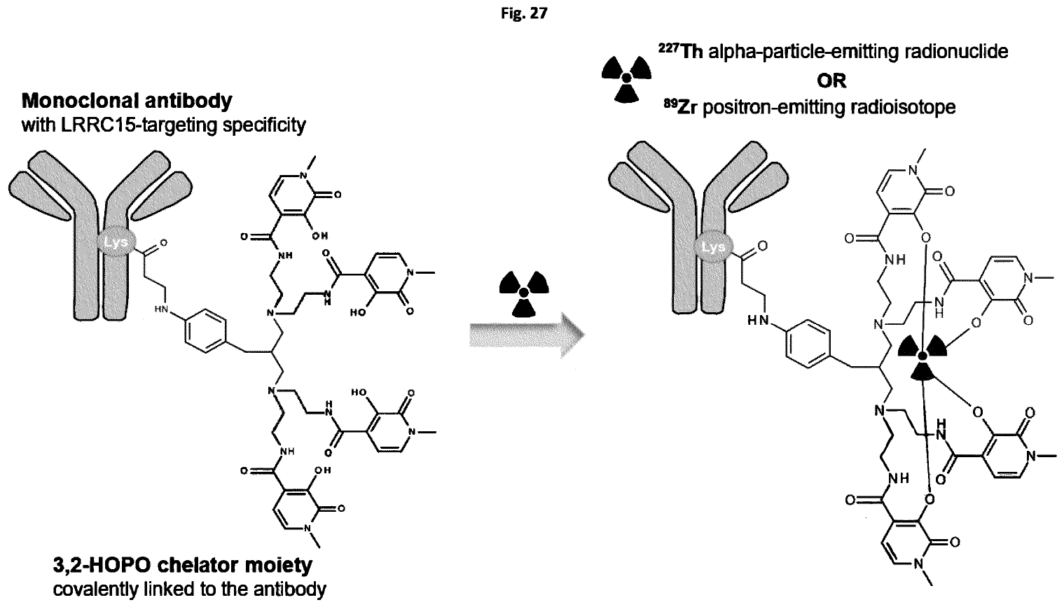

FIG. 1: Flow cytometry analysis of binding of TPP-12942, TPP-17078, and TPP-17421 to HEK293 cells transfected with EPHB6/ZsGreen1 or ZsGreen1-only. Plotted is the Median AF647 Fluorescence versus antibody concentration. Background binding of each antibody to the cells (i.e. binding to ZsGreen1-only transfectants) is subtracted from the LRRC15 transfected cells at each antibody dose. No binding is evident for human IgG1 isotype control TPP-754. The EC50 binding value of TPP-12942 to EPHB6 was determined to be 51.8 μg/ml. TPP-754 represents a human IgG1 isotype control.

FIG. 5: SPR data. Temperature dependence of TPP-12942 (A) compared to TPP-17421 (B).

FIG. 2: Thermodynamic parameters, free Gibbs energy (ΔG), enthalpy (ΔH) and entropy term (-TΔS) are plotted in kJ/mol for six different antibodies.

FIG. 7: Induction of DNA double strand breaks (A) and cell cycle arrest (B) in vitro by LRRC15-TTC (TPP-14389) in comparison to a radiolabeled isotype control on LRRC15-transfected human colorectal HT29 cells.

FIG. 8: IHC analysis of human breast cancer patient biopsies with detection of LRRC15 in the tumor stroma at a five-fold magnification.

FIG. 9: IHC analysis of human pancreatic cancer patient biopsies with detection of LRRC15 in the tumor stroma at a five-fold magnification.

FIG. 10: IHC analysis of human non-small cell lung cancer patient biopsies with detection of LRRC15 in the tumor stroma at a five-fold magnification.

FIG. 11: IHC analysis of human head and neck squamous cell cancer patient biopsies with detection of LRRC15 in the tumor stroma at a five-fold magnification.

FIG. 12: IHC analysis of human sarcoma patient derived xenograft models with detection of LRRC15 in the tumor stroma and partly on the tumor cells at a five-fold magnification.

FIG. 13: IHC analysis of human breast cancer cell line derived xenograft models with detection of LRRC15 in the tumor stroma at a five-fold magnification.

FIG. 143: IHC analysis of human breast cancer cell line derived xenograft models with detection of LRRC15 in the tumor stroma at a five-fold magnification.

FIG. 15: IHC analysis of human breast cancer cell line derived xenograft models with detection of LRRC15 in the tumor stroma at a five-fold magnification.

FIG. 16: IHC analysis of human lung cancer cell line derived xenograft models with detection of LRRC15 in the tumor stroma at a five-fold magnification.

FIG. 17: IHC analysis of human lung cancer cell line derived xenograft models with detection of LRRC15 in the tumor stroma at a five-fold magnification.

FIG. 18: IHC analysis of human lung cancer cell line derived xenograft models with detection of LRRC15 in the tumor stroma at a five-fold magnification.

FIG. 19: IHC analysis of human lung cancer cell line derived xenograft models with detection of LRRC15 in the tumor stroma at a five-fold magnification.

FIG. 20: IHC analysis of murine syngeneic cell line derived tumor models with detection of LRRC15 in the tumor stroma at a ten-fold magnification.

FIG. 21: IHC analysis of murine syngeneic cell line derived tumor models with detection of LRRC15 in the tumor stroma at a ten-fold magnification.

FIG. 22: Analysis of LRRC15 expression using immunohistochemistry staining in the different human tumor xenograft samples (A, B and C) as well as syngeneic mouse models (D). Tumor samples were stained for LRRC15 using a murine anti LRRC15 antibody. A, Calu-3 (NSCLC) xenograft. B, BxPC-3 (PancCa). C, SCC-15 (HNSCC). D, 4T1 (murine BrCa).

FIG. 23: Efficacy of LRRC15-TTC (with targeting moiety TPP-14389) in the human NSCLC xenograft model Calu-3. Presented are the tumor growth inhibition data over the course of 30 days after treatment, including statistical significance compared to vehicle.

FIG. 24: Efficacy of LRRC15-TTCs in the human PancCa xenograft model BxPC-3. LRRC15-TTCs are labeled based on the respective targeting moiety. (A) Tumor area over the course of 40 days after treatment. (B) Determined tumor weights at study end of respective treatment groups. The statistical significance (one way annova) compared to vehicle was as follows: isotype control, p<0.01; TPP-14389, p<0.0001; TPP-12942, p<0.001; TPP-17078, p<0.001; TPP-17421, p<0.0001.

FIG. 25 A4: Efficacy of LRRC15-TTC (with targeting moiety TPP-17421) in the human HNSCC xenograft model SCC-15. LRRC15-TTC as well as a radiolabeled isotype control were administered at a dose of 2×250 kBq/kg (interim of one week) using total antibody doses of 0.14, 1.5 and 3 mg/kg.

FIG. 25 B: Efficacy of LRRC15-TTC (with targeting moiety TPP-17421) in the murine breast cancer model 4T1 in immunocompetent mice. LRRC15-TTC as well as a radiolabeled isotype control were administered at a dose of 2×375 kBq/kg (interim of one week) using total antibody doses of 0.14 mg/kg. Anti PD-L1 antibody was administered at 10 mg/kg (i.p.; dosed every third or fourth day) in monotherapy or in combination with LRRC15-TTC or radiolabeled isotype control.

FIG. 265: Biodistribution of LRRC15-TTC (with targeting moiety TPP-17421) in the human HNSCC xenograft model SCC-15. LRRC15-TTC as well as a radiolabeled isotype control were administered at a dose of 2×250 kBq/kg (interim of one week) using total antibody doses of 0.14, 1.5 and 3 mg/kg. Thorium-227 accumulation is presented in % of ID/g as determined at the timepoints indicated. (A) Total antibody dose of 0.14 mg/kg. (B) Total antibody dose of 1.5 mg/kg. (C) Total antibody dose of 3 mg/kg.

FIG. 27. Generation of TTCs. Schematic representation of the generation of TTCs. Monoclonal antibodies as moieties with tumor-targeting specificity are covalently linked to octadentate 3,2-HOPO chelator through the ε-amino groups of lysine residues to generate the antibody-3,2-HOPO chelator conjugate. The binding of a radionuclide (227Th or 89Zr) to the chelator involves the formation of several bonds, resulting in a stable radionuclide-labeled antibody-3,2-HOPO chelator complex. 3,2-HOPO, 3,2-hydroxypyridinone; 227Th, thorium-227; TTCs, targeted thorium-227 conjugates, 89Zr, zirconium.

### BRIEF DESCRIPTION OF THE SEQUENCE IDs

The sequence listing provided with the application via electronic filing is included herein in its entirety.

| | | Sequence | Seq | |
|---|---|---|---|---|
| TPP ID | Sequence Name | Region | Type | SEQ ID |
| TPP-1633 | 060E-M016-G14-hIgG1 | VH | PRT | SEQ ID NO: 1 |
| TPP-1633 | 060E-M016-G14-hIgG1 | HCDR1 | PRT | SEQ ID NO: 2 |
| TPP-1633 | 060E-M016-G14-hIgG1 | HCDR2 | PRT | SEQ ID NO: 3 |
| TPP-1633 | 060E-M016-G14-hIgG1 | HCDR3 | PRT | SEQ ID NO: 4 |
| TPP-1633 | 060E-M016-G14-hIgG1 | VL | PRT | SEQ ID NO: 5 |
| TPP-1633 | 060E-M016-G14-hIgG1 | LCDR1 | PRT | SEQ ID NO: 6 |
| TPP-1633 | 060E-M016-G14-hIgG1 | LCDR2 | PRT | SEQ ID NO: 7 |
| TPP-1633 | 060E-M016-G14-hIgG1 | LCDR3 | PRT | SEQ ID NO: 8 |
| TPP-1633 | 060E-M016-G14-hIgG1 | Heavy Chain | PRT | SEQ ID NO: 9 |
| TPP-1633 | 060E-M016-G14-hIgG1 | Light Chain | PRT | SEQ ID NO: 10 |
| TPP-12942 | huM25-hIgG1Kappa | VH | PRT | SEQ ID NO: 11 |
| TPP-12942 | huM25-hIgG1Kappa | HCDR1 | PRT | SEQ ID NO: 12 |
| TPP-12942 | huM25-hIgG1Kappa | HCDR2 | PRT | SEQ ID NO: 13 |
| TPP-12942 | huM25-hIgG1Kappa | HCDR3 | PRT | SEQ ID NO: 14 |
| TPP-12942 | huM25-hIgG1Kappa | VL | PRT | SEQ ID NO: 15 |
| TPP-12942 | huM25-hIgG1Kappa | LCDR1 | PRT | SEQ ID NO: 16 |
| TPP-12942 | huM25-hIgG1Kappa | LCDR2 | PRT | SEQ ID NO: 17 |
| TPP-12942 | huM25-hIgG1Kappa | LCDR3 | PRT | SEQ ID NO: 18 |
| TPP-12942 | huM25-hIgG1Kappa | Heavy Chain | PRT | SEQ ID NO: 19 |
| TPP-12942 | huM25-hIgG1Kappa | Light Chain | PRT | SEQ ID NO: 20 |
| TPP-14389 | 13612-rec02-hIgG1Kappa | VH | PRT | SEQ ID NO: 21 |
| TPP-14389 | 13612-rec02-hIgG1Kappa | HCDR1 | PRT | SEQ ID NO: 22 |
| TPP-14389 | 13612-rec02-hIgG1Kappa | HCDR2 | PRT | SEQ ID NO: 23 |
| TPP-14389 | 13612-rec02-hIgG1Kappa | HCDR3 | PRT | SEQ ID NO: 24 |
| TPP-14389 | 13612-rec02-hIgG1Kappa | VL | PRT | SEQ ID NO: 25 |
| TPP-14389 | 13612-rec02-hIgG1Kappa | LCDR1 | PRT | SEQ ID NO: 26 |
| TPP-14389 | 13612-rec02-hIgG1Kappa | LCDR2 | PRT | SEQ ID NO: 27 |
| TPP-14389 | 13612-rec02-hIgG1Kappa | LCDR3 | PRT | SEQ ID NO: 28 |
| TPP-14389 | 13612-rec02-hIgG1Kappa | VH | DNA | SEQ ID NO: 29 |
| TPP-14389 | 13612-rec02-hIgG1Kappa | VL | DNA | SEQ ID NO: 30 |
| TPP-14389 | 13612-rec02-hIgG1Kappa | Heavy Chain | PRT | SEQ ID NO: 31 |
| TPP-14389 | 13612-rec02-hIgG1Kappa | Light Chain | PRT | SEQ ID NO: 32 |
| TPP-14389 | 13612-rec02-hIgG1Kappa | Heavy Chain | DNA | SEQ ID NO: 33 |
| TPP-14389 | 13612-rec02-hIgG1Kappa | Light Chain | DNA | SEQ ID NO: 34 |
| TPP-14392 | 13612-rec05-hIgG1Kappa | VH | PRT | SEQ ID NO: 35 |
| TPP-14392 | 13612-rec05-hIgG1Kappa | HCDR1 | PRT | SEQ ID NO: 36 |
| TPP-14392 | 13612-rec05-hIgG1Kappa | HCDR2 | PRT | SEQ ID NO: 37 |
| TPP-14392 | 13612-rec05-hIgG1Kappa | HCDR3 | PRT | SEQ ID NO: 38 |
| TPP-14392 | 13612-rec05-hIgG1Kappa | VL | PRT | SEQ ID NO: 39 |
| TPP-14392 | 13612-rec05-hIgG1Kappa | LCDR1 | PRT | SEQ ID NO: 40 |
| TPP-14392 | 13612-rec05-hIgG1Kappa | LCDR2 | PRT | SEQ ID NO: 41 |
| TPP-14392 | 13612-rec05-hIgG1Kappa | LCDR3 | PRT | SEQ ID NO: 42 |
| TPP-14392 | 13612-rec05-hIgG1Kappa | VH | DNA | SEQ ID NO: 43 |
| TPP-14392 | 13612-rec05-hIgG1Kappa | VL | DNA | SEQ ID NO: 44 |
| TPP-14392 | 13612-rec05-hIgG1Kappa | Heavy Chain | PRT | SEQ ID NO: 45 |
| TPP-14392 | 13612-rec05-hIgG1Kappa | Light Chain | PRT | SEQ ID NO: 46 |
| TPP-14392 | 13612-rec05-hIgG1Kappa | Heavy Chain | DNA | SEQ ID NO: 47 |
| TPP-14392 | 13612-rec05-hIgG1Kappa | Light Chain | DNA | SEQ ID NO: 48 |
| TPP-17073 | 438H-M113-N15-hIgG1 | VH | PRT | SEQ ID NO: 49 |
| TPP-17073 | 438H-M113-N15-hIgG1 | HCDR1 | PRT | SEQ ID NO: 50 |
| TPP-17073 | 438H-M113-N15-hIgG1 | HCDR2 | PRT | SEQ ID NO: 51 |
| TPP-17073 | 438H-M113-N15-hIgG1 | HCDR3 | PRT | SEQ ID NO: 52 |
| TPP-17073 | 438H-M113-N15-hIgG1 | VL | PRT | SEQ ID NO: 53 |
| TPP-17073 | 438H-M113-N15-hIgG1 | LCDR1 | PRT | SEQ ID NO: 54 |
| TPP-17073 | 438H-M113-N15-hIgG1 | LCDR2 | PRT | SEQ ID NO: 55 |
| TPP-17073 | 438H-M113-N15-hIgG1 | LCDR3 | PRT | SEQ ID NO: 56 |
| TPP-17073 | 438H-M113-N15-hIgG1 | Heavy Chain | PRT | SEQ ID NO: 57 |
| TPP-17073 | 438H-M113-N15-hIgG1 | Light Chain | PRT | SEQ ID NO: 58 |
| TPP-17074 | 438H-M161-K22-hIgG1 | VH | PRT | SEQ ID NO: 59 |
| TPP-17074 | 438H-M161-K22-hIgG1 | HCDR1 | PRT | SEQ ID NO: 60 |
| TPP-17074 | 438H-M161-K22-hIgG1 | HCDR2 | PRT | SEQ ID NO: 61 |
| TPP-17074 | 438H-M161-K22-hIgG1 | HCDR3 | PRT | SEQ ID NO: 62 |
| TPP-17074 | 438H-M161-K22-hIgG1 | VL | PRT | SEQ ID NO: 63 |
| TPP-17074 | 438H-M161-K22-hIgG1 | LCDR1 | PRT | SEQ ID NO: 64 |
| TPP-17074 | 438H-M161-K22-hIgG1 | LCDR2 | PRT | SEQ ID NO: 65 |
| TPP-17074 | 438H-M161-K22-hIgG1 | LCDR3 | PRT | SEQ ID NO: 66 |
| TPP-17074 | 438H-M161-K22-hIgG1 | Heavy Chain | PRT | SEQ ID NO: 67 |
| TPP-17074 | 438H-M161-K22-hIgG1 | Light Chain | PRT | SEQ ID NO: 68 |
| TPP-17078 | 438H-M308-H05-hIgGkappa | VH | PRT | SEQ ID NO: 69 |
| TPP-17078 | 438H-M308-H05-hIgGkappa | HCDR1 | PRT | SEQ ID NO: 70 |
| TPP-17078 | 438H-M308-H05-hIgGkappa | HCDR2 | PRT | SEQ ID NO: 71 |
| TPP-17078 | 438H-M308-H05-hIgGkappa | HCDR3 | PRT | SEQ ID NO: 72 |
| TPP-17078 | 438H-M308-H05-hIgGkappa | VL | PRT | SEQ ID NO: 73 |
| TPP-17078 | 438H-M308-H05-hIgGkappa | LCDR1 | PRT | SEQ ID NO: 74 |
| TPP-17078 | 438H-M308-H05-hIgGkappa | LCDR2 | PRT | SEQ ID NO: 75 |
| TPP-17078 | 438H-M308-H05-hIgGkappa | LCDR3 | PRT | SEQ ID NO: 76 |
| TPP-17078 | 438H-M308-H05-hIgGkappa | VH | DNA | SEQ ID NO: 77 |
| TPP-17078 | 438H-M308-H05-hIgGkappa | VL | DNA | SEQ ID NO: 78 |
| TPP-17078 | 438H-M308-H05-hIgGkappa | Heavy Chain | PRT | SEQ ID NO: 79 |
| TPP-17078 | 438H-M308-H05-hIgGkappa | Light Chain | PRT | SEQ ID NO: 80 |
| TPP-17078 | 438H-M308-H05-hIgGkappa | Heavy Chain | DNA | SEQ ID NO: 81 |
| TPP-17078 | 438H-M308-H05-hIgGkappa | Light Chain | DNA | SEQ ID NO: 82 |
| TPP-17405 | 438H-M345-F05-hIgG1Kappa | VH | PRT | SEQ ID NO: 83 |
| TPP-17405 | 438H-M345-F05-hIgG1Kappa | HCDR1 | PRT | SEQ ID NO: 84 |
| TPP-17405 | 438H-M345-F05-hIgG1Kappa | HCDR2 | PRT | SEQ ID NO: 85 |
| TPP-17405 | 438H-M345-F05-hIgG1Kappa | HCDR3 | PRT | SEQ ID NO: 86 |
| TPP-17405 | 438H-M345-F05-hIgG1Kappa | VL | PRT | SEQ ID NO: 87 |
| TPP-17405 | 438H-M345-F05-hIgG1Kappa | LCDR1 | PRT | SEQ ID NO: 88 |
| TPP-17405 | 438H-M345-F05-hIgG1Kappa | LCDR2 | PRT | SEQ ID NO: 89 |
| TPP-17405 | 438H-M345-F05-hIgG1Kappa | LCDR3 | PRT | SEQ ID NO: 90 |
| TPP-17405 | 438H-M345-F05-hIgG1Kappa | Heavy Chain | PRT | SEQ ID NO: 91 |
| TPP-17405 | 438H-M345-F05-hIgG1Kappa | Light Chain | PRT | SEQ ID NO: 92 |
| TPP-17418 | 438H-M308-H05_B-hIgG1Kappa | VH | PRT | SEQ ID NO: 93 |
| TPP-17418 | 438H-M308-H05_B-hIgG1Kappa | HCDR1 | PRT | SEQ ID NO: 94 |
| TPP-17418 | 438H-M308-H05_B-hIgG1Kappa | HCDR2 | PRT | SEQ ID NO: 95 |
| TPP-17418 | 438H-M308-H05_B-hIgG1Kappa | HCDR3 | PRT | SEQ ID NO: 96 |
| TPP-17418 | 438H-M308-H05_B-hIgG1Kappa | VL | PRT | SEQ ID NO: 97 |
| TPP-17418 | 438H-M308-H05_B-hIgG1Kappa | LCDR1 | PRT | SEQ ID NO: 98 |
| TPP-17418 | 438H-M308-H05_B-hIgG1Kappa | LCDR2 | PRT | SEQ ID NO: 99 |
| TPP-17418 | 438H-M308-H05_B-hIgG1Kappa | LCDR3 | PRT | SEQ ID NO: 100 |
| TPP-17418 | 438H-M308-H05_B-hIgG1Kappa | Heavy Chain | PRT | SEQ ID NO: 101 |
| TPP-17418 | 438H-M308-H05_B-hIgG1Kappa | Light Chain | PRT | SEQ ID NO: 102 |
| TPP-17419 | 438H-M307-H07-hIgG1Kappa | VH | PRT | SEQ ID NO: 103 |
| TPP-17419 | 438H-M307-H07-hIgG1Kappa | HCDR1 | PRT | SEQ ID NO: 104 |
| TPP-17419 | 438H-M307-H07-hIgG1Kappa | HCDR2 | PRT | SEQ ID NO: 105 |
| TPP-17419 | 438H-M307-H07-hIgG1Kappa | HCDR3 | PRT | SEQ ID NO: 106 |
| TPP-17419 | 438H-M307-H07-hIgG1Kappa | VL | PRT | SEQ ID NO: 107 |
| TPP-17419 | 438H-M307-H07-hIgG1Kappa | LCDR1 | PRT | SEQ ID NO: 108 |
| TPP-17419 | 438H-M307-H07-hIgG1Kappa | LCDR2 | PRT | SEQ ID NO: 109 |
| TPP-17419 | 438H-M307-H07-hIgG1Kappa | LCDR3 | PRT | SEQ ID NO: 110 |
| TPP-17419 | 438H-M307-H07-hIgG1Kappa | Heavy Chain | PRT | SEQ ID NO: 111 |
| TPP-17419 | 438H-M307-H07-hIgG1Kappa | Light Chain | PRT | SEQ ID NO: 112 |
| TPP-17421 | 438H-M306-C11-hIgG1Kappa | VH | PRT | SEQ ID NO: 113 |
| TPP-17421 | 438H-M306-C11-hIgG1Kappa | HCDR1 | PRT | SEQ ID NO: 114 |
| TPP-17421 | 438H-M306-C11-hIgG1Kappa | HCDR2 | PRT | SEQ ID NO: 115 |
| TPP-17421 | 438H-M306-C11-hIgG1Kappa | HCDR3 | PRT | SEQ ID NO: 116 |
| TPP-17421 | 438H-M306-C11-hIgG1Kappa | VL | PRT | SEQ ID NO: 117 |
| TPP-17421 | 438H-M306-C11-hIgG1Kappa | LCDR1 | PRT | SEQ ID NO: 118 |
| TPP-17421 | 438H-M306-C11-hIgG1Kappa | LCDR2 | PRT | SEQ ID NO: 119 |
| TPP-17421 | 438H-M306-C11-hIgG1Kappa | LCDR3 | PRT | SEQ ID NO: 120 |
| TPP-17421 | 438H-M306-C11-hIgG1Kappa | VH | DNA | SEQ ID NO: 121 |
| TPP-17421 | 438H-M306-C11-hIgG1Kappa | VL | DNA | SEQ ID NO: 122 |
| TPP-17421 | 438H-M306-C11-hIgG1Kappa | Heavy Chain | PRT | SEQ ID NO: 123 |
| TPP-17421 | 438H-M306-C11-hIgG1Kappa | Light Chain | PRT | SEQ ID NO: 124 |
| TPP-17421 | 438H-M306-C11-hIgG1Kappa | Heavy Chain | DNA | SEQ ID NO: 125 |
| TPP-17421 | 438H-M306-C11-hIgG1Kappa | Light Chain | DNA | SEQ ID NO: 126 |
| TPP-17422 | 438H-M313-J08_B-hIgG1Kappa | VH | PRT | SEQ ID NO: 127 |
| TPP-17422 | 438H-M313-J08_B-hIgG1Kappa | HCDR1 | PRT | SEQ ID NO: 128 |
| TPP-17422 | 438H-M313-J08_B-hIgG1Kappa | HCDR2 | PRT | SEQ ID NO: 129 |
| TPP-17422 | 438H-M313-J08_B-hIgG1Kappa | HCDR3 | PRT | SEQ ID NO: 130 |
| TPP-17422 | 438H-M313-J08_B-hIgG1Kappa | VL | PRT | SEQ ID NO: 131 |
| TPP-17422 | 438H-M313-J08_B-hIgG1Kappa | LCDR1 | PRT | SEQ ID NO: 132 |
| TPP-17422 | 438H-M313-J08_B-hIgG1Kappa | LCDR2 | PRT | SEQ ID NO: 133 |
| TPP-17422 | 438H-M313-J08_B-hIgG1Kappa | LCDR3 | PRT | SEQ ID NO: 134 |
| TPP-17422 | 438H-M313-J08_B-hIgG1Kappa | Heavy Chain | PRT | SEQ ID NO: 135 |
| TPP-17422 | 438H-M313-J08_B-hIgG1Kappa | Light Chain | PRT | SEQ ID NO: 136 |
| TPP-1545 | hLRRC15 CT-His | Chain 1 | PRT | SEQ ID NO: 137 |
| TPP-9045 | mLRRC15-ECD_His6 | Chain 1 | PRT | SEQ ID NO: 138 |
| TPP-9046 | macfasLRRC15-ECD_His6 | Chain 1 | PRT | SEQ ID NO: 139 |
| TPP-21468 | Human germline heavy chain | Chain 1 | PRT | SEQ ID NO: 140 |
| | (V-segment) - IGHV1-2-02 | | | |
| TPP-21469 | Human germline light chain - | Chain 1 | PRT | SEQ ID NO: 141 |
| | IGKV1-NL1-01-IGKJ4-01-02 | | | |
| TPP-21470 | Human germline heavy chain | Chain 1 | PRT | SEQ ID NO: 142 |
| | (J-segment) - HV3-23-J1 | | | |
| TPP-21479 | Human germline light chain - | Chain 1 | PRT | SEQ ID NO: 143 |
| | IGKV1-39-01-IGKJ4-01-02 | | | |
| TPP-21547 | Human germline heavy chain | Chain 1 | PRT | SEQ ID NO: 144 |
| | (V-segment) - IGHV3-23-01 | | | |

### DETAILED DESCRIPTION

### Definitions

Unless otherwise defined, all scientific and technical terms used in the description, figures and claims have their ordinary meaning as commonly understood by one of ordinary skill in the art. All publications, patent applications, patents, and other references mentioned herein are incorporated by reference in their entirety. In case of conflict, the present specification, including definitions, will prevail. If two or more documents incorporated by reference include conflicting and/or inconsistent disclosure with respect to each other, then the document having the later effective date shall control. The materials, methods, and examples are illustrative only and not intended to be limiting. Unless stated otherwise, the following terms used in this document, including the description and claims, have the definitions given below.

Singular forms such as “a”, “an” or “the” include plural references unless the context clearly indicates otherwise. Thus, for example, reference to an “antibody” includes a single antibody as well as a plurality of antibodies, either the same or different. Likewise reference to a “cell” includes a single cell as well as a plurality of cells.

The word “about” as used herein refers to a value being within an acceptable error range for the particular value as determined by one of ordinary skill in the art, which will depend in part on how the value is measured or determined, i.e., on the limitations of the measurement system. For example, “about” can mean within 1 or more than 1 standard deviation per the practice in the art. The term “about” is also used to indicate that the amount or value in question may be the value designated or some other value that is approximately the same. The phrase is intended to convey that similar values promote equivalent results or effects as described herein. In this context “about” may refer to a range above and/or below of up to 20% or 10%. Wherever the term “about” is specified for a certain assay or embodiment, that definition prevails for the particular context.

It is furthermore understood that slight variations above and below a stated range can be used to achieve substantially the same results as a value within the range. Also, unless indicated otherwise, the disclosure of ranges is intended as a continuous range including every value between the minimum and maximum values.

The terms “comprising”, “including”, “containing”, “having” etc. shall be read expansively or open-ended and without limitation. The term “comprising” when used in the specification includes “consisting of” and “essentially consisting of”.

Unless otherwise indicated, the term “at least” preceding a series of elements is to be understood to refer to every element in the series. The terms “at least one” and “at least one of” include for example, one, two, three, four, or five or more elements.

The term “isolated” when applied to a defined biological subject matter such as a nucleic acid, gene, polypeptide, protein or antibody, denotes that the biological subject matter is essentially free of other cellular components with which it is associated in the natural state. In particular, an isolated gene is separated from open reading frames that flank the gene and encode a protein other than the gene of interest. The isolated subject matter is preferably in a homogenous state and may be without limitation in a dry state, or in an aqueous solution.

A nucleic acid, polypeptide, protein, antibody or cell that is the predominant species present in a preparation is called “substantially purified”. Preferably, this means that the nucleic acid, polypeptide, protein, antibody or cell is at least 85% pure, more preferably at least 95% pure, and most preferably at least 99% pure. Purity and homogeneity are typically determined using analytical chemistry techniques such as polyacrylamide gel electrophoresis or high performance liquid chromatography, or fluorescence activated cell sorting for cells.

### Radioconjugates

A “radionuclide” (also: “radioactive nuclide”, “radioisotope” or “radioactive isotope”) is an atom that undergoes radioactive decay. Without limitation, for example the radionuclide may be a beta particle emitting radionuclide (beta emitter), an α-particle-emitting radionuclide (alpha emitter), or an Auger electron emitting radionuclide (Auger electron emitter).

“β-particle-emitting radionuclides” or “beta emitter” are radionuclides which emit beta particles. Examples of beta emitters include without limitation copper-67 (⁶⁷Cu), strontium-89 (⁸⁹Sr), yttrium-90 (⁹⁰Y), rhodium-105 (¹⁰⁵Rh), iodine-131 (¹³¹I), promethium-149 (¹⁴⁹Pm), holmium-166 (¹⁶⁶Ho), lutetium-177 (¹⁷⁷Lu), rhenium-186 (¹⁸⁶Re), rhenium-188 (¹⁸⁸Re), gold-198 (¹⁹⁸Au) and gold-199 (¹⁹⁹Au). Jongho has reviewed various techniques for chelation of various of these radioisotypes (Jeon, Jongho. “Review of therapeutic applications of radiolabeled functional nanomaterials.” International journal of molecular sciences 20.9 (2019): 2323.).

“Auger electron emitting radionuclides” or “Auger electron emitter” are radionuclides which emit Auger electrons. Examples of Auger electron emitters include without limitation bromine-77, indium-111, iodine-123, and iodine-125.

“α-particle-emitting radionuclides” or “alpha emitter” are radionuclides which emit alpha particles, i.e. 4He nuclei with a +2 charge. Non limiting examples of alpha emitters include bismuth-213 (²¹³Bi), characterized by a half-life of 45.6 min, actinium-225 (²²⁵Ac), characterized by a half-life of 9.9 d, astatine-211 (²¹¹At), characterized by a half-life of 7.2 h, radium-223 (²²³Ra), characterized by a half-life of 11.4 d, radium-224 (²²⁴Ra), characterized by a half-life of 3.7 d, and thorium-227 (²²⁷Th), characterized by a half-life of 18.7 d.

Radionuclides can be obtained as known in the art. For example, as described in Poty, Sophie, et al, ²¹¹At can be cyclotron-produced by bombarding natural bismuth with a medium energy α-particle beam using the ²⁰⁹Bi(α,2n)²¹¹At reaction (“Poty, Sophie, et al. α-Emitters for radiotherapy: from basic radiochemistry to clinical studies-part 1/2.” Journal of Nuclear Medicine 59.6/59.7 (2018): 878-884/1020-1027). ²²⁷Th and ²²³Ra are both available upon separation from their mutual parent, ²²⁷Ac. Clinical production of ²²³Ra uses ²²⁷Ac/²²⁷Th-based generators. Parent isotopes are loaded on actinide chromatographic resin and ²²³Ra chloride solution is obtained after elution with 1M HCl or HNO3, subsequent cation exchange column, evaporation, and dissolution in saline solution.

“Chelation” refers to the formation or presence of two or more separate coordinate bonds between a ligand and a single central metal atom. The ligands which are capable of forming these coordinate bonds are termed “chelators”, “chelating agents”, or “sequestering agents”.

“A chelator arranged for complexation of a radionuclide” is a chelator which can chelate a given radionuclide or group of radionuclides, such as, without limitation, alpha emitters, beta emitters or Auger electron emitter. Various chelators are known in the art and can be used according to the current invention, e.g. as described in Price, Eric W., and Chris Orvig. “Matching chelators to radiometals for radiopharmaceuticals.” Chemical Society Reviews 43.1 (2014): 260-290, incorporated herein in its entirety.

“A chelator arranged for complexation of an α-particle-emitting radionuclide” is a chelator which can chelate at least one α-particle-emitting radionuclide. Unlike Ra-223, Th-227 exists in the 4+ oxidation state and forms stable complexes with chelators such as 1,4,7,10-tetra-azacycloododecane-N,N′,N″,N′″-tetraacetic acid (DOTA). Table 1 lists some non-limiting examples of suitable chelators.

TABLE 1
Non-limiting examples of chelators arranged
for complexation of specific radionuclides

| Radioisotype | Radiochemistry |
|---|---|
| ²¹¹At | Tin precursor, prosthetic group |
| ²²⁵Ac | DOTA, DO3A chelator |
| ²¹³Bi | CHX-A″-DTPA, DOTA, NETA |
| ²²⁷Th | DOTA, Me-3,2-HOPO |
| ²¹²Pb | TCMC |
| ²¹²Bi | CHX-A″-DTPA, DOTA, NETA |

A “derivative” as used herein is a compound that is derived from a compound with the same core structure by chemical reaction and that is suitable for the same purpose (e.g. chelation of a radionuclide).

The term “DOTA” refers to 2,2′,2″,2′″-(1,4,7,10-tetraazacyclododecane-1,4,7,10-tetrayl)tetraaceticacid or 1,4,7,10-tetra-azacycloododecane-N,N′,N″,N′″-tetraacetic acid and derivatives thereof, which can chelate a radionuclide. DOTA is a chelator arranged for complexation of an α-particle-emitting radionuclide, such as ²¹²Bi, ²¹³Bi, ²²⁵Ac, ²²⁷Th. Chelators such as DOTA form stable complexes with a chelated Th-227, which exists in the 4+ oxidation state. In order to achieve sufficient labeling of DOTA-coupled antibodies, the complexation step should preferably be performed as a two-step process or directly at elevated temperatures.

The term “DO3A” refers to 2,2′,2″-(1,4,7,10-tetraazacyclododecane-1,4,7-triyl)triacetate and derivatives thereof, which can chelate a radionuclide. DO3A is a chelator arranged for complexation of an α-particle-emitting radionuclide, such as ²²⁵Ac.

The term “CHX-A″-DTPA” refers to 2-(p-isothiocyanatobenzyl)-cyclohexyldiethylenetriaminepentaaceticacid and derivatives thereof, which can chelate a radionuclide. CHX-A″-DTPA is a chelator arranged for complexation of an α-particle-emitting radionuclide, such as ²¹²Bi or ²¹³Bi.

The term “NETA” refers to {4-{2-(bis-carboxymethylamino)-ethyl]-7-carboxymethyl-[1,4,7]triazonan-1-yl}-aceticacid and derivatives thereof, which can chelate a radionuclide. NETA is a chelator arranged for complexation of an α-particle-emitting radionuclide, such as ²¹²Bi or ²¹³Bi.

The term “TCMC” refers to 1,4,7,10-tetrakis(carbamoylmethyl)-1,4,7,10-tetraazacyclododecane and derivatives thereof, which can chelate a radionuclide. TCMC is a chelator arranged for complexation of an α-particle-emitting radionuclide, such as ²¹²Pb.

The term “HOPO” refers to a hydroxypyridinone. HOPOs form 5-membered chelate rings in which the metal is coordinated by two vicinal oxygen atoms. There are at least three classes of metal chelating HOPO ligands, namely, 1-hydroxypyridin-2-one (1,2-HOPO), 3-hydroxypyridin-2-one (3,2-HOPO), and 3-hydroxypyridin-4-one (3,4-HOPO).

The term “Me-3,2-HOPO” refers to 3-hydroxy-N-methyl-2-pyridinone and derivatives thereof, which can chelate a radionuclide. The Me-3,2-HOPO groups are monoprotic acids that complex thorium-227 through the two oxygen atoms on each subunit. A detailed report on the synthesis and conjugation to monoclonal antibodies is provided in Ramdahl, Thomas, et al. “An efficient chelator for complexation of thorium-227.” Bioorganic & medicinal chemistry letters 26.17 (2016): 4318-4321, and is incorporated herein in its entirety. A chelator comprising Me-3,2-HOPO may preferably furthermore comprise a (symmetrical) polyamine scaffold to which the Me-3,2-HOPO moieties are coupled and a carboxylic acid group facilitating conjugation to biomolecules such as antibodies.

A “chelator of general formula (I)” as used herein is a chelator of the formula (I)

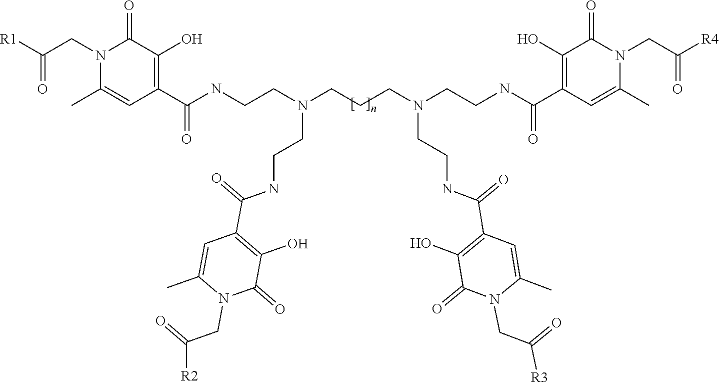

wherein:
n is 1, 2 or 3;
R1, R2, R3 and R4, independently represent OH or Q; and
Q represents a connection to a targeting moiety, e.g. to a targeting moiety binding LRRC15.

Where any of R1, R2, R3 and/or R4 is a targeting moiety binding LRRC15, the targeting moiety is considered as a separate entity which does not form part of the chelator. Chelation may occur, e.g. according to formula IA, wherein the chelator of general formula (I) is radiolabled with a radionuclide A selected from the group consisting of ⁴³Sc, ⁴⁴Sc, ⁴⁷Sc, ⁸⁹Zr, ⁹⁰Y, ¹¹¹In, ¹⁴⁹Tb, ¹⁵²Tb, ¹⁵⁵Tb, ¹⁶¹Tb, ¹⁶⁶Ho, ¹⁷⁷Lu, ¹⁸⁶Re, ¹⁸⁸Re, ²¹²Bi, ²¹³Bi, ²²⁵Ac, ²²⁷Th, and ²³²Th.

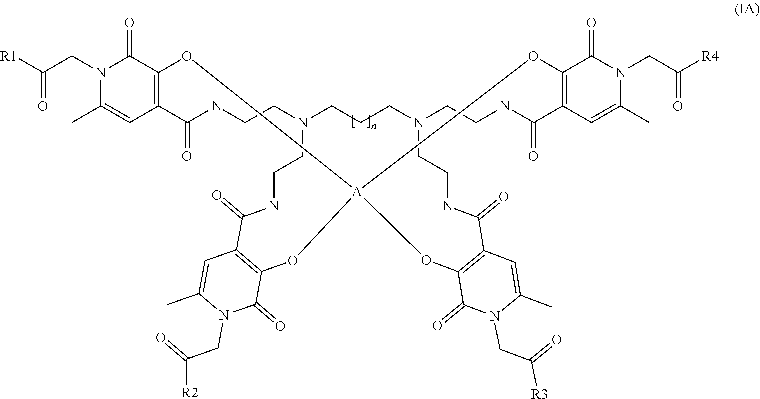

wherein:
n is 1, 2 or 3;
R1, R2, R3 and R4, independently represent OH or Q; and
Q represents a connection to a targeting moiety, e.g. to a targeting moiety binding LRRC15.

In a particular example, n is 1 and two of R1, R2, R3 and R4 represent OH and two of R1, R2, R3 and R4 represent Q. In a particular example, n is 1 and all of R1, R2, R3 and R4 represent Q. In a particular example n is 1 and one of R1, R2, R3 and R4 represents OH and three of R1, R2, R3 and R4 represent Q.

It is possible for the compounds of general formula (I) to exist as isotopic variants. The invention therefore includes conjugates comprising one or more isotopic variant(s) of the compounds of general formula (I), particularly deuterium-containing compounds of general formula (I).
The term “Isotopic variant” of a compound or a reagent is defined as a compound exhibiting an unnatural proportion of one or more of the isotopes that constitute such a compound.
The term “Isotopic variant of the compound of general formula (I)” is defined as a compound of general formula (I) exhibiting an unnatural proportion of one or more of the isotopes that constitute such a compound.

The expression “unnatural proportion” means a proportion of such isotope which is higher than its natural abundance. The natural abundances of isotopes to be applied in this context are described in “Isotopic Compositions of the Elements 1997”, Pure Appl. Chem., 70(1), 217-235, 1998.

Examples of such isotopes include stable and radioactive isotopes of hydrogen, carbon, nitrogen, oxygen, phosphorus, sulfur, fluorine, chlorine, bromine and iodine, such as 2H (deuterium), 3H (tritium), 11C, 13C, 14C, 15N, 17O, 18O, 32P, 33P, 33S, 34S, 35S, 36S, 18F, 36Cl, 82Br, 123I, 124I, 125I, 129I and 131I, respectively.

With respect to the treatment and/or prophylaxis of the disorders specified herein the isotopic variant(s) of the compounds of general formula (I) preferably contain deuterium (“deuterium-containing compounds of general formula (I)”). Isotopic variants of the compounds of general formula (I) in which one or more radioactive isotopes, such as 3H or 14C, are incorporated are useful e.g. in drug and/or substrate tissue distribution studies. These isotopes are particularly preferred for the ease of their incorporation and detectability. Positron emitting isotopes such as 18F or 11C may be incorporated into a compound of general formula (I). These isotopic variants of the compounds of general formula (I) are useful for in vivo imaging applications. Deuterium-containing and 13C-containing compounds of general formula (I) can be used in mass spectrometry analyses (H. J. Leis et al., Curr. Org. Chem., 1998, 2, 131) in the context of preclinical or clinical studies.

Isotopic variants of the compounds of general formula (I) can generally be prepared by methods known to a person skilled in the art, such as those described in the schemes and/or examples herein, by substituting a reagent for an isotopic variant of said reagent, preferably for a deuterium-containing reagent. Depending on the desired sites of deuteration, in some cases deuterium from D2O can be incorporated either directly into the compounds or into reagents that are useful for synthesizing such compounds (Esaki et al., Tetrahedron, 2006, 62, 10954; Esaki et al., Chem. Eur. J., 2007, 13, 4052). Deuterium gas is also a useful reagent for incorporating deuterium into molecules. Catalytic deuteration of olefinic bonds (H. J. Leis et al., Curr. Org. Chem., 1998, 2, 131; J. R. Morandi et al., J. Org. Chem., 1969, 34 (6), 1889) and acetylenic bonds (N. H. Khan, J. Am. Chem. Soc., 1952, 74 (12), 3018; S. Chandrasekhar et al., Tetrahedron Letters, 2011, 52, 3865) is a rapid route for incorporation of deuterium. Metal catalysts (i.e. Pd, Pt, and Rh) in the presence of deuterium gas can be used to directly exchange deuterium for hydrogen in functional groups containing hydrocarbons (J. G. Atkinson et al., U.S. Pat. No. 3,966,781). A variety of deuterated reagents and synthetic building blocks are commercially available from companies such as for example C/D/N Isotopes, Quebec, Canada; Cambridge Isotope Laboratories Inc., Andover, MA, USA; and CombiPhos Catalysts, Inc., Princeton, NJ, USA. Further information on the state of the art with respect to deuterium-hydrogen exchange is given for example in Hanzlik et al., J. Org. Chem. 55, 3992-3997, 1990; R. P. Hanzlik et al., Biochem. Biophys. Res. Commun. 160, 844, 1989; P. J. Reider et al., J. Org. Chem. 52, 3326-3334, 1987; M. Jarman et al., Carcinogenesis 16(4), 683-688, 1995; J. Atzrodt et al., Angew. Chem., Int. Ed. 2007, 46, 7744; K. Matoishi et al., Chem. Commun. 2000, 1519-1520; K. Kassahun et al., WO2012/112363.

The term “deuterium-containing compound of general formula (I)” is defined as a compound of general formula (I), in which one or more hydrogen atom(s) is/are replaced by one or more deuterium atom(s) and in which the abundance of deuterium at each deuterated position of the compound of general formula (I) is higher than the natural abundance of deuterium, which is about 0.015%. Particularly, in a deuterium-containing compound of general formula (I) the abundance of deuterium at each deuterated position of the compound of general formula (I) is higher than 10%, 20%, 30%, 40%, 50%, 60%, 70% or 80%, preferably higher than 90%, 95%, 96% or 97%, even more preferably higher than 98% or 99% at said position(s). It is understood that the abundance of deuterium at each deuterated position is independent of the abundance of deuterium at other deuterated position(s).

The selective incorporation of one or more deuterium atom(s) into a compound of general formula (I) may alter the physicochemical properties (such as for example acidity [C. L. Perrin, et al., J. Am. Chem. Soc., 2007, 129, 4490; A. Streitwieser et al., J. Am. Chem. Soc., 1963, 85, 2759;], basicity [C. L. Perrin et al., J. Am. Chem. Soc., 2005, 127, 9641; C. L. Perrin, et al., J. Am. Chem. Soc., 2003, 125, 15008; C. L. Perrin in Advances in Physical Organic Chemistry, 44, 144], lipophilicity [B. Testa et al., Int. J. Pharm., 1984, 19(3), 271]) and/or the metabolic profile of the molecule and may result in changes in the ratio of parent compound to metabolites or in the amounts of metabolites formed. Such changes may result in certain therapeutic advantages and hence may be preferred in some circumstances. Reduced rates of metabolism and metabolic switching, where the ratio of metabolites is changed, have been reported (A. E. Mutlib et al., Toxicol. Appl. Pharmacol., 2000, 169, 102; D. J. Kushner et al., Can. J. Physiol. Pharmacol., 1999, 77, 79). These changes in the exposure to parent drug and metabolites can have important consequences with respect to the pharmacodynamics, tolerability and efficacy of a deuterium-containing compound of general formula (I). In some cases deuterium substitution reduces or eliminates the formation of an undesired or toxic metabolite and enhances the formation of a desired metabolite (e.g. Nevirapine: A. M. Sharma et al., Chem. Res. Toxicol., 2013, 26, 410; Efavirenz: A. E. Mutlib et al., Toxicol. Appl. Pharmacol., 2000, 169, 102). In other cases the major effect of deuteration is to reduce the rate of systemic clearance. As a result, the biological half-life of the compound is increased. The potential clinical benefits would include the ability to maintain similar systemic exposure with decreased peak levels and increased trough levels. This could result in lower side effects and enhanced efficacy, depending on the particular compound's pharmacokinetic/pharmacodynamic relationship. ML-337 (C. J. Wenthur et al., J. Med. Chem., 2013, 56, 5208) and Odanacatib (K. Kassahun et al., WO2012/112363) are examples for this deuterium effect. Still other cases have been reported in which reduced rates of metabolism result in an increase in exposure of the drug without changing the rate of systemic clearance (e.g. Rofecoxib: F. Schneider et al., Arzneim. Forsch./Drug. Res., 2006, 56, 295; Telaprevir: F. Maltais et al., J. Med. Chem., 2009, 52, 7993). Deuterated drugs showing this effect may have reduced dosing requirements (e.g. lower number of doses or lower dosage to achieve the desired effect) and/or may produce lower metabolite loads.

A compound of general formula (I) may have multiple potential sites of attack for metabolism. To optimize the above-described effects on physicochemical properties and metabolic profile, deuterium-containing compounds of general formula (I) having a certain pattern of one or more deuterium-hydrogen exchange(s) can be selected. Particularly, the deuterium atom(s) of deuterium-containing compound(s) of general formula (I) is/are attached to a carbon atom and/or is/are located at those positions of the compound of general formula (I), which are sites of attack for metabolizing enzymes such as e.g. cytochrome P450.

The term “TETA” refers to macrocyclic chelator “1,4,8,11-tetraazacyclotetradecane-N,N′,N″,N′″-tetraacetic acid”.

“Desferrioxamine B” (Df) is a chelator which can be used for ⁸⁹Zr labeling of antibodies and can form a stable chelate with ⁸⁹Zr through 3 hydroxamate groups. Generally, mAbs are conjugated with a bifunctional derivative of Df via an amide linkage for subsequent labeling with ⁸⁹Zr. The hydroxamate groups within Df is preferably temporarily blocked with Fe(III) before mAb conjugation. Subsequently, Fe(III) is removed by transchelation to ethylenediaminetetraacetic acid (EDTA) before the conjugate is exposed to ⁸⁹Zr.

“Radioconjugates” are conjugates comprising a first moiety, at least one chelator or chelating group arranged for complexation of a radioisotope, and optionally a radioisotype. The first moiety can be, without limitation, a targeting moiety or a detectable moiety.

“Targeted thorium conjugates” are radioconjugates comprising a targeting moiety and at least one chelator or chelating group arranged for complexation of thorium, and optionally comprise thorium.

A “detectable moiety” is a moiety arranged for detection using a matching imaging technology. Examples of detectable moieties include various enzymes or enzymatic labels, prosthetic groups, fluorescent groups or materials, luminescent groups or materials, bioluminescent groups or materials, radioactive isotopes or materials, positron emitting metals, nonradioactive paramagnetic metal ions, and reactive moieties.

The detectable substance can be coupled or conjugated either directly to the conjugate, antibody or fragment thereof or indirectly, e.g. without limitation through a linker known in the art or through another moiety, using techniques known in the art.

Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, β-galactosidase, acetylcholinesterase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Examples of suitable prosthetic groups include streptavidin/biotin and avidin/biotin. Examples of suitable fluorescent groups or materials include umbelliferone, fluorescein, fluorescein isothiocyanate, rhodamine, dichlorotriazinylamine fluorescein, dansyl chloride or phycoerythrin. An example of a luminescent group or material includes luminol. Examples of bioluminescent groups or materials include luciferase, luciferin, and aequorin. Examples of suitable radioactive isotopes or materials include ¹²⁵I, ¹³¹I, ¹¹¹In or ^{99m}Tc.

A “targeting moiety” (also “tissue-targeting group” or “tissue-targeting moiety”) as used herein is any chemical structure binding to a biological target, such as LRRC15 or a cell expressing LRRC15. The targeting moiety localizes itself, e.g. as part of a bigger structure at a specific site where the presence is required to exert the intended effect, e.g. delivery of radioactive decay in case of a TAT. Thus, a tissue targeting group or moiety serves to provide greater localization of a molecule or conjugate to at least one desired site in the body of a subject in comparison with the concentration of an equivalent complex not comprising the targeting moiety. According to the current invention, the targeting moiety can be for example selected without limitation from the group consisting of nucleotides, DNA and RNA fragments, aptamers, peptides, proteins, antibodies or fragments thereof, nanoparticles, or small molecules, or any combination thereof.

A “targeting moiety binding to (human) LRRC15” is a chemical structure binding to (human) protein LRRC15. According to highly preferred embodiments of the current invention, the targeting moiety can be an antibody or antigen binding fragment thereof, as described herein.

The present invention includes all possible stereoisomers of the compounds disclosed herein as single stereoisomers, or as any mixture of said stereoisomers, e.g. (R)- or (S)-isomers, in any ratio. Isolation of a single stereoisomer, e.g. a single enantiomer or a single diastereomer, of a compound of the present invention is achieved by any suitable state of the art method, such as chromatography, especially chiral chromatography, for example.

The present invention includes all possible tautomers of the compounds of the present invention as single tautomers, or as any mixture of said tautomers, in any ratio.

Further, the compounds of the present invention can exist as N-oxides, which are defined in that at least one nitrogen of the compounds of the present invention is oxidised. The present invention includes all such possible N-oxides.

The present invention also covers useful forms of the compounds of the present invention, such as metabolites, hydrates, solvates, prodrugs, salts, in particular pharmaceutically acceptable salts, and/or co-precipitates.

The compounds disclosed herein can exist as a hydrate, or as a solvate, wherein the compounds contain polar solvents, in particular water, methanol or ethanol for example, as structural element of the crystal lattice of the compounds. It is possible for the amount of polar solvents, in particular water, to exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, it is possible for the compounds disclosed herein to exist in free form, e.g. as a free base, or as a free acid, or as a zwitterion, or to exist in the form of a salt. Said salt may be any salt, either an organic or inorganic addition salt, particularly any pharmaceutically acceptable organic or inorganic addition salt, which is customarily used in pharmacy, or which is used, for example, for isolating or purifying the compounds disclosed herein.

The term “pharmaceutically acceptable salt” refers to an inorganic or organic acid addition salt of a compound disclosed herein. For example, see S. M. Berge, et al. “Pharmaceutical Salts,” J. Pharm. Sci. 1977, 66, 1-19. A suitable pharmaceutically acceptable salt of the compounds disclosed herein may be, for example and without limitation, an acid-addition salt of a compound disclosed herein bearing a nitrogen atom, in a chain or in a ring, for example, which is sufficiently basic, such as an acid-addition salt with an inorganic acid, or “mineral acid”, such as hydrochloric, hydrobromic, hydroiodic, sulfuric, sulfamic, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, 3-phenylpropionic, pivalic, 2-hydroxyethanesulfonic, itaconic, trifluoromethanesulfonic, dodecylsulfuric, ethanesulfonic, benzenesulfonic, para-toluenesulfonic, methanesulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, orthiocyanic acid. Further, another suitably pharmaceutically acceptable salt of a compound disclosed herein which is sufficiently acidic, is an alkali metal salt, for example a sodium or potassium salt, an alkaline earth metal salt, for example a calcium, magnesium or strontium salt, or an aluminium or a zinc salt, or an ammonium salt derived from ammonia or from an organic primary, secondary or tertiary amine having 1 to 20 carbon atoms, such as ethylamine, diethylamine, triethylamine, ethyldiisopropylamine, monoethanolamine, diethanolamine, triethanolamine, dicyclohexylamine, dimethylaminoethanol, diethylaminoethanol, tris(hydroxymethyl)aminomethane, procaine, dibenzylamine, N-methylmorpholine, arginine, lysine, 1,2-ethylenediamine, N-methylpiperidine, N-methyl-glucamine, N,N-dimethyl-glucamine, N-ethyl-glucamine, 1,6-hexanediamine, glucosamine, sarcosine, serinol, 2-amino-1,3-propanediol, 3-amino-1,2-propanediol, 4-amino-1,2,3-butanetriol, or a salt with a quaternary ammonium ion having 1 to 20 carbon atoms, such as tetramethylammonium, tetraethylammonium, tetra(n-propyl)ammonium, tetra(n-butyl)ammonium, N-benzyl-N,N,N-trimethylammonium, choline or benzalkonium.

Those skilled in the art will further recognise that it is possible for acid addition salts of the claimed compounds to be prepared by reaction of the compounds with the appropriate inorganic or organic acid via any of a number of known methods. Alternatively, alkali and alkaline earth metal salts of acidic compounds disclosed herein are prepared by reacting the compounds of the present invention with the appropriate base via a variety of known methods. The present invention includes all possible salts of the compounds disclosed herein as single salts, or as any mixture of said salts, in any ratio.

In the present text, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown.

Furthermore, the present invention includes all possible crystalline forms, or polymorphs, of the compounds of the present invention, either as single polymorph, or as a mixture of more than one polymorph, in any ratio.

Moreover, the present invention also includes prodrugs of the compounds according to the invention. The term “prodrugs” here designates compounds which themselves can be biologically active or inactive, but are converted (for example metabolically or hydrolytically) into compounds according to the invention during their residence time in the body.

### Targets

The term “LRRC15” refers to the protein Leucine-rich repeat-containing protein 15. The human LRRC15 protein is encoded by the gene LRRC15 (NCBI gene ID 131578). A synonym for LRRC15 is LIB. The LRRC15 protein comprises human, murine, cynomolgus and further mammalian and non-mammalian homologues. Sequence(s) for human LRRC15 are accessible via UniProt Identifier Q8TF66 (LRC15_HUMAN), for instance human isoform Q8TF66-1 or Q8TF66-2 (Entry version 159, Jun. 17, 2020). Sequence(s) for murine LRRC15 are accessible via UniProt Identifier Q80X72 (LRRC15_MOUSE). Sequence(s) for cynomolgus (Macaca fascicularis) LRRC15 are accessible via UniProt Identifier G7NYR2 (G7NYR2_MACFA). Different isoforms and variants may exist for the different species and are all comprised by the term LRRC15. Also comprised are LRRC15 molecules before and after maturation, i.e., independent of cleavage of one or more pro-domains. In addition, synthetic variants of the LRRC15 protein may be generated and are comprised by the term LRRC15. The protein LRRC15 may furthermore be subject to various modifications, e.g., synthetic or naturally occurring modifications. Recombinant human LRRC15 (rh LRRC15) is commercially available or can be manufactured as known in the art.

The term “EPHB6” refers to the protein Ephrin type-B receptor 6. The human EPHB6 protein is encoded by the gene EPHB6 (NCBI gene ID 2051). This gene encodes a member of a family of transmembrane proteins that function as receptors for ephrin-B family proteins. Unlike other members of this family, the encoded protein does not contain a functional kinase domain. Activity of this protein can influence cell adhesion and migration. Expression of this gene is downregulated during tumor progression, suggesting that the protein may suppress tumor invasion and metastasis. Ephrin receptors and their ligands, the ephrins, mediate numerous developmental processes, particularly in the nervous system. A synonym for EPHB6 is HEP. Sequence(s) for human EPHB6 are accessible via UniProt Identifier 015197 (EPHB6_HUMAN), for instance human isoform 015197-1 or 015197-2.

### Biological Subject Matter

The terms “peptide”, “polypeptide”, and “protein” are used interchangeably herein, and refer to a compound which comprises at least two amino acid residues covalently linked by at least one peptide bond. No limitation is placed on the maximum number of amino acids that can comprise a peptide's sequence. A “peptide” may comprise without limitation modified amino acids, non naturally-occurring amino acids and/or D amino acids. Unless otherwise indicated, a particular peptide sequence also encompasses variants wherein at least one amino acid has been replaced by an amino acid which is characterized by similar structural properties. A “peptide” may be a natural peptide, a recombinant peptide, a synthetic peptide, or a combination thereof. A “peptide” may be, for example, a biologically active fragment, an oligopeptide, a homodimer, a heterodimer, a peptide variant, a modified peptide, a peptide derivative, a peptide analog, a fusion protein, among others.

The term “amino acid” or “amino acid residue” (“aa”) as used herein typically refers to a naturally-occurring amino acid but may also refer to a non naturally-occurring amino acid. The term typically refers to an L-amino acid but may also encompass a D-amino acid. An amino acid may or may not be modified as described elsewhere herein. The one letter code is used herein to refer to the respective amino acid. As used herein, a “charged amino acid” is an amino acid which is negatively charged or positively charged. “Negatively charged amino acids” are aspartic acid (D) and glutamic acid (E). “Positively charged amino acids” are arginine (R) lysine (K) and histidine (H). “Polar amino acids” are all amino acids that form hydrogen bonds as donors or acceptors. These are all charged amino acids and asparagine (N), glutamine (Q), serine (S), threonine (T), tyrosine (Y) and cysteine (C). “Polar uncharged amino acids” are asparagine (N), glutamine (Q), serine (S), threonine (T), tyrosine (Y) and cysteine (C). “Amphiphatic amino acids” are tryptophan (W), tyrosine (Y) and methionine (M). “Aromatic amino acids” are phenylalanine (F), tyrosine (Y), and tryptophan (W). “Hydrophobic amino acids” are glycine (G), alanine (A), valine (V), leucine (L), isoleucine (I), proline (P), phenylalanine (F), methionine (M) and cysteine. “Small amino acids” are glycine (G), alanine (A), serine (S), proline (P), threonine (T), aspartic acid (D) and asparagine (N).

Two amino acids are “characterized by similar structural properties” if (a) both are charged amino acids, preferably both are negatively charged amino or both are positively charged amino acids, (b) both are polar amino acids, (c) both are polar uncharged amino acids, (d) both are amphiphatic amino acids, (e) both are aromatic amino acids, (f) both are hydrophobic amino acids, or (g) both are small amino acids.

The term “nucleic acid” refers to deoxyribonucleotides or ribonucleotides and polymers thereof composed of monomers (nucleotides) containing a sugar, phosphate and a base that is either a purine or pyrimidine. For example and without limitation nucleic acids may occur in single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., (1991); Ohtsuka et al., (1985); Rossolini et al., (1994)).

The term “nucleotide sequence” refers to a polymer of DNA or RNA which can be single- or double-stranded, optionally containing synthetic, non-natural or altered nucleotide bases capable of incorporation into DNA or RNA polymers. The terms “nucleic acid”, “nucleic acid molecule”, “nucleic acid fragment”, “nucleic acid sequence or segment”, or “polynucleotide” are used interchangeably and may also be used interchangeably with gene, cDNA, DNA and RNA encoded by a gene.

“Sequence identity”, “percent identity” or “percent (%) sequence identity” is a number that describes how similar a query sequence is to a target sequence, more precisely how many characters in each sequence are identical after alignment. The most popular tool to calculate sequence identity is BLAST (basic local alignment search tool, NCBI), which performs comparisons between pairs of sequences, searching for regions of local similarity. Suitable alignment methods are known in the art, e.g. Needleman-Wunsch algorithm for global-global alignment, using BLOSUM62 matrix, with gap opening penalty of 11 and a gap extension penalty of 1. Afterwards, the pairs of aligned identical residues can be counted and then divided by the total length of the alignment (including gaps, internal as well as external) to arrive at the percent identity value. For “percent similarity” values, the same approach as for percent identity values can be used, except that what is counted, instead of pairs of identical residues, would be the aligned residue pairs with BLOSUM62 values that are not negative (i.e., ≥0). “Sequence homology” indicates the percentage of amino acids that are identical or that represent conservative amino acid substitutions.

A “host cell” is a cell that is used in to receive, maintain, reproduce and amplify a vector. A host cell also can be used to express the polypeptide encoded by the vector. The nucleic acid contained in the vector is replicated when the host cell divides, thereby amplifying the nucleic acids.

The term “vector”, as used herein, refers to a nucleic acid molecule capable of propagating a nucleic acid molecule to which it is linked. The term further comprises plasmids (non-viral) and viral vectors. Certain vectors are capable of directing the expression of nucleic acids or polynucleotides to which they are operatively linked. Such vectors are referred to herein as “expression vectors”. Expression vectors for eukaryotic use can be constructed by inserting a polynucleotide sequence encoding at least one protein of interest (POI) into a suitable vector backbone. The vector backbone can comprise the necessary elements to ensure maintenance of the vector and, if desirable, to provide amplification within the host. For viral vectors, e.g. lentiviral or retroviral vectors, further virus specific elements such as structural elements or other elements can be required and are well known in the art. These elements can be for instance provided in cis (on the same plasmid) or in trans (on a separate plasmid). Viral vectors may require helper viruses or packaging lines for large-scale transfection. Vectors may contain further elements such as e.g. enhancer elements (e.g. viral, eukaryotic), introns, and viral origins of plasmid replication for replication in mammalian cells. According to the current invention, expression vectors typically have a promoter sequence that drives expression of the POI. Expression of the POI and/or selective marker protein may be constitutive or regulated (e.g. inducible by addition or removal of small molecule inductors). Preferred regulatory sequences for mammalian host cell expression include viral elements that direct high levels of expression of a POI in mammalian cells, such as regulatory elements, promoters and/or enhancers derived from cytomegalovirus (CMV), Simian Virus 40 (SV40), adenovirus, (e.g., the adenovirus major late promoter Ad LP) or polyoma. For further description of viral regulatory elements, and sequences thereof, see e.g., U.S. Pat. No. 5,168,062 by Stinski, U.S. Pat. No. 4,510,245 by Bell et al. and U.S. Pat. No. 4,968,615 by Schaffner et al.

### Antibodies

The terms “(anti) LRRC15 antibody”, “antibody binding LRRC15” and “antibody that binds to LRRC15” are used synonymously herein and refer to an antibody that is capable of binding LRRC15, preferably with sufficient affinity such that the antibody is useful as a diagnostic and/or therapeutic agent in targeting LRRC15. In some embodiments, the extent of binding of an antibody binding LRRC15 to an unrelated protein different from LRRC15 is less than about 10%, less than about 5%, or preferably less than about 2%, and most preferably less than about 1% of the binding of the antibody to LRRC15 as measured, e.g. by surface plasmon resonance (SPR) or a further standard method such as ELISA assay.

In certain preferred embodiments, an antibody that binds to LRRC15 has a dissociation constant (K_{D}) of ≤1 μM, ≤500 nM, ≤200 nM, ≤100 nM, ≤10 nM, ≤1 nM, ≤0.1 nM, 0.01 nM, or ≤50.001 nM (e.g. 10⁻⁸ M or less, e.g. from 10⁻⁸ M to 10⁻¹³ M, e.g., from 10⁻⁹ M to 10⁻¹³ M). In certain embodiments, an antibody that binds to LRRC15 has a binding activity (EC50) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M 25 to 10⁻¹³ M, e.g., from 10⁻⁹ M to 10⁻¹³ M). In certain embodiments, an anti LRRC15 antibody binds to an epitope of LRRC15 that is conserved among LRRC15 from different species.

The term “antibody” includes, but is not limited to, an immunoglobulin molecule (e.g. without limitation human IgG1, IgG2, IgG3, IgG4, IgM, IgD, IgE, IgA1, IgA2, mouse IgG1, IgG2a, IgG2b, IgG2c, IgG3, IgA, IgD, IgE or IgM, rat IgG1, IgG2a, IgG2b, IgG2c, IgA, IgD, IgE or IgM, rabbit IgA1, IgA2, IgA3, IgE, IgG, IgM, goat IgA, IgE, IgG1, IgG2, IgE, IgM or chicken IgY) that binds to a particular antigen. The term also comprises bispecific antibodies as described elsewhere herein. Depending on the context, the term antibody may also refer to a functional fragment of a full length antibody as disclosed elsewhere herein. The term furthermore includes any proteinaceous binding molecule with immunoglobulin-like function. According to the current invention, the antibodies or fragments thereof are preferably characterized by an affinity for their target corresponding to a K_{D} of less than 10⁻⁷ M, more preferably of less than 10⁻⁸ M, even more preferably in the range from 10⁻¹¹ M to 10⁻⁹ M. In some embodiments the antibody may be a lama, camel, alpake (e.g. camelid-hcIgG or IgG), or shark (e.g. IgNAR) antibody.

An antibody may be composed of two identical pairs of polypeptide chains. In particular embodiments, antibodies may comprise four polypeptide chains, e.g. two “heavy chains” (H) (about 50-70 kDa) and two “light chains” (L) (about 25 kDa), which may be connected by disulfide bonds.

The amino-terminal portion of a polypeptide chain of an antibody usually comprises a “variable region”, e.g. of about 100 to 110 or more amino acids, which is primarily responsible for antigen recognition. The heavy chain variable region is abbreviated herein as “VH”, the light chain variable region is abbreviated herein as “VL”. The VH and VL regions can be further subdivided into regions of hypervariability, termed CDRs, interspersed with regions that are more conserved, termed “framework regions” or “FR”. Each VH and VL is typically composed of three CDRs and up to four FRs, arranged from amino-terminus to carboxy-terminus e.g., in the following order: FR1, CDR1, FR2, CDR2, FR3, CDR3, FR4.

The term “CDR” refers to the complementary determining region of an antibody. CDRs are crucial to the diversity of antigen specificities. A set of CDRs constitutes a paratope. There are usually three CDRs (CDR1, CDR2 and CDR3), arranged non-consecutively on the amino acid sequence of a variable domain of an antigen receptor. The CDRs are typically held together in close proximity by the FR regions and—e.g. with the CDRs from the other chain—contribute to the formation of the antigen binding site of antibodies. The CDRs of the light chain are termed LCDR1, LCDR2 and LCDR3. The CDRs of the heavy chain are termed HCDR1, HCDR2 and HCDR3. HCDR3 is the most variable CDR. As known in the art, the amino acid position/boundary delineating a hypervariable region of an antibody can vary, depending on the context and the various definitions known in the art. As used herein, numbering of antibody amino acid residues is done according to the immunoglobulin amino acid residue numbering system of Kabat.

The carboxy-terminal portion of each polypeptide chain of an antibody usually comprises a “constant region”, a portion of the antibody molecule that confers effector functions. The heavy chain constant region can comprise e.g. three domains CH1, CH2 and CH3. The light chain constant region is comprised of one domain (CL). The heavy chain constant region can for example be selected from any of the five isotypes: alpha (α), delta (δ), epsilon (ε), gamma (g), or mu (μ).

The term “fragment crystallizable region”, also “Fc domain”, “Fc region” or “Fc part” as used herein refers to a C-terminal region of an antibody heavy chain that contains at least a portion of the constant region. The Fc region may interact with Fc receptors and some proteins of the complement system, e.g. on immune effector cells. The Fc region defines the class of an antibody or isotype. For an IgG antibody, the Fc region consists of the C2H and C3H domains. The term includes native sequence Fc regions and variant Fc regions. For example, a human IgG heavy chain Fc region may extend from Cys226, or from Pro230, to the carboxyl-terminus of the heavy chain.

Antibodies or binding fragments according to the current invention may have been modified to alter at least one Fc region-mediated biological effector function, e.g. by reduced or improved binding to the Fc receptor (e.g. FcγR). FcγR binding may be reduced, e.g. by mutating the immunoglobulin constant region/Fc region of the antibody (See, e.g., Canfield and Morrison, 1991, J. Exp. Med. 173:1483-1491; and Lund et al., 1991, J. Immunol. 147:2657-2662), or may be enhanced, e.g. by afucosylation. Reducing or enhancing Fc(γ)R binding may also reduce or enhance other effector functions which rely on Fc(γ)R interactions, such as opsonization, phagocytosis, ADCP or ADCC.

Antibodies or antibody fragments can be produced synthetically or recombinantly. A number of technologies are available to produce antibodies. For example, phage-antibody technology can be used to generate antibodies (Knappik et al., J. Mol. Biol. 296:57-86, 2000). Another approach for obtaining antibodies is to screen a DNA library from B cells as described in WO 91/17271 and WO 92/01047. In these methods, libraries of phages are produced in which members display different antibodies on their outer surfaces. Antibodies are usually displayed as Fv or Fab fragments. Phage displaying antibodies are selected by affinity enrichment for binding to a selected protein. Antibodies can also be produced using trioma methodology (e.g., Oestberg et al., Hybridoma 2:361-367, 1983; U.S. Pat. Nos. 4,634,664; 4,634,666).

Antibodies can also be purified from any cell that expresses the antibodies, including host cells that have been transfected with antibody-encoding expression constructs. The host cells can be cultured under conditions whereby the antibodies are expressed. Purified antibody can be separated from other cellular components that can associate with the antibody in the cell, such as certain proteins, carbohydrates, or lipids, using methods well known in the art. Such methods include, but are not limited to, size exclusion chromatography, ammonium sulfate fractionation, ion exchange chromatography, affinity chromatography, and preparative gel electrophoresis. Purity of the preparations can be assessed by any means known in the art, such as SDS-polyacrylamide gel electrophoresis. A preparation of purified antibodies can contain more than one type of antibody.

Alternatively, antibodies according to the current invention can be produced using chemical methods to synthesize its amino acid sequence, such as by direct peptide synthesis using solid-phase techniques (e.g., Merrifield, J. Am. Chem. Soc. 85:2149-2154, 1963; Roberge et al., Science 269:202-204, 1995). Protein synthesis can be performed using manual techniques or by automation. Optionally, fragments of antibodies can be separately synthesized and combined using chemical methods to produce a full-length molecule.

A “proteinaceous binding molecule with immunoglobulin-like function” is a proteinaceous molecule which is not an immunoglobulin but binds to a particular antigen. An example of a proteinaceous binding molecule with immunoglobulin-like functions is a mutein based on a polypeptide of the lipocalin family (WO 03/029462, Beste et al., Proc. Natl. Acad. Sci. USA (1999) 96, 1898-1903). Lipocalins, such as the bilin binding protein, the human neutrophil gelatinase-associated lipocalin, human Apolipoprotein D or glycodelin, possess natural ligand-binding sites that can be modified so that they bind to selected small protein regions known as haptens. Examples of other proteinaceous binding molecules are glubodies (see e.g. WO 96/23879 or Napolitano, E. W., et al., Chemistry & Biology (1996) 3, 5, 359-367), proteins based on the ankyrin scaffold (Mosavi, L. K., et al., Protein Science (2004) 13, 6, 1435-1448) or crystalline scaffold (e.g. WO 01/04144), the proteins described in Skerra, J. Mol. Recognit. (2000) 13, 167-187, adnectins, tetranectins, avimers and peptoids. Adnectins, derived from a domain of human fibronectin, contain three loops that can be engineered for immunoglobulin-like binding to targets (Gill, D. S. & Damle, N. K., Current Opinion in Biotechnology (2006) 17, 653-658). Tetranectins, derived from the respective human homotrimeric protein, likewise contain loop regions in a C-type lectin domain that can be engineered for desired binding. Avimers contain so called A-domains that occur as strings of multiple domains in several cell surface receptors (Silverman, J., et al., Nature Biotechnology (2005) 23, 1556-1561). Peptoids, which can act as protein ligands, are oligo(N-alkyl) glycines that differ from peptides in that the side chain is connected to the amide nitrogen rather than the alpha carbon atom. Peptoids are typically resistant to proteases and other modifying enzymes and can have a much higher cell permeability than peptides (see e.g. Kwon, Y.-U., and Kodadek, T., J. Am. Chem. Soc. (2007) 129, 1508-1509).

### Antibody Fragments

A “fragment”, “functional fragment” or “antigen-binding fragment” of an antibody is required to retain the ability of the antibody to bind the particular antigen. Fragments of an antibody therefore typically comprise a functional portion of a full-length antibody, generally the antigen binding region or variable region thereof. Preferably, a fragment of an antibody as used herein substantially retains the affinity of the full-length antibody. As such, suitable fragments of an anti-LRRC15 antibody will retain the ability to bind to the target protein, e.g. to bind to LRRC15. Examples of antibody fragments include, but are not limited to, Fab, Fab′, F(ab′)2, and Fv fragments, single-chain antibody molecules, diabodies and domain antibodies, see Holt, L. J., et al., Trends Biotechnol. (2003), 21, 11, 484-490.

A “Fab fragment” contains the constant domain of the light chain and the first constant domain (CH2) of the heavy chain. “Fab′ fragments” differ from Fab fragments by the addition of a few residues at the carboxyl terminus of the heavy chain CH2 domain including one or more cysteines from the antibody hinge region. “F(ab′) fragments” are produced by cleavage of the disulfide bond at the hinge cysteines of the F(ab′)2 pepsin digestion product. Additional chemical couplings of antibody fragments are known to those of ordinary skill in the art. Fab and F(ab′)2 fragments lack the Fc fragment of intact antibody, clear more rapidly from the circulation of animals, and may have less non-specific tissue binding than an intact antibody, see, e.g., Wahl et al., 1983, J. Nucl. Med. 24:316.

An “Fv fragment” is the minimum fragment of an antibody that contains a complete target recognition and binding site. This region consists of a dimer of one heavy and one light chain variable domain in a tight, non-covalent association (VH-VL dimer). It is in this configuration that the three CDRs of each variable domain interact to define an antigen binding site on the surface of the VH-VL dimer. Often, the six CDRs confer antigen binding specificity upon the antibody. However, in some instances even a single variable domain (or half of an Fv comprising only three CDRs specific for a target) may have the ability to recognize and bind antigen, although at a lower affinity than the entire binding site.

“Single-chain Fv” or “scFv” antibody fragments comprise the VH and VL domains of an antibody in a single polypeptide chain. Generally, the Fv polypeptide further comprises a polypeptide linker between the VH and VL domains which enables the scFv to form the desired structure for antigen binding.

“Single domain antibodies” are composed of a single VH or VL domains which exhibit sufficient affinity to the target. In a specific embodiment, the single domain antibody is a camelized antibody, see, e.g., Riechmann, 1999, Journal of Immunological Methods 231:25-38.

### Binding

The term “affinity” or “binding affinity” is a term of the art and describes the strength of non-covalent binding between a single binding site of a molecule and its binding partner. The affinity of an antibody or fragment thereof for a target can be determined using techniques well known in the art, for example by ELISA, isothermal titration calorimetry (ITC), surface plasmon resonance (SPR), flow cytometry or fluorescent polarization assays. Preferably the affinity is provided as dissociation constant K_{D}, in the alternative the affinity is provided as an EC50 value.

The “dissociation constant” (K_{D}) has molar units (M) and corresponds to the concentration of the binder (e.g. antibody or fragment) at which half of the target proteins or binding partners are occupied at equilibrium. The smaller the dissociation constant is, the higher is the affinity between the binder and its target. The K_{D} values can be preferably measured by using surface plasmon resonance assays using suitable devices including but not limited to Biacore instruments like Biacore T100, Biacore T200, Biacore 2000, Biacore 4000, a Biacore 3000 (GE Healthcare Biacore, Inc., cf. e.g., Sjolander & Urbaniczky; Anal. Chem. 63:2338-2345, 1991; Szabo, et al., Curr. Opin. Struct. Biol. 5:699-705, 1995), or a ProteOn XPR36 instrument (Bio-Rad Laboratories, Inc.). In the alternative the affinity can be determined using any method known in the art including, for example immunoassays such as enzyme-linked immununospecific assay (ELISA) and fluorescence-activated cell sorting (FACS) for quantification of antibody binding to cells that express an antigen. Where assay conditions were found to influence the determined KD, the assay setup with the least standard deviation shall be used.

“Half maximal effective concentration” (EC50) refers to the concentration of a drug, modulator, antibody, fragment, conjugate or molecule which induces a response halfway between the baseline and maximum after a specified incubation time. In the context of affinity, the EC50 thus reflects the concentration of binder (e.g. antibody) that is needed for half-maximal binding. An EC50 can be determined if an inflection point can be determined by mathematical modeling (e.g., non-linear regression) of the dose-response curve describing the relationship between applied drug, antibody, fragment, conjugate or molecule concentration and signal. For example, if the dose-response curve follows a sigmoidal curve, an EC50 can be determined. Where the response is an inhibition, the EC50 is termed “half maximal inhibitory concentration” (IC50).

If two quantities are “in the same order of magnitude”, the larger value is less than ten times the smaller value.

As used herein, a binder or antibody that “binds specifically to”, is “specific to/for” or “specifically recognizes” an antigen of interest, e.g. LRRC15, is one that binds the antigen with sufficient affinity such that the binder or antibody is useful as a therapeutic agent in targeting a cell or tissue expressing the antigen, and does not significantly cross-react with proteins other than orthologs and variants (e.g. mutant forms, splice variants, or proteolytically truncated forms) of the aforementioned antigen target. The term “specifically recognizes” as used herein can be exhibited, for example, by a binder, an antibody, or antigen-binding fragment thereof, having a monovalent K_{D} for the antigen of less than about 10⁻⁴ M, alternatively less than about 10⁻⁵ M, alternatively less than about 10⁻⁶ M, alternatively less than about 10⁻⁷ M, alternatively less than about 10⁻⁸ M, alternatively less than about 10⁻⁹ M, alternatively less than about 10⁻¹⁰ M, alternatively less than about 10⁻¹¹ M, alternatively less than about 10⁻¹² M, or less.

In its most general form, “specific binding” is referring to the ability of a binder or antibody to discriminate between the antigen of interest and an unrelated antigen, as determined, for example, by surface plasmon resonance (SPR), Western blot, ELISA-, RIA-, ECL-, IRMA-test or peptide scans. For example, a standard ELISA assay can be carried out. The scoring may be carried out by standard color development (e.g. secondary antibody with horseradish peroxidase and tetramethyl benzidine with hydrogen peroxide). The reaction in certain wells is scored by the optical density, for example, at 450 nm. Typical background (=negative reaction) may be 0.1 OD; typical positive reaction may be 1 OD. This means the difference positive/negative is more than 5-fold, 10-fold, 50-fold, and preferably more than 100-fold. Typically, determination of binding specificity is performed by using not a single reference antigen, but a set of about three to five unrelated antigens, such as milk powder, BSA, transferrin or the like.

“Polyspecificity”, also “polyreactivity” or “unspecific binding” refers to the binders' or antibodies' ability to bind a defined set of unrelated antigens but the terms are not necessarily used interchangeably. Where the binder or antibody binds specifically to a target and binds unspecifically to at least one further unrelated antigen this is called “polyreactivity” or “unspecific binding”. Where a binder or antibody binds not only specifically to the intended target but also binds specifically to a further unrelated target this is called “polyspecificity”. Polyspecificity, polyreactivity and unspecific binding is substantial if the (therapeutic) applicability of the antibody is compromised. Binding of non-protein structures including without limitation target negative cell lines or tissues, baculo virus particle (BVP), insulin or DNA, may be evaluated as known in the art. In a first example, binding to target negative human cell lines can be determined e.g. by FACS analysis using mock transfected CHO or HEK293 cells. In a second example, unspecific binding to different tissues or cell populations can be analyzed by FACS analysis of a cell line or panel of cell lines derived from the respective tissue, or by FACS analysis of a sorted cell population. In a third example, unspecific binding to BVP, insulin or DNA can be analyzed using ELISA, e.g. as described in Hötzel, Isidro, et al. “A strategy for risk mitigation of antibodies with fast clearance.” MAbs. Vol. 4. No. 6. Taylor & Francis, 2012, Avery, Lindsay B., et al. “Establishing in vitro in vivo correlations to screen monoclonal antibodies for physicochemical properties related to favorable human pharmacokinetics.” MAbs. Vol. 10. No. 2. Taylor & Francis, 2018, and Jain, Tushar, et al. “Biophysical properties of the clinical-stage antibody landscape.” Proceedings of the National Academy of Sciences 114.5 (2017): 944-949, incorporated herein in their entirety and in particular with regards to the technical details necessary to analyze and quantify unspecific binding. The degree of unspecific binding of an antibody according to the current invention at least with regard to BVP, insulin and DNA, is preferably lower than the degree of unspecific binding of reference antibody Gantenerumab (Roche) and most preferably lower than the degree of unspecific binding of reference antibody Remicade (Janssen Biotech).

An antibody or fragment is termed “cross-reactive” or “cross reactive” if the antibody or fragment binds an antigen from a first species (e.g. human LRRC15) and a related antigen from at least one further species (e.g. cynomolgus LRRC15), e.g. without limitation both with a K_{D} value of less than 10⁻⁷ M, more preferably of less than 10⁻⁸ M, even more preferably in the range from 10⁻⁹ M to 10⁻¹¹ M.

As used herein, the term “epitope” refers to a structure that is specifically bound by an antibody or T-cell receptor. Epitopes may be characterized by specific three dimensional structures or charge patterns. For example, these three dimensional structures or charge patterns may be defined by amino acids or sugar residues. According to preferred embodiments an epitope may be a defined amino acid sequence, which may or may not be modified.

### FC Functions

An “activating Fc receptor” is an Fc receptor that elicits signaling events that stimulate the receptor-bearing cell to perform effector functions upon binding to an Fc domain of an antibody. Human activating Fc receptors include without limitation FcγRIIIa (CD16a), FcγRI (CD64), FcγRIIa (CD32), and FcαRI (CD89).

The term “effector functions” refers to those biological activities attributable to the Fc region of an antibody, which vary with the antibody isotype. Examples of antibody effector functions include without limitation: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

The term “effector cells” refers to a population of lymphocytes that display effector moiety receptors, e.g. cytokine receptors, and/or Fc receptors on their surface through which they bind an effector moiety, e.g. a cytokine, and/or an Fc region of an antibody and contribute to the destruction of target cells, such as tumor cells. Effector cells may for example mediate ADCC, ADCP or CDC. Effector cells include, but are not limited to, effector T cells such as CD8 positive cytotoxic Tcells, CD4 positive helperT cells, γδ Tcells, NK cells, lymphokine-activated killer (LAK) cells and macrophages/monocytes.

“Afucosylated” antibodies are antibodies engineered such that the oligosaccharides in the Fc region of the antibody do not have any fucose sugar units. Glycosylation of an antibody can alter its function. For example, if glycosylation at N297 in the CH2 domain of an IgG is completely eliminated, binding to FcγRs is lost. However, modulation of the specific carbohydrate composition at N297 can have the opposite effect and enhance the ADCC activity of the antibody. In brief, the affinity of an antibody for the activating FcγRs depends on the composition of the N297 N-linked oligosaccharide. There are 32 different possible combinations of oligosaccharides that can occur at this site. Naturally occurring human IgG and those produced by hybridomas or other common expression systems are usually composed of N-acetylglucosamine (GlcNAc) and three mannose residues that form a core carbohydrate. This core is attached to two additional GlcNAc groups to form biantennary branches. The addition of galactose at each branch can occur as well as the terminal addition of sialic acid to these galactose molecules. Fucose is often part of the core GlcNAc. This fucose, through steric hindrance, obstructs the interaction of the antibody with the FcγRIIIA. Thus, elimination of this fucose molecule while maintaining other forms of glycosylation at this site increases the binding of the antibody to the activating FcγRs, enhancing its ability to elicit ADCC and/or ADCP (Almagro 2017, Front Immunol. 2017; 8: 1751). Methods of preparing fucose-less antibodies include growth in rat myeloma YB2/0 cells (ATCC CRL 1662). YB2/0 cells express low levels of FUT8 mRNA, which encodes α-1,6-fucosyltransferase, an enzyme necessary for fucosylation of polypeptides. Afucosylated antibodies are preferred embodiments of the current invention.

“Antibody-dependent cellular cytotoxicity” (“ADCC”), also “antibody-dependent cell-mediated cytotoxicity”, is a mechanism of cell-mediated immune defense whereby an immune cell actively lyses a target cell, whose membrane-surface antigens have been bound by specific antibodies. ADCC is mediated via interaction of the antibody or fragment with FcγRIIIa. In humans, FcγRIII exists in two different forms: FcγRIIIa (CD16a) and FcγRIIIb (CD16b). While FcγRIIIa is expressed on monocytes, neutrophils, mast cells, macrophages, and natural killer cells as a transmembrane receptor, FcγRIIIb is only expressed on neutrophils. These receptors bind to the Fc portion of an antibody, which then activates antibody-dependent cell-mediated cytotoxicity (ADCC) mediated by the human effector cells. Different assay systems to determine ADCC induction in human subjects have been described in the literature and are suitable for characterization of the subject matter disclosed herein. For example, Yao-Te Hsieh et al. have studied different ADCC assay systems, namely assays based on (i) natural killer cells from human donors (FcγRIIIA+primary NK), (ii) FcγRIIIA engineered NK-92 cells and (iii) FcγRIIIA/NFAT-RE/luc2 engineered Jurkat T cells (Hsieh, Yao-Te, et al. “Characterization of FcγRIIIA effector cells used in in vitro ADCC bioassay: comparison of primary NK cells with engineered NK-92 and Jurkat T cells.” Journal of Immunological Methods 441 (2017): 56-66, incorporated herein in entirety; in particular, reference is made to the method description for these assays). In brief, all three effector cell systems differentially express FcγRIIIA and provide dose-dependent ADCC pathway activity, yet only primary NK and engineered NK-92 cells are capable of inducing ADCC-mediated cell lysis. For functional assessment of ADCC activity, primary NK or NK-92 (V-158) cells thus better reflect the physiologically relevant ADCC mechanism of action. As an engineered cell line, NK-92 cells may behave more reproducibly than primary NK and are therefore the preferred assay system to determine ADCC response in human subjects, e.g. in case of doubt.

An antibody “inducing ADCC” is an antibody which may elicit a substantial amount of lysis of target cells in the presence of FcγRIIIa expressing effector cells. Preferably, the ADCC induction results in the lysis of at least 2%, 5%, 10%, 15%, more preferably at least 20%, 25%, 30%, 35%, 40%, 45%, most preferably at least 50%, 55%, 60%, 65%, 70%, 75%, 80%, 85%, 90%, 95% or 99% of the target cells. Antibodies inducing ADCC are preferred embodiments of the current invention.

“Antibody-dependent cellular phagocytosis” (“ADCP”) is the mechanism by which antibody-opsonized target cells activate the FcγRs on the surface of macrophages to induce phagocytosis, resulting in internalization and degradation of the target cell. For ADCP, binding to macrophages as effector cells typically occurs via the interaction of the antibodies FC part with FcγRIIa (CD32a) expressed by macrophages.

An antibody “inducing ADCP” is an antibody which may elicit a substantial amount of phagocytosis of target cells in the presence of macrophages. Preferably, the ADCP induction results in the phagocytosis of at least 2%, 5%, 10%, 15%, more preferably at least 20%, 25%, 30%, 35%, 40%, 45%, most preferably at least 50%, 55%, 60%, 65%, 70%, 75%, 80%, 85%, 90%, 95% or 99% of the target cells. Antibodies inducing ADCP are preferred embodiments of the current invention.

“C1q binding and complement dependent cytotoxicity” also “Complement-dependent cytotoxicity” (“CDC”) is an effector function of IgG and IgM antibodies. When they are bound to a surface antigen on a target cell, the classical complement pathway is triggered by bonding protein C1q to these antibodies, resulting in formation of a membrane attack complex (MAC) and target cell lysis. Complement system is efficiently activated by human IgG1, IgG3 and IgM antibodies, weakly by IgG2 antibodies and is not activated by IgG4 antibodies. Several laboratory methods exist for determining the efficacy of CDC and are known in the art.

An antibody “inducing CDC” is an antibody which may elicit a substantial amount of formation of a membrane attack complex and lysis of target cells. Preferably, the CDC induction results in the lysis of at least 2%, 5%, 10%, more preferably at least 15%, 20%, 25%, 30%, 35%, 40%, 45%, most preferably at least 50%, 55%, 60%, 65%, 70%, 75%, 80%, 85%, 90%, 95% or 99% of the target cells. Antibodies inducing CDC are preferred embodiments of the current invention.

### Antibody Formats

“Bispecific antibodies” are monoclonal antibodies that have binding specificities for at least two different epitopes on the same target or different targets. In the present disclosure, one of the binding specificities can be directed towards LRRC15, the other can be for any other antigen, e.g., without limitation for a cell-surface protein, receptor, receptor subunit, tissue-specific antigen, virally derived protein, virally encoded envelope protein, bacterially derived protein, or bacterial surface protein. Bispecific antibody constructs according to the invention also encompass multispecific antibody constructs comprising multiple binding domains/binding sites, such as trispecific antibody constructs, where the construct comprises three binding domains.

Bispecific antibody formats comprise IgG-like and non-IgG-like antibodies (Fan et al (2015) Journal of Hematology & Oncology. 8: 130). IgG-like antibodies have a monoclonal antibody (mAb) structure of two Fab arms and one Fc region, wherein the two Fab sites bind different antigens. The most common IgG-like antibody types comprise two Fab regions, and the Fc region. Each heavy and light chain pair may be from a unique mAb. The Fc region is usually made from the two heavy chains. These BsABs can be manufactured for instance with the quadroma or the hybrid hybridoma method or another method known in the art. Non-IgG-like BsABs lack an Fc region. Non-IgG-like BsABs include chemically linked Fabs, comprising only the Fab regions, and various types of bivalent and trivalent single-chain variable fragments (scFvs). There are also fusion proteins mimicking the variable domains of two antibodies. These formats comprise bi-specific T-cell engagers (BiTEs).

Bispecific antibodies include but are not limited to multivalent single chain antibodies, diabodies and triabodies, and antibodies having the constant domain structure of full length antibodies to which further antigen-binding sites are linked via one or more linker or peptide-linker. Possible further antigen-binding sites comprise for example single chain Fv, VH domain and/or VL domain, Fab, (Fab)2, VHH nanobodies (Hamers-Casterman C et al., (1993) Nature 363(6428), 446-448), single domain antibodies, scFabs, or fragments of any of these.

Bispecific antibodies according to the current invention include but are not limited to Fc fusions to which further antigen-binding sites are linked via one or more linker or peptide-linker, for example N-terminal and/or C-terminal. Possible further antigen-binding sites comprise for example single chain Fv, VH domain and/or VL domain, Fab, (Fab)2, VHH nanobodies, single domain antibodies, scFabs, or fragments of any of these. Bispecific antibodies are highly preferred embodiments or form part of highly preferred embodiments of the different aspects of the current invention.

A “modification promoting the association of the first and the second subunit of the Fc domain” is a manipulation of the peptide backbone or the post-translational modifications of an Fc domain subunit that reduces or prevents the association of a polypeptide comprising the Fc domain subunit with an identical polypeptide to form a homodimer. A modification promoting association as used herein particularly includes separate modifications made to each of the two Fc domain subunits desired to associate (i.e. the first and the second subunit of the Fc domain), wherein the modifications are complementary to each other so as to promote association of the two Fc domain subunits. For example, a modification promoting association may alter the structure or charge of one or both of the Fc domain subunits so as to make their association sterically or electrostatically favorable. Thus, (hetero)dimerization occurs between a polypeptide comprising the first Fc domain subunit and a polypeptide comprising the second Fc domain subunit, which might be non-identical, e.g. in the sense that further components fused to each of the subunits (e.g. antigen binding moieties) are not the same. In some embodiments the modification promoting association comprises an amino acid mutation in the Fc domain, specifically an amino acid substitution. In a particular embodiment, the modification promoting association comprises a separate amino acid mutation, specifically an amino acid substitution, in each of the two subunits of the Fc domain. According to the current invention, antibodies comprising an Fc region may or may not comprise a modification promoting the association of the first and the second subunit of the Fc domain. Antibodies comprising a modification promoting the association of the first and the second subunit of the Fc domain are preferred embodiments of the current invention.

The term “chimeric antigen receptor” or “CAR” as used herein, refers to an artificial T cell surface receptor that is engineered to be expressed on an immune effector cell and specifically bind an antigen. CARs may be used as a therapy with adoptive cell transfer. Monocytes are removed from a patient (blood, tumor or ascites fluid) and modified so that they express the receptors specific to a particular form of antigen. In some embodiments, the CARs have been expressed with specificity to a tumor associated antigen. CARs may also comprise an intracellular activation domain, a transmembrane domain and an extracellular domain comprising a tumor associated antigen binding region. In some aspects, CARs comprise fusions of single-chain variable fragments (scFv) derived monoclonal antibodies, fused to CD3-zeta transmembrane and intracellular domain. The specificity of CAR designs may be derived from ligands of receptors (e.g., peptides). In some embodiments, a CAR can target cancers by redirecting a monocyte/macrophage expressing the CAR specific for tumor associated antigens. According to the current invention the CAR binds LRRC15.

An antibody may be monoclonal or polyclonal. The term “polyclonal” refers to antibodies that are heterogenous populations of antibodies, derived for example from the sera of animals immunized with an antigen or an antigenic functional derivative thereof. For the production of polyclonal immunoglobulins, one or more of various host animals may be immunized by injection with the antigen. Various adjuvants may be used to increase the immunological response, depending on the host species.

“Monoclonal antibodies” are substantially homogenous populations of antibodies binding a particular antigen. Monoclonal antibodies may be obtained by methods well known to those skilled in the art (see for example, Köhler et al., Nature (1975) 256, 495-497, and U.S. Pat. No. 4,376,110). An antibody or fragment with specific binding affinity can be isolated, enriched, or purified from a prokaryotic or eukaryotic organism. The antibodies according to the current invention are preferably monoclonal.

“Humanized antibodies” contain CDR regions derived from a non human species, such as mouse, that have, for example, been engrafted, along with any necessary framework back-mutations, into human sequence-derived V regions. Thus, for the most part, humanized antibodies are human immunoglobulins (recipient antibody) in which residues from a hypervariable region of the recipient are replaced by residues from a hypervariable region of a non-human species (donor antibody) such as mouse, rat, rabbit or non human primate having the desired specificity, affinity, and capacity. See, for example, U.S. Pat. Nos. 5,225,539; 5,585,089; 5,693,761; 5,693,762; 5,859,205, each herein incorporated by reference. In some instances, framework residues of the human immunoglobulin are replaced by corresponding non-human residues (see, for example, U.S. Pat. Nos. 5,585,089; 5,693,761; 5,693,762, each herein incorporated by reference). Furthermore, humanized antibodies may comprise residues that are not found in the recipient antibody or in the donor antibody. These modifications are made to further refine antibody performance (e.g., to obtain the desired affinity). In general, the humanized antibody will comprise substantially all of at least one, and typically two, variable domains, in which all or substantially all of the hypervariable regions correspond to those of a non-human immunoglobulin and all or substantially all of the framework regions are those of a human immunoglobulin sequence. The humanized antibody will optionally comprise at least a portion of an immunoglobulin constant region (Fc), typically that of a human immunoglobulin. For further details see Jones et al., Nature 331:522-25 (1986); Riechmann et al., Nature 332:323-27 (1988); and Presta, Curr. Opin. Struct. Biol. 2:593-96 (1992), each incorporated herein by reference.

Fully human antibodies (human antibodies) comprise human derived CDRs, i.e. CDRs of human origin. Preferably, a fully human antibody according to the current invention is an antibody having at least 90%, 91%, 92%, 93%, 94%, 95%, 96%, 97%, 98%, 99%, 99.5% or 100% sequence identity with the closest human VH germline gene (e.g. sequence extracted from recommended list and analyzed in IMGT/Domain-gap-align).

As accepted by usual nomenclature systems such as the INN species subsystem in force until 2017, fully human antibodies may comprise a low number of germline deviations compared with the closest human germline reference determined based on the IMGT database (http://www.imgt.org). For example, a fully human antibody according to the current invention may comprise up to 1, 2, 3, 4 or 5 germline deviations per CDR compared with the closest human germline reference. Fully human antibodies can be developed from human derived B cells by cloning techniques in combination with a cell enrichment or immortalization step. The majority of fully human antibodies in clinical use, however, were isolated either from immunized mice transgenic for the human IgG locus or from sophisticated combinatorial libraries by phage display (Bruggemann M., Osborn M. J., Ma B., Hayre J., Avis S., Lundstrom B. and Buelow R., Human Antibody Production in Transgenic Animals, Arch Immunol Ther Exp (Warsz.) 63 (2015), 101-108; Carter P. J., Potent antibody therapeutics by design, Nat Rev Immunol 6 (2006), 343-357; Frenzel A., Schirrmann T. and Hust M., Phage display-derived human antibodies in clinical development and therapy, MAbs 8 (2016), 1177-1194; Nelson A. L., Dhimolea E. and Reichert J. M., Development trends for human monoclonal antibody therapeutics, Nat Rev Drug Discov 9 (2010), 767-774.)).

Several techniques are available to generate fully human antibodies or to generate antibodies comprising human derived CDRs (cf. WO2008/112640 A3). Cambridge Antibody Technologies (CAT) and Dyax have obtained antibody cDNA sequences from peripheral B cells isolated from immunized humans and devised phage display libraries for the identification of human variable region sequences of a particular specificity. Briefly, the antibody variable region sequences are fused either with the Gene III or Gene VIII structure of the M13 bacteriophage. These antibody variable region sequences are expressed either as Fab or single chain Fv (scFv) structures at the tip of the phage carrying the respective sequences. Through rounds of a panning process using different levels of antigen binding conditions (stringencies), phages expressing Fab or scFv structures that are specific for the antigen of interest can be selected and isolated. The antibody variable region cDNA sequences of selected phages can then be elucidated using standard sequencing procedures. These sequences may then be used for the reconstruction of a full antibody having the desired isotype using established antibody engineering techniques. Antibodies constructed in accordance with this method are considered fully human antibodies (including the CDRs). In order to improve the immunoreactivity (antigen binding affinity and specificity) of the selected antibody, an in vitro maturation process can be performed, including a combinatorial association of different heavy and light chains, deletion/addition/mutation at the CDR3 of the heavy and light chains (to mimic V-J, and V-D-J recombination), and introduction of random mutations (to mimic somatic hypermutation). An example of a “fully human” antibody generated by this method is the anti-tumor necrosis factor α antibody, Humira (adalimumab).

“Derivatized antibodies” are typically modified by glycosylation, acetylation, pegylation, phosphorylation, sulfation, amidation, derivatization by known protecting/blocking groups, proteolytic cleavage, linkage to a cellular ligand or other protein. Any of numerous chemical modifications may be carried out by known techniques, including, but not limited to, specific chemical cleavage, acetylation, formylation, metabolic synthesis of tunicamycin, etc. Additionally, the derivative may contain one or more non-natural amino acids, e.g., using ambrx technology. See, e.g., Wolfson, 2006, Chem. Biol. 13(10):1011-2. Antibodies according to the current invention may be derivatized, e.g. sulfated.

The term “maturated antibodies” or “maturated antigen-binding fragments” such as maturated Fab variants or “optimized” variants includes without limitation derivatives of an antibody or antibody fragment exhibiting stronger binding—i.e. binding with increased affinity—to a given antigen such as the extracellular domain of a target protein. Maturation is the process of identifying a small number of mutations within the six CDRs of an antibody or antibody fragment leading to this affinity increase. The maturation process is the combination of molecular biology methods for introduction of mutations into the antibody and screening for identifying the improved binders.

The term “germlining” refers to replacement of residues in the variable domains of an antibody with those present in the pre-mutated germline genes to reduce the potential for immunogenicity.

### CONJUGATES

The term “conjugate” refers to a molecule comprising at least two moieties. For example, and without limitation, the moieties can be connected via a linker.

The term “antibody conjugate” refers to a conjugate comprising at least one antibody moiety and one or more further molecules or moieties. The one or more further molecules or moieties may be selected without limitation from a drug, a chelator, a radioactive element, a cytotoxic agent, a further antibody or antigen-binding fragment.

The term “antibody drug conjugate” refers to an antibody conjugate comprising at least one drug moiety.

The term “linker” as used herein refers to any molecule enabling a direct topological connection of different portions of a construct or conjugate. For example, a linker may connect a chelator and the targeting moiety. In the alternative, a linker may connect different parts of the chelator. For bispecific antibodies a linker may connect the different antigen-binding portions. Examples for linkers establishing a covalent connection between the different portions include peptide linker and non-proteinaceous polymers, including but not limited to polyethylene glycol (PEG), polypropylene glycol, polyoxyalkylenes, or copolymers of polyethylene glycol, polypropylene glycol.

The term “internalization” of an antibody, fragment or conjugate refers to the uptake of the antibody, fragment or conjugate into a cell. Preferably, internalization is determined for a cell line with endogenous target expression.

### Therapy

“Treating” a disease in a subject or “treating” a subject having a disease refers to subjecting the subject to a pharmaceutical treatment, e.g., the administration of a drug, such that at least one symptom of the disease is decreased or prevented from worsening.

The terms “prevent”, “preventing”, “prevention” and the like refer to reducing the probability of developing a disease, disorder, or condition in a subject, who does not have, but is at risk of or susceptible to developing a disease, disorder, or condition.

The term “effective amount” or “therapeutically effective amount” are used interchangeably herein and refer to an amount sufficient to achieve a particular biological result or to modulate or ameliorate a symptom in a subject, or the time of onset of a symptom, typically by at least about 10%; usually by at least about 20%, preferably at least about 30%, or more preferably at least about 50%. Efficacy of the use of an antibody in cancer therapy can be assessed based on the change in tumor burden. Both tumor shrinkage (objective response) and time to the development of disease progression are important endpoints in cancer clinical trials. Standardized response criteria, known as RECIST (Response Evaluation Criteria in Solid Tumors), were published in 2000. An update (RECIST 1.1) was released in 2009. RECIST criteria are typically used in clinical trials where objective response is the primary study endpoint, as well as in trials where assessment of stable disease, tumor progression or time to progression analyses are undertaken because these outcome measures are based on an assessment of anatomical tumor burden and its change over the course of the trial. An effective amount for a particular subject may vary depending on factors such as the condition being treated, the overall health of the subject, the method, route, and dose of administration and the severity of side effects. When in combination, an effective amount is in ratio to a combination of components and the effect is not limited to individual components alone.

If not defined otherwise, “Complete Response” (CR) is defined as disappearance of all target lesions. Any pathological lymph nodes (whether target or non-target) must have reduction in short axis to <10 mm. For “Partial Response” (PR) at least a 30% decrease in the sum of diameters of target lesions has to be reached, taking as reference the baseline sum diameters. For “Progressive Disease” (PD) at least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. In “Stable Disease” (SD) neither sufficient shrinkage to qualify for PR nor sufficient increase to qualify for PD is observed, taking as reference the smallest sum diameters while on study.

Secondary outcome measures that can be used to determine the therapeutic benefit of the inventive antibodies described herein include the following: “Objective Response Rate” (ORR) is defined as the proportion of subjects who achieve a complete response (CR) or partial response (PR). “Progression Free Survival” (PFS) is defined as the time from the first dose date of an antibody to either disease progression or death, whichever occurs first. “Overall Survival” (OS) is defined as the length of time from either the date of diagnosis or the start of treatment for a disease, that patients diagnosed with the disease are still alive. “Duration of Overall Response” (DOR) is defined as the time from the participant's initial CR or PR to the time of disease progression. “Depth of Response” (DpR) is defined as the percentage of tumor shrinkage observed at the maximal response point compared to baseline tumor load. Clinical endpoints for both ORR and PFS can be determined based on RECIST 1.1 criteria described above.

Typical “subjects” according to the current invention include human and non-human subjects. Subjects can be mammals such as mice, rats, cats, dogs, primates and/or humans.

“Pharmaceutical compositions” (also “therapeutic formulations”) of the antibody, fragment or conjugate can be prepared by mixing the antibody or conjugate having the desired degree of purity with optional physiologically acceptable carriers, excipients or stabilizers, e.g. according to Remington's Pharmaceutical Sciences (18th ed.; Mack Pub. Co.: Eaton, Pa., 1990), e.g. in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as Tween®, Pluronic® or polyethylene glycol (PEG).

A “fixed combination” in the present invention is used as known to persons skilled in the art and is defined as a combination wherein, for example, a first active ingredient according to the present invention, and a further active ingredient are present together in one unit dosage or in one single entity. One example of a “fixed combination” is a pharmaceutical composition wherein a first active ingredient and a further active ingredient are present in admixture for simultaneous administration, such as in a formulation. Another example of a “fixed combination” is a pharmaceutical combination wherein a first active ingredient and a further active ingredient are present in one unit without being in admixture.

### EMBODIMENTS

As described e.g. in examples 9, 11, 12 and 13 it was found according to the current invention that LRRC15 is a suitable target structure for radioconjugates, e.g. for therapeutic or diagnostic applications. This was particularly surprising, because LRRC15 is (i) a stromal protein, and (ii) a low internalizing target, such that the observed suitability could not be anticipated.

In addition, the LRRC15 binding antibodies and conjugates described herein show favorable binding profiles, clearance rates and pharmacokinetics, physicochemical characteristics, immunological behavior, stability during production and/or internalization behavior, as described elsewhere herein. They can be used in multiple further embodiments, e.g. as bispecific antibodies.

### LRRC15 Radioconjugates

According to a first aspect there is provided a conjugate targeting LRRC15, wherein the conjugate comprises at least one chelating group arranged for complexation of a radionuclide and at least one targeting moiety binding to LRRC15. The chelator may or may not comprise the radionuclide. Optionally, the conjugate targeting LRRC15 according to the first aspect may comprise a linker between the at least one chelating group and the at least one targeting moiety binding to LRRC15.

The conjugates disclosed according to the current aspect or other aspects herein are modular in nature as described elsewhere herein. As specific non-limiting examples, specific embodiments of antibodies or fragments thereof, linkers, chelating groups and radionuclides are described. It is intended that all of the specific embodiments described may be combined with each other as though each specific combination were explicitly described individually. Where the skilled person has doubts if a given chelating group may be used with a given radionuclide, the chelating properties of the chelating group for the radionuclide can be evaluated as known in the art.

### Radionuclide

According to the first aspect, the radionuclide may be an α-particle-emitting radionuclide, a 3-particle emitting radionuclide, an Auger electron emitting radionuclide, or a γ-particle-emitting radionuclide. For example, the radionuclide may be selected from the group consisting of ⁴³Sc, ⁴⁴Sc, ⁴⁷Sc, ⁸⁹Zr, ⁹⁰Y, ¹¹¹In, ¹⁴⁹Tb, ¹⁵²Tb, ¹⁵⁵Tb, ¹⁶¹Tb, ¹⁶⁶Ho, ¹⁷⁷Lu, ¹⁸⁶Re, ¹⁸⁸Re, ²¹²Bi, ²¹³Bi, ²²⁵Ac, ²²⁷Th, and ²³²Th.

In some embodiments, the radionuclide is a β-particle emitting radionuclide selected from ⁶⁷Cu, ⁸⁹Sr, ⁸⁹Zr, ⁹⁰Y, ¹⁰⁵Rh, 131I, ¹⁴⁹Pm, ¹⁶⁶Ho, ¹⁷Lu, ¹⁸⁶Re, ¹⁸⁸Re, ¹⁹⁸Au. In some preferred embodiments of the current invention, the radionuclide is ⁸⁹Zr.

In some embodiments, the radionuclide is an Auger electron emitting radionuclide selected from ⁶⁷Ga, ⁷¹Ge, ⁷⁷Br, ^{99m}Tc, ¹⁰³Pd, ¹¹¹In, ¹²³I, ¹²⁵I, ¹⁴⁰Nd, ¹⁷⁸Ta, ¹⁹³Pt, ^{195m}Pt, ¹⁹⁷Hg.

In some preferred embodiments, the radionuclide is an α-particle-emitting radionuclide. For example, the α-particle-emitting radionuclide may be selected from ²¹¹At, ²¹²Pb, ²¹³Bi, ²²³Ra, ²²⁴Ra, ²²⁵Ac, or ²²⁷Th. In most preferred embodiments of all aspects of the current invention, the radionuclide is ²²⁷Th.

### Chelating Group

According to the first aspect, the at least one chelating group may be any chelator arranged for complexation of the radionuclide, such as a chelator comprising desferrioxamine, DOTA, DO3A, CHX-A″-DTPA, NETA, HOPO, 3,2-HOPO, Me-3,2-HOPO, TCMC, a chelator according to formula I or a derivative of any of these, suitable for chelation of the radionuclide. A chelator as used herein may comprise one, two, three, four or more chelating groups which may be either identical or different.

According some most preferred embodiments, the chelator comprises at least one, two, three, or four Me-3,2-HOPO group(s) or at least one structure according to general formula I.

Table E1 shows preferred chelating groups for some selected α-particle emitting radionuclides or beta particle emitting radionuclides.

| Radionuclide | Radiochemistry |
|---|---|
| 211At | Tin precursor, prosthetic group |
| 225Ac | DOTA, DO3A chelator, formula I |
| 213Bi | CHX-A″-DTPA, DOTA, NETA, formula I |
| 227Th | DOTA, HOPO, 3,2-HOPO, Me-3,2-HOPO, formula I |
| 212Pb | TCMC, formula I |
| 212Bi | CHX-A″-DTPA, DOTA, NETA, formula I |
| 89Zr | desferrioxamine (DFO), HOPO, 3,2-HOPO, Me-3,2-HOPO, formula I |

The number of chelating groups linked to the targeting moiety (e.g. chelating group-to-targeting moiety ratio such as chelating group-to-antibody ratio) can vary. Chelating groups may be linked directly to the targeting moiety or may be connected via a linker or scaffold. Typically, a linker will link multiple chelating groups to the targeting moiety. In embodiments which include more than a single chelating group, each chelating group may be the same or different. As long as no unacceptable levels of aggregation under the conditions of use and/or storage are observed, chelating group-to-targeting moiety ratios of 1, 2, 3, 4, 5, 6, 7, 8 or even higher, are contemplated. In some embodiments, conjugates described herein may have a chelator-to-antibody ratio in the range of about 1 to 10, 1 to 8, 1 to 6, 1 to 4 or 1 to 2. In certain specific embodiments, the conjugate may have a chelator-to-antibody ratio of 2, 4 or 6.

### Linker Connecting Different Chelating Groups

Where the conjugate comprises at least two chelating groups, the chelating groups may be connected via a linker or scaffold. The linker connecting different chelating groups may be any suitable linker known in the art or described herein. In some preferred embodiments, the linker is a polyamine linker. In some of these embodiments the conjugate comprises four 3-hydroxy-N-methyl-2-pyridinone moieties, e.g. on a (symmetrical) polyamine scaffold.

The total number of atoms joining two chelating groups (counting by the shortest path if more than one path exists) will generally be limited, so as to constrain the chelating groups in a suitable arrangement for complex formation. Thus, linkers will typically be chosen to provide no more than 15 atoms between chelating groups, preferably, 1 to 12 atoms, and more preferably 1 to 10 atoms between chelating groups. Where a linker joins two chelating groups directly, the linker will typically be 1 to 12 atoms in length, preferably 2 to 10 (such as ethyl, propyl, n-butyl etc).

### Linker Between Chelating Group(s) and Targeting Moiety

Conjugation to a targeting moiety can be achieved as known in the art and as described elsewhere herein, e.g. through amide bond formation with the ε-amino groups of lysine residues.

The linker between chelating group(s) and targeting moiety may be the same or different from a linker connecting at least two chelating groups. Should two or more coupling moieties be used, each can be attached to any of the available sites such as on any linker or chelating group.

### Targeting Moiety

According to the first aspect, the LRRC15 may be from any species e.g. human, monkey, Macaca fascicularis (cynomolgus monkey), Macaca mulatta (rhesus macaque), rodent, mouse, rat, horse, bovine, pig, dog, cat and camel LRRC15. Preferably, the LRRC15 is human LRRC15 and/or cynomolgus and/or murine LRRC15.

According to the first aspect, the targeting moiety binding to LRRC15 may be selected without limitation from the group consisting of peptides, proteins, antibodies or antigen-binding fragments, nanoparticles, polynucleotides, DNA and RNA fragments, aptamers, or small molecules.

According to some highly preferred embodiments, the targeting moiety binding to LRRC15 is an antibody or antigen-binding fragment binding to human LRRC15, e.g. as described elsewhere herein.

In some highly preferred embodiments A of the first aspect, the targeting moiety binding to LRRC15 is an antibody or antigen-binding fragment binding to LRRC15, comprising at least one, two, three, four, five and preferably six CDR sequences having at least 90%, 95%, 98%, 99% or 100% sequence identity with at least one of
a) SEQ ID NO:2, SEQ ID NO:3, SEQ ID NO:4, SEQ ID NO:6, SEQ ID NO:7 and SEQ ID NO:8 (TPP-1633), or
b) SEQ ID NO:22, SEQ ID NO:23, SEQ ID NO:24, SEQ ID NO:26, SEQ ID NO:27 and SEQ ID NO:28 (TPP-14389), or
c) SEQ ID NO:36, SEQ ID NO:37, SEQ ID NO:38, SEQ ID NO:40, SEQ ID NO:41 and SEQ ID NO:42 (TPP-14392), or
d) SEQ ID NO:50, SEQ ID NO:51, SEQ ID NO:52, SEQ ID NO:54, SEQ ID NO:55, and SEQ ID NO:56 (TPP-17073), or
e) SEQ ID NO:60, SEQ ID NO:61, SEQ ID NO:62, SEQ ID NO:64, SEQ ID NO:65, and SEQ ID NO:66 (TPP-17074), or
f) SEQ ID NO:70, SEQ ID NO:71, SEQ ID NO:72, SEQ ID NO:74, SEQ ID NO:75, and SEQ ID NO:76 (TPP-17078), or
g) SEQ ID NO:84, SEQ ID NO:85, SEQ ID NO:86, SEQ ID NO:88, SEQ ID NO:89, and SEQ ID NO:90 (TPP-17405), or
h) SEQ ID NO:94, SEQ ID NO:95, SEQ ID NO:96, SEQ ID NO:98, SEQ ID NO:99, and SEQ ID NO:100 (TPP-17418), or
i) SEQ ID NO:104, SEQ ID NO:105, SEQ ID NO:106, SEQ ID NO:108, SEQ ID NO:109, and SEQ ID NO:110 (TPP-17419), or
j) SEQ ID NO:114, SEQ ID NO:115, SEQ ID NO:116, SEQ ID NO:118, SEQ ID NO:119, and SEQ ID NO:120 (TPP-17421), or
k) SEQ ID NO:128, SEQ ID NO:129, SEQ ID NO:130, SEQ ID NO:132, SEQ ID NO:133, and SEQ ID NO:134 (TPP-17422).

In some highly preferred embodiments, the targeting moiety binding to LRRC15 is an antibody or antigen-binding fragment binding to LRRC15, comprising at least one, two, three, four, five and preferably six CDR sequences having at least 90%, 95%, 98%, 99% or 100% sequence identity with at least one of SEQ ID NO:2, SEQ ID NO:3, SEQ ID NO:4, SEQ ID NO:6, SEQ ID NO:7 and SEQ ID NO:8 (TPP-1633). In some highly preferred embodiments, the targeting moiety binding to LRRC15 is an antibody or antigen-binding fragment binding to LRRC15, comprising at least one, two, three, four, five and preferably six CDR sequences having at least 90%, 95%, 98%, 99% or 100% sequence identity with at least one of SEQ ID NO:22, SEQ ID NO:23, SEQ ID NO:24, SEQ ID NO:26, SEQ ID NO:27 and SEQ ID NO:28 (TPP-14389). In some highly preferred embodiments, the targeting moiety binding to LRRC15 is an antibody or antigen-binding fragment binding to LRRC15, comprising at least one, two, three, four, five and preferably six CDR sequences having at least 90%, 95%, 98%, 99% or 100% sequence identity with at least one of SEQ ID NO:36, SEQ ID NO:37, SEQ ID NO:38, SEQ ID NO:40, SEQ ID NO:41 and SEQ ID NO:42 (TPP-14392). In some highly preferred embodiments, the targeting moiety binding to LRRC15 is an antibody or antigen-binding fragment binding to LRRC15, comprising at least one, two, three, four, five and preferably six CDR sequences having at least 90%, 95%, 98%, 99% or 100% sequence identity with at least one of SEQ ID NO:50, SEQ ID NO:51, SEQ ID NO:52, SEQ ID NO:54, SEQ ID NO:55, and SEQ ID NO:56 (TPP-17073). In some highly preferred embodiments, the targeting moiety binding to LRRC15 is an antibody or antigen-binding fragment binding to LRRC15, comprising at least one, two, three, four, five and preferably six CDR sequences having at least 90%, 95%, 98%, 99% or 100% sequence identity with at least one of SEQ ID NO:60, SEQ ID NO:61, SEQ ID NO:62, SEQ ID NO:64, SEQ ID NO:65, and SEQ ID NO:66 (TPP-17074). In some highly preferred embodiments, the targeting moiety binding to LRRC15 is an antibody or antigen-binding fragment binding to LRRC15, comprising at least one, two, three, four, five and preferably six CDR sequences having at least 90%, 95%, 98%, 99% or 100% sequence identity with at least one of SEQ ID NO:70, SEQ ID NO:71, SEQ ID NO:72, SEQ ID NO:74, SEQ ID NO:75, and SEQ ID NO:76 (TPP-17078). In some highly preferred embodiments, the targeting moiety binding to LRRC15 is an antibody or antigen-binding fragment binding to LRRC15, comprising at least one, two, three, four, five and preferably six CDR sequences having at least 90%, 95%, 98%, 99% or 100% sequence identity with at least one of SEQ ID NO:84, SEQ ID NO:85, SEQ ID NO:86, SEQ ID NO:88, SEQ ID NO:89, and SEQ ID NO:90 (TPP-17405). In some highly preferred embodiments, the targeting moiety binding to LRRC15 is an antibody or antigen-binding fragment binding to LRRC15, comprising at least one, two, three, four, five and preferably six CDR sequences having at least 90%, 95%, 98%, 99% or 100% sequence identity with at least one of SEQ ID NO:94, SEQ ID NO:95, SEQ ID NO:96, SEQ ID NO:98, SEQ ID NO:99, and SEQ ID NO:100 (TPP-17418). In some highly preferred embodiments, the targeting moiety binding to LRRC15 is an antibody or antigen-binding fragment binding to LRRC15, comprising at least one, two, three, four, five and preferably six CDR sequences having at least 90%, 95%, 98%, 99% or 100% sequence identity with at least one of SEQ ID NO:104, SEQ ID NO:105, SEQ ID NO:106, SEQ ID NO:108, SEQ ID NO:109, and SEQ ID NO:110 (TPP-17419). In some highly preferred embodiments, the targeting moiety binding to LRRC15 is an antibody or antigen-binding fragment binding to LRRC15, comprising at least one, two, three, four, five and preferably six CDR sequences having at least 90%, 95%, 98%, 99% or 100% sequence identity with at least one of SEQ ID NO:114, SEQ ID NO:115, SEQ ID NO:116, SEQ ID NO:118, SEQ ID NO:119, and SEQ ID NO:120 (TPP-17421). In some highly preferred embodiments, the targeting moiety binding to LRRC15 is an antibody or antigen-binding fragment binding to LRRC15, comprising at least one, two, three, four, five and preferably six CDR sequences having at least 90%, 95%, 98%, 99% or 100% sequence identity with at least one of SEQ ID NO:128, SEQ ID NO:129, SEQ ID NO:130, SEQ ID NO:132, SEQ ID NO:133, and SEQ ID NO:134 (TPP-17422).

In some highly preferred embodiments B of the first aspect, the targeting moiety binding to LRRC15 is an antibody or antigen-binding fragment binding to LRRC15 comprising
a) a variable heavy chain having at least 90%, 95%, 98%, 99% or 100% sequence identity with SEQ ID NO:1 and/or
   a variable light chain having at least 90%, 95%, 98%, 99% or 100% sequence identity with SEQ ID NO:5 (TPP-1633), or
b) a variable heavy chain having at least 90%, 95%, 98%, 99% or 100% sequence identity with SEQ ID NO:21 and/or
   a variable light chain having at least 90%, 95%, 98%, 99% or 100% sequence identity with SEQ ID NO:25 (TPP-14389), or
c) a variable heavy chain having at least 90%, 95%, 98%, 99% or 100% sequence identity with SEQ ID NO:35 and/or
   a variable light chain having at least 90%, 95%, 98%, 99% or 100% sequence identity with SEQ ID NO:39 (TPP-14392), or
d) a variable heavy chain having at least 90%, 95%, 98%, 99% or 100% sequence identity with SEQ ID NO:49 and/or
   a variable light chain having at least 90%, 95%, 98%, 99% or 100% sequence identity with SEQ ID NO:53 (TPP-17073), or
e) a variable heavy chain having at least 90%, 95%, 98%, 99% or 100% sequence identity with SEQ ID NO:59 and/or
   a variable light chain having at least 90%, 95%, 98%, 99% or 100% sequence identity with SEQ ID NO:63 (TPP-17074), or
f) a variable heavy chain having at least 90%, 95%, 98%, 99% or 100% sequence identity with SEQ ID NO:69 and/or
   a variable light chain having at least 90%, 95%, 98%, 99% or 100% sequence identity with SEQ ID NO:73 (TPP-17078), or
g) a variable heavy chain having at least 90%, 95%, 98%, 99% or 100% sequence identity with SEQ ID NO:83 and/or
   a variable light chain having at least 90%, 95%, 98%, 99% or 100% sequence identity with SEQ ID NO:87 (TPP-17405), or
h) a variable heavy chain having at least 90%, 95%, 98%, 99% or 100% sequence identity with SEQ ID NO:93 and/or
   a variable light chain having at least 90%, 95%, 98%, 99% or 100% sequence identity with SEQ ID NO:97 (TPP-17418), or
i) a variable heavy chain having at least 90%, 95%, 98%, 99% or 100% sequence identity with SEQ ID NO:103 and/or
   a variable light chain having at least 90%, 95%, 98%, 99% or 100% sequence identity with SEQ ID NO:107 (TPP-17419), or
j) a variable heavy chain having at least 90%, 95%, 98%, 99% or 100% sequence identity with SEQ ID NO:113 and/or
   a variable light chain having at least 90%, 95%, 98%, 99% or 100% sequence identity with SEQ ID NO:117 (TPP-17421), or
k) a variable heavy chain having at least 90%, 95%, 98%, 99% or 100% sequence identity with SEQ ID NO:127 and/or
   a variable light chain having at least 90%, 95%, 98%, 99% or 100% sequence identity with SEQ ID NO:131 (TPP-17422).

In some highly preferred embodiments, the targeting moiety binding to LRRC15 is an antibody or antigen-binding fragment binding to LRRC15 comprising a variable heavy chain having at least 90%, 95%, 98%, 99% or 100% sequence identity with SEQ ID NO:1 and/or a variable light chain having at least 90%, 95%, 98%, 99% or 100% sequence identity with SEQ ID NO:5 (TPP-1633).

In some highly preferred embodiments, the targeting moiety binding to LRRC15 is an antibody or antigen-binding fragment binding to LRRC15 comprising a variable heavy chain having at least 90%, 95%, 98%, 99% or 100% sequence identity with SEQ ID NO:21 and/or a variable light chain having at least 90%, 95%, 98%, 99% or 100% sequence identity with SEQ ID NO:25 (TPP-14389).

In some highly preferred embodiments, the targeting moiety binding to LRRC15 is an antibody or antigen-binding fragment binding to LRRC15 comprising a variable heavy chain having at least 90%, 95%, 98%, 99% or 100% sequence identity with SEQ ID NO:35 and/or a variable light chain having at least 90%, 95%, 98%, 99% or 100% sequence identity with SEQ ID NO:39 (TPP-14392).

In some highly preferred embodiments, the targeting moiety binding to LRRC15 is an antibody or antigen-binding fragment binding to LRRC15 comprising a variable heavy chain having at least 90%, 95%, 98%, 99% or 100% sequence identity with SEQ ID NO:49 and/or a variable light chain having at least 90%, 95%, 98%, 99% or 100% sequence identity with SEQ ID NO:53 (TPP-17073).

In some highly preferred embodiments, the targeting moiety binding to LRRC15 is an antibody or antigen-binding fragment binding to LRRC15 comprising a variable heavy chain having at least 90%, 95%, 98%, 99% or 100% sequence identity with SEQ ID NO:59 and/or a variable light chain having at least 90%, 95%, 98%, 99% or 100% sequence identity with SEQ ID NO:63 (TPP-17074).

In some highly preferred embodiments, the targeting moiety binding to LRRC15 is an antibody or antigen-binding fragment binding to LRRC15 comprising a variable heavy chain having at least 90%, 95%, 98%, 99% or 100% sequence identity with SEQ ID NO:69 and/or a variable light chain having at least 90%, 95%, 98%, 99% or 100% sequence identity with SEQ ID NO:73 (TPP-17078).

In some highly preferred embodiments, the targeting moiety binding to LRRC15 is an antibody or antigen-binding fragment binding to LRRC15 comprising a variable heavy chain having at least 90%, 95%, 98%, 99% or 100% sequence identity with SEQ ID NO:83 and/or a variable light chain having at least 90%, 95%, 98%, 99% or 100% sequence identity with SEQ ID NO:87 (TPP-17405).

In some highly preferred embodiments, the targeting moiety binding to LRRC15 is an antibody or antigen-binding fragment binding to LRRC15 comprising a variable heavy chain having at least 90%, 95%, 98%, 99% or 100% sequence identity with SEQ ID NO:93 and/or a variable light chain having at least 90%, 95%, 98%, 99% or 100% sequence identity with SEQ ID NO:97 (TPP-17418).

In some highly preferred embodiments, the targeting moiety binding to LRRC15 is an antibody or antigen-binding fragment binding to LRRC15 comprising a variable heavy chain having at least 90%, 95%, 98%, 99% or 100% sequence identity with SEQ ID NO:103 and/or a variable light chain having at least 90%, 95%, 98%, 99% or 100% sequence identity with SEQ ID NO:107 (TPP-17419).

In some highly preferred embodiments, the targeting moiety binding to LRRC15 is an antibody or antigen-binding fragment binding to LRRC15 comprising a variable heavy chain having at least 90%, 95%, 98%, 99% or 100% sequence identity with SEQ ID NO:113 and/or a variable light chain having at least 90%, 95%, 98%, 99% or 100% sequence identity with SEQ ID NO:117 (TPP-17421).

In some highly preferred embodiments, the targeting moiety binding to LRRC15 is an antibody or antigen-binding fragment binding to LRRC15 comprising a variable heavy chain having at least 90%, 95%, 98%, 99% or 100% sequence identity with SEQ ID NO:127 and/or a variable light chain having at least 90%, 95%, 98%, 99% or 100% sequence identity with SEQ ID NO:131 (TPP-17422).

In some highly preferred embodiments C of the first aspect, the targeting moiety binding to LRRC15 is an antibody or antigen-binding fragment binding to LRRC15 comprising
a) a heavy chain region having at least 90%, 95%, 98%, 99% or 100% sequence identity with SEQ ID NO:9 and/or
   a light chain region having at least 90%, 95%, 98%, 99% or 100% sequence identity with SEQ ID NO:10 (TPP-1633), or
b) a heavy chain region having at least 90%, 95%, 98%, 99% or 100% sequence identity with SEQ ID NO:31 and/or
   a light chain region having at least 90%, 95%, 98%, 99% or 100% sequence identity with SEQ ID NO:32 (TPP-14389), or
c) a heavy chain region having at least 90%, 95%, 98%, 99% or 100% sequence identity with SEQ ID NO:45 and/or
   a light chain region having at least 90%, 95%, 98%, 99% or 100% sequence identity with SEQ ID NO:46 (TPP-14392), or
d) a heavy chain region having at least 90%, 95%, 98%, 99% or 100% sequence identity with SEQ ID NO:57 and/or
   a light chain region having at least 90%, 95%, 98%, 99% or 100% sequence identity with SEQ ID NO:58 (TPP-17073), or
e) a heavy chain region having at least 90%, 95%, 98%, 99% or 100% sequence identity with SEQ ID NO:67 and/or
   a light chain region having at least 90%, 95%, 98%, 99% or 100% sequence identity with SEQ ID NO:68 (TPP-17074), or
f) a heavy chain region having at least 90%, 95%, 98%, 99% or 100% sequence identity with SEQ ID NO:79 and/or
   a light chain region having at least 90%, 95%, 98%, 99% or 100% sequence identity with SEQ ID NO:80 (TPP-17078), or
g) a heavy chain region having at least 90%, 95%, 98%, 99% or 100% sequence identity with SEQ ID NO:91 and/or
   a light chain region having at least 90%, 95%, 98%, 99% or 100% sequence identity with SEQ ID NO:92 (TPP-17405), or
h) a heavy chain region having at least 90%, 95%, 98%, 99% or 100% sequence identity with SEQ ID NO:101 and/or
   a light chain region having at least 90%, 95%, 98%, 99% or 100% sequence identity with SEQ ID NO:102 (TPP-17418), or
i) a heavy chain region having at least 90%, 95%, 98%, 99% or 100% sequence identity with SEQ ID NO:111 and/or
   a light chain region having at least 90%, 95%, 98%, 99% or 100% sequence identity with SEQ ID NO:112 (TPP-17419), or
j) a heavy chain region having at least 90%, 95%, 98%, 99% or 100% sequence identity with SEQ ID NO:123 and/or
   a light chain region having at least 90%, 95%, 98%, 99% or 100% sequence identity with SEQ ID NO:124 (TPP-17421), or
k) a heavy chain region having at least 90%, 95%, 98%, 99% or 100% sequence identity with SEQ ID NO:135 and/or
   a light chain region having at least 90%, 95%, 98%, 99% or 100% sequence identity with SEQ ID NO:136 (TPP-17422).

In some highly preferred embodiments, the targeting moiety binding to LRRC15 is an antibody or antigen-binding fragment binding to LRRC15 comprising a heavy chain region having at least 90%, 95%, 98%, 99% or 100% sequence identity with SEQ ID NO:9 and/or a light chain region having at least 90%, 95%, 98%, 99% or 100% sequence identity with SEQ ID NO:10 (TPP-1633).

In some highly preferred embodiments, the targeting moiety binding to LRRC15 is an antibody or antigen-binding fragment binding to LRRC15 comprising a heavy chain region having at least 90%, 95%, 98%, 99% or 100% sequence identity with SEQ ID NO:31 and/or a light chain region having at least 90%, 95%, 98%, 99% or 100% sequence identity with SEQ ID NO:32 (TPP-14389).

In some highly preferred embodiments, the targeting moiety binding to LRRC15 is an antibody or antigen-binding fragment binding to LRRC15 comprising a heavy chain region having at least 90%, 95%, 98%, 99% or 100% sequence identity with SEQ ID NO:45 and/or a light chain region having at least 90%, 95%, 98%, 99% or 100% sequence identity with SEQ ID NO:46 (TPP-14392).

In some highly preferred embodiments, the targeting moiety binding to LRRC15 is an antibody or antigen-binding fragment binding to LRRC15 comprising a heavy chain region having at least 90%, 95%, 98%, 99% or 100% sequence identity with SEQ ID NO:57 and/or a light chain region having at least 90%, 95%, 98%, 99% or 100% sequence identity with SEQ ID NO:58 (TPP-17073).

In some highly preferred embodiments, the targeting moiety binding to LRRC15 is an antibody or antigen-binding fragment binding to LRRC15 comprising a heavy chain region having at least 90%, 95%, 98%, 99% or 100% sequence identity with SEQ ID NO:67 and/or a light chain region having at least 90%, 95%, 98%, 99% or 100% sequence identity with SEQ ID NO:68 (TPP-17074).

In some highly preferred embodiments, the targeting moiety binding to LRRC15 is an antibody or antigen-binding fragment binding to LRRC15 comprising a heavy chain region having at least 90%, 95%, 98%, 99% or 100% sequence identity with SEQ ID NO:79 and/or a light chain region having at least 90%, 95%, 98%, 99% or 100% sequence identity with SEQ ID NO:80 (TPP-17078).

In some highly preferred embodiments, the targeting moiety binding to LRRC15 is an antibody or antigen-binding fragment binding to LRRC15 comprising a heavy chain region having at least 90%, 95%, 98%, 99% or 100% sequence identity with SEQ ID NO:91 and/or a light chain region having at least 90%, 95%, 98%, 99% or 100% sequence identity with SEQ ID NO:92 (TPP-17405).

In some highly preferred embodiments, the targeting moiety binding to LRRC15 is an antibody or antigen-binding fragment binding to LRRC15 comprising a heavy chain region having at least 90%, 95%, 98%, 99% or 100% sequence identity with SEQ ID NO:101 and/or a light chain region having at least 90%, 95%, 98%, 99% or 100% sequence identity with SEQ ID NO:102 (TPP-17418).

In some highly preferred embodiments, the targeting moiety binding to LRRC15 is an antibody or antigen-binding fragment binding to LRRC15 comprising a heavy chain region having at least 90%, 95%, 98%, 99% or 100% sequence identity with SEQ ID NO:111 and/or a light chain region having at least 90%, 95%, 98%, 99% or 100% sequence identity with SEQ ID NO:112 (TPP-17419).

In some highly preferred embodiments, the targeting moiety binding to LRRC15 is an antibody or antigen-binding fragment binding to LRRC15 comprising a heavy chain region having at least 90%, 95%, 98%, 99% or 100% sequence identity with SEQ ID NO:123 and/or a light chain region having at least 90%, 95%, 98%, 99% or 100% sequence identity with SEQ ID NO:124 (TPP-17421).

In some highly preferred embodiments, the targeting moiety binding to LRRC15 is an antibody or antigen-binding fragment binding to LRRC15 comprising a heavy chain region having at least 90%, 95%, 98%, 99% or 100% sequence identity with SEQ ID NO:135 and/or a light chain region having at least 90%, 95%, 98%, 99% or 100% sequence identity with SEQ ID NO:136 (TPP-17422).

### RADIOCONJUGATE EMBODIMENTS

According to some preferred embodiments, the conjugate targeting LRRC15 according to the first aspect comprises
a) a chelator arranged for complexation of an α-particle-emitting radionuclide and
b) a targeting moiety binding to LRRC15.

According to some of these preferred embodiments, the α-particle-emitting radionuclide is thorium-227. According to some of these embodiments, the conjugate targeting LRRC15 comprises thorium-227.

According to some of the aforementioned embodiments, the chelator comprises
a) hydroxypyridinone (HOPO),
b) 3-hydroxypyridin-2-one (3,2-HOPO),
c) 3-hydroxy-N-methyl-2-pyridinone (Me-3,2-HOPO),
d) 1,4,7,10-tetra-azacycloododecane-N,N′,N″,N′″-tetraacetic acid (DOTA), and/or
e) a chelator according to formula I,
   or a derivative thereof.

For some most preferred of the aforementioned embodiments, the LRRC15 is human, cynomolgus and/or murine LRRC15, e.g. human and/or cynomolgus LRRC15.

For some further most preferred of the aforementioned embodiments, the targeting moiety binding to LRRC15 is an antibody or antigen-binding fragment thereof. In particular, the antibody or fragment thereof can be and is suggested to be an antibody according to any of the embodiments listed elsewhere herein for the first aspect or for the second aspect. In particular, the antibody or fragment thereof can be an IgG1 antibody such as a human or humanized IgG1 antibody or fragment thereof.

In some preferred embodiments, the conjugate is a conjugate comprising structural formula (II):

[C]n-L-Ab (II)
or a salt thereof, where
each “C” represents, independently of the others, a chelating group arranged for complexation of a radionuclide;
n represents the number of chelating groups attached to a single linker “L” and is preferably 1, 2, 3, 4, 5, or 6;
“Ab” represents an LRRC15 targeting moiety, such as an anti LRRC15 antibody or antigen-binding fragment, e.g. according to the current invention.

In a specific exemplary embodiment, the conjugate is a compound according to structural formula (II) in which each “C” is the same and is either a 3,2 HOPO group or a Me-3,2 HOPO group; L is preferably a polyamine linker; “Ab” is an antibody or fragment thereof comprising six CDRs corresponding to the six CDRs of an anti LRRC15 antibody according to the current invention.

### Conjugates Chelating α-Particle-Emitting Radionuclide

In some embodiments, the radionuclide is ²²⁵Ac, the chelator comprises DOTA, HOPO, Me-3,2-HOPO or a chelator according to formula I or a derivative of any of these, and/or the targeting moiety binding to human LRRC15 is an antibody or antigen-binding fragment thereof, such as one of those according to embodiments A, B or C of the current aspect.

In some embodiments, the radionuclide is ²¹²Bi or ²¹³Bi, the chelator comprises CHX-A″-DTPA, DOTA, NETA or a chelator according to formula I or a derivative of any of these, and/or the targeting moiety binding to human LRRC15 is an antibody or antigen-binding fragment thereof, such as one of those according to embodiments A, B or C of the current aspect.

In some embodiments, the radionuclide is ²¹²Pb, the chelator comprises TCMC or a chelator according to formula I or a derivative of any of these, and/or the targeting moiety binding to human LRRC15 is an antibody or antigen-binding fragment thereof, such as one of those according to embodiments A, B or C of the current aspect.

### Conjugates Chelating ²²⁷Th

In some preferred embodiments, the radionuclide is ²²⁷Th, the chelator comprises HOPO, Me-3,2-HOPO, DOTA, a chelator according to formula I or a derivative of any of these, and/or the targeting moiety binding to human LRRC15 is an antibody or antigen-binding fragment thereof, such as an antibody or antigen-binding fragment according to embodiments A, B or C of the current aspect. For example, the chelator comprises DOTA or a derivative thereof. For example, the chelator comprises HOPO or a derivative thereof. For example, the chelator comprises Me-3,2-HOPO or a derivative thereof. For example, the chelator comprises a structure according to formula I or a derivative thereof. Preferably, the chelating group-to-antibody ratio is 4.

### TPP-1633

In some of these preferred embodiments, the radionuclide is ²²⁷Th, the chelator comprises Me-3,2-HOPO or a derivative thereof, and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:2, SEQ ID NO:3, SEQ ID NO:4, SEQ ID NO:6, SEQ ID NO:7 and SEQ ID NO:8 (TPP-1633). In some of these examples, the antibody furthermore comprises a variable heavy chain having at least 90%, 95%, 98%, 99% or 100% sequence identity with SEQ ID NO:1 and/or a variable light chain having at least 90%, 95%, 98%, 99% or 100% sequence identity with SEQ ID NO:5. In some of the examples, the antibody or antigen-binding fragment thereof is a human Fab′, F(ab′)2, Fab, Fv, rIgG, or scFv fragment. In some examples, the antibody comprises a heavy chain region having at least 90%, 95%, 98%, 99% or 100% sequence identity with SEQ ID NO:9 and/or a light chain region having at least 90%, 95%, 98%, 99% or 100% sequence identity with SEQ ID NO:10. In some of these examples, the antibody or antigen-binding fragment thereof is a human IgG1 antibody.

In some other of the preferred embodiments, the radionuclide is ²²⁷Th, the chelator comprises a structure according to formula I or a derivative thereof, and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:2, SEQ ID NO:3, SEQ ID NO:4, SEQ ID NO:6, SEQ ID NO:7 and SEQ ID NO:8 (TPP-1633). In some of these examples, the antibody furthermore comprises a variable heavy chain having at least 90%, 95%, 98%, 99% or 100% sequence identity with SEQ ID NO:1 and/or a variable light chain having at least 90%, 95%, 98%, 99% or 100% sequence identity with SEQ ID NO:5. In some of the examples, the antibody or antigen-binding fragment thereof is a human Fab′, F(ab′)2, Fab, Fv, rIgG, or scFv fragment. In some examples, the antibody comprises a heavy chain region having at least 90%, 95%, 98%, 99% or 100% sequence identity with SEQ ID NO:9 and/or a light chain region having at least 90%, 95%, 98%, 99% or 100% sequence identity with SEQ ID NO:10. In some of these examples, the antibody or antigen-binding fragment thereof is a human IgG1 antibody.

In some of the preferred embodiments, the radionuclide is ²²⁷Th, the chelator comprises 3,2-HOPO, or a derivative thereof and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:2, SEQ ID NO:3, SEQ ID NO:4, SEQ ID NO:6, SEQ ID NO:7 and SEQ ID NO:8 (TPP-1633). In some of these examples, the antibody furthermore comprises a variable heavy chain having at least 90%, 95%, 98%, 99% or 100% sequence identity with SEQ ID NO:1 and/or a variable light chain having at least 90%, 95%, 98%, 99% or 100% sequence identity with SEQ ID NO:5. In some of the examples, the antibody or antigen-binding fragment thereof is a human Fab′, F(ab′)2, Fab, Fv, rIgG, or scFv fragment. In some examples, the antibody comprises a heavy chain region having at least 90%, 95%, 98%, 99% or 100% sequence identity with SEQ ID NO:9 and/or a light chain region having at least 90%, 95%, 98%, 99% or 100% sequence identity with SEQ ID NO:10. In some of these examples, the antibody or antigen-binding fragment thereof is a human IgG1 antibody.

### TPP-14389

In some of these preferred embodiments, the radionuclide is ²²⁷Th, the chelator comprises Me-3,2-HOPO or a derivative thereof, and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:22, SEQ ID NO:23, SEQ ID NO:24, SEQ ID NO:26, SEQ ID NO:27 and SEQ ID NO:28 (TPP-14389). In some of these examples, the antibody furthermore comprises a variable heavy chain having at least 90%, 95%, 98%, 99% or 100% sequence identity with SEQ ID NO:21 and/or a variable light chain having at least 90%, 95%, 98%, 99% or 100% sequence identity with SEQ ID NO:25. In some of the examples, the antibody or antigen-binding fragment thereof is a human Fab′, F(ab′)2, Fab, Fv, rIgG, or scFv fragment. In some examples, the antibody comprises a heavy chain region having at least 90%, 95%, 98%, 99% or 100% sequence identity with SEQ ID NO:31 and/or a light chain region having at least 90%, 95%, 98%, 99% or 100% sequence identity with SEQ ID NO:32. In some of these examples, the antibody or antigen-binding fragment thereof is a human IgG1 antibody.

In some other of the preferred embodiments, the radionuclide is ²²⁷Th, the chelator comprises a structure according to formula I or a derivative thereof, and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:22, SEQ ID NO:23, SEQ ID NO:24, SEQ ID NO:26, SEQ ID NO:27 and SEQ ID NO:28 (TPP-14389). In some of these examples, the antibody furthermore comprises a variable heavy chain having at least 90%, 95%, 98%, 99% or 100% sequence identity with SEQ ID NO:21 and/or a variable light chain having at least 90%, 95%, 98%, 99% or 100% sequence identity with SEQ ID NO:25. In some of the examples, the antibody or antigen-binding fragment thereof is a human Fab′, F(ab′)2, Fab, Fv, rIgG, or scFv fragment. In some examples, the antibody comprises a heavy chain region having at least 90%, 95%, 98%, 99% or 100% sequence identity with SEQ ID NO:31 and/or a light chain region having at least 90%, 95%, 98%, 99% or 100% sequence identity with SEQ ID NO:32. In some of these examples, the antibody or antigen-binding fragment thereof is a human IgG1 antibody.

In some of the preferred embodiments, the radionuclide is ²²⁷Th, the chelator comprises 3,2-HOPO, or a derivative thereof and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:22, SEQ ID NO:23, SEQ ID NO:24, SEQ ID NO:26, SEQ ID NO:27 and SEQ ID NO:28 (TPP-14389). In some of these examples, the antibody furthermore comprises a variable heavy chain having at least 90%, 95%, 98%, 99% or 100% sequence identity with SEQ ID NO:21 and/or a variable light chain having at least 90%, 95%, 98%, 99% or 100% sequence identity with SEQ ID NO:25. In some of the examples, the antibody or antigen-binding fragment thereof is a human Fab′, F(ab′)2, Fab, Fv, rIgG, or scFv fragment. In some examples, the antibody comprises a heavy chain region having at least 90%, 95%, 98%, 99% or 100% sequence identity with SEQ ID NO:31 and/or a light chain region having at least 90%, 95%, 98%, 99% or 100% sequence identity with SEQ ID NO:32. In some of these examples, the antibody or antigen-binding fragment thereof is a human IgG1 antibody.

### TPP-14392

In some other of these preferred embodiments, the radionuclide is ²²⁷Th, the chelator comprises Me-3,2-HOPO, or a derivative thereof and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:36, SEQ ID NO:37, SEQ ID NO:38, SEQ ID NO:40, SEQ ID NO:41 and SEQ ID NO:42 (e.g. TPP-14392). In some of these examples, the antibody furthermore comprises a variable heavy chain having at least 90%, 95%, 98%, 99% or 100% sequence identity with SEQ ID NO:35 and/or a variable light chain having at least 90%, 95%, 98%, 99% or 100% sequence identity with SEQ ID NO:39. In some of the examples, the antibody or antigen-binding fragment thereof is a human Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:45 and/or a light chain region having at least 90%, 95%, 98%, 99% or 100% sequence identity with SEQ ID NO:46. In some of these examples, the antibody or antigen-binding fragment thereof is a human IgG1 antibody. In some other of these preferred embodiments, the radionuclide is ²²⁷Th, the chelator comprises a chelator according to formula I or a derivative thereof, and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:36, SEQ ID NO:37, SEQ ID NO:38, SEQ ID NO:40, SEQ ID NO:41 and SEQ ID NO:42 (e.g. TPP-14392). In some of these examples, the antibody furthermore comprises a variable heavy chain having at least 90%, 95%, 98%, 99% or 100% sequence identity with SEQ ID NO:35 and/or a variable light chain having at least 90%, 95%, 98%, 99% or 100% sequence identity with SEQ ID NO:39. In some of the examples, the antibody or antigen-binding fragment thereof is a human Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:45 and/or a light chain region having at least 90%, 95%, 98%, 99% or 100% sequence identity with SEQ ID NO:46. In some of these examples, the antibody or antigen-binding fragment thereof is a human IgG1 antibody.

In some other of these preferred embodiments, the radionuclide is ²²⁷Th, the chelator comprises 3,2-HOPO, or a derivative thereof and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:36, SEQ ID NO:37, SEQ ID NO:38, SEQ ID NO:40, SEQ ID NO:41 and SEQ ID NO:42 (e.g. TPP-14392). In some of these examples, the antibody furthermore comprises a variable heavy chain having at least 90%, 95%, 98%, 99% or 100% sequence identity with SEQ ID NO:35 and/or a variable light chain having at least 90%, 95%, 98%, 99% or 100% sequence identity with SEQ ID NO:39. In some of the examples, the antibody or antigen-binding fragment thereof is a human Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:45 and/or a light chain region having at least 90%, 95%, 98%, 99% or 100% sequence identity with SEQ ID NO:46. In some of these examples, the antibody or antigen-binding fragment thereof is a human IgG1 antibody.

### TPP-17073

In some other of these preferred embodiments, the radionuclide is ²²⁷Th, the chelator comprises Me-3,2-HOPO, or a derivative thereof and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:50, SEQ ID NO:51, SEQ ID NO:52, SEQ ID NO:54, SEQ ID NO:55, and SEQ ID NO:56 (TPP-17073). In some of these examples, the antibody furthermore comprises a variable heavy chain having at least 90%, 95%, 98%, 99% or 100% sequence identity with SEQ ID NO:49 and/or a variable light chain having at least 90%, 95%, 98%, 99% or 100% sequence identity with SEQ ID NO:53. In some of the examples, the antibody or antigen-binding fragment thereof is a human Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:57 and/or a light chain region having at least 90%, 95%, 98%, 99% or 100% sequence identity with SEQ ID NO:58. In some of these examples, the antibody or antigen-binding fragment thereof is a human IgG1 antibody. In some other of the preferred embodiments, the radionuclide is ²²⁷Th, the chelator comprises a structure according to formula I or a derivative thereof, and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:50, SEQ ID NO:51, SEQ ID NO:52, SEQ ID NO:54, SEQ ID NO:55, and SEQ ID NO:56 (TPP-17073). In some of these examples, the antibody furthermore comprises a variable heavy chain having at least 90%, 95%, 98%, 99% or 100% sequence identity with SEQ ID NO:49 and/or a variable light chain having at least 90%, 95%, 98%, 99% or 100% sequence identity with SEQ ID NO:53. In some of the examples, the antibody or antigen-binding fragment thereof is a human Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:57 and/or a light chain region having at least 90%, 95%, 98%, 99% or 100% sequence identity with SEQ ID NO:58. In some of these examples, the antibody or antigen-binding fragment thereof is a human IgG1 antibody.

In some other of these preferred embodiments, the radionuclide is ²²⁷Th, the chelator comprises 3,2-HOPO, or a derivative thereof and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:50, SEQ ID NO:51, SEQ ID NO:52, SEQ ID NO:54, SEQ ID NO:55, and SEQ ID NO:56 (TPP-17073). In some of these examples, the antibody furthermore comprises a variable heavy chain having at least 90%, 95%, 98%, 99% or 100% sequence identity with SEQ ID NO:49 and/or a variable light chain having at least 90%, 95%, 98%, 99% or 100% sequence identity with SEQ ID NO:53. In some of the examples, the antibody or antigen-binding fragment thereof is a human Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:57 and/or a light chain region having at least 90%, 95%, 98%, 99% or 100% sequence identity with SEQ ID NO:58. In some of these examples, the antibody or antigen-binding fragment thereof is a human IgG1 antibody.

### TPP-17074

In some other of these preferred embodiments, the radionuclide is ²²⁷Th, the chelator comprises Me-3,2-HOPO, or a derivative thereof and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:60, SEQ ID NO:61, SEQ ID NO:62, SEQ ID NO:64, SEQ ID NO:65, and SEQ ID NO:66 (TPP-17074). In some of these examples, the antibody furthermore comprises a variable heavy chain having at least 90%, 95%, 98%, 99% or 100% sequence identity with SEQ ID NO:59 and/or a variable light chain having at least 90%, 95%, 98%, 99% or 100% sequence identity with SEQ ID NO:63. In some of the examples, the antibody or antigen-binding fragment thereof is a human Fab′, F(ab′)2, Fab, Fv, rIgG, or scFv fragment. In some examples, the antibody furthermore comprises a e) a heavy chain region having at least 90%, 95%, 98%, 99% or 100% sequence identity with SEQ ID NO:67 and/or a light chain region having at least 90%, 95%, 98%, 99% or 100% sequence identity with SEQ ID NO:68. In some of these examples, the antibody or antigen-binding fragment thereof is a human IgG1 antibody. In some other of the preferred embodiments, the radionuclide is ²²⁷Th, the chelator comprises a structure according to formula I or a derivative thereof, and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:60, SEQ ID NO:61, SEQ ID NO:62, SEQ ID NO:64, SEQ ID NO:65, and SEQ ID NO:66 (TPP-17074). In some of these examples, the antibody furthermore comprises a variable heavy chain having at least 90%, 95%, 98%, 99% or 100% sequence identity with SEQ ID NO:59 and/or a variable light chain having at least 90%, 95%, 98%, 99% or 100% sequence identity with SEQ ID NO:63. In some of the examples, the antibody or antigen-binding fragment thereof is a human Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:67 and/or a light chain region having at least 90%, 95%, 98%, 99% or 100% sequence identity with SEQ ID NO:68. In some of these examples, the antibody or antigen-binding fragment thereof is a human IgG1 antibody.

In some other of these preferred embodiments, the radionuclide is ²²⁷Th, the chelator comprises 3,2-HOPO, or a derivative thereof and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:60, SEQ ID NO:61, SEQ ID NO:62, SEQ ID NO:64, SEQ ID NO:65, and SEQ ID NO:66 (TPP-17074). In some of these examples, the antibody furthermore comprises a variable heavy chain having at least 90%, 95%, 98%, 99% or 100% sequence identity with SEQ ID NO:59 and/or a variable light chain having at least 90%, 95%, 98%, 99% or 100% sequence identity with SEQ ID NO:63. In some of the examples, the antibody or antigen-binding fragment thereof is a human Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:67 and/or a light chain region having at least 90%, 95%, 98%, 99% or 100% sequence identity with SEQ ID NO:68. In some of these examples, the antibody or antigen-binding fragment thereof is a human IgG1 antibody.

### TPP-17078

In some other of these preferred embodiments, the radionuclide is ²²⁷Th, the chelator comprises Me-3,2-HOPO, or a derivative thereof and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:70, SEQ ID NO:71, SEQ ID NO:72, SEQ ID NO:74, SEQ ID NO:75, and SEQ ID NO:76 (TPP-17078). In some of these examples, the antibody furthermore comprises a variable heavy chain having at least 90%, 95%, 98%, 99% or 100% sequence identity with SEQ ID NO:69 and/or a variable light chain having at least 90%, 95%, 98%, 99% or 100% sequence identity with SEQ ID NO:73. In some of the examples, the antibody or antigen-binding fragment thereof is a human Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:79 and/or a light chain region having at least 90%, 95%, 98%, 99% or 100% sequence identity with SEQ ID NO:80. In some of these examples, the antibody or antigen-binding fragment thereof is a human IgG1 antibody. In some other of these preferred embodiments, the radionuclide is ²²⁷Th, the chelator comprises a chelator according to formula I or a derivative thereof, and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:70, SEQ ID NO:71, SEQ ID NO:72, SEQ ID NO:74, SEQ ID NO:75, and SEQ ID NO:76 (TPP-17078). In some of these examples, the antibody furthermore comprises a variable heavy chain having at least 90%, 95%, 98%, 99% or 100% sequence identity with SEQ ID NO:69 and/or a variable light chain having at least 90%, 95%, 98%, 99% or 100% sequence identity with SEQ ID NO:73. In some of the examples, the antibody or antigen-binding fragment thereof is a human Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:79 and/or a light chain region having at least 90%, 95%, 98%, 99% or 100% sequence identity with SEQ ID NO:80. In some of these examples, the antibody or antigen-binding fragment thereof is a human IgG1 antibody.

In some other of these preferred embodiments, the radionuclide is ²²⁷Th, the chelator comprises 3,2-HOPO, or a derivative thereof and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:70, SEQ ID NO:71, SEQ ID NO:72, SEQ ID NO:74, SEQ ID NO:75, and SEQ ID NO:76 (TPP-17078). In some of these examples, the antibody furthermore comprises a variable heavy chain having at least 90%, 95%, 98%, 99% or 100% sequence identity with SEQ ID NO:69 and/or a variable light chain having at least 90%, 95%, 98%, 99% or 100% sequence identity with SEQ ID NO:73. In some of the examples, the antibody or antigen-binding fragment thereof is a human Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:79 and/or a light chain region having at least 90%, 95%, 98%, 99% or 100% sequence identity with SEQ ID NO:80. In some of these examples, the antibody or antigen-binding fragment thereof is a human IgG1 antibody.

### TPP-17405

In some other of these preferred embodiments, the radionuclide is ²²⁷Th, the chelator comprises Me-3,2-HOPO, or a derivative thereof and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:84, SEQ ID NO:85, SEQ ID NO:86, SEQ ID NO:88, SEQ ID NO:89, and SEQ ID NO:90 (TPP-17405). In some of these examples, the antibody furthermore comprises a variable heavy chain having at least 90%, 95%, 98%, 99% or 100% sequence identity with SEQ ID NO:83 and/or a variable light chain having at least 90%, 95%, 98%, 99% or 100% sequence identity with SEQ ID NO:87. In some of the examples, the antibody or antigen-binding fragment thereof is a Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:91 and/or a light chain region having at least 90%, 95%, 98%, 99% or 100% sequence identity with SEQ ID NO:92. In some of these examples, the antibody or antigen-binding fragment thereof is a humanized IgG1 antibody.

In some other of these preferred embodiments, the radionuclide is ²²⁷Th, the chelator comprises a chelator according to formula I or a derivative thereof, and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:84, SEQ ID NO:85, SEQ ID NO:86, SEQ ID NO:88, SEQ ID NO:89, and SEQ ID NO:90 (TPP-17405). In some of these examples, the antibody furthermore comprises a variable heavy chain having at least 90%, 95%, 98%, 99% or 100% sequence identity with SEQ ID NO:83 and/or a variable light chain having at least 90%, 95%, 98%, 99% or 100% sequence identity with SEQ ID NO:87. In some of the examples, the antibody or antigen-binding fragment thereof is a Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:91 and/or a light chain region having at least 90%, 95%, 98%, 99% or 100% sequence identity with SEQ ID NO:92. In some of these examples, the antibody or antigen-binding fragment thereof is a humanized IgG1 antibody.

In some other of these preferred embodiments, the radionuclide is ²²⁷Th, the chelator comprises 3,2-HOPO, or a derivative thereof and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:84, SEQ ID NO:85, SEQ ID NO:86, SEQ ID NO:88, SEQ ID NO:89, and SEQ ID NO:90 (TPP-17405). In some of these examples, the antibody furthermore comprises a variable heavy chain having at least 90%, 95%, 98%, 99% or 100% sequence identity with SEQ ID NO:83 and/or a variable light chain having at least 90%, 95%, 98%, 99% or 100% sequence identity with SEQ ID NO:87. In some of the examples, the antibody or antigen-binding fragment thereof is a Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:91 and/or a light chain region having at least 90%, 95%, 98%, 99% or 100% sequence identity with SEQ ID NO:92. In some of these examples, the antibody or antigen-binding fragment thereof is a humanized IgG1 antibody.

### TPP-17418

In some other of these preferred embodiments, the radionuclide is ²²⁷Th, the chelator comprises Me-3,2-HOPO, or a derivative thereof and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:94, SEQ ID NO:95, SEQ ID NO:96, SEQ ID NO:98, SEQ ID NO:99, and SEQ ID NO:100 (TPP-17418). In some of these examples, the antibody furthermore comprises a variable heavy chain having at least 90%, 95%, 98%, 99% or 100% sequence identity with SEQ ID NO:93 and/or a variable light chain having at least 90%, 95%, 98%, 99% or 100% sequence identity with SEQ ID NO:97. In some of the examples, the antibody or antigen-binding fragment thereof is a Fab′, F(ab′)2, Fab, Fv, rIgG, or scFv fragment. In some examples, the antibody furthermore comprises heavy chain region having at least 90%, 95%, 98%, 99% or 100% sequence identity with SEQ ID NO:101 and/or a light chain region having at least 90%, 95%, 98%, 99% or 100% sequence identity with SEQ ID NO:102. In some of these examples, the antibody or antigen-binding fragment thereof is a humanized IgG1 antibody.

In some other of these preferred embodiments, the radionuclide is ²²⁷Th, the chelator comprises a chelator according to formula I or a derivative thereof, and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:94, SEQ ID NO:95, SEQ ID NO:96, SEQ ID NO:98, SEQ ID NO:99, and SEQ ID NO:100 (TPP-17418). In some of these examples, the antibody furthermore comprises a variable heavy chain having at least 90%, 95%, 98%, 99% or 100% sequence identity with SEQ ID NO:93 and/or a variable light chain having at least 90%, 95%, 98%, 99% or 100% sequence identity with SEQ ID NO:97. In some of the examples, the antibody or antigen-binding fragment thereof is a Fab′, F(ab′)2, Fab, Fv, rIgG, or scFv fragment. In some examples, the antibody furthermore comprises heavy chain region having at least 90%, 95%, 98%, 99% or 100% sequence identity with SEQ ID NO:101 and/or a light chain region having at least 90%, 95%, 98%, 99% or 100% sequence identity with SEQ ID NO:102. In some of these examples, the antibody or antigen-binding fragment thereof is a humanized IgG1 antibody.

In some other of these preferred embodiments, the radionuclide is ²²⁷Th, the chelator comprises 3,2-HOPO, or a derivative thereof and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:94, SEQ ID NO:95, SEQ ID NO:96, SEQ ID NO:98, SEQ ID NO:99, and SEQ ID NO:100 (TPP-17418). In some of these examples, the antibody furthermore comprises a variable heavy chain having at least 90%, 95%, 98%, 99% or 100% sequence identity with SEQ ID NO:93 and/or a variable light chain having at least 90%, 95%, 98%, 99% or 100% sequence identity with SEQ ID NO:97. In some of the examples, the antibody or antigen-binding fragment thereof is a Fab′, F(ab′)2, Fab, Fv, rIgG, or scFv fragment. In some examples, the antibody furthermore comprises heavy chain region having at least 90%, 95%, 98%, 99% or 100% sequence identity with SEQ ID NO:101 and/or a light chain region having at least 90%, 95%, 98%, 99% or 100% sequence identity with SEQ ID NO:102. In some of these examples, the antibody or antigen-binding fragment thereof is a humanized IgG1 antibody.

### TPP-17419

In some other of these preferred embodiments, the radionuclide is ²²⁷Th, the chelator comprises Me-3,2-HOPO, or a derivative thereof and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:104, SEQ ID NO:105, SEQ ID NO:106, SEQ ID NO:108, SEQ ID NO:109, and SEQ ID NO:110 (TPP-17419). In some of these examples, the antibody furthermore comprises a variable heavy chain having at least 90%, 95%, 98%, 99% or 100% sequence identity with SEQ ID NO:103 and/or a variable light chain having at least 90%, 95%, 98%, 99% or 100% sequence identity with SEQ ID NO:107. In some of the examples, the antibody or antigen-binding fragment thereof is a Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:111 and/or a light chain region having at least 90%, 95%, 98%, 99% or 100% sequence identity with SEQ ID NO:112. In some of these examples, the antibody or antigen-binding fragment thereof is a humanized IgG1 antibody.

In some other of these preferred embodiments, the radionuclide is ²²⁷Th, the chelator comprises a chelator according to formula I or a derivative thereof, and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:104, SEQ ID NO:105, SEQ ID NO:106, SEQ ID NO:108, SEQ ID NO:109, and SEQ ID NO:110 (TPP-17419). In some of these examples, the antibody furthermore comprises a variable heavy chain having at least 90%, 95%, 98%, 99% or 100% sequence identity with SEQ ID NO:103 and/or a variable light chain having at least 90%, 95%, 98%, 99% or 100% sequence identity with SEQ ID NO:107. In some of the examples, the antibody or antigen-binding fragment thereof is a Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:111 and/or a light chain region having at least 90%, 95%, 98%, 99% or 100% sequence identity with SEQ ID NO:112. In some of these examples, the antibody or antigen-binding fragment thereof is a humanized IgG1 antibody.

In some other of these preferred embodiments, the radionuclide is ²²⁷Th, the chelator comprises 3,2-HOPO, or a derivative thereof and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:104, SEQ ID NO:105, SEQ ID NO:106, SEQ ID NO:108, SEQ ID NO:109, and SEQ ID NO:110 (TPP-17419). In some of these examples, the antibody furthermore comprises a variable heavy chain having at least 90%, 95%, 98%, 99% or 100% sequence identity with SEQ ID NO:103 and/or a variable light chain having at least 90%, 95%, 98%, 99% or 100% sequence identity with SEQ ID NO:107. In some of the examples, the antibody or antigen-binding fragment thereof is a Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:111 and/or a light chain region having at least 90%, 95%, 98%, 99% or 100% sequence identity with SEQ ID NO:112. In some of these examples, the antibody or antigen-binding fragment thereof is a humanized IgG1 antibody.

### TPP-17421

In some other of these preferred embodiments, the radionuclide is ²²⁷Th, the chelator comprises Me-3,2-HOPO, or a derivative thereof and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:114, SEQ ID NO:115, SEQ ID NO:116, SEQ ID NO:118, SEQ ID NO:119, and SEQ ID NO:120 (TPP-17421). In some of these examples, the antibody furthermore comprises a variable heavy chain having at least 90%, 95%, 98%, 99% or 100% sequence identity with SEQ ID NO:113 and/or a variable light chain having at least 90%, 95%, 98%, 99% or 100% sequence identity with SEQ ID NO:117. In some of the examples, the antibody or antigen-binding fragment thereof is a Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:123 and/or a light chain region having at least 90%, 95%, 98%, 99% or 100% sequence identity with SEQ ID NO:124. In some of these examples, the antibody or antigen-binding fragment thereof is a humanized IgG1 antibody.

In some other of these preferred embodiments, the radionuclide is ²²⁷Th, the chelator comprises a chelator according to formula I or a derivative thereof, and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:114, SEQ ID NO:115, SEQ ID NO:116, SEQ ID NO:118, SEQ ID NO:119, and SEQ ID NO:120 (TPP-17421). In some of these examples, the antibody furthermore comprises a variable heavy chain having at least 90%, 95%, 98%, 99% or 100% sequence identity with SEQ ID NO:113 and/or a variable light chain having at least 90%, 95%, 98%, 99% or 100% sequence identity with SEQ ID NO:117. In some of the examples, the antibody or antigen-binding fragment thereof is a Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:123 and/or a light chain region having at least 90%, 95%, 98%, 99% or 100% sequence identity with SEQ ID NO:124. In some of these examples, the antibody or antigen-binding fragment thereof is a humanized IgG1 antibody.

In some other of these preferred embodiments, the radionuclide is ²²⁷Th, the chelator comprises 3,2-HOPO, or a derivative thereof and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:114, SEQ ID NO:115, SEQ ID NO:116, SEQ ID NO:118, SEQ ID NO:119, and SEQ ID NO:120 (TPP-17421). In some of these examples, the antibody furthermore comprises a variable heavy chain having at least 90%, 95%, 98%, 99% or 100% sequence identity with SEQ ID NO:113 and/or a variable light chain having at least 90%, 95%, 98%, 99% or 100% sequence identity with SEQ ID NO:117. In some of the examples, the antibody or antigen-binding fragment thereof is a Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:123 and/or a light chain region having at least 90%, 95%, 98%, 99% or 100% sequence identity with SEQ ID NO:124. In some of these examples, the antibody or antigen-binding fragment thereof is a humanized IgG1 antibody.

### TPP-17422

In some other of these preferred embodiments, the radionuclide is ²²⁷Th, the chelator comprises Me-3,2-HOPO, or a derivative thereof and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:128, SEQ ID NO:129, SEQ ID NO:130, SEQ ID NO:132, SEQ ID NO:133, and SEQ ID NO:134 (TPP-17422). In some of these examples, the antibody furthermore comprises a variable heavy chain having at least 90%, 95%, 98%, 99% or 100% sequence identity with SEQ ID NO:127 and/or a variable light chain having at least 90%, 95%, 98%, 99% or 100% sequence identity with SEQ ID NO:131. In some of the examples, the antibody or antigen-binding fragment thereof is a Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:135 and/or a light chain region having at least 90%, 95%, 98%, 99% or 100% sequence identity with SEQ ID NO:136. In some of these examples, the antibody or antigen-binding fragment thereof is a humanized IgG1 antibody.

In some other of these preferred embodiments, the radionuclide is ²²⁷Th, the chelator comprises a chelator according to formula I or a derivative thereof, and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:128, SEQ ID NO:129, SEQ ID NO:130, SEQ ID NO:132, SEQ ID NO:133, and SEQ ID NO:134 (TPP-17422). In some of these examples, the antibody furthermore comprises a variable heavy chain having at least 90%, 95%, 98%, 99% or 100% sequence identity with SEQ ID NO:127 and/or a variable light chain having at least 90%, 95%, 98%, 99% or 100% sequence identity with SEQ ID NO:131. In some of the examples, the antibody or antigen-binding fragment thereof is a Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:135 and/or a light chain region having at least 90%, 95%, 98%, 99% or 100% sequence identity with SEQ ID NO:136. In some of these examples, the antibody or antigen-binding fragment thereof is a humanized IgG1 antibody.

In some other of these preferred embodiments, the radionuclide is ²²⁷Th, the chelator comprises 3,2-HOPO, or a derivative thereof and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:128, SEQ ID NO:129, SEQ ID NO:130, SEQ ID NO:132, SEQ ID NO:133, and SEQ ID NO:134 (TPP-17422). In some of these examples, the antibody furthermore comprises a variable heavy chain having at least 90%, 95%, 98%, 99% or 100% sequence identity with SEQ ID NO:127 and/or a variable light chain having at least 90%, 95%, 98%, 99% or 100% sequence identity with SEQ ID NO:131. In some of the examples, the antibody or antigen-binding fragment thereof is a Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:135 and/or a light chain region having at least 90%, 95%, 98%, 99% or 100% sequence identity with SEQ ID NO:136. In some of these examples, the antibody or antigen-binding fragment thereof is a humanized IgG1 antibody.

### Conjugates Chelating ⁸⁹Zr

In some further preferred embodiments, the radionuclide is ⁸⁹Zr, the chelator arranged for complexation of ⁸⁹Zr comprises desferrioxamine (DFO), HOPO, 3,2-HOPO, Me-3,2-HOPO, a chelator according to formula I or a derivative of any of these for chelation of zirconium-89 and/or the targeting moiety binding to human LRRC15 is an antibody or antigen-binding fragment thereof, such as one of those according to embodiments A, B or C. Zirconium-89 forms complexes in which zirconium is present in the +4 oxidation state.

### TPP-1633

In some of these preferred embodiments, the radionuclide is ⁸⁹Zr, the chelator comprises Me-3,2-HOPO, or a derivative thereof and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:2, SEQ ID NO:3, SEQ ID NO:4, SEQ ID NO:6, SEQ ID NO:7 and SEQ ID NO:8 (TPP-1633). In some of these examples, the antibody furthermore comprises a variable heavy chain having at least 90%, 95%, 98%, 99% or 100% sequence identity with SEQ ID NO:1 and/or a variable light chain having at least 90%, 95%, 98%, 99% or 100% sequence identity with SEQ ID NO:5. In some of the examples, the antibody or antigen-binding fragment thereof is a human Fab′, F(ab′)2, Fab, Fv, rIgG, or scFv fragment. In some examples, the antibody comprises a heavy chain region having at least 90%, 95%, 98%, 99% or 100% sequence identity with SEQ ID NO:9 and/or a light chain region having at least 90%, 95%, 98%, 99% or 100% sequence identity with SEQ ID NO:10. In some of these examples, the antibody or antigen-binding fragment thereof is a human IgG1 antibody.

In some other of these preferred embodiments, the radionuclide is ⁸⁹Zr, the chelator comprises a chelator according to formula I or a derivative thereof, and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:2, SEQ ID NO:3, SEQ ID NO:4, SEQ ID NO:6, SEQ ID NO:7 and SEQ ID NO:8 (TPP-1633). In some of these examples, the antibody furthermore comprises a variable heavy chain having at least 90%, 95%, 98%, 99% or 100% sequence identity with SEQ ID NO:1 and/or a variable light chain having at least 90%, 95%, 98%, 99% or 100% sequence identity with SEQ ID NO:5. In some of the examples, the antibody or antigen-binding fragment thereof is a human Fab′, F(ab′)2, Fab, Fv, rIgG, or scFv fragment. In some examples, the antibody comprises a heavy chain region having at least 90%, 95%, 98%, 99% or 100% sequence identity with SEQ ID NO:9 and/or a light chain region having at least 90%, 95%, 98%, 99% or 100% sequence identity with SEQ ID NO:10. In some of these examples, the antibody or antigen-binding fragment thereof is a human IgG1 antibody.

In some of these preferred embodiments, the radionuclide is ⁸⁹Zr, the chelator comprises 3,2-HOPO, or a derivative thereof and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:2, SEQ ID NO:3, SEQ ID NO:4, SEQ ID NO:6, SEQ ID NO:7 and SEQ ID NO:8 (TPP-1633). In some of these examples, the antibody furthermore comprises a variable heavy chain having at least 90%, 95%, 98%, 99% or 100% sequence identity with SEQ ID NO:1 and/or a variable light chain having at least 90%, 95%, 98%, 99% or 100% sequence identity with SEQ ID NO:5. In some of the examples, the antibody or antigen-binding fragment thereof is a human Fab′, F(ab′)2, Fab, Fv, rIgG, or scFv fragment. In some examples, the antibody comprises a heavy chain region having at least 90%, 95%, 98%, 99% or 100% sequence identity with SEQ ID NO:9 and/or a light chain region having at least 90%, 95%, 98%, 99% or 100% sequence identity with SEQ ID NO:10. In some of these examples, the antibody or antigen-binding fragment thereof is a human IgG1 antibody.

### TPP-14389

In some other of these preferred embodiments, the radionuclide is ⁸⁹Zr, the chelator comprises Me-3,2-HOPO, or a derivative thereof and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:22, SEQ ID NO:23, SEQ ID NO:24, SEQ ID NO:26, SEQ ID NO:27 and SEQ ID NO:28 (TPP-14389). In some of these examples, the antibody furthermore comprises a variable heavy chain having at least 90%, 95%, 98%, 99% or 100% sequence identity with SEQ ID NO:21 and/or a variable light chain having at least 90%, 95%, 98%, 99% or 100% sequence identity with SEQ ID NO:25. In some of the examples, the antibody or antigen-binding fragment thereof is a human Fab′, F(ab′)2, Fab, Fv, rIgG, or scFv fragment. In some examples, the antibody comprises a heavy chain region having at least 90%, 95%, 98%, 99% or 100% sequence identity with SEQ ID NO:31 and/or a light chain region having at least 90%, 95%, 98%, 99% or 100% sequence identity with SEQ ID NO:32. In some of these examples, the antibody or antigen-binding fragment thereof is a human IgG1 antibody.

In some other of these preferred embodiments, the radionuclide is ⁸⁹Zr, the chelator comprises a chelator according to formula I or a derivative thereof, and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:22, SEQ ID NO:23, SEQ ID NO:24, SEQ ID NO:26, SEQ ID NO:27 and SEQ ID NO:28 (TPP-14389). In some of these examples, the antibody furthermore comprises a variable heavy chain having at least 90%, 95%, 98%, 99% or 100% sequence identity with SEQ ID NO:21 and/or a variable light chain having at least 90%, 95%, 98%, 99% or 100% sequence identity with SEQ ID NO:25. In some of the examples, the antibody or antigen-binding fragment thereof is a human Fab′, F(ab′)2, Fab, Fv, rIgG, or scFv fragment. In some examples, the antibody comprises a heavy chain region having at least 90%, 95%, 98%, 99% or 100% sequence identity with SEQ ID NO:31 and/or a light chain region having at least 90%, 95%, 98%, 99% or 100% sequence identity with SEQ ID NO:32. In some of these examples, the antibody or antigen-binding fragment thereof is a human IgG1 antibody. In some other of these preferred embodiments, the radionuclide is ⁸⁹Zr, the chelator comprises 3,2-HOPO, or a derivative thereof and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:22, SEQ ID NO:23, SEQ ID NO:24, SEQ ID NO:26, SEQ ID NO:27 and SEQ ID NO:28 (TPP-14389). In some of these examples, the antibody furthermore comprises a variable heavy chain having at least 90%, 95%, 98%, 99% or 100% sequence identity with SEQ ID NO:21 and/or a variable light chain having at least 90%, 95%, 98%, 99% or 100% sequence identity with SEQ ID NO:25. In some of the examples, the antibody or antigen-binding fragment thereof is a human Fab′, F(ab′)2, Fab, Fv, rIgG, or scFv fragment. In some examples, the antibody comprises a heavy chain region having at least 90%, 95%, 98%, 99% or 100% sequence identity with SEQ ID NO:31 and/or a light chain region having at least 90%, 95%, 98%, 99% or 100% sequence identity with SEQ ID NO:32. In some of these examples, the antibody or antigen-binding fragment thereof is a human IgG1 antibody.

### TPP-14392

In some other of these preferred embodiments, the radionuclide is ⁸⁹Zr, the chelator comprises Me-3,2-HOPO, or a derivative thereof and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:36, SEQ ID NO:37, SEQ ID NO:38, SEQ ID NO:40, SEQ ID NO:41 and SEQ ID NO:42 (e.g. TPP-14392). In some of these examples, the antibody furthermore comprises a variable heavy chain having at least 90%, 95%, 98%, 99% or 100% sequence identity with SEQ ID NO:35 and/or a variable light chain having at least 90%, 95%, 98%, 99% or 100% sequence identity with SEQ ID NO:39. In some of the examples, the antibody or antigen-binding fragment thereof is a human Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:45 and/or a light chain region having at least 90%, 95%, 98%, 99% or 100% sequence identity with SEQ ID NO:46. In some of these examples, the antibody or antigen-binding fragment thereof is a human IgG1 antibody.

In some other of these preferred embodiments, the radionuclide is ⁸⁹Zr, the chelator comprises a chelator according to formula I or a derivative thereof, and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:36, SEQ ID NO:37, SEQ ID NO:38, SEQ ID NO:40, SEQ ID NO:41 and SEQ ID NO:42 (e.g. TPP-14392). In some of these examples, the antibody furthermore comprises a variable heavy chain having at least 90%, 95%, 98%, 99% or 100% sequence identity with SEQ ID NO:35 and/or a variable light chain having at least 90%, 95%, 98%, 99% or 100% sequence identity with SEQ ID NO:39. In some of the examples, the antibody or antigen-binding fragment thereof is a human Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:45 and/or a light chain region having at least 90%, 95%, 98%, 99% or 100% sequence identity with SEQ ID NO:46. In some of these examples, the antibody or antigen-binding fragment thereof is a human IgG1 antibody.

In some other of these preferred embodiments, the radionuclide is ⁸⁹Zr, the chelator comprises 3,2-HOPO, or a derivative thereof and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:36, SEQ ID NO:37, SEQ ID NO:38, SEQ ID NO:40, SEQ ID NO:41 and SEQ ID NO:42 (e.g. TPP-14392). In some of these examples, the antibody furthermore comprises a variable heavy chain having at least 90%, 95%, 98%, 99% or 100% sequence identity with SEQ ID NO:35 and/or a variable light chain having at least 90%, 95%, 98%, 99% or 100% sequence identity with SEQ ID NO:39. In some of the examples, the antibody or antigen-binding fragment thereof is a human Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:45 and/or a light chain region having at least 90%, 95%, 98%, 99% or 100% sequence identity with SEQ ID NO:46. In some of these examples, the antibody or antigen-binding fragment thereof is a human IgG1 antibody.

### TPP-17073

In some other of these preferred embodiments, the radionuclide is ⁸⁹Zr, the chelator comprises Me-3,2-HOPO, or a derivative thereof and the targeting moiety binding to human LRRC15 is an antibody or antigen-binding fragment thereof, the chelator comprises Me-3,2-HOPO, or a derivative thereof and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:50, SEQ ID NO:51, SEQ ID NO:52, SEQ ID NO:54, SEQ ID NO:55, and SEQ ID NO:56 (TPP-17073). In some of these examples, the antibody furthermore comprises a variable heavy chain having at least 90%, 95%, 98%, 99% or 100% sequence identity with SEQ ID NO:49 and/or a variable light chain having at least 90%, 95%, 98%, 99% or 100% sequence identity with SEQ ID NO:53. In some of the examples, the antibody or antigen-binding fragment thereof is a human Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:57 and/or a light chain region having at least 90%, 95%, 98%, 99% or 100% sequence identity with SEQ ID NO:58. In some of these examples, the antibody or antigen-binding fragment thereof is a human IgG1 antibody.

In some other of these preferred embodiments, the radionuclide is ⁸⁹Zr, the chelator comprises a chelator according to formula I or a derivative thereof, and the targeting moiety binding to human LRRC15 is an antibody or antigen-binding fragment thereof, the chelator comprises Me-3,2-HOPO, or a derivative thereof and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:50, SEQ ID NO:51, SEQ ID NO:52, SEQ ID NO:54, SEQ ID NO:55, and SEQ ID NO:56 (TPP-17073). In some of these examples, the antibody furthermore comprises a variable heavy chain having at least 90%, 95%, 98%, 99% or 100% sequence identity with SEQ ID NO:49 and/or a variable light chain having at least 90%, 95%, 98%, 99% or 100% sequence identity with SEQ ID NO:53. In some of the examples, the antibody or antigen-binding fragment thereof is a human Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:57 and/or a light chain region having at least 90%, 95%, 98%, 99% or 100% sequence identity with SEQ ID NO:58. In some of these examples, the antibody or antigen-binding fragment thereof is a human IgG1 antibody. In some other of these preferred embodiments, the radionuclide is ⁸⁹Zr, the chelator comprises 3,2-HOPO, or a derivative thereof and the targeting moiety binding to human LRRC15 is an antibody or antigen-binding fragment thereof, the chelator comprises Me-3,2-HOPO, or a derivative thereof and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:50, SEQ ID NO:51, SEQ ID NO:52, SEQ ID NO:54, SEQ ID NO:55, and SEQ ID NO:56 (TPP-17073). In some of these examples, the antibody furthermore comprises a variable heavy chain having at least 90%, 95%, 98%, 99% or 100% sequence identity with SEQ ID NO:49 and/or a variable light chain having at least 90%, 95%, 98%, 99% or 100% sequence identity with SEQ ID NO:53. In some of the examples, the antibody or antigen-binding fragment thereof is a human Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:57 and/or a light chain region having at least 90%, 95%, 98%, 99% or 100% sequence identity with SEQ ID NO:58. In some of these examples, the antibody or antigen-binding fragment thereof is a human IgG1 antibody.

### TPP-17074

In some other of these preferred embodiments, the radionuclide is ⁸⁹Zr, the chelator comprises Me-3,2-HOPO, or a derivative thereof and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:60, SEQ ID NO:61, SEQ ID NO:62, SEQ ID NO:64, SEQ ID NO:65, and SEQ ID NO:66 (TPP-17074). In some of these examples, the antibody furthermore comprises a variable heavy chain having at least 90%, 95%, 98%, 99% or 100% sequence identity with SEQ ID NO:59 and/or a variable light chain having at least 90%, 95%, 98%, 99% or 100% sequence identity with SEQ ID NO:63. In some of the examples, the antibody or antigen-binding fragment thereof is a human Fab′, F(ab′)2, Fab, Fv, rIgG, or scFv fragment. In some examples, the antibody furthermore comprises a e) a heavy chain region having at least 90%, 95%, 98%, 99% or 100% sequence identity with SEQ ID NO:67 and/or a light chain region having at least 90%, 95%, 98%, 99% or 100% sequence identity with SEQ ID NO:68. In some of these examples, the antibody or antigen-binding fragment thereof is a human IgG1 antibody. In some other of these preferred embodiments, the radionuclide is ⁸⁹Zr, the chelator comprises a chelator according to formula I or a derivative thereof, and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:60, SEQ ID NO:61, SEQ ID NO:62, SEQ ID NO:64, SEQ ID NO:65, and SEQ ID NO:66 (TPP-17074). In some of these examples, the antibody furthermore comprises a variable heavy chain having at least 90%, 95%, 98%, 99% or 100% sequence identity with SEQ ID NO:59 and/or a variable light chain having at least 90%, 95%, 98%, 99% or 100% sequence identity with SEQ ID NO:63. In some of the examples, the antibody or antigen-binding fragment thereof is a human Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:67 and/or a light chain region having at least 90%, 95%, 98%, 99% or 100% sequence identity with SEQ ID NO:68. In some of these examples, the antibody or antigen-binding fragment thereof is a human IgG1 antibody.

In some other of these preferred embodiments, the radionuclide is ⁸⁹Zr, the chelator comprises 3,2-HOPO, or a derivative thereof and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:60, SEQ ID NO:61, SEQ ID NO:62, SEQ ID NO:64, SEQ ID NO:65, and SEQ ID NO:66 (TPP-17074). In some of these examples, the antibody furthermore comprises a variable heavy chain having at least 90%, 95%, 98%, 99% or 100% sequence identity with SEQ ID NO:59 and/or a variable light chain having at least 90%, 95%, 98%, 99% or 100% sequence identity with SEQ ID NO:63. In some of the examples, the antibody or antigen-binding fragment thereof is a human Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:67 and/or a light chain region having at least 90%, 95%, 98%, 99% or 100% sequence identity with SEQ ID NO:68. In some of these examples, the antibody or antigen-binding fragment thereof is a human IgG1 antibody.

### TPP-17078

In some other of these preferred embodiments, the radionuclide is ⁸⁹Zr, the chelator comprises Me-3,2-HOPO, or a derivative thereof and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:70, SEQ ID NO:71, SEQ ID NO:72, SEQ ID NO:74, SEQ ID NO:75, and SEQ ID NO:76 (TPP-17078). In some of these examples, the antibody furthermore comprises a variable heavy chain having at least 90%, 95%, 98%, 99% or 100% sequence identity with SEQ ID NO:69 and/or a variable light chain having at least 90%, 95%, 98%, 99% or 100% sequence identity with SEQ ID NO:73. In some of the examples, the antibody or antigen-binding fragment thereof is a human Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:79 and/or a light chain region having at least 90%, 95%, 98%, 99% or 100% sequence identity with SEQ ID NO:80. In some of these examples, the antibody or antigen-binding fragment thereof is a human IgG1 antibody. In some other of these preferred embodiments, the radionuclide is ⁸⁹Zr, the chelator comprises a chelator according to formula I or a derivative thereof, and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:70, SEQ ID NO:71, SEQ ID NO:72, SEQ ID NO:74, SEQ ID NO:75, and SEQ ID NO:76 (TPP-17078). In some of these examples, the antibody furthermore comprises a variable heavy chain having at least 90%, 95%, 98%, 99% or 100% sequence identity with SEQ ID NO:69 and/or a variable light chain having at least 90%, 95%, 98%, 99% or 100% sequence identity with SEQ ID NO:73. In some of the examples, the antibody or antigen-binding fragment thereof is a human Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:79 and/or a light chain region having at least 90%, 95%, 98%, 99% or 100% sequence identity with SEQ ID NO:80. In some of these examples, the antibody or antigen-binding fragment thereof is a human IgG1 antibody.

In some other of these preferred embodiments, the radionuclide is ⁸⁹Zr, the chelator comprises 3,2-HOPO, or a derivative thereof and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:70, SEQ ID NO:71, SEQ ID NO:72, SEQ ID NO:74, SEQ ID NO:75, and SEQ ID NO:76 (TPP-17078). In some of these examples, the antibody furthermore comprises a variable heavy chain having at least 90%, 95%, 98%, 99% or 100% sequence identity with SEQ ID NO:69 and/or a variable light chain having at least 90%, 95%, 98%, 99% or 100% sequence identity with SEQ ID NO:73. In some of the examples, the antibody or antigen-binding fragment thereof is a human Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:79 and/or a light chain region having at least 90%, 95%, 98%, 99% or 100% sequence identity with SEQ ID NO:80. In some of these examples, the antibody or antigen-binding fragment thereof is a human IgG1 antibody.

### TPP-17405

In some other of these preferred embodiments, the radionuclide is ⁸⁹Zr, the chelator comprises Me-3,2-HOPO, or a derivative thereof and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:84, SEQ ID NO:85, SEQ ID NO:86, SEQ ID NO:88, SEQ ID NO:89, and SEQ ID NO:90 (TPP-17405). In some of these examples, the antibody furthermore comprises a variable heavy chain having at least 90%, 95%, 98%, 99% or 100% sequence identity with SEQ ID NO:83 and/or a variable light chain having at least 90%, 95%, 98%, 99% or 100% sequence identity with SEQ ID NO:87. In some of the examples, the antibody or antigen-binding fragment thereof is a Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:91 and/or a light chain region having at least 90%, 95%, 98%, 99% or 100% sequence identity with SEQ ID NO:92. In some of these examples, the antibody or antigen-binding fragment thereof is a humanized IgG1 antibody.

In some other of these preferred embodiments, the radionuclide is ⁸⁹Zr, the chelator comprises a chelator according to formula I or a derivative thereof, and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:84, SEQ ID NO:85, SEQ ID NO:86, SEQ ID NO:88, SEQ ID NO:89, and SEQ ID NO:90 (TPP-17405). In some of these examples, the antibody furthermore comprises a variable heavy chain having at least 90%, 95%, 98%, 99% or 100% sequence identity with SEQ ID NO:83 and/or a variable light chain having at least 90%, 95%, 98%, 99% or 100% sequence identity with SEQ ID NO:87. In some of the examples, the antibody or antigen-binding fragment thereof is a Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:91 and/or a light chain region having at least 90%, 95%, 98%, 99% or 100% sequence identity with SEQ ID NO:92. In some of these examples, the antibody or antigen-binding fragment thereof is a humanized IgG1 antibody.

In some other of these preferred embodiments, the radionuclide is ⁸⁹Zr, the chelator comprises 3,2-HOPO, or a derivative thereof and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:84, SEQ ID NO:85, SEQ ID NO:86, SEQ ID NO:88, SEQ ID NO:89, and SEQ ID NO:90 (TPP-17405). In some of these examples, the antibody furthermore comprises a variable heavy chain having at least 90%, 95%, 98%, 99% or 100% sequence identity with SEQ ID NO:83 and/or a variable light chain having at least 90%, 95%, 98%, 99% or 100% sequence identity with SEQ ID NO:87. In some of the examples, the antibody or antigen-binding fragment thereof is a Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:91 and/or a light chain region having at least 90%, 95%, 98%, 99% or 100% sequence identity with SEQ ID NO:92. In some of these examples, the antibody or antigen-binding fragment thereof is a humanized IgG1 antibody.

### TPP-17418

In some other of these preferred embodiments, the radionuclide is ⁸⁹Zr, the chelator comprises Me-3,2-HOPO, or a derivative thereof and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:94, SEQ ID NO:95, SEQ ID NO:96, SEQ ID NO:98, SEQ ID NO:99, and SEQ ID NO:100 (TPP-17418). In some of these examples, the antibody furthermore comprises a variable heavy chain having at least 90%, 95%, 98%, 99% or 100% sequence identity with SEQ ID NO:93 and/or a variable light chain having at least 90%, 95%, 98%, 99% or 100% sequence identity with SEQ ID NO:97. In some of the examples, the antibody or antigen-binding fragment thereof is a Fab′, F(ab′)2, Fab, Fv, rIgG, or scFv fragment. In some examples, the antibody furthermore comprises heavy chain region having at least 90%, 95%, 98%, 99% or 100% sequence identity with SEQ ID NO:101 and/or a light chain region having at least 90%, 95%, 98%, 99% or 100% sequence identity with SEQ ID NO:102. In some of these examples, the antibody or antigen-binding fragment thereof is a humanized IgG1 antibody.

In some other of these preferred embodiments, the radionuclide is ⁸⁹Zr, the chelator comprises a chelator according to formula I or a derivative thereof, and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:94, SEQ ID NO:95, SEQ ID NO:96, SEQ ID NO:98, SEQ ID NO:99, and SEQ ID NO:100 (TPP-17418). In some of these examples, the antibody furthermore comprises a variable heavy chain having at least 90%, 95%, 98%, 99% or 100% sequence identity with SEQ ID NO:93 and/or a variable light chain having at least 90%, 95%, 98%, 99% or 100% sequence identity with SEQ ID NO:97. In some of the examples, the antibody or antigen-binding fragment thereof is a Fab′, F(ab′)2, Fab, Fv, rIgG, or scFv fragment. In some examples, the antibody furthermore comprises heavy chain region having at least 90%, 95%, 98%, 99% or 100% sequence identity with SEQ ID NO:101 and/or a light chain region having at least 90%, 95%, 98%, 99% or 100% sequence identity with SEQ ID NO:102. In some of these examples, the antibody or antigen-binding fragment thereof is a humanized IgG1 antibody.

In some other of these preferred embodiments, the radionuclide is ⁸⁹Zr, the chelator comprises 3,2-HOPO, or a derivative thereof and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:94, SEQ ID NO:95, SEQ ID NO:96, SEQ ID NO:98, SEQ ID NO:99, and SEQ ID NO:100 (TPP-17418). In some of these examples, the antibody furthermore comprises a variable heavy chain having at least 90%, 95%, 98%, 99% or 100% sequence identity with SEQ ID NO:93 and/or a variable light chain having at least 90%, 95%, 98%, 99% or 100% sequence identity with SEQ ID NO:97. In some of the examples, the antibody or antigen-binding fragment thereof is a Fab′, F(ab′)2, Fab, Fv, rIgG, or scFv fragment. In some examples, the antibody furthermore comprises heavy chain region having at least 90%, 95%, 98%, 99% or 100% sequence identity with SEQ ID NO:101 and/or a light chain region having at least 90%, 95%, 98%, 99% or 100% sequence identity with SEQ ID NO:102. In some of these examples, the antibody or antigen-binding fragment thereof is a humanized IgG1 antibody.

### TPP-17419

In some other of these preferred embodiments, the radionuclide is ⁸⁹Zr, the chelator comprises Me-3,2-HOPO, or a derivative thereof and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:104, SEQ ID NO:105, SEQ ID NO:106, SEQ ID NO:108, SEQ ID NO:109, and SEQ ID NO:110 (TPP-17419). In some of these examples, the antibody furthermore comprises a variable heavy chain having at least 90%, 95%, 98%, 99% or 100% sequence identity with SEQ ID NO:103 and/or a variable light chain having at least 90%, 95%, 98%, 99% or 100% sequence identity with SEQ ID NO:107. In some of the examples, the antibody or antigen-binding fragment thereof is a Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:111 and/or a light chain region having at least 90%, 95%, 98%, 99% or 100% sequence identity with SEQ ID NO:112. In some of these examples, the antibody or antigen-binding fragment thereof is a humanized IgG1 antibody.

In some other of these preferred embodiments, the radionuclide is ⁸⁹Zr, the chelator comprises a chelator according to formula I or a derivative thereof, and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:104, SEQ ID NO:105, SEQ ID NO:106, SEQ ID NO:108, SEQ ID NO:109, and SEQ ID NO:110 (TPP-17419). In some of these examples, the antibody furthermore comprises a variable heavy chain having at least 90%, 95%, 98%, 99% or 100% sequence identity with SEQ ID NO:103 and/or a variable light chain having at least 90%, 95%, 98%, 99% or 100% sequence identity with SEQ ID NO:107. In some of the examples, the antibody or antigen-binding fragment thereof is a Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:111 and/or a light chain region having at least 90%, 95%, 98%, 99% or 100% sequence identity with SEQ ID NO:112. In some of these examples, the antibody or antigen-binding fragment thereof is a humanized IgG1 antibody.

In some other of these preferred embodiments, the radionuclide is ⁸⁹Zr, the chelator comprises 3,2-HOPO, or a derivative thereof and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:104, SEQ ID NO:105, SEQ ID NO:106, SEQ ID NO:108, SEQ ID NO:109, and SEQ ID NO:110 (TPP-17419). In some of these examples, the antibody furthermore comprises a variable heavy chain having at least 90%, 95%, 98%, 99% or 100% sequence identity with SEQ ID NO:103 and/or a variable light chain having at least 90%, 95%, 98%, 99% or 100% sequence identity with SEQ ID NO:107. In some of the examples, the antibody or antigen-binding fragment thereof is a Fab′, F(ab′)2, Fab, Fv, rIgG, or scFv fragment.

In some examples, the antibody furthermore comprises a heavy chain region having at least 90%, 95%, 98%, 99% or 100% sequence identity with SEQ ID NO:111 and/or a light chain region having at least 90%, 95%, 98%, 99% or 100% sequence identity with SEQ ID NO:112. In some of these examples, the antibody or antigen-binding fragment thereof is a humanized IgG1 antibody.

### TPP-17421

In some other of these preferred embodiments, the radionuclide is ⁸⁹Zr, the chelator comprises Me-3,2-HOPO, or a derivative thereof and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:114, SEQ ID NO:115, SEQ ID NO:116, SEQ ID NO:118, SEQ ID NO:119, and SEQ ID NO:120 (TPP-17421). In some of these examples, the antibody furthermore comprises a variable heavy chain having at least 90%, 95%, 98%, 99% or 100% sequence identity with SEQ ID NO:113 and/or a variable light chain having at least 90%, 95%, 98%, 99% or 100% sequence identity with SEQ ID NO:117. In some of the examples, the antibody or antigen-binding fragment thereof is a Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:123 and/or a light chain region having at least 90%, 95%, 98%, 99% or 100% sequence identity with SEQ ID NO:124. In some of these examples, the antibody or antigen-binding fragment thereof is a humanized IgG1 antibody.

In some other of these preferred embodiments, the radionuclide is ⁸⁹Zr, the chelator comprises a chelator according to formula I or a derivative thereof, and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:114, SEQ ID NO:115, SEQ ID NO:116, SEQ ID NO:118, SEQ ID NO:119, and SEQ ID NO:120 (TPP-17421). In some of these examples, the antibody furthermore comprises a variable heavy chain having at least 90%, 95%, 98%, 99% or 100% sequence identity with SEQ ID NO:113 and/or a variable light chain having at least 90%, 95%, 98%, 99% or 100% sequence identity with SEQ ID NO:117. In some of the examples, the antibody or antigen-binding fragment thereof is a Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:123 and/or a light chain region having at least 90%, 95%, 98%, 99% or 100% sequence identity with SEQ ID NO:124. In some of these examples, the antibody or antigen-binding fragment thereof is a humanized IgG1 antibody.

In some other of these preferred embodiments, the radionuclide is ⁸⁹Zr, the chelator comprises 3,2-HOPO, or a derivative thereof and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:114, SEQ ID NO:115, SEQ ID NO:116, SEQ ID NO:118, SEQ ID NO:119, and SEQ ID NO:120 (TPP-17421). In some of these examples, the antibody furthermore comprises a variable heavy chain having at least 90%, 95%, 98%, 99% or 100% sequence identity with SEQ ID NO:113 and/or a variable light chain having at least 90%, 95%, 98%, 99% or 100% sequence identity with SEQ ID NO:117. In some of the examples, the antibody or antigen-binding fragment thereof is a Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:123 and/or a light chain region having at least 90%, 95%, 98%, 99% or 100% sequence identity with SEQ ID NO:124. In some of these examples, the antibody or antigen-binding fragment thereof is a humanized IgG1 antibody.

### TPP-17422

In some other of these preferred embodiments, the radionuclide is ⁸⁹Zr, the chelator comprises Me-3,2-HOPO, or a derivative thereof and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:128, SEQ ID NO:129, SEQ ID NO:130, SEQ ID NO:132, SEQ ID NO:133, and SEQ ID NO:134 (TPP-17422). In some of these examples, the antibody furthermore comprises a variable heavy chain having at least 90%, 95%, 98%, 99% or 100% sequence identity with SEQ ID NO:127 and/or a variable light chain having at least 90%, 95%, 98%, 99% or 100% sequence identity with SEQ ID NO:131. In some of the examples, the antibody or antigen-binding fragment thereof is a Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:135 and/or a light chain region having at least 90%, 95%, 98%, 99% or 100% sequence identity with SEQ ID NO:136. In some of these examples, the antibody or antigen-binding fragment thereof is a humanized IgG1 antibody.

In some other of these preferred embodiments, the radionuclide is ⁸⁹Zr, the chelator comprises a chelator according to formula I or a derivative thereof, and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:128, SEQ ID NO:129, SEQ ID NO:130, SEQ ID NO:132, SEQ ID NO:133, and SEQ ID NO:134 (TPP-17422). In some of these examples, the antibody furthermore comprises a variable heavy chain having at least 90%, 95%, 98%, 99% or 100% sequence identity with SEQ ID NO:127 and/or a variable light chain having at least 90%, 95%, 98%, 99% or 100% sequence identity with SEQ ID NO:131. In some of the examples, the antibody or antigen-binding fragment thereof is a Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:135 and/or a light chain region having at least 90%, 95%, 98%, 99% or 100% sequence identity with SEQ ID NO:136. In some of these examples, the antibody or antigen-binding fragment thereof is a humanized IgG1 antibody.

In some other of these preferred embodiments, the radionuclide is ⁸⁹Zr, the chelator comprises 3,2-HOPO, or a derivative thereof and the targeting moiety binding to human LRRC15 is an antibody or antigen-binding fragment thereof, e.g. comprising at least one, two, three, four, five and preferably six CDR sequences having at least 90%, 95%, 98%, 99% or 100% sequence identity with at least one of SEQ ID NO:128, SEQ ID NO:129, SEQ ID NO:130, SEQ ID NO:132, SEQ ID NO:133, and SEQ ID NO:134 (TPP-17422). In some of these examples, the antibody furthermore comprises a variable heavy chain having at least 90%, 95%, 98%, 99% or 100% sequence identity with SEQ ID NO:127 and/or a variable light chain having at least 90%, 95%, 98%, 99% or 100% sequence identity with SEQ ID NO:131. In some of the examples, the antibody or antigen-binding fragment thereof is a Fab′, F(ab′)2, Fab, Fv, rIgG, or scFv fragment. In some examples, the antibody furthermore comprises a heavy chain region having at least 90%, 95%, 98%, 99% or 100% sequence identity with SEQ ID NO:135 and/or a light chain region having at least 90%, 95%, 98%, 99% or 100% sequence identity with SEQ ID NO:136. In some of these examples, the antibody or antigen-binding fragment thereof is a humanized IgG1 antibody.

FIG. 27 shows some exemplary embodiments of the current aspect, wherein the radionuclide is ²²⁷Th or ⁸⁹Zr, wherein the chelator furthermore comprises 3,2 HOPO and wherein the targeting moiety binding to human LRRC15 is an antibody or antigen-binding fragment thereof. Further embodiments are described within the example section.

### LRRC15 Antibodies Defined by Sequence

According to a second aspect of the current invention, there is provided an isolated antibody or antigen-binding fragment thereof binding to LRRC15.

The antibody can be, for example, an IgG antibody, such as a human IgG1, IgG2, IgG3, or IgG4, or a mouse IgG1, IgG2a, IgG2b or IgG2c. In some highly preferred embodiments, the isolated antibody or antigen-binding fragment thereof according to the current aspect is a human or humanized IgG1 antibody. In some further preferred embodiments, the antibody or antigen-binding fragment thereof according to the current aspect is a Fab′, F(ab′)2, Fab, Fv, rIgG, or scFv fragment.

If not explicitly stated otherwise, the LRRC15 may be from any species e.g. human, monkey, Macaca fascicularis (cynomolgus monkey), Macaca mulatta (rhesus macaque), rodent, mouse, rat, horse, bovine, pig, dog, cat and camel LRRC15.

In some preferred embodiments, the isolated antibody or antigen-binding fragment thereof according to the current aspect is binding to human LRRC15 and/or cynomolgus LRRC15 and/or murine LRRC15. In some of these preferred embodiments, the isolated antibody or antigen-binding fragment thereof according to the current aspect is binding to human LRRC15 and/or cynomolgus LRRC15.

According to some preferred embodiments, the antibody or antigen-binding fragment has a binding affinity KD for human LRRC15 which is below 2E-07 M⁻¹, preferably below 1E-08 M⁻¹, 1E-09 M⁻¹, 10E-10 M⁻¹ or 1E-10 M⁻¹. According to some embodiments, which may be the same or different, the antibody or antigen-binding fragment has a binding affinity KD to cynomolgus LRRC15 which is below 2E-07 M⁻¹, preferably below 1E-08 M⁻¹, 1E-09 M⁻¹, 10E-10 M⁻¹ or 1E-10 M⁻¹. According to some embodiments, which may be the same or different, the antibody or antigen-binding fragment has a binding affinity KD to murine LRRC15 which is below 2E-07 M⁻¹, preferably below 1E-08 M⁻¹, 1E-09 M⁻¹, 10E-10 M⁻¹ or 1E-10 M⁻¹.

Preferably, the antibody or antigen-binding fragment binds human and cynomolgus LRRC15, and optionally murine LRRC15 with a KD in the same order of magnitude, e.g. with a KD below 2E-07 M⁻¹, 1E-08 M⁻¹, 1E-09 M⁻¹, 10E-10 M⁻¹ or 1E-10 M⁻¹.

As will be appreciated by skilled artisans, antibodies and/or binding fragments are “modular” in nature. Throughout the disclosure, various specific aspects and embodiments of the various “modules” composing the antibodies and/or binding fragments are described. As specific non-limiting examples, various specific embodiments of VH CDRs, VH chains, VL CDRs and VL chains are described. It is intended that all of the specific embodiments may be combined with each other as though each specific combination were explicitly described individually. As specific non-limiting examples, various specific functional embodiments are described. It is intended that all of the specific embodiments may be combined with each other as though each specific combination were explicitly described individually.

Furthermore, each of the antibodies or antigen-binding fragments described according to the current aspect can be and is suggested to be used as targeting moiety for a conjugate according to the first aspect or for a conjugate according to the third aspect. Furthermore, in some most preferred embodiments, the antibody is a bispecific antibody.

### TPP-1633

In some preferred embodiments, the antibody or antigen-binding fragment thereof comprises at least one, two, three, four, five and preferably six CDR sequences having at least 90%, 95%, 98%, 99% or 100% sequence identity with at least one of SEQ ID NO:2, SEQ ID NO:3, SEQ ID NO:4, SEQ ID NO:6, SEQ ID NO:7 and SEQ ID NO:8 (TPP-1633). In some of these examples, the antibody comprises a variable heavy chain having at least 90%, 95%, 98%, 99% or 100% sequence identity with SEQ ID NO:1 and/or a variable light chain having at least 90%, 95%, 98%, 99% or 100% sequence identity with SEQ ID NO:5. In some of the examples, the antibody or antigen-binding fragment thereof is a human Fab′, F(ab′)2, Fab, Fv, rIgG, or scFv fragment. In some examples, the antibody comprises a heavy chain region having at least 90%, 95%, 98%, 99% or 100% sequence identity with SEQ ID NO:9 and/or a light chain region having at least 90%, 95%, 98%, 99% or 100% sequence identity with SEQ ID NO:10. In some of these examples, the antibody or antigen-binding fragment thereof is a human IgG1 antibody. In some most preferred embodiments, the antibody is a bispecific antibody.

### TPP-14389

In some preferred embodiments, the antibody or antigen-binding fragment thereof comprises at least one, two, three, four, five and preferably six CDR sequences having at least 90%, 95%, 98%, 99% or 100% sequence identity with at least one of SEQ ID NO:22, SEQ ID NO:23, SEQ ID NO:24, SEQ ID NO:26, SEQ ID NO:27 and SEQ ID NO:28 (TPP-14389). In some of these examples, the antibody comprises a variable heavy chain having at least 90%, 95%, 98%, 99% or 100% sequence identity with SEQ ID NO:21 and/or a variable light chain having at least 90%, 95%, 98%, 99% or 100% sequence identity with SEQ ID NO:25. In some of the examples, the antibody or antigen-binding fragment thereof is a human Fab′, F(ab′)2, Fab, Fv, rIgG, or scFv fragment. In some examples, the antibody comprises a heavy chain region having at least 90%, 95%, 98%, 99% or 100% sequence identity with SEQ ID NO:31 and/or a light chain region having at least 90%, 95%, 98%, 99% or 100% sequence identity with SEQ ID NO:32. In some of these examples, the antibody or antigen-binding fragment thereof is a human IgG1 antibody. In some most preferred embodiments, the antibody is a bispecific antibody.

### TPP-14392

In some preferred embodiments, the antibody or antigen-binding fragment thereof comprises at least one, two, three, four, five and preferably six CDR sequences having at least 90%, 95%, 98%, 99% or 100% sequence identity with at least one of SEQ ID NO:36, SEQ ID NO:37, SEQ ID NO:38, SEQ ID NO:40, SEQ ID NO:41 and SEQ ID NO:42 (e.g. TPP-14392). In some of these examples, the antibody comprises a variable heavy chain having at least 90%, 95%, 98%, 99% or 100% sequence identity with SEQ ID NO:35 and/or a variable light chain having at least 90%, 95%, 98%, 99% or 100% sequence identity with SEQ ID NO:39. In some of the examples, the antibody or antigen-binding fragment thereof is a human Fab′, F(ab′)2, Fab, Fv, rIgG, or scFv fragment. In some examples, the antibody comprises a heavy chain region having at least 90%, 95%, 98%, 99% or 100% sequence identity with SEQ ID NO:45 and/or a light chain region having at least 90%, 95%, 98%, 99% or 100% sequence identity with SEQ ID NO:46. In some of these examples, the antibody or antigen-binding fragment thereof is a human IgG1 antibody. In some most preferred embodiments, the antibody is a bispecific antibody.

### TPP-17073

In some preferred embodiments, the antibody or antigen-binding fragment thereof comprises at least one, two, three, four, five and preferably six CDR sequences having at least 90%, 95%, 98%, 99% or 100% sequence identity with at least one of SEQ ID NO:50, SEQ ID NO:51, SEQ ID NO:52, SEQ ID NO:54, SEQ ID NO:55, and SEQ ID NO:56 (TPP-17073). In some of these examples, the antibody comprises a variable heavy chain having at least 90%, 95%, 98%, 99% or 100% sequence identity with SEQ ID NO:49 and/or a variable light chain having at least 90%, 95%, 98%, 99% or 100% sequence identity with SEQ ID NO:53. In some of the examples, the antibody or antigen-binding fragment thereof is a human Fab′, F(ab′)2, Fab, Fv, rIgG, or scFv fragment. In some examples, the antibody comprises a heavy chain region having at least 90%, 95%, 98%, 99% or 100% sequence identity with SEQ ID NO:57 and/or a light chain region having at least 90%, 95%, 98%, 99% or 100% sequence identity with SEQ ID NO:58. In some of these examples, the antibody or antigen-binding fragment thereof is a human IgG1 antibody. In some most preferred embodiments, the antibody is a bispecific antibody.

### TPP-17074

In some preferred embodiments, the antibody or antigen-binding fragment thereof comprises at least one, two, three, four, five and preferably six CDR sequences having at least 90%, 95%, 98%, 99% or 100% sequence identity with at least one of SEQ ID NO:60, SEQ ID NO:61, SEQ ID NO:62, SEQ ID NO:64, SEQ ID NO:65, and SEQ ID NO:66 (TPP-17074). In some of these examples, the antibody comprises a variable heavy chain having at least 90%, 95%, 98%, 99% or 100% sequence identity with SEQ ID NO:59 and/or a variable light chain having at least 90%, 95%, 98%, 99% or 100% sequence identity with SEQ ID NO:63. In some of the examples, the antibody or antigen-binding fragment thereof is a human Fab′, F(ab′)2, Fab, Fv, rIgG, or scFv fragment. In some examples, the antibody comprises a heavy chain region having at least 90%, 95%, 98%, 99% or 100% sequence identity with SEQ ID NO:67 and/or a light chain region having at least 90%, 95%, 98%, 99% or 100% sequence identity with SEQ ID NO:68. In some of these examples, the antibody or antigen-binding fragment thereof is a human IgG1 antibody. In some most preferred embodiments, the antibody is a bispecific antibody.

### TPP-17078

In some preferred embodiments, the antibody or antigen-binding fragment thereof comprises at least one, two, three, four, five and preferably six CDR sequences having at least 90%, 95%, 98%, 99% or 100% sequence identity with at least one of SEQ ID NO:70, SEQ ID NO:71, SEQ ID NO:72, SEQ ID NO:74, SEQ ID NO:75, and SEQ ID NO:76 (TPP-17078). In some of these examples, the antibody comprises a variable heavy chain having at least 90%, 95%, 98%, 99% or 100% sequence identity with SEQ ID NO:69 and/or a variable light chain having at least 90%, 95%, 98%, 99% or 100% sequence identity with SEQ ID NO:73. In some of the examples, the antibody or antigen-binding fragment thereof is a human Fab′, F(ab′)2, Fab, Fv, rIgG, or scFv fragment. In some examples, the antibody comprises a heavy chain region having at least 90%, 95%, 98%, 99% or 100% sequence identity with SEQ ID NO:79 and/or a light chain region having at least 90%, 95%, 98%, 99% or 100% sequence identity with SEQ ID NO:80. In some of these examples, the antibody or antigen-binding fragment thereof is a human IgG1 antibody. In some most preferred embodiments, the antibody is a bispecific antibody.

### TPP-17405

In some preferred embodiments, the antibody or antigen-binding fragment thereof comprises at least one, two, three, four, five and preferably six CDR sequences having at least 90%, 95%, 98%, 99% or 100% sequence identity with at least one of SEQ ID NO:84, SEQ ID NO:85, SEQ ID NO:86, SEQ ID NO:88, SEQ ID NO:89, and SEQ ID NO:90 (TPP-17405). In some of these examples, the antibody comprises a variable heavy chain having at least 90%, 95%, 98%, 99% or 100% sequence identity with SEQ ID NO:83 and/or a variable light chain having at least 90%, 95%, 98%, 99% or 100% sequence identity with SEQ ID NO:87. In some of the examples, the antibody or antigen-binding fragment thereof is a Fab′, F(ab′)2, Fab, Fv, rIgG, or scFv fragment. In some examples, the antibody comprises a heavy chain region having at least 90%, 95%, 98%, 99% or 100% sequence identity with SEQ ID NO:91 and/or a light chain region having at least 90%, 95%, 98%, 99% or 100% sequence identity with SEQ ID NO:92. In some of these examples, the antibody or antigen-binding fragment thereof is a humanized IgG1 antibody. In some most preferred embodiments, the antibody is a bispecific antibody.

### TPP-17418

In some preferred embodiments, the antibody or antigen-binding fragment thereof comprises at least one, two, three, four, five and preferably six CDR sequences having at least 90%, 95%, 98%, 99% or 100% sequence identity with at least one of SEQ ID NO:94, SEQ ID NO:95, SEQ ID NO:96, SEQ ID NO:98, SEQ ID NO:99, and SEQ ID NO:100 (TPP-17418). In some of these examples, the antibody comprises a variable heavy chain having at least 90%, 95%, 98%, 99% or 100% sequence identity with SEQ ID NO:93 and/or a variable light chain having at least 90%, 95%, 98%, 99% or 100% sequence identity with SEQ ID NO:97. In some of the examples, the antibody or antigen-binding fragment thereof is a Fab′, F(ab′)2, Fab, Fv, rIgG, or scFv fragment. In some examples, the antibody comprises heavy chain region having at least 90%, 95%, 98%, 99% or 100% sequence identity with SEQ ID NO:101 and/or a light chain region having at least 90%, 95%, 98%, 99% or 100% sequence identity with SEQ ID NO:102. In some of these examples, the antibody or antigen-binding fragment thereof is a humanized IgG1 antibody. In some most preferred embodiments, the antibody is a bispecific antibody.

### TPP-17419

In some preferred embodiments, the antibody or antigen-binding fragment thereof comprises at least one, two, three, four, five and preferably six CDR sequences having at least 90%, 95%, 98%, 99% or 100% sequence identity with at least one of SEQ ID NO:104, SEQ ID NO:105, SEQ ID NO:106, SEQ ID NO:108, SEQ ID NO:109, and SEQ ID NO:110 (TPP-17419). In some of these examples, the antibody comprises a variable heavy chain having at least 90%, 95%, 98%, 99% or 100% sequence identity with SEQ ID NO:103 and/or a variable light chain having at least 90%, 95%, 98%, 99% or 100% sequence identity with SEQ ID NO:107. In some of the examples, the antibody or antigen-binding fragment thereof is a Fab′, F(ab′)2, Fab, Fv, rIgG, or scFv fragment. In some examples, the antibody comprises a heavy chain region having at least 90%, 95%, 98%, 99% or 100% sequence identity with SEQ ID NO:111 and/or a light chain region having at least 90%, 95%, 98%, 99% or 100% sequence identity with SEQ ID NO:112. In some of these examples, the antibody or antigen-binding fragment thereof is a humanized IgG1 antibody. In some most preferred embodiments, the antibody is a bispecific antibody.

### TPP-17421

In some preferred embodiments, the antibody or antigen-binding fragment thereof comprises at least one, two, three, four, five and preferably six CDR sequences having at least 90%, 95%, 98%, 99% or 100% sequence identity with at least one of SEQ ID NO:114, SEQ ID NO:115, SEQ ID NO:116, SEQ ID NO:118, SEQ ID NO:119, and SEQ ID NO:120 (TPP-17421). In some of these examples, the antibody comprises a variable heavy chain having at least 90%, 95%, 98%, 99% or 100% sequence identity with SEQ ID NO:113 and/or a variable light chain having at least 90%, 95%, 98%, 99% or 100% sequence identity with SEQ ID NO:117. In some of the examples, the antibody or antigen-binding fragment thereof is a Fab′, F(ab′)2, Fab, Fv, rIgG, or scFv fragment. In some examples, the antibody comprises a heavy chain region having at least 90%, 95%, 98%, 99% or 100% sequence identity with SEQ ID NO:123 and/or a light chain region having at least 90%, 95%, 98%, 99% or 100% sequence identity with SEQ ID NO:124. In some of these examples, the antibody or antigen-binding fragment thereof is a humanized IgG1 antibody. In some most preferred embodiments, the antibody is a bispecific antibody.

### TPP-17422

In some preferred embodiments, the antibody or antigen-binding fragment thereof comprises at least one, two, three, four, five and preferably six CDR sequences having at least 90%, 95%, 98%, 99% or 100% sequence identity with at least one of SEQ ID NO:128, SEQ ID NO:129, SEQ ID NO:130, SEQ ID NO:132, SEQ ID NO:133, and SEQ ID NO:134 (TPP-17422). In some of these examples, the antibody comprises a variable heavy chain having at least 90%, 95%, 98%, 99% or 100% sequence identity with SEQ ID NO:127 and/or a variable light chain having at least 90%, 95%, 98%, 99% or 100% sequence identity with SEQ ID NO:131. In some of the examples, the antibody or antigen-binding fragment thereof is a Fab′, F(ab′)2, Fab, Fv, rIgG, or scFv fragment. In some examples, the antibody comprises a heavy chain region having at least 90%, 95%, 98%, 99% or 100% sequence identity with SEQ ID NO:135 and/or a light chain region having at least 90%, 95%, 98%, 99% or 100% sequence identity with SEQ ID NO:136. In some of these examples, the antibody or antigen-binding fragment thereof is a humanized IgG1 antibody. In some most preferred embodiments, the antibody is a bispecific antibody.

In some highly preferred embodiments, the isolated antibody or antigen-binding fragment according to the current aspect comprises at least one, two, three, four, five and preferably six CDR sequences, wherein each of said CDR sequences has at least 90%, 95%, 98%, 99% or 100% sequence identity with one or more of
a) SEQ ID NO:2, SEQ ID NO:3, SEQ ID NO:4, SEQ ID NO:6, SEQ ID NO:7 and SEQ ID NO:8 (TPP-1633), or
b) SEQ ID NO:22, SEQ ID NO:23, SEQ ID NO:24, SEQ ID NO:26, SEQ ID NO:27 and SEQ ID NO:28 (TPP-14389), or
c) SEQ ID NO:36, SEQ ID NO:37, SEQ ID NO:38, SEQ ID NO:40, SEQ ID NO:41 and SEQ ID NO:42 (TPP-14392), or
d) SEQ ID NO:50, SEQ ID NO:51, SEQ ID NO:52, SEQ ID NO:54, SEQ ID NO:55, and SEQ ID NO:56 (TPP-17073), or
e) SEQ ID NO:60, SEQ ID NO:61, SEQ ID NO:62, SEQ ID NO:64, SEQ ID NO:65, and SEQ ID NO:66 (TPP-17074), or
f) SEQ ID NO:70, SEQ ID NO:71, SEQ ID NO:72, SEQ ID NO:74, SEQ ID NO:75, and SEQ ID NO:76 (TPP-17078), or
g) SEQ ID NO:84, SEQ ID NO:85, SEQ ID NO:86, SEQ ID NO:88, SEQ ID NO:89, and SEQ ID NO:90 (TPP-17405), or
h) SEQ ID NO:94, SEQ ID NO:95, SEQ ID NO:96, SEQ ID NO:98, SEQ ID NO:99, and SEQ ID NO:100 (TPP-17418), or
i) SEQ ID NO:104, SEQ ID NO:105, SEQ ID NO:106, SEQ ID NO:108, SEQ ID NO:109, and SEQ ID NO:110 (TPP-17419), or
j) SEQ ID NO:114, SEQ ID NO:115, SEQ ID NO:116, SEQ ID NO:118, SEQ ID NO:119, and SEQ ID NO:120 (TPP-17421), or
k) SEQ ID NO:128, SEQ ID NO:129, SEQ ID NO:130, SEQ ID NO:132, SEQ ID NO:133, and SEQ ID NO:134 (TPP-17422).

In some of these embodiments, the antibody or antigen-binding fragment thereof is a Fab′, F(ab′)2, Fab, Fv, rIgG, or scFv fragment. In some embodiments, the antibody or antigen-binding fragment thereof is an IgG antibody, preferably a human or humanized IgG1 binding human LRRC15. In some most preferred embodiments, the antibody is a bispecific antibody.

In some highly preferred embodiments, the isolated antibody or antigen-binding fragment thereof comprises at least one, two, three, four, five and preferably six CDR sequences according to
a) SEQ ID NO:2, SEQ ID NO:3, SEQ ID NO:4, SEQ ID NO:6, SEQ ID NO:7 and SEQ ID NO:8 (TPP-1633), or
b) SEQ ID NO:22, SEQ ID NO:23, SEQ ID NO:24, SEQ ID NO:26, SEQ ID NO:27 and SEQ ID NO:28 (TPP-14389), or
c) SEQ ID NO:36, SEQ ID NO:37, SEQ ID NO:38, SEQ ID NO:40, SEQ ID NO:41 and SEQ ID NO:42 (TPP-14392), or
d) SEQ ID NO:50, SEQ ID NO:51, SEQ ID NO:52, SEQ ID NO:54, SEQ ID NO:55, and SEQ ID NO:56 (TPP-17073), or
e) SEQ ID NO:60, SEQ ID NO:61, SEQ ID NO:62, SEQ ID NO:64, SEQ ID NO:65, and SEQ ID NO:66 (TPP-17074), or
f) SEQ ID NO:70, SEQ ID NO:71, SEQ ID NO:72, SEQ ID NO:74, SEQ ID NO:75, and SEQ ID NO:76 (TPP-17078), or
g) SEQ ID NO:84, SEQ ID NO:85, SEQ ID NO:86, SEQ ID NO:88, SEQ ID NO:89, and SEQ ID NO:90 (TPP-17405), or
h) SEQ ID NO:94, SEQ ID NO:95, SEQ ID NO:96, SEQ ID NO:98, SEQ ID NO:99, and SEQ ID NO:100 (TPP-17418), or
i) SEQ ID NO:104, SEQ ID NO:105, SEQ ID NO:106, SEQ ID NO:108, SEQ ID NO:109, and SEQ ID NO:110 (TPP-17419), or
j) SEQ ID NO:114, SEQ ID NO:115, SEQ ID NO:116, SEQ ID NO:118, SEQ ID NO:119, and SEQ ID NO:120 (TPP-17421), or
k) SEQ ID NO:128, SEQ ID NO:129, SEQ ID NO:130, SEQ ID NO:132, SEQ ID NO:133, and SEQ ID NO:134 (TPP-17422).

In some most preferred embodiments, the antibody is a bispecific antibody.

In some highly preferred embodiments, the isolated antibody or antigen-binding fragment thereof comprises at least
a) a variable heavy chain having at least 90%, 95%, 98%, 99% or 100% sequence identity with SEQ ID NO:1 and/or
   a variable light chain having at least 90%, 95%, 98%, 99% or 100% sequence identity with SEQ ID NO:5 (TPP-1633), or
b) a variable heavy chain having at least 90%, 95%, 98%, 99% or 100% sequence identity with SEQ ID NO:21 and/or
   a variable light chain having at least 90%, 95%, 98%, 99% or 100% sequence identity with SEQ ID NO:25 (TPP-14389), or
c) a variable heavy chain having at least 90%, 95%, 98%, 99% or 100% sequence identity with SEQ ID NO:35 and/or
   a variable light chain having at least 90%, 95%, 98%, 99% or 100% sequence identity with SEQ ID NO:39 (TPP-14392), or
d) a variable heavy chain having at least 90%, 95%, 98%, 99% or 100% sequence identity with SEQ ID NO:49 and/or
   a variable light chain having at least 90%, 95%, 98%, 99% or 100% sequence identity with SEQ ID NO:53 (TPP-17073), or
e) a variable heavy chain having at least 90%, 95%, 98%, 99% or 100% sequence identity with SEQ ID NO:59 and/or
   a variable light chain having at least 90%, 95%, 98%, 99% or 100% sequence identity with SEQ ID NO:63 (TPP-17074), or
f) a variable heavy chain having at least 90%, 95%, 98%, 99% or 100% sequence identity with SEQ ID NO:69 and/or
   a variable light chain having at least 90%, 95%, 98%, 99% or 100% sequence identity with SEQ ID NO:73 (TPP-17078), or
g) a variable heavy chain having at least 90%, 95%, 98%, 99% or 100% sequence identity with SEQ ID NO:83 and/or
   a variable light chain having at least 90%, 95%, 98%, 99% or 100% sequence identity with SEQ ID NO:87 (TPP-17405), or
h) a variable heavy chain having at least 90%, 95%, 98%, 99% or 100% sequence identity with SEQ ID NO:93 and/or
   a variable light chain having at least 90%, 95%, 98%, 99% or 100% sequence identity with SEQ ID NO:97 (TPP-17418), or
i) a variable heavy chain having at least 90%, 95%, 98%, 99% or 100% sequence identity with SEQ ID NO:103 and/or
   a variable light chain having at least 90%, 95%, 98%, 99% or 100% sequence identity with SEQ ID NO:107 (TPP-17419), or
j) a variable heavy chain having at least 90%, 95%, 98%, 99% or 100% sequence identity with SEQ ID NO:113 and/or
   a variable light chain having at least 90%, 95%, 98%, 99% or 100% sequence identity with SEQ ID NO:117 (TPP-17421), or
k) a variable heavy chain having at least 90%, 95%, 98%, 99% or 100% sequence identity with SEQ ID NO:127 and/or
   a variable light chain having at least 90%, 95%, 98%, 99% or 100% sequence identity with SEQ ID NO:131 (TPP-17422).

In some of these embodiments, the antibody or antigen-binding fragment thereof is a Fab′, F(ab′)2, Fab, Fv, rIgG, or scFv fragment. In some embodiments, the antibody or antigen-binding fragment thereof is an IgG antibody, preferably a human or humanized IgG1 binding human LRRC15. In some most preferred embodiments, the antibody is a bispecific antibody.

In some highly preferred embodiments, the isolated antibody or antigen-binding fragment thereof comprises at least
a) a variable heavy chain sequence according to SEQ ID NO:1 and/or
   a variable light chain sequence according to SEQ ID NO:5 (TPP-1633), or
b) a variable heavy chain sequence according to SEQ ID NO:21 and/or
   a variable light chain sequence according to SEQ ID NO:25 (TPP-14389), or
c) a variable heavy chain sequence according to SEQ ID NO:35 and/or
   a variable light chain sequence according to SEQ ID NO:39 (TPP-14392), or
d) a variable heavy chain sequence according to SEQ ID NO:49 and/or
   a variable light chain sequence according to SEQ ID NO:53 (TPP-17073), or
e) a variable heavy chain sequence according to SEQ ID NO:59 and/or
   a variable light chain sequence according to SEQ ID NO:63 (TPP-17074), or
f) a variable heavy chain sequence according to SEQ ID NO:69 and/or
   a variable light chain sequence according to SEQ ID NO:73 (TPP-17078), or
g) a variable heavy chain sequence according to SEQ ID NO:83 and/or
   a variable light chain sequence according to SEQ ID NO:87 (TPP-17405), or
h) a variable heavy chain sequence according to SEQ ID NO:93 and/or
   a variable light chain sequence according to SEQ ID NO:97 (TPP-17418), or
i) a variable heavy chain sequence according to SEQ ID NO:103 and/or
   a variable light chain sequence according to SEQ ID NO:107 (TPP-17419), or
j) a variable heavy chain sequence according to SEQ ID NO:113 and/or
   a variable light chain sequence according to SEQ ID NO:117 (TPP-17421), or
k) a variable heavy chain sequence according to SEQ ID NO:127 and/or
   a variable light chain sequence according to SEQ ID NO:131 (TPP-17422).

In some highly preferred embodiments, the isolated antibody or antigen-binding fragment thereof comprises at least
a) a heavy chain region having at least 90%, 95%, 98%, 99% or 100% sequence identity with SEQ ID NO:9 and/or
   a light chain region having at least 90%, 95%, 98%, 99% or 100% sequence identity with SEQ ID NO:10 (TPP-1633), or
b) a heavy chain region having at least 90%, 95%, 98%, 99% or 100% sequence identity with SEQ ID NO:31 and/or
   a light chain region having at least 90%, 95%, 98%, 99% or 100% sequence identity with SEQ ID NO:32 (TPP-14389), or
c) a heavy chain region having at least 90%, 95%, 98%, 99% or 100% sequence identity with SEQ ID NO:45 and/or
   a light chain region having at least 90%, 95%, 98%, 99% or 100% sequence identity with SEQ ID NO:46 (TPP-14392), or
d) a heavy chain region having at least 90%, 95%, 98%, 99% or 100% sequence identity with SEQ ID NO:57 and/or
   a light chain region having at least 90%, 95%, 98%, 99% or 100% sequence identity with SEQ ID NO:58 (TPP-17073), or
e) a heavy chain region having at least 90%, 95%, 98%, 99% or 100% sequence identity with SEQ ID NO:67 and/or
   a light chain region having at least 90%, 95%, 98%, 99% or 100% sequence identity with SEQ ID NO:68 (TPP-17074), or
f) a heavy chain region having at least 90%, 95%, 98%, 99% or 100% sequence identity with SEQ ID NO:79 and/or
   a light chain region having at least 90%, 95%, 98%, 99% or 100% sequence identity with SEQ ID NO:80 (TPP-17078), or
g) a heavy chain region having at least 90%, 95%, 98%, 99% or 100% sequence identity with SEQ ID NO:91 and/or
   a light chain region having at least 90%, 95%, 98%, 99% or 100% sequence identity with SEQ ID NO:92 (TPP-17405), or
h) a heavy chain region having at least 90%, 95%, 98%, 99% or 100% sequence identity with SEQ ID NO:101 and/or
   a light chain region having at least 90%, 95%, 98%, 99% or 100% sequence identity with SEQ ID NO:102 (TPP-17418), or
i) a heavy chain region having at least 90%, 95%, 98%, 99% or 100% sequence identity with SEQ ID NO:111 and/or
   a light chain region having at least 90%, 95%, 98%, 99% or 100% sequence identity with SEQ ID NO:112 (TPP-17419), or
j) a heavy chain region having at least 90%, 95%, 98%, 99% or 100% sequence identity with SEQ ID NO:123 and/or
   a light chain region having at least 90%, 95%, 98%, 99% or 100% sequence identity with SEQ ID NO:124 (TPP-17421), or
k) a heavy chain region having at least 90%, 95%, 98%, 99% or 100% sequence identity with SEQ ID NO:135 and/or
   a light chain region having at least 90%, 95%, 98%, 99% or 100% sequence identity with SEQ ID NO:136 (TPP-17422).

In some of these embodiments, the antibody or antigen-binding fragment thereof is a Fab′, F(ab′)2, Fab, Fv, rIgG, or scFv fragment. In some embodiments, the antibody or antigen-binding fragment thereof is an IgG antibody, preferably a human or humanized IgG1 binding human LRRC15. In some most preferred embodiments, the antibody is a bispecific antibody.

In some highly preferred embodiments, the isolated antibody or antigen-binding fragment thereof comprises at least
a) a heavy chain region according to SEQ ID NO:9 and/or
   a light chain region according to SEQ ID NO:10 (TPP-1633), or
b) a heavy chain region according to SEQ ID NO:31 and/or
   a light chain region according to SEQ ID NO:32 (TPP-14389), or
c) a heavy chain region according to SEQ ID NO:45 and/or
   a light chain region according to SEQ ID NO:46 (TPP-14392), or
d) a heavy chain region according to SEQ ID NO:57 and/or
   a light chain region according to SEQ ID NO:58 (TPP-17073), or
e) a heavy chain region according to SEQ ID NO:67 and/or
   a light chain region according to SEQ ID NO:68 (TPP-17074), or
f) a heavy chain region according to SEQ ID NO:79 and/or
   a light chain region according to SEQ ID NO:80 (TPP-17078), or
g) a heavy chain region according to SEQ ID NO:91 and/or
   a light chain region according to SEQ ID NO:92 (TPP-17405), or
h) a heavy chain region according to SEQ ID NO:101 and/or
   a light chain region according to SEQ ID NO:102 (TPP-17418), or
i) a heavy chain region according to SEQ ID NO:111 and/or
   a light chain region according to SEQ ID NO:112 (TPP-17419), or
j) a heavy chain region according to SEQ ID NO:123 and/or
   a light chain region according to SEQ ID NO:124 (TPP-17421), or
k) a heavy chain region according to SEQ ID NO:135 and/or
   a light chain region according to SEQ ID NO:136 (TPP-17422).

According to some preferred embodiments of the current aspect, the temperature dependent loss in binding affinity to LRRC15 of the antibody or antigen-binding fragment is <10 for 37° C. relative to 25° C.

According to some preferred embodiments of the current aspect, the antibody or antigen-binding fragment is specific for LRRC15 and/or shows less polyreactivity. For example, the antibody or fragment according to the current invention does not bind EPHB6 or binds EPHB6 with a lower affinity than prior art antibody TPP-12942. According to some preferred embodiments of the current aspect, the antibody or antigen-binding fragment is characterized by a clearance rate in cynomolgus monkey <0.5 ml kg⁻¹ h⁻¹, even more preferably <0.4 ml kg⁻¹ h⁻¹, 0.3 ml kg⁻¹ h⁻¹, or 0.2 ml kg⁻¹ h⁻¹.

According to some preferred embodiments, the antibody or antigen-binding fragment comprises ≤15, 14, 13, 12, 11 or 10 germline deviations in the light chain and 10 germline deviations in the heavy chain, compared with the respective closest human germline.

According to some preferred embodiments, the antibody or antigen-binding fragment is stable at low pH, e.g. <pH 5. For example, incubation at pH 3.8 for 270 min yields >95%, 97%, 98%, 99% or 100% of intact antibody or fragment. All tested antibodies according to the current invention had a superior stability in downstream processing.

### Specific Antibody-Defined Conjugates

In addition to naked antibodies, various further antibody- or antibody fragment-based conjugates can be designed using the antibodies or antigen binding fragments disclosed herein. According to a third aspect of the current invention, there is provided a conjugate targeting LRRC15 comprising an antibody or antigen-binding fragment according to the second aspect. These conjugates may be conjugates for diagnosis, therapy, research applications, and various other purposes. Provided are for example antibodies or antigen binding fragments thereof conjugated to radionuclides, cytotoxic agents, organic compounds, protein toxins, immunomodulators such as cytokines, fluorescent moieties, cells, further antibodies or antigen binding fragments thereof.

The conjugates disclosed herein, e.g. antibody drug conjugates (ADCs), targeted thorium conjugates (TTCs), bispecific antibodies etc., are “modular” in nature. Throughout the disclosure, various specific embodiments of the various “modules” composing the conjugates are described. As specific non-limiting examples, specific embodiments of antibodies or fragments thereof, linkers, and cytotoxic and/or cytostatic agents that may compose the ADCs are described. It is intended that all of the specific embodiments described may be combined with each other as though each specific combination were explicitly described individually.

According to some preferred embodiments the conjugate comprises (a) a radioactive element, (b) a cytotoxic agent, such as an auristatin, a maytansinoid, a kinesin-spindle protein (KSP) inhibitor, a nicotinamide phosphoribosyltransferase (NAMPT) inhibitor or a pyrrolobenzodiazepine derivative, (c) a further antibody or antigen-binding fragment, or (d) a chimeric antigen receptor (CAR).

Preferably, the conjugate according to the current aspect comprises
a. an α-particle-emitting radionuclide such as ²At, ²¹²Pb, ²¹³Bi, ²²³Ra, ²²⁴Ra, ²²⁵Ac, or ²²⁷Th, and/or
b. a beta-particle-emitting radionuclide such as ⁶⁷Cu, ⁸⁹Sr, ⁸⁹Zr, ⁹⁰Y, ¹⁰⁵Rh, ¹³¹I, ¹⁴⁹Pm, ¹⁶⁶Ho, ¹⁷⁷Lu, ¹⁸⁶Re, ¹⁸⁸Re, ¹⁹⁸Au and/or
c. a cytotoxic agent, such as such as an auristatin, a maytansinoid, a kinesin-spindle protein inhibitor, a nicotinamide phosphoribosyltransferase inhibitor or a pyrrolobenzodiazepine derivative, and/or
d. a detectable moiety and/or
e. a CAR.

### Radioconjugates

According to some first embodiments of the 3rd aspect, the conjugate comprises a radionuclide, such as a beta particle, an alpha particle, a gamma particle or an Auger electron emitter. The conjugate targeting LRRC15 according to the first embodiments of the 3^{rd} aspect may or may not be or comprise a conjugate according to the first aspect.

For example, the radionuclide may be selected from the group consisting of of ⁴³Sc, ⁴⁴Sc, ⁴⁷Sc, ⁸⁹Zr, ⁹⁰Y, ¹¹¹In, ¹⁴⁹Tb, ¹⁵²Tb, ¹⁵⁵Tb, ¹⁶¹Tb, ¹⁶⁶Ho, ¹⁷⁷Lu, ¹⁸⁶Re, ¹⁸⁸Re, ²¹²Bi, ²¹³Bi, ²²⁵Ac, ²²⁷Th, and ²³²Th.

Suitable beta emitters according to the current invention are for example ⁶⁷Cu, ⁸⁹Sr, ⁸⁹Zr, ⁹⁰Y, ¹⁰⁵Rh, ¹³¹I, ¹⁴⁹Pm, ¹⁶⁶Ho, ¹⁷⁷Lu, ¹⁸⁶Re, ¹⁸⁸Re, ¹⁹⁸Au. In some preferred embodiments of the current invention, the radionuclide is ⁸⁹Zr.

Suitable Auger electron emitting radionuclides are for example ⁶⁷Ga, ⁷¹Ge, ⁷⁷Br, ^{99m}Tc, ¹⁰³Pd, ¹¹¹In, ¹²³I, ¹²⁵I, ¹⁴⁰Nd, ¹⁷⁸Ta, ¹⁹³Pt, ^{195m}Pt, ¹⁹⁷Hg.

Suitable alpha emitters, according to the current invention are for example ²¹¹At, ²¹²Pb, ²¹³Bi, ²²³Ra, ²²⁴Ra, ²²⁵Ac, or ²²⁷Th. In some most preferred embodiments, the radionuclide is an α-particle-emitting radionuclide such as ²²⁷Th.

In some highly preferred of the first embodiments of the 3rd aspect, the conjugate furthermore comprises a chelator or a synthetic group for immobilization of the radionuclide, e.g. as described elsewhere herein.

### ADCs

According to some second embodiments of the 3rd aspect, the conjugate comprises a cytotoxic agent, e.g. to form an antibody drug conjugate (ADC).

In some preferred embodiments, the cytotoxic agent is an auristatin, a maytansinoid, a kinesin-spindle protein (KSP) inhibitor, a nicotinamide phosphoribosyltransferase (NAMPT) inhibitor or a pyrrolobenzodiazepine derivative. Generation of conjugates comprising maytansinoid may occur as described in EP2424569 B1, incorporated herein in their entirety. Generation of conjugates comprising kinesin-spindle protein (KSP) inhibitors may occur as described in WO2019/243159 A1, incorporated herein in its entirety. Generation of conjugates comprising a nicotinamide phosphoribosyltransferase (NAMPT) inhibitor may occur as described in WO2019/149637 A1, incorporated herein in its entirety. Conjugates comprising a pyrrolobenzodiazepine may be obtained as described in EP3355935 A1, incorporated herein in its entirety.

The cytotoxic and/or cytostatic agent of the ADC may be any agent known to inhibit the growth and/or replication of, and/or kill cells. Numerous agents having cytotoxic and/or cytostatic properties are known in the literature. Non-limiting examples of classes of cytotoxic and/or cytostatic agents include, by way of example and not limitation, cell cycle modulators, apoptosis regulators, kinase inhibitors, protein synthesis inhibitors, alkylating agents, DNA cross-linking agents, intercalating agents, mitochondria inhibitors, nuclear export inhibitors, topoisomerase I inhibitors, topoisomerase II inhibitors, RNA/DNA antimetabolites and antimitotic agents.

The linkers linking the cytotoxic and/or cytostatic agent(s) to the targeting moiety of an ADC may be long, short, flexible, rigid, hydrophilic or hydrophobic in nature, or may comprise segments that have different characteristics, such as segments of flexibility, segments of rigidity, etc. The linker may be chemically stable to extracellular environments, for example, chemically stable in the blood stream, or may include linkages that are not stable and release the cytotoxic and/or cytostatic agents in the extracellular milieu. In some embodiments, the linkers include linkages that are designed to release the cytotoxic and/or cytostatic agents upon internalization of the LRRC15 targeting ADC, within the cell. In some specific embodiments, the linkers include linkages designed to cleave and/or immolate or otherwise breakdown specifically or non-specifically inside cells. A wide variety of linkers useful for linking drugs to antigen binding moieties such as antibodies in the context of ADCs are known in the art. Any of these linkers, as well as other linkers, may be used to link the cytotoxic and/or cytostatic agents to the antigen binding moiety of the LRRC15 targeting ADCs described herein.

The number of cytotoxic and/or cytostatic agents linked to the targeting moiety of an anti LRRC15 ADC (drug-to-antibody ratio: DAR) can vary and will be limited only by the number of available attachments sites on the targeting moiety and the number of agents linked to a single linker. Typically, a linker will link a single cytotoxic and/or cytostatic agent to the targeting moiety of the ADC. In embodiments of ADCs which include more than a single cytotoxic and/or cytostatic agent, each agent may be the same or different. As long as the ADC, does not exhibit unacceptable levels of aggregation under the conditions of use and/or storage, ADCs, with DARs of twenty, or even higher, are contemplated. In some embodiments, the ADCs, described herein may have a DAR in the range of about 1-10, 1-8, 1-6, or 1-4. In certain specific embodiments, the LRRC15 targeting ADC may have a DAR of 2, 3 or 4.

In some embodiments, the ADCs, are compounds according to structural formula (III):

[D-L-XY]n-Ab (III)

or salts thereof, where each “D” represents, independently of the others, a cytotoxic and/or cytostatic agent; each “L” represents, independently of the others, a linker; “Ab” represents a LRRC15 targeting moiety, e.g. an anti LRRC15 antibody according to the current invention; each “XY” represents a linkage formed between a functional group Rx on the linker and a “complementary” functional group Ry on the LRRC15 targeting moiety; and n represents the DAR of the LRRC15 targeting ADC.

In a specific exemplary embodiment, the ADCs are compounds according to structural formula (III) in which each “D” is the same and is either a cell-permeating auristatin (for example, dolastatin-10 or MMAE) or a cell-permeating minor groove-binding DNA cross-linking agent; each “L” is the same and is a linker cleavable by a lysosomal enzyme; each “XY” is a linkage formed between a maleimide and a sulfhydryl group; “Ab” is an antibody or fragment thereof comprising six CDRs corresponding to the six CDRs of an LRRC15 antibody according to the current invention; and n is 2, 3 or 4.

Cytotoxic and cytostatic agents are agents known to inhibit the growth and/or replication of and/or kill cells and in particular tumor cells. These compounds may be used in a combination therapy with an LRRC15 antibody, or as part of an LRRC15 targeting ADC as described herein: In some embodiments, the cytotoxic agent is selected from radionuclides, alkylating agents, DNA cross-linking agents, DNA intercalating agents (e.g., groove binding agents such as minor groove binders), cell cycle modulators, apoptosis regulators, kinase inhibitors, protein synthesis inhibitors, mitochondria inhibitors, nuclear export inhibitors, topoisomerase I inhibitors, topoisomerase II inhibitors, RNA/DNA antimetabolites and antimitotic agents.

In some embodiments, the cytotoxic agent is an alkylating agent selected from asaley (L-Leucine, N-[N-acetyl-4-[bis-(2-chloroethyl)amino]-DL-phenylalanyl]-, ethylester); AZQ (1,4-cyclohexadiene-1,4-dicarbamic acid, 2,5-bis(1-aziridinyl)-3,6-dioxo-, diethyl ester); BCNU (N,N′-Bis(2-chloroethyl)-N-nitrosourea); busulfan (1,4-butanediol dimethanesulfonate); (carboxyphthalato)platinum; CBDCA (cis-(1,1-cyclobutanedicarboxylato)diammineplatinum(II))); CCNU (N-(2-chloroethyl)-N′-cyclohexyl-N-nitrosourea); CHIP (iproplatin; NSC 256927); chlorambucil; chlorozotocin (2-[[[(2-chloroethyl) nitrosoamino]carbonyl]amino]-2-deoxy-D-glucopyranose); cis-platinum (cisplatin); clomesone; cyanomorpholinodoxorubicin; cyclodisone; dianhydrogalactitol (5,6-diepoxydulcitol); fluorodopan ((5-[(2-chloroethyl)-(2-fluoroethyl)amino]-6-methyl-uracil); hepsulfam; hycanthone; indolinobenzodiazepine dimer DGN462; melphalan; methyl CCNU ((1-(2-chloroethyl)-3-(trans-4-methylcyclohexane)-1-nitrosourea); mitomycin C; mitozolamide; nitrogen mustard ((bis(2-chloroethyl) methylamine hydrochloride); PCNU ((1-(2-chloroethyl)-3-(2,6-dioxo-3-piperidyl)-1-nitrosourea)); piperazine alkylator ((1-(2-chloroethyl)-4-(3-chloropropyl)-piperazine dihydrochloride)); piperazinedione; pipobroman (N,N′-bis(3-bromopropionyl) piperazine); porfiromycin (N-methylmitomycin C); spirohydantoin mustard; teroxirone (triglycidylisocyanurate); tetraplatin; thio-tepa (N,N′,N″-tri-1,2-ethanediylthio phosphoramide); triethylenemelamine; uracil nitrogen mustard (desmethyldopan); Yoshi-864 ((bis(3-mesyloxy propyl)amine hydrochloride).

In some embodiments, the cytotoxic agent is a DNA alkylating-like agent selected from Cisplatin; Carboplatin; Nedaplatin; Oxaliplatin; Satraplatin; Triplatin tetranitrate; Procarbazine; altretamine; dacarbazine; mitozolomide; temozolomide.

In some embodiments, the cytotoxic agent is an alkylating antineoplastic agents selected from Carboquone; Carmustine; Chlornaphazine; Chlorozotocin; Duocarmycin; Evofosfamide; Fotemustine; Glufosfamide; Lomustine; Mannosulfan; Nimustine; Phenanthriplatin; Pipobroman; Ranimustine; Semustine; Streptozotocin; ThioTEPA; Treosulfan; Triaziquone; Triethylenemelamine; Triplatin tetranitrate.

In some embodiments, the cytotoxic agent is a DNA replication and repair inhibitor selected from Altretamine; Bleomycin; Dacarbazine; Dactinomycin; Mitobronitol; Mitomycin; Pingyangmycin; Plicamycin; Procarbazine; Temozolomide; ABT-888 (veliparib); olaparib; KU-59436; AZD-2281; AG-014699; BSI-201; BGP-15; INO-1001; ONO-2231.

In some embodiments, the cytotoxic agent is a cell cycle modulator, such as Paclitaxel; Nab-Paclitaxel; Docetaxel; Vincristine; Vinblastine; ABT-348; AZD-1152; MLN-8054; VX-680; Aurora A-specific kinase inhibitors; Aurora B-specific kinase inhibitors and pan-Aurora kinase inhibitors; AZD-5438; BMI-1040; BMS-032; BMS-387; CVT-2584; flavopyridol; GPC-286199; MCS-5A; PD0332991; PHA-690509; seliciclib (CYC-202, R-roscovitine); ZK-304709; AZD4877, ARRY-520: GSK923295A.

In some embodiments, the cytotoxic agent is an apoptosis regulator such as AT-101 ((-)gossypol); G3139 or oblimersen (Bcl-2-targeting antisense oligonucleotide); IPI-194; IPI-565; N-(4-(4-((4′-chloro(1,1′-biphenyl)-2-yl)methyl)piperazin-1-ylbenzoyl)-4-(((1R)-3-(dimethylamino)-1-((phenylsulfanyl)methyl)propyl)amino)-3-nitrobenzenesulfonamide); N-(4-(4-((2-(4-chlorophenyl)-5,5-dimethyl-1-cyclohex-1-en-1-yl)methyl)piperazin-1-yl)benzoyl)-4-(((1R)-3-(morpholin-4-yl)-1-((phenylsulfanyl)methyl)propyl)amino)-3-((trifluoromethyl)sulfonyl)benzenesulfonamide; GX-070 (Obatoclax®; 1H-Indole, 2-(2-((3,5-dimethyl-1H-pyrrol-2-yl)methylene)-3-methoxy-2H-pyrrol-5-yl)-)); HGS1029; GDC-0145; GDC-0152; LCL-161; LBW-242; venetoclax; agents that target TRAIL or death receptors (e.g., DR4 and DR5) such as ETR2-ST01, GDC0145, HGS-1029, LBY-135, PRO-1762; drugs that target caspases, caspase-regulators, BCL-2 family members, death domain proteins, TNF family members, Toll family members, and/or NF-kappa-B proteins.

In some embodiments, the cytotoxic agent is an angiogenesis inhibitor such as ABT-869; AEE-788; axitinib (AG-13736); AZD-2171; CP-547,632; IM-862; pegaptamib; sorafenib; BAY43-9006; pazopanib (GW-786034); vatalanib (PTK-787, ZK-222584); sunitinib; SU-11248; VEGF trap; vandetanib; ABT-165; ZD-6474; DLL4 inhibitors.

In some embodiments, the cytotoxic agent is a proteasome inhibitor such as Bortezomib; Carfilzomib; Epoxomicin; Ixazomib; Salinosporamide A.

In some embodiments, the cytotoxic agent is a kinase inhibitor such as Afatinib; Axitinib; Bosutinib; Crizotinib; Dasatinib; Erlotinib; Fostamatinib; Gefitinib; Ibrutinib; Imatinib; Lapatinib; Lenvatinib; Mubritinib; Nilotinib; Pazopanib; Pegaptanib; Sorafenib; Sunitinib; SU6656; Vandetanib; Vemurafenib; CEP-701 (lesaurtinib); XL019; INCB018424 (ruxolitinib); ARRY-142886 (selemetinib); ARRY-438162 (binimetinib); PD-325901; PD-98059; AP-23573; CCI-779; everolimus; RAD-001; rapamycin; temsirolimus; ATP-competitive TORC1/TORC2 inhibitors including PI-103, PP242, PP30, Torin 1; LY294002; XL-147; CAL-120; ONC-21; AEZS-127; ETP-45658; PX-866; GDC-0941; BGT226; BEZ235; XL765.

In some embodiments, the cytotoxic agent is a protein synthesis inhibitor such as Streptomycin; Dihydrostreptomycin; Neomycin; Framycetin; Paromomycin; Ribostamycin; Kanamycin; Amikacin; Arbekacin; Bekanamycin; Dibekacin; Tobramycin; Spectinomycin; Hygromycin B; Paromomycin; Gentamicin; Netilmicin; Sisomicin; Isepamicin; Verdamicin; Astromicin; Tetracycline; Doxycycline; Chlortetracycline; Clomocycline; Demeclocycline; Lymecycline; Meclocycline; Metacycline; Minocycline; Oxytetracycline; Penimepicycline; Rolitetracycline; Tetracycline; Glycylcyclines; Tigecycline; Oxazolidinone; Eperezolid; Linezolid; Posizolid; Radezolid; Ranbezolid; Sutezolid; Tedizolid; Peptidyl transferase inhibitors; Chloramphenicol; Azidamfenicol; Thiamphenicol; Florfenicol; Pleuromutilins; Retapamulin; Tiamulin; Valnemulin; Azithromycin; Clarithromycin; Dirithromycin; Erythromycin; Flurithromycin; Josamycin; Midecamycin; Miocamycin; Oleandomycin; Rokitamycin; Roxithromycin; Spiramycin; Troleandomycin; Tylosin; Ketolides; Telithromycin; Cethromycin; Solithromycin; Clindamycin; Lincomycin; Pirlimycin; Streptogramins; Pristinamycin; Quinupristin/dalfopristin; Virginiamycin. In some embodiments, the drug moiety of the anti-chemokine receptor or anti LRRC15 ADC is a histone deacetylase inhibitor such as Vorinostat; Romidepsin; Chidamide; Panobinostat; Valproic acid; Belinostat; Mocetinostat; Abexinostat; Entinostat; SB939 (pracinostat); Resminostat; Givinostat; Quisinostat; thioureidobutyronitrile (Kevetrin™); CUDC-10; CHR-2845 (tefinostat); CHR-3996; 4SC-202; CG200745; ACY-1215 (rocilinostat); ME-344; sulforaphane.

In some embodiments, the cytotoxic agent is a topoisomerase I inhibitor such as camptothecin; various camptothecin derivatives and analogs (for example, NSC 100880, NSC 603071, NSC 107124, NSC 643833, NSC 629971, NSC 295500, NSC 249910, NSC 606985, NSC 74028, NSC 176323, NSC 295501, NSC 606172, NSC 606173, NSC 610458, NSC 618939, NSC 610457, NSC 610459, NSC 606499, NSC 610456, NSC 364830, and NSC 606497); morpholinisoxorubicin; SN-38.

In some embodiments, the cytotoxic agent is a topoisomerase 11 inhibitor such as doxorubicin; amonafide (benzisoquinolinedione); m-AMSA (4′-(9-acridinylamino)-3′-methoxymethanesulfonanilide); anthrapyrazole derivative ((NSC 355644); etoposide (VP-16); pyrazoloacridine ((pyrazolo[3,4,5-kl]acridine-2(6H)-propanamine, 9-methoxy-N, N-dimethyl-5-nitro-, monomethanesulfonate); bisantrene hydrochloride; daunorubicin; deoxydoxorubicin; mitoxantrone; menogaril; N,N-dibenzyl daunomycin; oxanthrazole; rubidazone; teniposide.

In some embodiments, the cytotoxic agent is a DNA intercalating agent such as anthramycin; chicamycin A; tomaymycin; DC-81; sibiromycin; pyrrolobenzodiazepine derivative; SGD-1882 ((S)-2-(4-aminophenyl)-7-methoxy-8-(3S)-7-methoxy-2-(4-methoxyphenyl)-5-oxo-5,11a -dihydro-1H-benzo[e]pyrrolo[1,2-a][1,4]diazepin-8-yl)oxy)propoxy)-1H-benzo[e]pyrrolo[1,2-a][1,4]diazepin-5(11aH)-one); SG2000 (SJG-136; (11aS,11a'S)-8,8′-(propane-1,3-diylbis(oxy))bis(7-methoxy-2-methylene-2,3-dihydro-1H-benzo[e]pyrrolo[1,2-a][1,4]diazepin-5(11aH)-one)).

In some embodiments, the cytotoxic agent is a RNA/DNA antimetabolite such as L-alanosine; 5-azacytidine; 5-fluorouracil; acivicin; aminopterin derivative N-[2-chloro-5[[(2,4-diamino-5-methyl-6-quinazolinyl)methyl]amino]benzoyl]L-aspartic acid (NSC 132483); aminopterin derivative N-[4-[[(2,4-diamino-5-ethyl-6-quinazolinyl)methyl]amino]benzoyl]L-aspartic acid; aminopterin derivative N-[2-chloro-4-[[(2,4-diamino-6-pteridinyl)methyl]amino]benzoyl]L-aspartic acid monohydrate; antifolate PT523 ((Na-(4-amino-4-deoxypteroyl)-Ny-hemiphthaloyl-L-ornithine)); Baker's soluble antifol (NSC 139105); dichlorallyl lawsone ((2-(3,3-dichloroallyl)-3-hydroxy-1,4-naphthoquinone); brequinar; ftorafur ((pro-drug; 5-fluoro-1-(tetrahydro-2-furyl)-uracil); 5,6-dihydro-5-azacytidine; methotrexate; methotrexate derivative (N-[[4-[[(2,4-diamino-6-pteridinyl)methyl]methylamino]-1-naphthalenyl]carbonyl]L-glutamic acid); PALA ((N-(phosphonoacetyl)-L-aspartate); pyrazofurin; trimetrexate.

In some embodiments, the cytotoxic agent is a DNA antimetabolite such as 3-HP; 2′-deoxy-5-fluorouridine; 5-HP; α-TGDR (α-2′-deoxy-6-thioguanosine); aphidicolin glycinate; ara C (cytosine arabinoside); 5-aza-2′-deoxycytidine; β-TGDR (β-2′-deoxy-6-thioguanosine); cyclocytidine; guanazole; hydroxyurea; inosine glycodialdehyde; macbecin II; pyrazoloimidazole; thioguanine; thiopurine.

In some embodiments, the cytotoxic agent is a mitochondria inhibitor such as pancratistatin; phenpanstatin; rhodamine-123; edelfosine; d-alpha-tocopherol succinate; compound 11S; aspirin; ellipticine; berberine; cerulenin; GX015-070 (Obatoclax®; 1H-Indole, 2-(2-((3,5-dimethyl-1H-pyrrol-2-yl)methylene)-3-methoxy-2H-pyrrol-5-yl)-); celastrol (tripterine); metformin; Brilliant green; ME-344.

In some embodiments, the cytotoxic agent is an antimitotic agent such as allocolchicine; auristatins, such as MMAE (monomethyl auristatin E) and MMAF (monomethyl auristatin F); halichondrin B; cemadotin; colchicine; cholchicine derivative (N-benzoyl-deacetyl benzamide); dolastatin-10; dolastatin-15; maytansine; maytansinoids, such as DM1 (N2′-deacetyl-N2′-(3-mercapto-1-oxopropyl)-maytansine); rhozoxin; paclitaxel; paclitaxel derivative ((2′-N-[3-(dimethylamino)propyl]glutaramate paclitaxel); docetaxel; thiocolchicine; trityl cysteine; vinblastine sulfate; vincristine sulfate.

In some embodiments, the cytotoxic agent is a nuclear export inhibitor such as callystatin A; delactonmycin; KPT-185 (propan-2-yl (Z)-3-[3-[3-methoxy-5-(trifluoromethyl)phenyl]-1,2,4-triazol-1-yl]prop-2-enoate); kazusamycin A; leptolstatin; leptofuranin A; leptomycin B; ratjadone; Verdinexor ((Z)-3-[3-[3,5-bis(trifluoromethyl)phenyl]-1,2,4-triazol-1-yl]-N-pyridin-2-ylprop-2-enehydrazide).

In some embodiments, the cytotoxic agent is a hormonal therapeutics such as anastrozole; exemestane; arzoxifene; bicalutamide; cetrorelix; degarelix; deslorelin; trilostane; dexamethasone; flutamide; raloxifene; fadrozole; toremifene; fulvestrant; letrozole; formestane; glucocorticoids; doxercalciferol; sevelamer carbonate; lasofoxifene; leuprolide acetate; megesterol; mifepristone; nilutamide; tamoxifen citrate; abarelix; prednisone; finasteride; rilostane; buserelin; luteinizing hormone releasing hormone (LHRH); Histrelin; trilostane or modrastane; fosrelin; goserelin.

Any of these agents that include, or that may be modified to include, a site of attachment to an antibody and/or binding fragment can be included in an LRRC15 targeting ADC according to the current invention.

### CAR T Cells

According to some third embodiments of the 3rd aspect, the conjugate is a chimeric antigen receptor conjugate engineered for LRRC15 targeting. Recently, CART cells have gained attention from their clinical successes and expedited FDA approvals, cf. WO2020/102240, incorporated herein in its entirety. In the CAR T cell approach, T cells are engineered to express CARs that are specific for an antigen present on tumor cells. These engineered T cells are then re-administered to the same patient. Upon injection, CAR T cells recognize the targeted antigen on target cells to induce target cell death. T cells expressing chimeric antigen receptors (CAR T cells) thus constitute a novel modality for medical uses such as tumor treatment. The chimeric antigen receptor (CAR) is a genetically engineered receptor that is designed to target a specific antigen, for example, a tumor antigen. This targeting can result in cytotoxicity against the tumor, for example, such that CAR T cells expressing CARs can target and kill tumors via the specific tumor antigens.

According to the present invention, the antibodies or antigen binding fragments provided for LRRC15 can be used to engineer CAR T cells for specific recognition of LRRC15 expressing cells. CARs according to the current invention may comprise
(i) a recognition region, e.g., a single chain fragment variable (scFv) region derived from a provided anti LRRC15 antibody for recognition and binding to the LRRC15 or chemokine receptor expressed by the target cell, and
(ii) an activation signaling domain, e.g., the CD3 chain of T cells, which can serve as a T cell activation signal in CARs.

Preferably, the CARs according to the current invention comprise a co-stimulation domain (e.g., CD137, CD28 or CD134) to achieve prolonged activation of T cells in vivo. Addition of a co-stimulation domain enhances the in vivo proliferation and survival of T cells containing CARs, and initial clinical data have shown that such constructs are promising therapeutic agents in the treatment of diseases, such as cancer. According to the current invention, the CAR T cells can be used to treat any disease with local or systemic aberrant presence of cells expressing LRRC15.

### Bispecific Antibodies

According to some fourth, highly preferred embodiments of the 3rd aspect, the conjugate is or comprises a bispecific antibody or a multispecific antibody. In some preferred embodiments the bispecific antibody comprises at least one Fc domain.

In some preferred embodiments, the first binding moiety of the bispecific antibody is an antibody or antigen binding fragment according to the 2nd aspect and the second binding moiety of the bispecific antibody is the same or a different antibody or antigen binding fragment according to the 2nd aspect.

In some further embodiments, the first binding moiety of the bispecific antibody is an antibody or antigen binding fragment according to the 2nd aspect and the second binding moiety of the bispecific antibody is an antibody or antigen binding fragment binding to a cell-surface protein such as a cell type-specific antigen. In some of these embodiments, the second binding moiety of the bispecific antibody is an antibody or antigen binding fragment targeting a checkpoint protein, such as an anti PD1 antibody, an anti PD-L1 antibody, or a CTLA-4 antibody. Suitable checkpoint protein targeting antibodies include Nivolumab, Pembrolizumab, Atezolizumab, Avelumab, Durvalumab, Cemiplimab, Dostarlimab, or Ipilimumab. In some other of these embodiments, the second binding moiety of the bispecific antibody is HER2 targeting antibody, such as Trastuzumab, Pertuzumab and/or Margetuximab.

Techniques for making bi- or multispecific antibodies include, but are not limited to, recombinant co-expression of two immunoglobulin heavy chain-light chain pairs having different specificities (see Milstein and Cuello, Nature 305: 537 (1983), WO 93/08829, and Traunecker et al., EMBO J. 10: 3655 (1991)), and chemical conjugation of two different monoclonal antibodies (see Staerz et al. (1985) Nature 314(6012): 628-31). Multispecific antibodies may also be made by cross-linking two or more antibodies or fragments (see, e.g., U.S. Pat. No. 4,676,980, and Brennan et al., Science, 229: 81 (1985)), using leucine zippers to produce bi-specific antibodies (see, e.g., Kostelny et al., J. Immunol, 148(5): 1547-1553 (1992)), using diabody technology for making bispecific antibody fragments (see, e.g., Hollinger et al., Proc. Natl. Acad. Sci. USA, 90:6444-6448 (1993)), using single-chain Fv (sFv) dimers (see, e.g. Gruber et al., J. Immunol, 152:5368 (1994)), by preparing trispecific antibodies as described, e.g., in Tutt et al. J. Immunol. 147: 60 (1991) and by controlled Fab arm exchange (cFAE) according to Labrijn A F et al. Proc Natl Acad Sci USA 2013; 110:5145-50.

### Conjugates for Diagnosis and Research Applications

According to some fifth embodiments of the 3rd aspect, the conjugate comprises a detectable moiety. Examples of detectable moieties include various enzymes, prosthetic groups, fluorescent materials, luminescent materials, bioluminescent materials, radioactive materials, positron emitting metals, nonradioactive paramagnetic metal ions and reactive moieties. The detectable substance can be coupled or conjugated either directly to the antibody or fragment thereof or indirectly, e.g. through a linker known in the art or another moiety, using techniques known in the art. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, 3-galactosidase, acetylcholinesterase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like.

Examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include 125I, 131I, 111In or 99mTc.

Detection of expression of LRRC15 generally involves contacting a biological sample (tumor, cells, tissue, or body fluid of an individual) with one or more antibodies or fragments according to the current invention (optionally conjugated to a detectable moiety), and detecting whether or not the sample is positive for LRRC15, or whether the sample has altered (e.g., reduced or increased) expression as compared to a control sample.

### Conjugates Comprising IL2v

According to some sixth embodiments of the 3rd aspect, the conjugate comprises an IL-2 variant (IL2v) with abolished CD25 binding. Preferably, the IL-2 variant is fused to the C-terminus of the LRRC15 antibody described herein with a heterodimeric Fc-part.

### Pharmaceutical Composition

According to a fourth aspect, there is provided a pharmaceutical composition comprising a conjugate, antibody or antigen-binding fragment according to the current invention, or a combination thereof.

For example, the pharmaceutical composition comprises a conjugate according to the first aspect in a therapeutically effective amount. For example, the pharmaceutical composition comprises an antibody or antigen-binding fragment according to the second aspect in a therapeutically effective amount. For example, the pharmaceutical composition comprises a conjugate according to the third aspect in a therapeutically effective amount.

Preferably, the pharmaceutical composition furthermore comprises a pharmaceutically acceptable carrier, excipient, or stabilizer.

Pharmaceutical compositions can be prepared by mixing the antibody, fragment, or conjugate having the desired degree of purity with optional physiologically acceptable carriers, excipients or stabilizers (Remington's Pharmaceutical Sciences (18th ed.; Mack Pub. Co.: Eaton, Pa., 1990). Pharmaceutical compositions may be for example in the form of lyophilized formulations or aqueous solutions.

### Carriers, Excipients, Stabilizers

Acceptable carriers, excipients, or stabilizers are nontoxic to recipients at the dosages and concentrations employed, and include buffers such as phosphate buffer (e.g. PBS), citrate buffer, and other organic acid buffer; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (e.g. less than about 10 residues) polypeptide; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as Tween®, Pluronic® or polyethylene glycol (PEG).

Pharmaceutically suitable excipients include, inter alia,
fillers and carriers (for example cellulose, microcrystalline cellulose (such as, for example, Avicel®), lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat),
solvents (for example water, ethanol, isopropanol, glycerol, propylene glycol, medium chain-length triglycerides fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxethylene fatty acid esters, polyoxyethylene fatty alcohol ethers, glycerol fatty acid esters, poloxamers (such as, for example, Pluronic®),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (for example highly-disperse silicas),
viscosity-increasing agents, gel formers, thickeners and/or binders (for example polyvinylpyrrolidone, methylcellulose, hydroxypropylmethylcellulose, hydroxypropyl-cellulose, carboxymethylcellulose-sodium, starch, carbomers, polyacrylic acids (such as, for example, Carbopol®); alginates, gelatine),
disintegrants (for example modified starch, carboxymethylcellulose-sodium, sodium starch glycolate (such as, for example, Explotab®), cross-linked polyvinylpyrrolidone, croscarmellose-sodium (such as, for example, AcDiSol®)),
flow regulators, lubricants, glidants and mould release agents (for example magnesium stearate, stearic acid, talc, highly-disperse silicas (such as, for example, Aerosil®)),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropyl-methylcellulose phthalate, cellulose acetate, cellulose acetate phthalate, polyacrylates, polymethacrylates such as, for example, Eudragit®)),
capsule materials (for example gelatine, hydroxypropylmethylcellulose),
synthetic polymers (for example polylactides, polyglycolides, polyacrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate, para-aminobenzoic acid (pABA)),
colourants (for example inorganic pigments such as, for example, iron oxides, titanium dioxide),
flavourings, sweeteners, flavour- and/or odour-masking agents.

In a highly preferred embodiment, the pharmaceutical composition comprises
a. conjugate or antibody according to the current invention, e.g. 0.1 to 10 mg/ml or 2.5 mg/ml, and
b. citrate, e.g. 1 mM to 100 mM or 30 mM, and/or
c. sucrose, e.g. 1 mM to 100 mM or 50 mM, and/or
d. EDTA, e.g. 0.1 mM to 20 mM or 2 mM, and/or
e. pABA, e.g. 0.01 to 10 mg/ml or 0.5 mg/ml, and/or
f. polysorbate, e.g. 0.1 to 20% or 7%.

### Multiple Therapeutically Active Compounds

According to the current invention a pharmaceutical composition may contain more than one active compound, e.g. as necessary or beneficial for the particular indication being treated.

According to some preferred embodiments, the pharmaceutical composition comprises one or more further therapeutically active compounds.

In some preferred of these embodiments, the one or more further therapeutically active compound(s) comprise(s)
a) an antibody or a small molecule targeting a checkpoint protein, such as PD1, PD-L1 or CTLA-4, and/or
b) an antibody or a small molecule targeting HER2 and/or EGFR, and/or
c) a chemotherapeutic agent, such as a taxane, doxorubicin, cis-platin, carboplatin or oxaliplatin, and/or
d) a targeted kinase inhibitor, such as Sorafinib, Regorafenib, or MEKi-1.

### Combination with Checkpoint Inhibitors

According to some preferred embodiments, the further therapeutically active compound(s) comprise an antibody or a small molecule targeting a checkpoint protein, such as PD1, PD-L1 or CTLA-4. Suitable antibodies targeting a checkpoint protein include Nivolumab (PD1; Human IgG4), Pembrolizumab (PD1; Humanized IgG4), Atezolizumab (PD-L1; Humanized IgG1), Avelumab (PD-L1; Human IgG1), Durvalumab (PD-L1; Human IgG1), Cemiplimab, cemiplimab-rwlc (PD-1; Human mAb), Dostarlimab (TSR-042) (PD-1; Humanized IgG4), or Ipilimumab (CTLA-4; Human IgG1).

In some embodiments, the antibody or a small molecule targeting a checkpoint protein targets CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof.

### Combination with HER2 or EGFR Targeting Antibodies or Molecules

According to some preferred embodiments, the further therapeutically active compound(s) comprise an antibody or a small molecule targeting HER2 and/or EGFR. Suitable antibodies targeting HER2 are Trastuzumab (HER2; Humanized IgG1), Pertuzumab (HER2; humanized IgG1), Ado-trastuzumab emtansine (HER2; humanized IgG1; ADC), [fam-]trastuzumab deruxtecan, fam-trastuzumab deruxtecan-nxki (HER2; Humanized IgG1 ADC), Sacituzumab govitecan; sacituzumab govitecan-hziy (TROP-2; Humanized IgG1 ADC) and/or Margetuximab (HER2; Chimeric IgG1). Suitable antibodies targeting EGFR are Cetuximab (EGFR; Chimeric IgG1), Panitumumab (EGFR; Human IgG2), and Necitumumab (EGFR; Human IgG1).

### Combination with Therapeutic Antibodies

According to some embodiments, the further therapeutically active compound(s) comprise a therapeutic antibody selected from Muromonab-CD3 (CD3; Murine IgG2a), Efalizumab (CD11a; Humanized IgG1), Tositumomab-I131 (CD20; Murine IgG2a), Nebacumab (Endotoxin; Human IgM), Edrecolomab (EpCAM; Murine IgG2a), Catumaxomab (EPCAM/CD3; Rat/mouse bispecific mAb), Daclizumab (IL-2R; Humanized IgG1), Abciximab (GPIIb/IIIa; Chimeric IgG1 Fab), Rituximab (CD20; Chimeric IgG1), Basiliximab (IL-2R; Chimeric IgG1), Palivizumab (RSV; Humanized IgG1), Infliximab (TNF; Chimeric IgG1), Trastuzumab (HER2; Humanized IgG1), Adalimumab (TNF; Human IgG1), Ibritumomab tiuxetan (CD20; Murine IgG1), Omalizumab (IgE; Humanized IgG1), Cetuximab (EGFR; Chimeric IgG1), Bevacizumab (VEGF; Humanized IgG1), Natalizumab (a4 integrin; Humanized IgG4), Panitumumab (EGFR; Human IgG2), Ranibizumab (VEGF; Humanized IgG1 Fab), Eculizumab (C5; Humanized IgG2/4), Certolizumab pegol (TNF; Humanized Fab, pegylated), Ustekinumab (IL-12/23; Human IgG1), Canakinumab (IL-1S; Human IgG1), Golimumab (TNF; Human IgG1), Ofatumumab (CD20; Human IgG1), Tocilizumab (IL-6R; Humanized IgG1), Denosumab (RANK-L; Human IgG2), Belimumab (BLyS; Human IgG1), Ipilimumab (CTLA-4; Human IgG1), Brentuximab vedotin (CD30; Chimeric IgG1; ADC), Pertuzumab (HER2; humanized IgG1), Ado-trastuzumab emtansine (HER2; humanized IgG1; ADC), Raxibacumab (B. anthrasis PA; Human IgG1), Obinutuzumab (CD20; Humanized IgG1 Glycoengineered), Siltuximab (IL-6; Chimeric IgG1), Ramucirumab (VEGFR2; Human IgG1), Vedolizumab (α4β7 integrin; humanized IgG1), Nivolumab (PD1; Human IgG4), Pembrolizumab (PD1; Humanized IgG4), Blinatumomab (CD19, CD3; Murine bispecific tandem scFv), Alemtuzumab (CD52; Humanized IgG1), Evolocumab (PCSK9; Human IgG2), Idarucizumab (Dabigatran; Humanized Fab), Necitumumab (EGFR; Human IgG1), Dinutuximab (GD2; Chimeric IgG1), Secukinumab (IL-17a; Human IgG1), Mepolizumab (IL-5; Humanized IgG1), Alirocumab (PCSK9; Human IgG1), Daratumumab (CD38; Human IgG1), Elotuzumab (SLAMF7; Humanized IgG1), Ixekizumab (IL-17a; Humanized IgG4), Reslizumab (IL-5; Humanized IgG4), Olaratumab (PDGFRα; Human IgG1), Bezlotoxumab (Clostridium difficile enterotoxin B; Human IgG1), Atezolizumab (PD-L1; Humanized IgG1), Obiltoxaximab (B. anthrasis PA; Chimeric IgG1), Brodalumab (IL-17R; Human IgG2), Dupilumab (IL-4R a; Human IgG4), Inotuzumab ozogamicin (CD22; Humanized IgG4; ADC), Guselkumab (IL-23 p19; Human IgG1), Sarilumab (IL-6R; Human IgG1), Avelumab (PD-L1; Human IgG1), Emicizumab (Factor Ixa, X; Humanized IgG4, bispecific), Ocrelizumab (CD20; Humanized IgG1), Benralizumab (IL-5R a; Humanized IgG1), Durvalumab (PD-L1; Human IgG1), Gemtuzumab ozogamicin (CD33; Humanized IgG4; ADC), Erenumab, erenumab-aooe (CGRP receptor; Human IgG2), Galcanezumab, galcanezumab-gnlm (CGRP; Humanized IgG4), Burosumab, burosumab-twza (FGF23; Human IgG1), Lanadelumab, lanadelumab-flyo (Plasma kallikrelin; Human IgG1), Mogamulizumab, mogamulizumab-kpkc (CCR4; Humanized IgG1), Tildrakizumab; tildrakizumab-asmn (IL-23 p19; Humanized IgG1), Fremanezumab, fremanezumab-vfrm (CGRP; Humanized IgG2), Ravulizumab, ravulizumab-cwvz (C5; Humanized IgG2/4), Cemiplimab, cemiplimab-rwlc (PD-1; Human mAb), Ibalizumab, ibalizumab-uiyk (CD4; Humanized IgG4), Emapalumab, emapalumab-lzsg (IFNg; Human IgG1), Moxetumomab pasudotox, moxetumomab pasudotox-tdfk (CD22; Murine IgG1 dsFv immunotoxin), Caplacizumab, caplacizumab-yhdp (von Willebrand factor; Humanized Nanobody), Risankizumab, risankizumab-rzaa (IL-23 p19; Humanized IgG1), Polatuzumab vedotin, polatuzumab vedotin-piiq (CD79b; Humanized IgG1 ADC), Romosozumab, romosozumab-aqqg (Sclerostin; Humanized IgG2), Brolucizumab, brolucizumab-dbll (VEGF-A; Humanized scFv), Crizanlizumab; crizanlizumab-tmca (CD62 (aka P-selectin); Humanized IgG2), Enfortumab vedotin, enfortumab vedotin-ejfv (Nectin-4; Human IgG1 ADC), [fam-]trastuzumab deruxtecan, fam-trastuzumab deruxtecan-nxki (HER2; Humanized IgG1 ADC), Teprotumumab, teprotumumab-trbw (IGF-1R; Human IgG1), Eptinezumab, eptinezumab-jjmr (CGRP; Humanized IgG1), Isatuximab, isatuximab-irfc (CD38; Chimeric IgG1), Sacituzumab govitecan; sacituzumab govitecan-hziy (TROP-2; Humanized IgG1 ADC), Inebilizumab (CD19; Humanized IgG1), Leronlimab (CCR5; Humanized IgG4), Satralizumab (IL-6R; Humanized IgG2), Narsoplimab (MASP-2, Human IgG4), Tafasitamab (CD19; Humanized IgG1), REGNEB3 (Ebola virus; mixture of 3 human IgG1), Naxitamab (GD2; Humanized IgG1), Oportuzumab monatox (EpCAM; Humanized scFv immunotoxin), Belantamab mafodotin (B-cell maturation antigen; Humanized IgG1 ADC), Margetuximab (HER2; Chimeric IgG1), Tanezumab (Nerve growth factor; Humanized IgG2), Dostarlimab (TSR-042) (PD-1; Humanized IgG4), Teplizumab (CD3; Humanized IgG1), Aducanumab (Amyloid beta; Human IgG1), Sutimlimab (BIVV009) (C1s; Humanized IgG4), Evinacumab (Angiopoietin-like 3; Human IgG4).

### Combination with Cytotoxic or Cytostatic Agents

According to some embodiments, the further therapeutically active compound(s) comprise a cytostatic and/or cytotoxic agent selected from radionuclides, alkylating agents, DNA cross-linking agents, DNA intercalating agents (e.g., groove binding agents such as minor groove binders), cell cycle modulators, apoptosis regulators, kinase inhibitors, protein synthesis inhibitors, mitochondria inhibitors, nuclear export inhibitors, topoisomerase I inhibitors, topoisomerase II inhibitors, RNA/DNA antimetabolites and antimitotic agents.

In some preferred embodiments, the cytotoxic agent is an auristatin, a maytansinoid, a kinesin-spindle protein (KSP) inhibitor, a nicotinamide phosphoribosyltransferase (NAMPT) inhibitor or a pyrrolobenzodiazepine derivative.

Generally, the use of chemotherapeutic agents and/or anti-cancer agents in combination with a compound or pharmaceutical composition of the present invention serves to:
1. yield better efficacy in reducing the growth of a tumour or even eliminate the tumour as compared to administration of either agent alone,
2. provide for the administration of lesser amounts of the administered chemotherapeutic agents,
3. provide for a chemotherapeutic treatment that is well tolerated in the patient with fewer deleterious pharmacological complications than observed with single agent chemotherapies and certain other combined therapies,
4. provide for treating a broader spectrum of different cancer types in mammals, especially humans,
5. provide for a higher response rate among treated patients,
6. provide for a longer survival time among treated patients compared to standard chemotherapy treatments,
7. provide a longer time for tumour progression, and/or
8. yield efficacy and tolerability results at least as good as those of the agents used alone, compared to known instances where other cancer agent combinations produce antagonistic effects.

### Med Use/Methods of Treatment

According to a 5^{th} aspect, there is provided the conjugate according to the first or third aspect or the antibody or antigen binding fragment according to the 2nd aspect, or the pharmaceutical composition according to the 4th aspect for use as a medicament.

Also, there is provided the use of the conjugate according to the first or third aspect or the antibody or antigen binding fragment according to the 2nd aspect, or the pharmaceutical composition according to the 4th aspect for the manufacture of a medicament, e.g. for the treatment of a tumor or a disease involving cells expressing LRRC15.

Furthermore, there is provided a method of treating a disease, the method comprising administering an effective dose of the the conjugate according to the first or third aspect or the antibody or antigen binding fragment according to the 2nd aspect, or the pharmaceutical composition according to the 4th aspect to a patient in need thereof.

In some first preferred embodiments, there is provided the conjugate according to the first aspect for use as a medicament. In some second preferred embodiments, there is provided the antibody or antigen-binding fragment according to the second aspect for use as a medicament.

In some third preferred embodiments, there is provided the conjugate according to the third aspect for use as a medicament. In some fourth preferred embodiments, there is provided the pharmaceutical formulation according to the fourth aspect for use as a medicament.

For therapeutic applications, the antibody or antigen binding fragment or the conjugate or the pharmaceutical composition according to the invention, can be administered to a patient, e.g. to a human or non-human subject, in a pharmaceutically acceptable dosage form. For example, administration may occur intravenously as a bolus or by continuous infusion over a period of time, by intramuscular, intraperitoneal, intra-cerebrospinal, subcutaneous, intra-articular, intrasynovial, intrathecal, oral, topical, or inhalation routes. The antibodies, fragments, conjugates and pharmaceutical compositions according to the current invention are particularly suitable to be administered by intra-tumoral, peri-tumoral, intra-lesional, or peri-lesional routes, to exert local as well as systemic therapeutic effects.

Possible administration routes include parenteral (e.g., intramuscular, intravenous, intra-arterial, intraperitoneal, or subcutaneous), intrapulmonary and intranasal. In addition, the antibodies, fragments, conjugates and pharmaceutical compositions might be administered by pulse infusion, with, e.g., declining doses of the antibody, fragment or conjugate. Preferably, the dosing is given by injections, most preferably intravenous or subcutaneous injections, depending in part on whether the administration is brief or chronic. The amount to be administered may depend on a variety of factors such as the clinical symptoms, weight of the patient or subject, and whether other drugs are administered. The skilled artisan will recognize that the route of administration will vary depending on the disorder or condition to be treated. Dosing frequency of the antibody may range from once every 3-6 months to weekly or daily dosing. Similarly, dose levels range from a low mg fixed dose (daily, weekly, biweekly, or monthly, depending on antibody) up to approximately 1 g doses. Dosing frequency depends on a variety of factors including the concentration and turnover rate of the target, biodistribution of the target, half-life of the antibodies, fragments or conjugates and potential pharmacodynamic effects that may enhance the biological effects of the antibodies, fragments, conjugates and pharmaceutical compositions beyond its presence in pharmacologically relevant levels.

For the prevention or treatment of disease, the appropriate dosage of antibody or conjugate will depend on the type of disease to be treated, the severity and course of the disease, whether the antibody is administered for preventive, diagnostic or therapeutic purposes, previous therapy, the subject's clinical history and response to the antibody variant, and the discretion of the attending physician or health veterinary professional. The antibody is suitably administered to the subject at one time or over a series of treatments.

When administered intravenously, the pharmaceutical composition comprising the antibodies, fragments or conjugates can be administered by infusion over a period of about 0.5 to about 5 hours. In some embodiments, infusion may occur over a period of about 0.5 to about 2.5 hours, over a period of about 0.5 to about 2.0 hours, over a period of about 0.5 to about 1.5 hours, or over a period of about 1.5 hours, depending upon the antibodies, fragments, conjugates and pharmaceutical compositions being administered and the amount of antibody, fragment or conjugate being administered.

For TTCs the radiopharmaceutical is preferably administered at a dosage level of thorium-227 dosage of 500 kBq/kg to 2 MBq/kg bodyweight, preferably 1.5 MBq/kg. Correspondingly, a single dosage may comprise around any of these ranges multiplied by a suitable bodyweight, such as 30 to 150 kg, preferably 40 to 100 kg. The specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

Preferably, the antibody or antibody conjugate according to the current invention is administered at a total dose of ≥0.15 mg/kg, ≥1 mg/kg, ≥1.5 mg/kg, ≥3 mg/kg, ≥5 mg/kg, ≥10 mg/kg. It was surprisingly found that a dose about 1.5 mg/kg conjugate circumvented potential target mediated drug deposition. Target-mediated drug disposition (TMDD) corresponds to a special case wherein a significant proportion of a drug (relative to dose) is bound with high affinity to a pharmacological target, such that this interaction is reflected in the pharmacokinetic properties of the drug.

Preferably, the conjugate according to the current invention is furthermore loaded with radioisotope and administered at about ≥200 kBq/kg, ≥250 kBq/kg, ≥300 kBq/kg, ≥350 kBq/kg, 400 kBq/kg, ≥450 kBq/kg, ≥500 kBq/kg, ≥550 kBq/kg, ≥600 kBq/kg, ≥650 kBq/kg, ≥700 kBq/kg, ≥750 kBq/kg, ≥800 kBq/kg, ≥850 kBq/kg, ≥900 kBq/kg, ≥950 kBq/kg, ≥1000 kBq/kg, ≥1100 kBq/kg, ≥1200 kBq/kg, ≥1300 kBq/kg, ≥1400 kBq/kg, ≥1500 kBq/kg.

For example, the conjugate according to the current invention is administered at a total dose of 1.5 mg/kg and at 200 kBq/kg, ≥250 kBq/kg, ≥300 kBq/kg, ≥350 kBq/kg, ≥400 kBq/kg, ≥450 kBq/kg or ≥500 kBq/kg.

Preferably, the antibody, fragment or antibody conjugate according to the current invention is administered at an antibody dose of at least 1.5 mg/kg and/or at a radioactive dose of at least 250 kBq/kg.

### Specific Indications

According to the TCGA dataset, LRRC15 may be overexpressed in patients with glioma, thyroid cancer, lung cancer, colorectal cancer, head and neck cancer, stomach cancer, liver cancer, pancreatic cancer, renal cancer, urothelial cancer, prostate cancer, testis cancer, breast cancer, cervical cancer, endometrial cancer and melanoma.

In principle, the antibodies, fragments, conjugates and pharmaceutical compositions according to the current invention can be used in the treatment of any cancer involving LRRC15 expressing cells.

For example, the cancer may comprise cells expressing LRRC15, such as breast cancer, head and neck cancer, squamous cell cancer, (squamous) lung cancer, pancreatic cancer, diffuse large B-cell carcinoma, lung adenocarcinoma, colorectal cancer, gastric cancer, and sarcoma. In some examples, the cancer comprises stroma cells expressing LRRC15. In some other or the same examples, the cancer comprises tumor cells expressing LRRC15.

In some preferred embodiments, the conjugate, antibody or fragment according to the current invention is used as a medicament in the treatment of sarcoma, breast cancer, lung cancer, colorectal cancer, non small cell lung carcinoma (NSCLC), testicular cancer or melanoma.

### Combination Treatment

According to some preferred embodiments according to the 5th aspect the medical use is a use in simultaneous, separate, or sequential combination with one or more further therapeutically active compounds.

LRRC15 antibodies, fragments or conjugates thereof, may be used adjunctive to—or with—other agents or treatments having anti-cancer properties. When used adjunctively, the antibodies, fragments or conjugates and other agent(s) may be formulated together in a single, combination pharmaceutical composition or formulation, as described elsewhere herein, or may be formulated and administered separately, either on a single coordinated dosing regimen or on different dosing regimens. Agents administered adjunctively with LRRC15 antibodies, fragments or conjugates thereof may have complementary activities to the LRRC15 antibodies, fragments or conjugates thereof, such that the LRRC15 antibodies, fragments or conjugates thereof and other agents do not adversely affect each other.

Agents that may be used adjunctively with the LRRC15 antibodies, fragments or conjugates according to the current invention can be those described elsewhere herein as further therapeutically active compounds for pharmaceutical composition. For example, agents that may be used adjunctively with the LRRC15 antibodies, fragments or conjugates according to the current invention can be alkylating agents, angiogenesis inhibitors, antibodies, antimetabolites, antimitotics, antiproliferatives, antivirals, aurora kinase inhibitors, apoptosis promoters (for example, Bcl-2 family inhibitors), activators of death receptor pathway, Bcr-Abl kinase inhibitors, BiTE (Bi-Specific T cell Engager) antibodies, antibody drug conjugates, biologic response modifiers, cyclin-dependent kinase inhibitors, cell cycle inhibitors, cyclooxygenase-2 inhibitors, DVDs, leukemia viral oncogene homolog (ErbB2) receptor inhibitors, growth factor inhibitors, heat shock protein (HSP)-90 inhibitors, histone deacetylase (HDAC) inhibitors, hormonal therapies, immunologicals, inhibitors of inhibitors of apoptosis proteins (IAPs), intercalating antibiotics, kinase inhibitors, kinesin inhibitors, Jak2 inhibitors, mammalian target of rapamycin inhibitors, microRNAs, mitogen-activated extracellular signal-regulated kinase inhibitors, multivalent binding proteins, non-steroidal anti-inflammatory drugs (NSAIDs), poly ADP (adenosine diphosphate)-ribose polymerase (PARP) inhibitors, platinum chemotherapeutics, polo-like kinase (Plk) inhibitors, phosphoinositide-3 kinase (PI3K) inhibitors, proteasome inhibitors, purine analogs, pyrimidine analogs, receptor tyrosine kinase inhibitors, retinoids/deltoids plant alkaloids, small inhibitory ribonucleic acids (siRNAs), topoisomerase inhibitors, ubiquitin ligase inhibitors, and the like, as well as combinations of one or more of these agents.

### Diagnostic Uses

The LRRC15 antibodies, fragments and conjugates as described herein may be used for a variety of purposes, e.g. for in vitro, in vivo and ex vivo applications and/or in vitro, in vivo and ex vivo diagnostics.

According to a 6^{th} aspect, there is provided the conjugate according to the first or third aspect or the antibody or antigen binding fragment according to the 2nd aspect, or the pharmaceutical composition according to the 4th aspect for use as a diagnostic agent.

For example, LRRC15 antibodies or antigen-binding fragments thereof can be used for detecting the presence of LRRC15-expressing tumors. The presence or level of LRRC15-expressing cells or shed LRRC15 within various biological samples, including serum, and tissue biopsy specimens, may be analyzed.

In addition, LRRC15 targeting conjugates may be used in various imaging methodologies such as immunoscintigraphy, e.g. with 99Tc (or a different isotope). For example, an imaging protocol similar to the one described using a 1111n conjugated anti-PSMA antibody may be used to detect tumors (Sodee et al., Clin. Nuc. Med. 21: 759-766, 1997).

Positron emission tomography (PET) with radiolabeled monoclonal antibodies sometimes termed as “immunoPET”, is an attractive method for non-invasive tumor detection as well as treatment planning (Zhang, Yin, Hao Hong, and Weibo Cai. “PET tracers based on Zirconium-89.” Current radiopharmaceuticals 4.2 (2011): 131-139.). For PET applications, the conjugates according to the current invention can be loaded with 89Zr (t1/2=3.3 d), 124I (t1/2=4.2 d), 64Cu (t1/2=12.7 h), 86Y (t1/2=14.7 h), or any other beta particle emitting radionuclide suitable for PET applications (see also Herzog et al., J. Nucl. Med. 34:2222-2226, 1993). Although zirconium-89 has a relatively low probability of positron emission (23%), good-quality images can be obtained in PET because of its relatively low energy (397 keV).

In the alternative, conjugates comprising chelators arranged for complexation of gamma emitters according to the current invention may be used for SPECT applications.

Based on these findings LRRC15 has been suggested as a novel marker for cancer associated fibroblast (CAF) and mesenchymal cells.

### DNA/RNA for LRRC15 Antibody

According to a 7^{th} aspect, there is provided a polynucleotide encoding an antibody or antigen-binding fragment according to the 2^{nd} aspect. Sequences of exemplary polynucleotides are provided with the sequence listing.

### Vector for Antibody

According to an 8^{th} aspect, there is provided a vector comprising a polynucleotide according to the 7^{th} aspect.

### Production Cell for Antibody

According to a 9^{th} aspect, there is provided an isolated cell arranged for production of an antibody or antigen-binding fragment according to the 2^{nd} aspect. Preferably the isolated cell is a mammalian host cell such as a CHO cell or a HEK293 cell.

### Production Method for Antibody

According to a 10^{th} aspect, there is provided a method of producing an antibody or antigen-binding fragment according to the 2^{nd} aspect, or a conjugate according to the 1^{st} or 3^{rd} aspect.

In some preferred embodiments, which may be the same or different, the method comprises coupling of the at least one chelating group arranged for complexation of a radionuclide to the at least one targeting moiety binding LRRC15, to obtain a tissue-targeting chelator complex.

In some preferred embodiments, the radionuclide is ²²⁷Th, and the coupling of the at least one chelating group arranged for complexation of a radionuclide to the at least one targeting moiety binding LRRC15 is followed by contacting the obtained tissue-targeting chelator complex with an aqueous solution comprising 4+ ions of the radionuclide.

In some preferred embodiments, the method comprises culturing a cell according to the 9^{th} aspect to obtain an antibody or antigen-binding fragment according to the second aspect and optionally comprises purification of the antibody or antigen-binding fragment.

In some preferred embodiments, the method comprises making a polynucleotide according to the 7^{th} aspect.

### Kit of Parts

According to an 11^{th} aspect, there is provided a kit comprising the antibody or antigen-binding fragment according to the second aspect, or a conjugate according to the 1^{st} or 3^{rd} aspect, or the pharmaceutical composition according to the 4^{th} aspect, with instructions for use.

The antibodies, fragments, conjugates or pharmaceutical compositions of the present invention can be provided in a kit, i.e., a packaged combination of reagents in predetermined amounts in one or more containers with instructions. For example, where the antibody, fragment or conjugate is a therapeutic antibody, fragment or conjugate, the instructions for use may comprise the package insert.

For example, where the antibody is labeled with an enzyme, the kit may include substrates and cofactors required by the enzyme (e.g., a substrate precursor which provides the detectable chromophore or fluorophore). In addition, other additives may be included such as stabilizers, buffers (e.g., a block buffer or lysis buffer) and the like. The relative amounts of the various reagents may be varied widely to provide for concentrations in solution of the reagents which substantially optimize the sensitivity of the assay. Particularly, the reagents may be provided as dry powders, usually lyophilized, including excipients which on dissolution will provide a reagent solution having the appropriate concentration.

### Further Uses

The LRRC15 antibodies, fragments and conjugates as described herein may be used for a variety of purposes, e.g. for in vitro and ex vivo applications and/or in vitro and ex vivo diagnostics.

In a specific example, the antibodies or conjugates can be used for purification or immobilization of LRRC15 or LRRC15 expressing cells.

In another specific example, the antibodies or conjugates can be used for qualitatively and/or quantitatively measuring levels of LRRC15 or LRRC15 expressing cells in biological samples, e.g. in immunoassays, see, e.g., Harlow et al., Antibodies: A Laboratory Manual, Second Edition (Cold Spring Harbor Laboratory Press, 1988).

### General Procedures

Radionuclides can be obtained as known in the art. The most widely used cyclotron production method of zirconium-89 is from yttrium-89 using a (p,n) reaction.

### Conjugation of Chelator and Targeting Moiety

A chelator or chelating group can be covalently coupled (conjugated) to a targeting moiety using reactive functional groups either directly or indirectly using a linker. Common bioconjugation techniques utilize functional groups such as carboxylic acids or activated esters (e.g. N-hydroxysuccinimide NHS-ester, tetrafluorophenyl TFP-ester) for amide couplings, isothiocyanates for thiourea couplings, and maleimides for thiol couplings. Click chemistry may also be used, e.g. with traditional copper(I) catalyzed azide-alkyne Huisgen 1,3-dipolar cycloaddition “click” reaction (forming a 1,2,3-triazole-ring linkage), or copper-free reactions like strain-promoted azide-alkyne cycloadditions (e.g. dibenzocyclooctyne/azide reaction) and Diels-Alder click reactions (e.g. transcyclooctene/1,2,4,5-tetrazine).

### Introduction of the Radionuclide

Introduction of the radionuclide into the conjugate may occur prior to or after administration of the conjugate in a therapeutic or diagnostic setup. Synthesis of the conjugate prior to the radiolabeling and one-step radiolabeling is preferred, especially with short half-life radionuclides. However, the development of α-particle radioimmunoconjugates may require more complex procedures.

Maguire et al. have proposed a 1-step method for ²²⁵Ac radiolabeling of monoclonal antibodies that allows for radiochemical yields of up to 80% (Maguire, William F., et al. “Efficient 1-step radiolabeling of monoclonal antibodies to high specific activity with 225Ac for α-particle radioimmunotherapy of cancer.” Journal of Nuclear Medicine 55.9 (2014): 1492-1498). Ramdahl et al. reported superior properties with respect to ²²⁷Th radiolabeling and stability using Me-3,2-HOPO compared with the DOTA chelator (Ramdahl, Thomas, et al. “An efficient chelator for complexation of thorium-227.” Bioorganic & medicinal chemistry letters 26.17 (2016): 4318-4321.).

Further approaches are known in the art, such as pretargeting, which separates the administration of the targeting conjugate from the radioisotope (Altai, Mohamed, et al. “Pretargeted imaging and therapy.” Journal of Nuclear Medicine 58.10 (2017): 1553-1559.).

### Synthesis of Compounds According to Formula I

The schemes and procedures described below illustrate synthetic routes to the compounds of formula (I) of the invention and are not intended to be limiting. It is obvious to the person skilled in the art that the order of transformations as exemplified in the Schemes can be modified in various ways. The order of transformations exemplified in the Schemes is therefore not intended to be limiting. In addition, interconversion of any of the substituents, R1, R2, R3, R4, can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3rd edition, Wiley 1999).

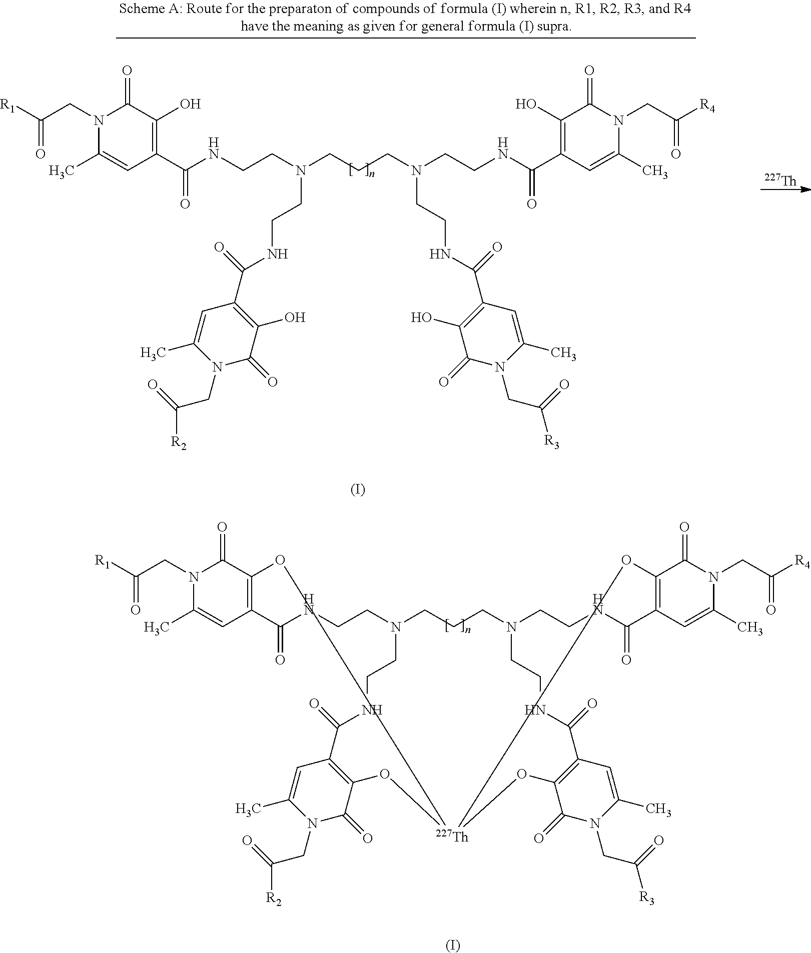

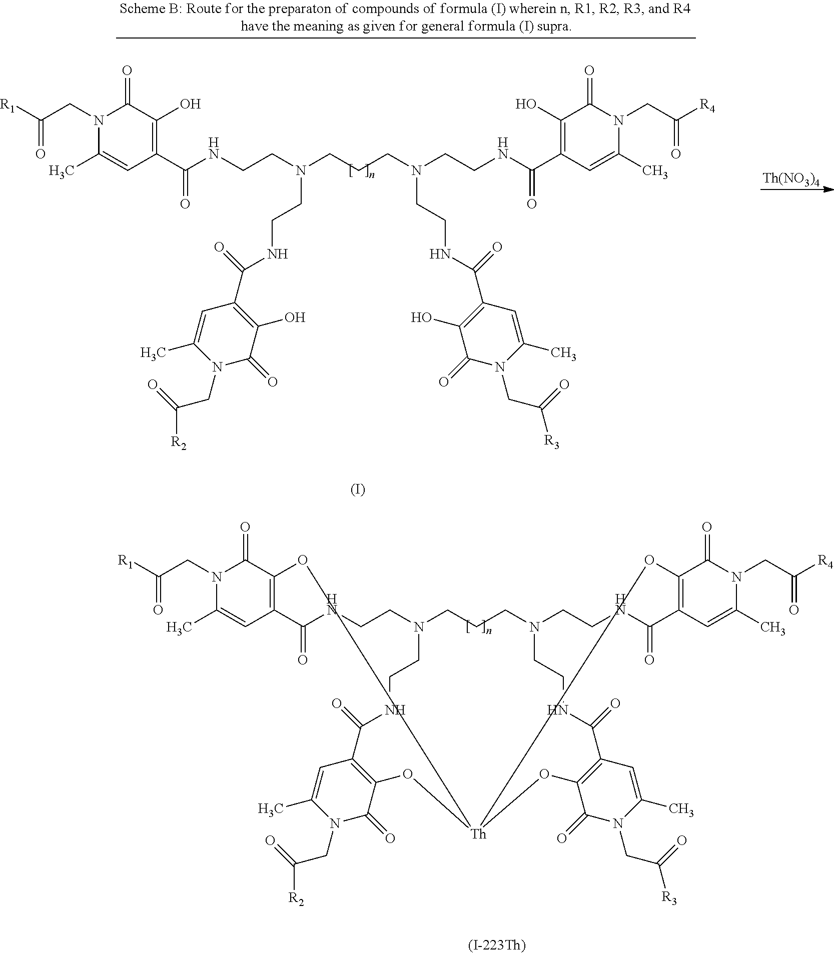

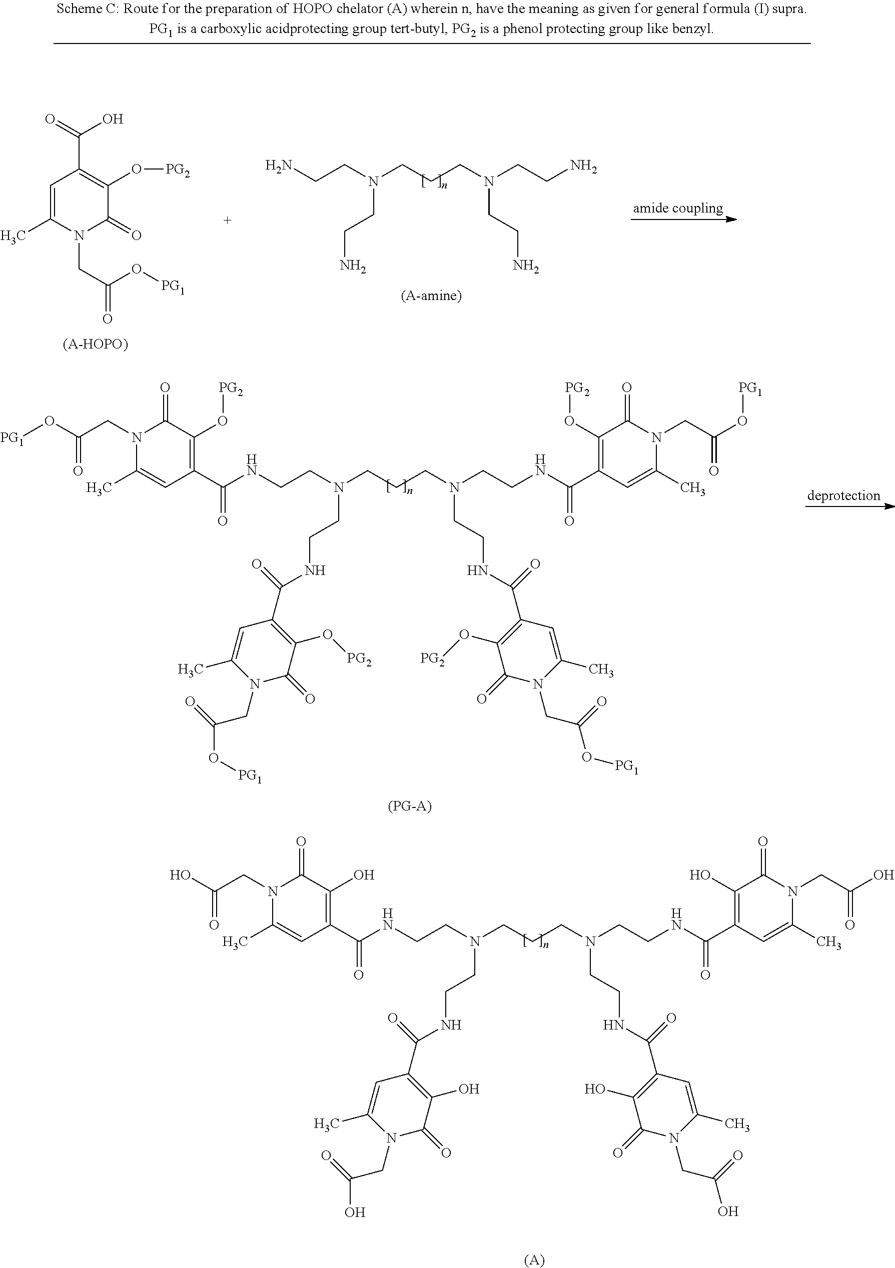

Suitably protected hydroxypyridone A-HOPO is coupled to tetraamine A-amine under amide coupling conditions known to those skilled in the art. Possible reaction conditions include but are not limited to amide coupling reagents like HATU (1-[Bis(dimethylamino)methylene]-1H-1,2,3-triazolo[4,5-b]pyridinium 3-oxide hexafluorophosphate). In the following step the protecting groups are removed by conditions known to those skilled in the art for the respective protecting groups. Possible reaction conditions include but are not limited to cleavage by hydrochloric acid, hydrobromic acid, hydrogen bromide in acetic acid or trifluoroacetic acid.

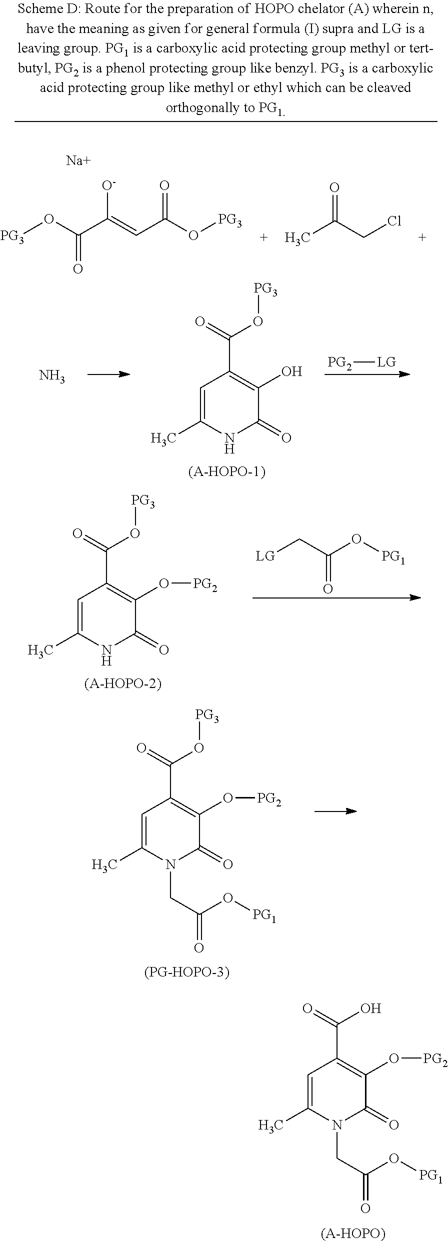

PG₃-protected oxalacetate sodium salt is reacted with chloroacetone and ammonia under suitable conditions to give protected hydroxypyridone A-HOPO-1. These reaction conditions include but are not limited to heating, elevated pressure or the use of a Lewis acid like aluminium trichloride. A-HOPO-1 is then protected at the phenol position by reaction with PG₂-X to give A-HOPO-2. After that A-HOPO-2 is reacted with an activated protected acetic acid equivalent like tert-butyl bromoacetate to give PG-HOPO-3. Finally PG₃ is cleaved selectively by conditions known to those skilled in the art like for example lithium hydroxide for the cleavage of ethyl or methyl esters to give A-HOPO.

The order of the second and third step in this synthesis can be exchanged meaning to alkylate the pyridine NH before protection of the phenol.

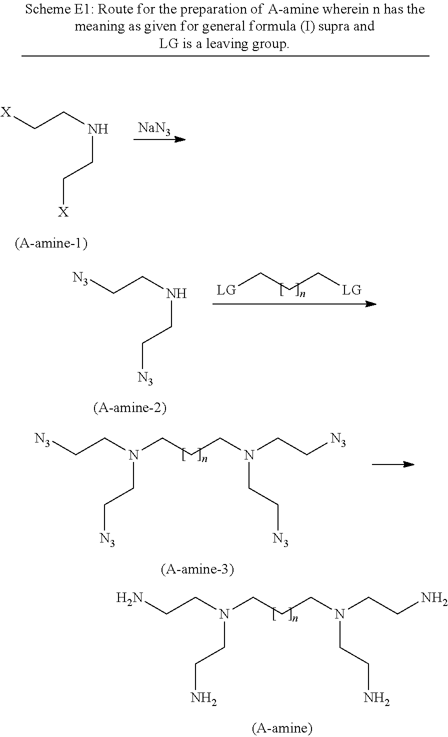

Bis-reactive A-amine-1 is reacted with an appropriate azide like sodium azide under conditions for alkylic nucleophilic displacement known to those skilled in the art to give bis azide A-amine-2. This is then further reacted with an appropriate bis-reactive alkane like 1,3-dibromopropoane under conditions for alkylic nucleophilic displacement known to those skilled in the art to give tetraazide A-amine-3. Tetraazide A-amine-3 is the reduced to tetraamien A-amine under conditions typical for the reduced of azides to the corresponding amine like catalytic hydrogenation with palladium on charcoal or with triphenyl phosphine.

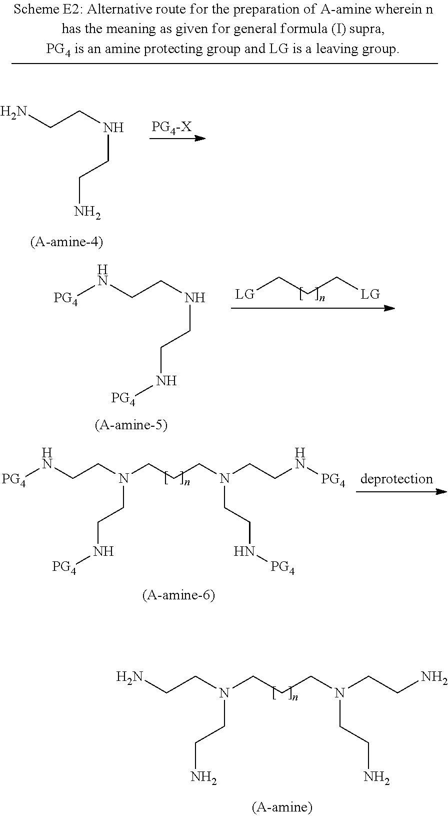

Trisamine A-amine-4 is protected at the terminal primary amines with a suitable protecting group like Boc, Fmoc, Cbz, ortrityl to give bis-protected trisamine A-amine-5. This is then further reacted with an appropriate bis-reactive alkane like 1,3-dibromopropoane under conditions for alkylic nucleophilic displacement known to those skilled in the art to give tetrakis-protected hexaamine A-amine-6. A-amine-6 is then deprotected under conditions known to those skilled in the art to give A-amine.

### EXAMPLES

All examples were carried out using standard techniques, except where described otherwise herein. Routine molecular biology techniques of the following examples can be carried out as described in standard laboratory manuals, such as Sambrook et al., Molecular Cloning: A Laboratory Manual, 2nd Ed.; Cold Spring Harbor Laboratory Press, Cold Spring Harbor, N.Y., 1989.

Generation of ²²⁷Th

227Th was selectively isolated from an ²²⁷Ac mixture, which had been growing in daughters for two weeks, by adding 0.25 ml of 7 M HNO₃ to the actinium mixture (which had been evaporated to dryness) and eluting the solution through an anion exchange column. The column had an inner diameter of 2 mm and a length of 30 mm containing approximately 70 mg of AG-1×8 anion exchange resin (Biorad Laboratories, Hercules, Calif., USA) (nitrate form). The column was washed with 2-4 ml of 7 M HNO₃ to remove ²²⁷Ac, ²²³Ra and Ra daughters while retaining ²²⁷Th. Subsequently ²²⁷Th was stripped from the column with a few ml of 12M HCl. Finally the HCl was evaporated to dryness and the ²²⁷Th re-dissolved in 0.05 M HCl.

### Example 1: Generation of Antibodies, Antigens and Reference Compounds

All antibodies were expressed in HEK293 cells using standard transient transfection procedures and purified from the cell culture supernatant via Protein-A and size exclusion chromatography. In some examples, prior art antibody sequences such as those of TPP-12942 (huM25) were randomly altered. Only few of the resulting antibodies showed improved behaviour. Only examples with improved characteristics are included herein.

In other examples, a fully human antibody phage display library was used to isolate LRRC15-specific, human monoclonal antibodies such as TPP-1633 (heavy and light chain provided in SEQ ID NO:19 and SEQ ID NO:20) by protein panning (see Hoogenboom H. R., Nat Biotechnol 2005; 23(3):1105-16) with extracellular domains of human LRRC15 and murine LRRC15 as immobilized targets.

Protein sequences for LRRC15 were retrieved from the UniProtKB/TrEMBL database using the following identifier: Q8TF66 for human LRRC15, Q80X72 for murine LRRC15 and G7NYR2 for cynomolgus (Macaca fascicularis) LRRC15.

To obtain recombinant extracellular domains of LRRC15 protein, extracellular domains were C-terminally appended with a His Tag and expressed in HEK293 cells using standard transient transfection procedures (cf. SEQ ID NO:137 for human LRRC15, SEQ ID NO:138 for murine LRRC15 and SEQ ID NO:139 for Macaca fascicularis LRRC15). Proteins were purified from the cell culture supernatant via Ni-IMAC and size exclusion chromatography.

Different Fab-phages were identified using phage display, and the corresponding antibodies were re-cloned into a mammalian IgG1 expression vector which provides the missing CH2-CH3 domains not present in the soluble Fab. The resulting IgGs were transiently expressed in mammalian cells, purified by Protein A chromatography and further characterized by their binding abilities to human and murine LRRC15. The antibody TPP-1633 was found to be cross-reactive to both human and mouse LRRC15 with monovalent affinities (KD) in the 200 nM range. Antibodies TPP-1633 and TPP-12942 were furthermore subjected to sequence germlining and further alterations were introduced. The resulting antibodies (TPP-14389, TPP-14392, TPP-17073, TPP-17074 for TPP-1633 and TPP-17078, TPP-17405, TPP-17418, TPP-17419, TPP-17421, TPP-17422 for TPP-12942) were characterized with regard to monovalent binding affinity (K_{D}) and off-rate (k_{d}) as assessed by surface plasmon resonance (SPR). Surprisingly, variants of both families were found to be superior not only with regard to affinity but also with regard to multiple other properties as listed in Table 1, and as described elsewhere herein.

### Example 1: Assessment of Binding to Off-Targets

To assess potential off-target binding activities to unrelated human targets, antibodies TPP-12942 and TPP-1633 were subjected to Retrogenix′ (High Peak, United Kingdom) cell microarray for off-target profiling. Briefly, each test antibody was screened at a fixed antibody dose for binding against fixed HEK293 cells on slides expressing 4575 different human plasma membrane proteins individually. Hits were subsequently confirmed by flow cytometry on living HEK293 cells transfected with the respective off-target in dose response.

Surprisingly, when determining the fixed dose for TPP-12942 and TPP-1633, it was found that TPP-1633 did not give any background signal on fixed, untransfected HEK293 cells up to an antibody concentration of 20 μg/ml. Therefore, the fixed dose for TPP-1633 was set to 20 μg/ml. In contrast, TPP-12942 showed substantial background staining at 20 μg/ml. Therefore, the concentration for the primary screen was reduced to 5 μg/ml for huM25.

Upon screening for binding against fixed HEK293 cells on slides at fixed dose, both antibodies specifically and reproducibly bound LRRC15 with strong intensity. Four putative off-target hits were evaluated by flow cytometry on living HEK293-transfected cells in dose response. An off target effect could be confirmed between TPP-12942 and Ephrin type-B receptor 6 (EPHB6) at ≥1 μg/ml on live cells (Table 2, FIG. 1).

TABLE 1
Overview of the properties of the inventive antibodies
in comparison with prior art antibody TPP-12942.

| | | | | clearance | Binding |
|---|---|---|---|---|---|
| | | Temperature | | rate in | Affinity KD |
| | | dependent | Driver of | cynomolgus | [M⁻¹] to |
| | Off target | loss in binding | binding | monkeys CL | human |
| | binding | affinity | reaction | [ml kg⁻¹ h⁻¹] | LRRC15 |
| TPP-ID No: | (Example 2) | (Example 3) | (Example 4) | (Example 5) | (Example 6) |
| TPP-1633 | none | >9 fold | nd | nd | 1.5E−07 |
| TPP-12942 | Binding to | 13.8 fold | Enthalpy | 0.58 | 2.5E−08 |
| [huM25] | EPHB6 | | | | |
| TPP-14389 | none | 108.2 fold | Enthalpy | 0.23 | 5.9E−09 |
| TPP-14392 | none | 56.9 fold | Enthalpy | 0.33 | 5.9E−09 |
| TPP-17073 | nd | nd | nd | nd | 2.1E−10 |
| TPP-17074 | nd | 33.3 fold | Enthalpy | nd | 1.9E−10 |
| TPP-17078 | strongly | 30.9 fold | Enthalpy | 0.31 | 1.7E−09 |
| | reduced | | | | |
| | binding to | | | | |
| | EPHB6 | | | | |
| TPP-17405 | nd | nd | nd | nd | 8.6E−10 |
| TPP-17418 | nd | nd | nd | nd | 1.1E−09 |
| TPP-17419 | nd | nd | nd | nd | 3.4E−09 |
| TPP-17421 | none | 5.2 fold | Entropy | 0.50 | 9.6E−10 |
| TPP-17422 | nd | nd | nd | nd | 1.3E−09 |
| | Binding | Binding | | | |
| | Affinity KD | Affinity KD | | | Low pH |
| | [M⁻¹] to | [M⁻¹] to | Germline | Germline | stability in |
| | cynomolgus | mouse | Deviations | Deviations | downstream |
| | LRRC15 | LRRC15 | Light Chain | Heavy Chain | processing |
| TPP-ID No: | (Example 6) | (Example 6) | (Example 7) | (Example 7) | (Example 8) |
| TPP-1633 | 1.9E−07 | 2.7E−07 | 15 | 11 | nd |
| TPP-12942 | 3.6E−08 | 4.9E−08 | 16 | 24 | reduced |
| [huM25] | | | | | |
| TPP-14389 | 9.0E−09 | 5.2E−08 | 13 | 9 | given |
| TPP-14392 | 1.0E−08 | 7.5E−09 | 14 | 9 | given |
| TPP-17073 | 1.8E−10 | 2.5E−09 | 14 | 10 | nd |
| TPP-17074 | 1.6E−10 | 2.1E−09 | 14 | 10 | nd |
| TPP-17078 | 1.8E−09 | 2.9E−09 | 9 | 24 | given |
| TPP-17405 | 7.8E−10 | 2.7E−09 | 12 | 24 | nd |
| TPP-17418 | 9.9E−10 | 8.9E−10 | 9 | 24 | nd |
| TPP-17419 | 2.5E−09 | 2.3E−09 | 11 | 24 | nd |
| TPP-17421 | 9.1E−10 | 1.6E−09 | 10 | 24 | given |
| TPP-17422 | 1.2E−09 | 2.3E−09 | 9 | 24 | nd |

nd: not determined.

TABLE 2
Binding of TPP-12942 (huM25) to HEK293 cells transfected with either LRRC15/ZsGreen1,
EPHB6/ZsGreen1, PIK3AP1/ZsGreen1, or ZsGreen1-only (ZS HEK). ZsGreen1
is a commercially available bright green fluorescent protein derived from
a Zoanthus sp. Shown is the Median Fluorescence of Alexa Fluor 647 (AF647)
labeled secondary antibody versus antibody concentration as determined by flow
cytometry. The EC₅₀ binding value of TPP-12942 to LRRC15 was determined
to be 1.4 +/− 0.5 μg/ml. The elevated binding of TPP-12942
to EPHB6-transfected cells is evident.

| TPP-12942 (μg/ml) | 0 | 0.004 | 0.02 | 0.1 | 0.5 | 2.5 | 12.5 | 62.5 |
|---|---|---|---|---|---|---|---|---|
| LRRC15 | 1088 | 16446 | 44092 | 85883 | 135497 | 227762 | 321803 | 377487 |
| EPHB6 | 1602 | 2686 | 5544 | 9576 | 16827 | 22976 | 37470 | 62443 |
| PIK3AP1 | 1380 | 2386 | 3738 | 5993 | 8034 | 11283 | 23597 | 32741 |
| ZsGreen-only | 1337 | 2563 | 3488 | 5082 | 5995 | 8771 | 13050 | 22103 |
| transfectants | | | | | | | | |

A further Retrogenix cell microarray screen was performed for antibodies TPP-14389 and TPP-14392, this time covering 5647 human plasma membrane proteins on fixed cells using 20 μg/ml of antibody. Both antibodies recognized their primary target LRRC15 as indicated by strong median fluorescence. No other specific cell surface interactions were observed for TPP-14392, indicating a high specificity for the primary target LRRC15. For TPP-14389, an initial hit for Cathepsin S (CTSS) was not confirmed by flow cytometry analysis with living cells (Table 3, FIG. 2). In summary, comparison with TPP-12942 showed a superior profile in off-target binding for TPP-14389 and TPP-14392.

TABLE 3
Binding of TPP-14389 to HEK293 cell transfected with LRRC15/ZsGreen1, CTSS/ZsGreen1,
or ZsGreen1-only (ZS HEK). Shown is the Median Fluorescence of AF647-labeled secondary
antibody versus antibody concentration as determined by flow cytometry. The EC₅₀ binding
value of TPP-14389 to LRRC15 was determined to be 0.26 +/− 0.03 μg/ml.
No binding of TPP-14389 to Cathepsin S (CTSS)-transfected cells is evident.

| TPP-14389 (μg/ml) | 0 | 0.004 | 0.02 | 0.1 | 0.5 | 2.5 | 12.5 | 62.5 |
|---|---|---|---|---|---|---|---|---|
| LRRC15 | 999 | 10687 | 34853 | 108786 | 244506 | 312705 | 366135 | 365927 |
| CTSS | 1017 | 2029 | 5077 | 5479 | 5446 | 6115 | 7092 | 8823 |
| ZsGreen-only | 937 | 1899 | 3621 | 4706 | 4825 | 5227 | 6109 | 7183 |
| transfectants | | | | | | | | |

TABLE 4
Binding of TPP-12942 (huM25), TPP-17078, and TPP-17421 as well as IgG1 isotype control
TPP-754 to HEK293 cells transfected with LRRC15/ZsGreen1, EPHB6/ZsGreen1 or ZsGreen1
only. Shown is the median fluorescence of AF647 labeled secondary antibody versus antibody
concentration as determined by flow cytometry. Background binding of each antibody to the
cells (i.e. binding to ZsGreen1 only transfectants) can be subtracted from the LRRC15
transfected cells for each antibody concentration. The EC₅₀ binding value of TPP-12942,
TPP-17078, and TPP-17421 to LRRC15 was determined to be 0.20 μg/ml, 0.15 μg/ml,
and 0.42 μg/ml, respectively. No binding is evident for human IgG1 isotype
control TPP-754. The EC₅₀ binding value of TPP-12942 to EPHB6 was determined to be
51.8 μg/ml. TPP-754 is a human IgG1 isotype control.

| | 0 | 0.004 | 0.02 | 0.1 | 0.5 | 2.5 | 12.5 | 62.5 |
|---|---|---|---|---|---|---|---|---|
| TPP-12942 (μg/ml) | | | | | | | | |
| LRRC15 | 594 | 19779 | 63361 | 212724 | 523633 | 746380 | 726283 | 620200 |
| EPHB6 | 591 | 1243 | 2118 | 4307 | 6121 | 9005 | 11869 | 17228 |
| ZsGreen-only | 542 | 1029 | 1557 | 2031 | 2352 | 2718 | 3192 | 4185 |
| transfectants | | | | | | | | |
| TPP-17078 (μg/ml) | | | | | | | | |
| LRRC15 | 597 | 27568 | 83672 | 284457 | 527120 | 776573 | 706781 | 621085 |
| EPHB6 | 633 | 1777 | 2545 | 3190 | 3679 | 5482 | 7969 | 14572 |
| ZsGreen-only | 510 | 1338 | 1836 | 2227 | 2364 | 3179 | 3923 | 8279 |
| transfectants | | | | | | | | |
| TPP-17421 (μg/ml) | | | | | | | | |
| LRRC15 | 588 | 13093 | 35177 | 101968 | 390531 | 649195 | 735662 | 650310 |
| EPHB6 | 628 | 1252 | 1774 | 2811 | 3151 | 4151 | 5394 | 9054 |
| ZsGreen-only | 492 | 964 | 1375 | 1940 | 2592 | 2972 | 3992 | 6970 |
| transfectants | | | | | | | | |
| TPP-754 (μg/ml) | | | | | | | | |
| LRRC15 | 618 | 607 | 582 | 595 | 554 | 663 | 1252 | 682 |
| EPHB6 | 588 | 622 | 553 | 599 | 571 | 643 | 620 | 631 |
| ZsGreen-only | 573 | 598 | 550 | 583 | 565 | 579 | 711 | 651 |
| transfectants | | | | | | | | |

Each test antibody showed a significant (and approximately equivalent) level of binding to the primary target, LRRC15. As observed before, a secondary off-target interaction to EPHB6 was observed with TPP-12942. This interaction to EPHB6 was largely reduced for TPP-17078 and absent for TPP-17421. In summary, the antibodies according to the current invention, in particular TPP-14389, TPP-14392, TPP-17421 and TPP-17078 show an improved off-target binding compared with prior art antibody TPP-12942. In addition, these antibodies do not show any polyreactivity (as assessed by binding to a panel of LRRC15 negative cells in FACS).

The absence of off-target binding is an important feature for a therapeutic antibody destined for human patients as this can lead to unexpected safety problems or pharmacokinetic insufficiencies. This is illustrated by results from early clinical trial with the anti-PD1 antibody SHR-1210 (also known as camrelizumab) that demonstrated the expected biological activity, but also had the unusual toxicity profile of causing capillary hemangioma. As this highly specific side-effect has not been reported for other anti-PD1 antibodies, the toxicity is attributed to off-target binding activities of this antibody (Finlay et al., MAbs. 2019 January; 11(1):26-44).

### Example 3: Temperature-Depending Binding of Antibodies to LRRC15

To assess if anti-LRRC-15 antibodies show a difference in binding at different temperatures, binding assays were conducted using surface plasmon resonance (SPR). Binding assays were performed on a Biacore T200 instrument at temperatures of 10° C., 20° C., 25° C. and 37° C. with assay buffer HBS EP+, 1 mg/ml BSA (bovine serum albumine), 300 mM NaCl, 0.05% NaN₃. Antibodies were captured via anti-human Fc IgGs covalently amine coupled to a CM5 sensor chip and human LRRC15 was used as an analyte in a concentration series from 1.56-200 nM in multi cycle kinetics mode. Pre-experiments were conducted to have an equal capture level at each temperature. Obtained sensorgrams were fitted to a 1:1 Langmuir binding model to derive kinetic data. Results are shown in Table 5.

TABLE 5
Summary of kinetic data acquired at different
temperatures for TPP-12942, TPP-17078, TPP-17074,
TPP-17421, TPP-1633, TPP-14389, TPP-14392.

| TPP No. | Temperature | kₐ [1/Ms] | k_{d} [1/s] | K_{D} [M] |
|---|---|---|---|---|
| TPP-12942 | 10° C. | 3.7E+04 | 2.0E−04 | 5.2E−09 |
| | 20° C. | 6.7E+04 | 8.8E−04 | 1.3E−08 |
| | 25° C. | 9.0E+04 | 1.9E−03 | 2.1E−08 |
| | 37° C. | 1.0E+05 | 7.2E−03 | 7.2E−08 |
| TPP-17078 | 10° C. | 4.0E+04 | 1.3E−05 | 3.2E−10 |
| | 20° C. | 5.2E+04 | 4.6E−05 | 8.8E−10 |
| | 25° C. | 5.8E+04 | 9.1E−05 | 1.6E−09 |
| | 37° C. | 6.7E+04 | 6.6E−04 | 9.9E−09 |
| TPP-17074 | 10° C. | 3.2E+05 | 1.8E−05 | 5.7E−11 |
| | 20° C. | 4.5E+05 | 6.1E−05 | 1.4E−10 |
| | 25° C. | 5.5E+05 | 1.2E−04 | 2.2E−10 |
| | 37° C. | 7.9E+05 | 1.5E−03 | 1.9E−09 |
| TPP-17421 | 10° C. | 4.2E+04 | 2.0E−05 | 4.6E−10 |
| | 20° C. | 5.2E+04 | 4.3E−05 | 8.2E−10 |
| | 25° C. | 6.0E+04 | 5.3E−05 | 8.8E−10 |
| | 37° C. | 7.6E+04 | 1.8E−04 | 2.4E−09 |
| TPP-1633 | 10° C. | 8.0E+04 | 1.4E−03 | 1.8E−08 |
| | 20° C. | 1.2E+05 | 9.3E−03 | 8.0E−08 |
| | 25° C. | 1.4E+05 | 2.2E−02 | 1.6E−07 |
| | 37° C. | | Not | |
| | | | determinable | |
| TPP-14389 | 10° C. | 1.9E+05 | 1.2E−04 | 6.1E−10 |
| | 20° C. | 2.3E+05 | 4.7E−04 | 2.0E−09 |
| | 25° C. | 2.4E+05 | 1.3E−03 | 5.4E−09 |
| | 37° C. | 3.0E+05 | 1.9E−02 | 6.6E−08 |
| TPP-14392 | 10° C. | 3.7E+04 | 6.0E−05 | 1.6E−09 |
| | 20° C. | 4.5E+04 | 1.3E−04 | 2.9E−09 |
| | 25° C. | 5.0E+04 | 3.4E−04 | 6.8E−09 |
| | 37° C. | 5.5E+04 | 5.0E−03 | 9.1E−08 |

As compared to TPP-12942, both, TPP-17078 and TPP-17421 show only a minor loss of affinity with increasing temperature from 10° C. to 37° C. (see also FIG. 5), primarily driven by a slower decrease of the dissociation rate constant k_{d}. Importantly, half-lives of the antibody-antigen complexes at 37° C. differ significantly (1.6 min for TPP-12942, 17.5 min for TPP-17078 and 64.2 min for TPP-17421) (Table 6).

TABLE 6
Summary of antibody-antigen complex half life calculated based
on K_{D} values at different temperatures for TPP-1633, TPP-14389,
TPP-14392, TPP-12942, TPP-17421, TPP-17074, TPP-17078.

| TPP | Temperature | k_{d} [1/s] | Complex Half Life [min] |
|---|---|---|---|
| 1633 | 10° C. | 1.40E−03 | 8.3 |
| | 20° C. | 9.30E−03 | 1.2 |
| | 25° C. | 2.20E−02 | 0.5 |
| | 37° C. | n.d. | |
| 14389 | 10° C. | 1.20E−04 | 96.3 |
| | 20° C. | 4.70E−04 | 24.6 |
| | 25° C. | 1.30E−03 | 8.9 |
| | 37° C. | 1.90E−02 | 0.6 |
| 14392 | 10° C. | 6.00E−05 | 192.5 |
| | 20° C. | 1.30E−04 | 88.9 |
| | 25° C. | 3.40E−04 | 34.0 |
| | 37° C. | 5.00E−03 | 2.3 |
| 12942 | 10° C. | 2.00E−04 | 57.8 |
| | 20° C. | 8.80E−04 | 13.1 |
| | 25° C. | 1.90E−03 | 6.1 |
| | 37° C. | 7.20E−03 | 1.6 |
| 17421 | 10° C. | 2.00E−05 | 577.6 |
| | 20° C. | 4.30E−05 | 268.7 |
| | 25° C. | 5.30E−05 | 218.0 |
| | 37° C. | 1.80E−04 | 64.2 |
| 17074 | 10° C. | 1.80E−05 | 641.8 |
| | 20° C. | 6.10E−05 | 189.4 |
| | 25° C. | 1.20E−04 | 96.3 |
| | 37° C. | 1.50E−03 | 7.7 |
| 17078 | 10° C. | 1.30E−05 | 888.7 |
| | 20° C. | 4.60E−05 | 251.1 |
| | 25° C. | 9.10E−05 | 127.0 |
| | 37° C. | 6.60E−04 | 17.5 |

This feature is especially relevant for therapeutic interventions as the compound needs to act at ˜ 37° C. in a human patient and thus antibodies with a prolonged half-life of the binding complex will be retained at the tumor site and target a TTC for a prolonged time. TPP-17074 shows a decrease in binding only at 37° C., but not below, also showing that introduction of the specific mutations into the CDRs leads to a surprising stabilization of the dissociation rate constant in a temperature gradient.

### Example 2: Conversion of Enthalpic Binder to Entropic Binder

Thermodynamics help to explain why an interaction is happening and what the driving forces for the interaction are. To assess the thermodynamic parameters for anti-LRRC15 antibodies binding assays were conducted using surface plasmon resonance (SPR). Binding assays were performed on a Biacore T200 instrument at temperatures of 10° C., 20° C., 25° C. and 37° C. with assay buffer HBS EP+, 1 mg/ml BSA, 300 mM NaCl, 0.05% NaN3. Antibodies were captured via anti-human Fc IgGs covalently amine coupled to a CM5 sensor chip and human LRRC15 was used as an analyte in a concentration series from 1.56-200 nM in multi cycle kinetics mode. Pre-experiments were conducted to have an equal capture level at each temperature. Obtained sensorgrams were fitted to a 1:1 Langmuir binding model to derive kinetic data. Thermodynamic parameters were obtained using the integrated thermodynamics wizard in the Biacore T200 software. Van′t Hoff and Eyring plots were calculated and plotted and thermodynamic parameters were derived. Thermodynamic parameters are shown in FIG. 2.

TABLE 7
Thermodynamic parameters, free Gibbs energy (ΔG), enthalpy
(ΔH) and entropy term (−TΔS) are shown in kJ/mol
for six different antibodies

| | ΔH⁰ [kJ/mol] | −TΔS [kJ/mol] | ΔG⁰ [kJ/mol] |
|---|---|---|---|
| TPP-14392 | −110 | 64 | −46 |
| TPP-14389 | −130 | 81 | −47 |
| TPP-17074 | −95 | 40 | −54 |
| TPP-12942 | −71 | 28 | −44 |
| TPP-17078 | −93 | 43 | −50 |
| TPP-17421 | −44 | −7.6 | −51 |

Any spontaneous biological interaction is driven by a negative change in the Gibbs free energy ΔG which can be further dissected in enthalpy (ΔH) and entropy (ΔS) terms. An interaction which is driven by enthalpy is caused by non-covalent interactions like hydrogen bonding, van der Waals or electrostatic interactions like salt bridges. Vice versa, entropy driven reactions are based on the change of the system in terms of conformational changes in the antibody, antigen or both or the reorganization of solvent molecules interacting with the involved binding partners.

Furthermore, there is also the concept of an enthalpy/entropy compensation effect. For example, a gain in enthalpy by new non-covalent interactions leads to a higher constraint in the complex in terms of conformational freedom and thus the entropy of the system decreases.

As can be seen from Table 7 and FIG. 7, all anti-LRRC15 antibodies are driven by enthalpic terms and have an entropic burden. Strikingly, TPP-17421 exhibit a negative entropic term compared to antibody TPP-12942. Here, the alterations have surprisingly led to a completely different thermodynamic fingerprint by lowering the enthalpic term, but on the other hand completely abolishing the entropic barrier and introducing entropic driving forces. Thus, to reach the same or equal affinity as other improved TPP-12942 versions, TPP-17421 does not need high enthalpy values, but makes use of combined smaller changes. This fingerprint nicely balances the interaction between high specificity by non-covalent interactions, but on the other hand also introduces e.g. more flexibility into the antibody-antigen complex additionally leading to specificity utilizing this entropic effect not present in TPP-12942.

### Example 3: Clearance Rates in Cynomolgus Monkeys

The pharmacokinetic properties of some of the antibodies were assessed in female cynomolgus monkeys (M. fascicularis) (n=2, each). The animals were dosed with a single bolus of antibody solution in PBS buffer intravenously at 1 mg/kg body weight. Blood samples were taken after different time points (more than 10 time points), including terminal time points covering at least 336 h up to maximal 672 hours after dosing. Blood was collected in K3 EDTA tubes, and blood plasma was obtained by centrifugation, and the plasma was subsequently frozen at −20° C.

The plasma concentrations of test antibodies in plasma were determined using a generic IgG ELISA. Briefly, ELISA plates were coated with anti-human IgG-Fc from goat. After incubation with test samples, plates were washed and incubated using anti-human-IgG(H+L) antibody from donkey conjugated to Horseradish Peroxidase (HRP). After another washing step, the HRP-substrate OPD was added and development product was monitored by absorption at 490 nm. Standard samples of known concentration were included, and values obtained were fitted by a 4-parameter equation. Unknown concentrations between the LLOQ (Lower Limit Of Quantitation) and ULOQ (Upper Limit Of Quantitation) were determined by interpolation. Pharmacokinetic parameters like CL (clearance) were calculated by non-compartmental analysis (NCA). The algorithms for calculating the parameters based on rules published in general textbooks of pharmacokinetics, with CL=dose/AUC. The clearance CL values for different LRRC15 antibodies are shown in table 8:

TABLE 8
Clearance (CL) values determined for different antibodies
in female cynomolgus monkeys (n = 2, each).

| Protein-ID | CL [ml kg⁻¹ h⁻¹] |
|---|---|
| TPP-12942 | 0.58 |
| TPP-14389 | 0.23 |
| TPP-14392 | 0.33 |
| TPP-17078 | 0.31 |
| TPP-17421 | 0.50 |

The clearance values CL for TPP-14389, TPP-14392 and TPP-17078 are significantly lower than the CL value of TPP-12942. The residence time of an antibody molecule in the body will increase with a lower clearance rate CL and the longer residence is expected to result in a better accumulation of the antibody at the target site. Thus, it can be expected that antibodies with a low clearance value CL have in general a greater therapeutic potential as they are expected to accumulate better at target sites such as LRRC15-positive tumors. In addition, a less frequent dosing in a therapeutic application is conceivable. In summary, the antibodies according to the current invention show a superior clearance behavior compared to prior art antibody TPP-12942.

### Example 4: Determination of Affinity and Species Cross-Reactivity

To assess the binding kinetics and affinity of anti-LRRC15 antibodies as well as their species cross-reactivity profile, binding assays were conducted using surface plasmon resonance (SPR). Binding assays were performed on a Biacore T200 instrument at 25° C. using assay buffer HBS EP+, 1 mg/ml BSA, 300 mM NaCl, 0.05% NaN3. Antibodies were captured via anti-human Fc IgGs covalently amine coupled to a CM5 sensor chip and human, mouse and cynomolgus LRRC15 were used as analytes in a concentration series from 1.56-200 nM in multi cycle kinetics mode. To obtain reliable dissociation rate constants (k_{d}) the dissociation rate was prolonged from 2.000 seconds to 12.000 seconds for two concentrations of antigen injection (25 nM and 200 nM). Obtained sensorgrams were fitted to a 1:1 Langmuir binding model to derive kinetic data. Results are shown in Table 9.

TABLE 9
Kinetic data of profiled anti-LRRC15 antibodies using SPR

| | | Human | | | Cyno | | | Mouse | | |
|---|---|---|---|---|---|---|---|---|---|---|
| | | LRRC15 | | | LRRC15 | | | LRRC15 | | |
| | | kₐ | k_{d} | K_{D} | kₐ | k_{d} | K_{D} | kₐ | k_{d} | K_{D} |
| Ligand | | [1/Ms] | [1/s] | [M] | [1/Ms] | [1/s] | [M] | [1/Ms] | [1/s] | [M] |
| TPP-1633-family | TPP-1633 | 1.1E+05 | 1.7E−02 | 1.5E−07 | 1.5E+05 | 2.9E−02 | 1.9E−07 | 4.5E+04 | 1.2E−02 | 2.7E−07 |
| | TPP-17074 | 3.7E+05 | 6.8E−05 | 1.9E−10 | 5.3E+05 | 8.4E−05 | 1.6E−10 | 7.7E+04 | 1.6E−04 | 2.1E−09 |
| | TPP-17073 | 4.4E+05 | 9.1E−05 | 2.1E−10 | 6.0E+05 | 1.1E−04 | 1.8E−10 | 9.7E+04 | 2.4E−04 | 2.5E−09 |
| | TPP-14389 | 3.2E+05 | 1.9E−03 | 5.9E−09 | 1.8E+05 | 1.6E−03 | 9.0E−09 | 3.7E+04 | 1.9E−03 | 5.2E−08 |
| | TPP-14392 | 3.7E+04 | 2.1E−04 | 5.9E−09 | 3.9E+04 | 4.0E−04 | 1.0E−08 | 1.6E+05 | 1.2E−03 | 7.5E−09 |
| TPP-12942-family | TPP-12942 | 4.2E+04 | 1.1E−03 | 2.5E−08 | 5.5E+04 | 2.0E−03 | 3.6E−08 | 1.3E+04 | 6.1E−04 | 4.9E−08 |
| | TPP-17078 | 4.7E+04 | 8.1E−05 | 1.7E−09 | 5.9E+04 | 1.1E−04 | 1.8E−09 | 4.2E+04 | 1.2E−04 | 2.9E−09 |
| | TPP-17421 | 3.9E+04 | 3.7E−05 | 9.6E−10 | 4.6E+04 | 4.2E−05 | 9.1E−10 | 2.9E+04 | 4.5E−05 | 1.6E−09 |
| | TPP-17405 | 7.2E+04 | 6.1E−05 | 8.6E−10 | 8.8E+04 | 6.9E−05 | 7.8E−10 | 4.2E+04 | 1.1E−04 | 2.7E−09 |
| | TPP-17418 | 3.3E+04 | 3.5E−05 | 1.1E−09 | 3.9E+04 | 3.9E−05 | 9.9E−10 | 2.9E+04 | 2.6E−05 | 8.9E−10 |
| | TPP-17422 | 5.7E+04 | 7.1E−05 | 1.3E−09 | 6.9E+04 | 8.4E−05 | 1.2E−09 | 3.3E+04 | 7.4E−05 | 2.3E−09 |
| | TPP-17419 | 2.2E+04 | 7.6E−05 | 3.4E−09 | 4.4E+04 | 1.1E−04 | 2.5E−09 | 2.4E+04 | 5.6E−05 | 2.3E−09 |

As can be seen from the kinetic data the affinity was significantly improved for all shown variants derived from either TPP-1633 or TPP-12942. Affinities were even improved into the subnanomolar range for certain variants. The improvement is largely driven by a decrease in the dissociation rate constant k_{d} from the 10⁻² s⁻¹ range for TPP-1633 and 10⁻³ s⁻¹ range for TPP-12942 up into the 10⁻⁵ s⁻¹ range. This improvement results in a shift of antibody-antigen half-lives from minutes into the range of hours which is a strong benefit for therapeutic use as the antibodies will most likely be longer retained on the tumor and can act longer.

Surprisingly, improvement was not only observed for human LRRC15 but also for the cynomolgus and the mouse LRRC15 protein. Thus, improving the affinity on human LRRC15 did not lead to a loss of binding on cynomolgus or mouse protein, but instead resulted in an overall improvement on all species. This also highlights the cross-reactive binding of the anti-LRRC15 antibodies tested.

### Example 5: Germlining of Antibody Sequences

Prior art antibody TPP-12942 carries multiple deviations from the human germline as described in the IMGT database (cf. http://www.imgt.org/).

TPP-12942 carries 16 deviations from the closest germline light chain identified (KV1-39-J4, TPP-21469; consecutive numbering; SEQ ID NO:141): S28D, S31N, K42G, P44V, L46F, A50Y, A51T, S54R, Q56H, F71Y, Y87F, S91G, Y92E, S93A, T94L, L96W.

TPP-12942 carries 24 deviations from the closest germline heavy chain identified (V1-2-02.1-J4, TPP-21468 (SEQ-ID NO: 140)/TPP-21470 (SEQ-ID NO: 142); consecutive numbering): Q1E, T28K, T30S, G31S, Y33W, M341, H35E, R38K, M481, W50E, N52L, N54G, G56D, G57T, A61N, Q62E, Q65K, G66D, V68A, M70F, R72S, S77N, Y106W and D108G.

Compared to the TPP-12942 antibody, the antibodies according to the current invention carry a reduced number of amino acid deviations from germline.

A lower number of germline deviations of an antibody in comparison to its closest human germline sequence, reduces the murine content of the antibody or a phage-display derived human antibody without affecting antigen binding, thereby increasing the overall “humanness” and leading to a reduced risk of immunogenicity caused by those molecules (Hwang et al.; Methods, Volume 36, Issue 1, 2005, Pages 35-42, ISSN 1046-2023). Details are described in Table 10 and Table Table 11.

TABLE 10
Number of germline deviations in Light and Heavy Chain and respective
mutations as compared to human reference sequence for TPP-12942 family.

| | | # of heavy | | |
|---|---|---|---|---|
| | | chain | | |
| | # of light | deviations | | |
| | chain | (Reference | | |
| | deviations | sequence | | |
| | (Reference | (V1-2-02.1- | | |
| | sequence | J4, TPP- | | |
| | KV1-39-J4, | 21468 & | | |
| TPP- | TPP-21469) | 21470) | | |
| ID | 0 | 0 | mutations V_{L} | mutations V_{H} |
| 12942 | 16 | 24 | S28D, S31N, K42G, P44V, | Q1E, T28K, T30S, G31S, Y33W, M34I, |
| | | | L46F, A50Y, A51T, S54R, | H35E, R38K, M48I, W50E, N52L, N54G, |
| | | | Q56H, F71Y, Y87F, S91G, | G56D, G57T, A61N, Q62E, Q65K, G66D, |
| | | | Y92E, S93A, T94L, L96W | V68A, M70F, R72S, S77N, Y106W, D108G |
| 17078 | 9 | 24 | K42G, L46F, A50Y, F71Y, | Q1E, T28K, T30S, G31S, Y33W, M34I, |
| | | | Y87F, S91G, Y92F, T94L, | H35E, R38K, M48I, W50E, N52L, N54G, |
| | | | L96W | G56D, G57T, A61N, Q62E, Q65K, G66D, |
| | | | | V68A, M70F, R72S, S77N, Y106W, D108G |
| 17405 | 12 | 24 | S28R, K42G, L46F, A50Y, | Q1E, T28K, T30S, G31S, Y33W, M34I, |
| | | | F71Y, Y87F, Q89D, S91G, | H35E, R38K, M48I, W50E, N52L, N54G, |
| | | | Y92L, S93E, T94L, L96W | G56D, G57T, A61N, Q62E, Q65K, G66D, |
| | | | | V68A, M70F, R72S, S77N, Y106W, D108Q |
| 17418 | 9 | 24 | K42G, L46F, A50Y, F71Y, | Q1E, T28K, T30S, G31S, Y33W, M34I, |
| | | | Y87F, S91G, Y92F, T94L, | H35E, R38K, M48I, W50E, N52L, N54G, |
| | | | L96W | G56D, G57W, A61N, Q62E, Q65K, G66D, |
| | | | | V68A, M70F, R72S, S77N, Y106W, D108Q |
| 17419 | 11 | 24 | A25I, K42G, L46F, A50Y, | Q1E, T28K, T30S, G31S, Y33W, M34I, |
| | | | F71Y, Y87F, S91G, Y92L, | H35E, R38K, M48I, W50E, N52L, N54G, |
| | | | S93R, T94L, L96W | G56D, G57W, A61N, Q62E, Q65K, G66D, |
| | | | | V68A, M70F, R72S, S77N, Y106W, D108Q |
| 17421 | 10 | 24 | K42G, L46F, A50Y, F71Y, | Q1E, T28K, T30S, G31S, Y33W, M34I, |
| | | | Y87F, S91G, Y92L, S93E, | H35E, R38K, M48I, W50E, N52L, N54G, |
| | | | T94L, L96W | G56D, G57W, A61N, Q62E, Q65K, G66D, |
| | | | | V68A, M70F, R72S, S77N, Y106W, D108Q |
| 17422 | 9 | 24 | K42G, L46F, A50Y, F71Y, | Q1E, T28K, T30S, G31S, Y33W, M34I, |
| | | | Y87F, S91G, Y92L, T94L, | H35E, R38K, M48I, W50E, N52L, N54G, |
| | | | L96W | G56D, G57T, A61N, Q62E, Q65K, G66D, |
| | | | | V68A, M70F, R72S, S77N, Y106W, D108Q |

TABLE 11
Number of germline deviations in Light and Heavy Chain and respective
mutations as compared to human reference sequence for TPP-1633 family

| | | # of heavy chain | | |
|---|---|---|---|---|
| | # of light chain | deviations | | |
| | deviations | (Reference | | |
| | (Reference | Sequence HV3-23- | | |
| | Sequence KV1-39- | J1, TPP-21547 & | | |
| TPP- | J4, TPP-21479) | TPP-21470) | | |
| ID | 0 | 0 | mutations V_{L} | mutations V_{H} |
| 14389 | 13 | 9 | S11D, S30D, I31V, S32D, | S31G, A33M, A50G, |
| | | | Y34W, N36A, Y51F, Q57E, | S52Y, G53P, G55P, S57Y, |
| | | | V60I, S93A, Y92N, S93G, T94F | Y111A, F112L |
| 14392 | 14 | 9 | S11D, S30D, I31V, S32D, | S31G, A33M, A50G, |
| | | | Y34W, N36A, Y51F, S55Y, | S52Y, G53P, G55P, S57Y, |
| | | | Q57E, V60I, S93A, Y92N, | Y111A, F112L |
| | | | S93G, T94F | |
| 17073 | 14 | 10 | S11D, S30D, I31V, S32D, | S31G, A33M, A50G, |
| | | | Y34W, N36A, Y51F, S55Y, | S52Y, G53P, G55A, S57Y, |
| | | | Q57E, V60I, S93A, Y92N, | Y59L, Y111A, F112L |
| | | | S93G, T94F | |
| 17074 | 14 | 10 | S11D, S30D, I31V, S32D, | S31G, A33M, A50G, |
| | | | Y34W, N36A, Y51F, S55Y, | S52Y, G53P, S57Y, T58A, |
| | | | Q57E, V60I, S93A, Y92N, | Y59L, Y111A, F112L |
| | | | S93G, T94F | |

In summary, TPP-17078, TPP-17405, TPP-17418, TPP-17419, TPP-17421 and TPP-17421 have fewer number of germline deviations than their humanized parent TPP-12942. The antibodies of the human antibody TPP-1633 family TPP-14389, TPP-14392, TPP-17073, TPP-17074, TPP-17075, and TPP-17076 have even lower number of germline deviations, thereby decreasing the risk of immunogenic reaction upon use in human therapy.

### Example 6: Improved pH Stability for Downstream Processing

To facilitate manufacturing therapeutic antibodies efficiently and cost effectively for a human therapeutic use, the antibodies must display certain ‘drug-like’ properties to withstand the challenges of the requirements of a manufacturing process. Typically, there is an elution step using a low pH buffer after Protein A affinity chromatography. Similarly, a low pH hold step for several hours for virus inactivation is integrated in such a typical manufacturing process. Any shortcoming in the ability of an antibody to withstand such more extreme conditions will make development and manufacturing more difficult and costly, since individual solutions for the issues need to be found.

In order to check the stability of the antibodies at low pH, the storage buffer of the antibodies was exchanged to a low pH buffer (50 mM sodium acetate and 500 mM NaCl, pH 3.8) using a PD10 Mini column according to manufacturer's protocol. After buffer exchange the samples had a concentration between 1 and 2 mg/ml. The samples were incubated for 270 min at room temperature and small aliquots were taken at several points in time followed by analytical size exclusion chromatography (SEC) analysis. The column (Superdex 200 Increase 10/300 GL column) was run in low pH buffer at room temperature; flow rate 0.7 ml/min, sample injection volume 50 μl.

No significant changes were detected in the SEC elution profile after incubating at low pH for 270 min for TPP-14389, TPP-14392, TPP-17078 and TPP-17421 (Table 12). In contrast, the level of intact antibody of TPP-12942 was reduced to only 94.5% after incubation at low pH 3.8 over time. In summary, TPP-14389, TPP-14392, TPP-17078 and TPP-17421 were able to withstand low pH conditions at pH 3.8 much better than TPP-12942. The reason for this difference is currently not clear.

TABLE 12
Percent intact antibody determined by analytical size exclusion
chromatography after incubating TPP-12942, TPP-14389, TPP-
14392, TPP-17078 and TPP-17421 at pH 3.8 for 270 min.

| | TPP-12942 | TPP-14389 | TPP-14392 | TPP-17078 | TPP-17421 |
|---|---|---|---|---|---|
| % intact antibody | 94.5% | 100% | 99.8% | 99.1% | 100% |

### Example 9: Assessment of LRRC15 Antibody Internalization Capability

LRRC15 has been previously described as an ADC (antibody drug conjugate) target for the killing of cancer cells. However suitability of a target highly depends on the type of ADC. For example, quick and effective internalization of a binding antibody into the targeted cell may or may not be desirable depending for example on the mode of action of a drug. Available data from a non-TTC approach, wherein a murine LRRC15 antibody was conjugated to the microtubule toxin auristatin E have shown that the internalization time course is significantly slower for LRRC15 compared to other ADC targets, which internalize completely within 2 hours of incubation (U.S. Pat. No. 7,399,469). Where a TTC approach according to the current invention is designed, the slow rate of internalization results in a comparably long residence time of the TTC on the cell surface, making the suitability of the target unpredictable: For TTCs, the internalization ability of a target/antibody combination may define in which ratio the radioactivity hits the tumor cell, the stroma cells surrounding the tumor cell, or both. Until today only fast internalizing targets have been addressed with TTC approaches. According to the current invention it is shown for the first time, that a low internalizing target such as LRRC15 can be used for a TTC approach and gives highly encouraging results in various tumor models.

### Example 10: Preparation of Targeted Thorium Conjugate (TTC)

The disclosure of WO2016096843 is incorporated herein by reference in its entirety and in particular with regard to the production of the conjugate as described in this example.

Conjugation of the 3,2-hydroxypiridonone (3,2-HOPO) chelator to the antibodies TPP-14389, TPP-12942, TPP-17078, TPP-17421 and TPP-17421, was conducted as previously described in patent application WO2016096843. Briefly, to activate the chelator, the 3,2-HOPO chelator, dissolved in DMA at a 1:1 ratio with 0.1 M MES buffer pH 5.4, NHS and EDC, both dissolved in 0.1 M MES buffer pH 5.4, were mixed at a ratio of 1/1/3. For conjugation to the antibodies, a molar ratio of 7.5/7.5/22.5/1 (chelator/NHS/EDC/mAb) of the activated chelator was charged to mAb. After 20-60 min, the reaction was quenched with 12% v/v 0.3 M citric acid to adjust pH to 5.5. The protein concentration was determined by HPLC, integrating the peak area at an absorbance of 280 nm. The solution was then buffer exchanged into 30 mM Citrate, 50 mg/ml sucrose, 2 mM EDTA, 0.5 mg/ml pABA, pH 5.5 by Tangential Flow Filtration (TFF) at constant volume. At the end of the diafiltration, the solution was discharged to a formulation container. The product was formulated with TFF buffer (30 mM Citrate, 50 mg/ml M Sucrose, 2 mM EDTA, 0.5 mg/ml pABA, pH 5.5) and 7% w/v polysorbate 80 to obtain 2.5 mg/ml of respective LRRC15-antibody-chelator conjugates (LRRC15-ACCs). All LRRC15-ACCs were filtered through a 0.2 μm filter into sterile vials.

LRRC15-ACCs were radiolabeled with thorium-227 as described in WO2016096843. Briefly, 5 μl of LRRC15-ACCs were mixed with 32 μl of thorium-227 (activity of 3.875 MBq/ml) and 13 μl of citrate buffer, resulting in LRRC15-targeted thorium-227 conjugates (LRRC15-TTCs) at specific activities of 10 kBq/μg. The sample was incubated for 60 min at room temperature to allow for stable radiolabeling of thorium-227 into the 3,2-HOPO chelator. An aliquot of the sample was analyzed by instant thin layer chromatography (iTLC). The radiochemical purity (RCP) was determined to be ≥95% for all respective LRRC15-TTCs.

### Example 11: In Vitro Cytotoxicity and Induction of DNA Double Strand Breaks by LRRC15-TTCs

In vitro cytotoxicity of LRRC15-TTCs was tested using
(i) human osteosarcoma cell line Saos-2, which endogenously expresses LRRC15,
(ii) human colon cancer cell line HT29, which is negative for LRRC15 and
(iii) cell line HT29-LRRC15, derived from HT29 by transfection with human LRRC15.

For this purpose, cells were seeded in 384-well plates and incubated in presence of the respective LRRC15-TTC, starting at a concentration of 20 kBq/ml, radiolabeled at a specific activity of 40 kBq/μg. For each case a matching radiolabeled isotype control was included for comparison. After 5 days, the decrease in viability was assessed using Cell Titer Glo assay (Promega). Resulting IC50 values in kBq/ml are summarized in Table 13.

Specific reduction of cell viability was observed when LRRC15-TTCs were incubated on LRRC15-positive cell lines Saos-2 and HT29-LRRC15 with ˜25-fold and ˜37-fold specificity over the radiolabeled isotype control. In contrast, no difference to the radiolabeled isotype control was observed on the target negative cell line HT29.

TABLE 13
Summary of in vitro cytotoxicity of LRRC15-TTCs treatment
of LRRC15 expressing cell lines Saos-2 and HT29-LRRC15
as well as treatment of LRRC15-negative cell line HT29.
IC₅₀ values were determined using Cell Titer Glo; a radiolabeled
isotype control was included for comparison.

| IC₅₀ (kBq/ml) | Saos-2 | HT29-LRRC15 | HT29 |
|---|---|---|---|
| Radiolabeled | 7.6 ± 3.8 | 3.35 ± 0.5 | 4.4 ± 0.2 |
| Isotype Control | | | |
| TPP-14389 | 0.33 ± 0.2 | 0.09 ± 0.0 | 4.7 ± 0.0 |
| TPP-12942 | 0.35 ± 0.3 | 0.08 ± 0.0 | 4.8 ± 1.1 |
| TPP-17078 | 0.30 ± 0.2 | 0.09 ± 0.0 | 4.6 ± 0.4 |
| TPP-17421 | 0.34 ± 0.3 | 0.1 ± 0.0 | 5.3 ± 0.4 |

Phosphorylated histone H2AX (gH2AX) reflects the presence of double-strand breaks in DNA. The reduction in cell viability was therefore further shown to be based on induction of DNA double strand breaks by immunofluorescence staining of Saos-2 cells for gH2AX upon treatment with LRRC15-TTC (TPP-14389). For this purpose, cells were exposed to either cell culture medium, non-radiolabeled LRRC15-antibody-chelator conjugate, a radiolabeled isotype control (0.5 and 5 kBq/ml) or LRRC15-TTC (0.5 and 5 kBq/ml). After 96 hours, cells were washed with PBS and fixed using 4% paraformaldehyde. LRRC15-antigen was visualized using a human anti human LRRC15 antibody, followed by incubation with an anti-human secondary antibody labeled with Alexa 647. DNA double strand breaks were visualized using a gH2AX specific antibody (rabbit; Cell Signaling), followed by incubation with an Alexa 647 labeled secondary antibody (anti-rabbit; Invitrogen) and analyzed by flow.

As shown in FIG. 7, treatment of LRRC15-positive HT29-LRRC15 positive cells resulted in specific induction of gH2AX and cell cycle arrest reflecting the presence of double-strand breaks after 96 h.

### Example 12: LRRC15 Expression in Different Tumor Types

The expression of LRRC15 was confirmed by RNAseq data, available via the “the cancer genome atlas (TCGA) database” on one hand, and by immunohistochemistry (IHC) analysis on human biopsies on the other hand. LRRC15 RNA levels are high in several cancer tissues, including breast cancer>head and neck squamous cell cancer>squamous lung cancer>pancreatic cancer>diffused large B-cell carcinoma>lung adenocarcinoma>colorectal cancer>gastric cancer, as well as Sarcoma.

For IHC on human tissues, a murine antibody targeting human LRRC15 was incubated at a concentration of 0.1 μg/ml on paraffin embedded tissue slices for 1 h at room temperature. Samples were washed with Tris buffer saline (TBS) and incubated with labeled polymer-HRP anti-mouse (Dako) for 30 min. Slices were washed with TBS buffer and incubated with 3,3′ diaminobenzidine tetrahydrochloride (DAB) solution for 2-6 mins for development and visualization. The reaction was stopped by adding tap water. Respective stainings are presented in FIG. 8 to 11.

For IHC on tissues isolated from xenograft or syngeneic models, a murine antibody targeting human LRRC15 was incubated at a concentration of 0.1 μg/ml on paraffin embedded, blocked tissue slices for 1 h at room temperature. Samples were washed with TBS buffer and incubated with labeled polymer-horse raddish peroxidase anti-mouse (Dako) for 30 min. Slices were washed with TBS buffer and incubated with DAB solution for 2-6 mins for development and visualization. The reaction was stopped by adding tap water. Respective stainings are presented in FIGS. 12 to 21. A summary of all IHC stainings of xenograft and murine syngeneic models is presented in Table 14 below.

TABLE 14
Summary of LRRC15-stained xenograft and murine syngeneic models,
listed by respective tissue type. LRRC15 staining intensities
were scored from 1+ to 3+ upon visual inspection.

| | | IHC Score | | |
|---|---|---|---|---|
| Tissue | Model | 1+ | 2+ | 3+ |
| Sarcoma | Sarc 4183 | | | 3+ |
| | Sarc 9503 | | | 3+ |
| | Sarc 10751 | | | 3+ |
| Breast Cancer | KPL-4 | 1+ | | |
| | T47D | | | 3+ |
| | MX-1 | | 2+ | |
| | MDA-MB-231 | | 2+ | |
| | BT20 | 1+ | | |
| | BT-474 | 1+ | | |
| | MCF-7 | | | 3+ |
| | MFM-223 | 1+ | | |
| NSCLC | NCl-H292 | | | 3+ |
| | NCl-H1975 | 1+ | | |
| | NCl-H460 | 1+ | | |
| | A549 | | 2+ | |
| | NCl-H322 | | | 3+ |
| | NCl-H522 | 1+ | | |
| | NCl-H1993 | 1+ | | |
| | NCl-H441 | | 2+ | |
| | NCl-H228 | | 2+ | |
| | NCl-H82 | | 2+ | |
| | NCl-H226 | | 2+ | |
| Murine Breast | 4T1 | | 2+ | |
| Cancer | | | | |
| Murine Lung | Lewis Lung | | 2+ | |
| Cancer | | | | |
| Murine Colorectal | MC38 | | | |
| Cancer | CT26 | 1+ | | |
| Murine Testicular | F9 | 1+ | | |
| Teratoma | | | | |
| Murine Melanoma | B16F10 | 1+ | | |

### Example 13: In Vivo Efficacy of LRRC15-TTC Treatment in Various Tumor Indications

The in vivo efficacy of LRRC15-TTCs was evaluated in several xenograft models including the human NSCLC model Calu-3, the human pancreatic cancer model BxPC-3, the human HNSCC model SCC-15 as well as the murine syngeneic breast cancer model 4T1. The expression of LRRC15 in these models was investigated by IHC using a murine antibody targeting human LRRC15. Respective IHC pictures are shown in FIG. 22. The LRRC15 antigen staining intensity was determined to be high (3+) for the Calu-3, moderate (2+) for BxPC-3 and SCC-15 and medium (2+) for 4T1. It is also noteworthy that in all respective tumors, the LRRC15 expression is rather homogeneous.

The efficacy of LRRC15-TTCs was tested in the different models as outlined above. The administered doses of the LRRC15-TTCs ranged between 250 and 750 kBq/kg at a total antibody dose of 0.14 mg/kg, if not indicated differently. A radiolabeled isotype control with matching activity at the highest dose was included for comparison. Doses were administered once, if not indicated differently. In case of the Calu-3 and the SCC-15 model, tumor accumulation as well as normal organ distribution was investigated by ex vivo analysis by counting the accumulated thorium-227 activity using a high purity germanium detector.

### Example 13.1 NSCLC Model Calu-3

In the human NSCLC xenograft model Calu-3, specific tumor growth inhibition of an LRRC15-TTC comprising TPP-14389 as targeting moiety compared to vehicle was observed after a single dose administration of 250 kBq/kg and 500 kBq/kg (0.14 mg/kg). The treatment was further shown to be specific in comparison to a radiolabeled isotype control at an administered dose level of 250 kBq/kg (0.14 mg/kg). Treatment further resulted in higher number of complete and partial responses in comparison to vehicle and radiolabeled isotype control treated animals. The results are presented in FIG. 23 and matching Table 15.

TABLE 15
Percentage of progressing diseases (PDs), stable diseases (SDs),
partial responses (PRs) and complete responses (CRs) evaluated
based on RECIST criteria in Calu-3 tumor bearing mixe after
single dose administration of LRRC15-TTC (TPP-14389).

| | | Isotype Ctrl | | TPP-14389 | |
|---|---|---|---|---|---|
| RECIST | Vehicle | 250 | 500 | 250 | 500 |
| Dose | — | kBq/kg | kBq/kg | kBq/kg | kBq/kg |
| PD | 100% | 90% | 90% | 80% | 10% |
| SD | | 10% | 10% | 10% | 20% |
| PR | | | | 10% | 50% |
| CR | | | | | 20% |

The biodistribution of LRRC15-TTCs (TTCs with targeting moieties TPP-14389, TPP-13612, TPP-17074, TPP-17078, TPP-17421 and TPP-12942) was studied in parallel in the same model. Tumors and organs were isolated at the timepoints indicated and the accumulated thorium-227 activity was measured using a high purity germanium detector. Specific tumor accumulation of LRRC15-TTCs (TPP-14389, TPP-13612, TPP-17074, TPP-17078, TPP-17421 and TPP-12942) was observed compared to a radiolabeled isotype control with a determined injected dose per gram around 25% at t=336 h. No major accumulation in other healthy organs was observed. The results are presented in Table 16.1, 16.2 and 16.3.

TABLE 16.1
Biodistribution of LRRC15-TTCs in the human NSCLC xenograft model Calu-3 in
tumor or blood. LRRC15-TTCs are labeled based on the respective targeting
moiety (TPP). Tumors and organs were isolated at the respective timepoints.
Accumulated thorium-227 is given in % of injected dose per gram (ID/g).

| Tumor | t in hours | 168 | 336 | 504 | Blood | t in hours | 168 | 336 | 504 |
|---|---|---|---|---|---|---|---|---|---|
| Isotype Control | Animal 1 | 6.0 | 5.0 | 4.4 | | Animal 1 | 6.1 | 4.6 | 2.6 |
| | Animal 2 | 6.0 | 6.4 | 5.4 | | Animal 2 | 4.4 | 5.0 | 1.2 |
| | Animal 3 | 7.7 | 8.9 | 8.2 | | Animal 3 | 6.8 | 3.4 | 2.0 |
| TPP-14389 | Animal 1 | 23.3 | 14.6 | 12.0 | | Animal 1 | 0.3 | 0.1 | 0.1 |
| | Animal 2 | 15.1 | 15.4 | 24.8 | | Animal 2 | 0.3 | 0.1 | |
| | Animal 3 | 12.6 | 13.5 | 9.9 | | Animal 3 | 0.2 | 0.1 | |
| TPP-13612 | Animal 1 | 15.0 | 9.3 | 15.4 | | Animal 1 | 0.6 | 0.2 | 0.1 |
| | Animal 2 | 20.1 | 9.5 | 24.4 | | Animal 2 | 0.8 | 0.1 | 0.1 |
| | Animal 3 | 10.5 | 17.0 | 25.4 | | Animal 3 | 0.4 | 0.1 | 0.1 |
| TPP-17074 | Animal 1 | 7.9 | 21.9 | 10.4 | | Animal 1 | 0.1 | 0.1 | 0.1 |
| | Animal 2 | 11.5 | 16.0 | 9.6 | | Animal 2 | 0.1 | 0.0 | 0.1 |
| | Animal 3 | 12.8 | 14.1 | 4.8 | | Animal 3 | 0.1 | 0.0 | 0.0 |
| TPP-12942 | Animal 1 | 8.1 | 9.1 | 7.7 | | Animal 1 | 0.1 | 0.0 | 0.0 |
| | Animal 2 | 13.2 | 8.6 | 5.4 | | Animal 2 | 0.1 | 0.1 | |
| | Animal 3 | 11.0 | 5.8 | 5.7 | | Animal 3 | 0.1 | 0.1 | |
| TPP-17078 | Animal 1 | 15.3 | 6.9 | 9.0 | | Animal 1 | 0.1 | 0.1 | |
| | Animal 2 | 8.0 | 5.6 | 7.5 | | Animal 2 | 0.2 | 0.1 | 0.1 |
| | Animal 3 | 7.9 | 5.8 | 9.3 | | Animal 3 | 0.1 | 0.1 | |
| TPP-17421 | Animal 1 | 7.8 | 11.0 | 13.7 | | Animal 1 | 0.2 | 0.1 | 0.1 |
| | Animal 2 | 8.5 | 11.2 | 5.7 | | Animal 2 | 0.2 | 0.1 | 0.0 |
| | Animal 3 | 10.6 | 9.9 | 8.2 | | Animal 3 | 0.2 | 0.1 | 0.0 |

TABLE 16.2
Biodistribution of LRRC15-TTCs in the human NSCLC xenograft model Calu-3 in
liver or spleen. LRRC15-TTCs are labeled based on the respective targeting
moiety (TPP). Tumors and organs were isolated at the respective timepoints.
Accumulated thorium-227 is given in % of injected dose per gram (ID/g).

| Liver | t in hours | 168 | 336 | 504 | Spleen | t in hours | 168 | 336 | 504 |
|---|---|---|---|---|---|---|---|---|---|
| Isotype Control | Animal 1 | 3.7 | 4.7 | 4.6 | | Animal 1 | 4.0 | 4.2 | 6.5 |
| | Animal 2 | 5.7 | 5.3 | 4.5 | | Animal 2 | 4.7 | 5.5 | 6.2 |
| | Animal 3 | 4.1 | 5.5 | 4.5 | | Animal 3 | 4.8 | 5.4 | 6.8 |
| TPP-14389 | Animal 1 | 3.2 | 2.2 | 2.7 | | Animal 1 | 2.0 | 3.0 | 3.5 |
| | Animal 2 | 1.8 | 2.0 | 2.5 | | Animal 2 | 2.0 | 2.3 | 3.3 |
| | Animal 3 | 2.2 | 2.0 | 1.8 | | Animal 3 | 2.4 | 2.7 | 3.0 |
| TPP-13612 | Animal 1 | 1.2 | 2.2 | 1.4 | | Animal 1 | 2.1 | 2.0 | 2.5 |
| | Animal 2 | 1.5 | 1.9 | 1.6 | | Animal 2 | 2.0 | 1.8 | 2.4 |
| | Animal 3 | 1.1 | 2.5 | 1.6 | | Animal 3 | 1.2 | 3.2 | 3.0 |
| TPP-17074 | Animal 1 | 1.3 | 1.7 | 1.9 | | Animal 1 | 1.3 | 2.4 | 2.9 |
| | Animal 2 | 1.7 | 2.8 | 1.7 | | Animal 2 | 1.6 | 2.7 | 2.0 |
| | Animal 3 | 2.0 | 2.1 | 1.6 | | Animal 3 | 1.6 | 2.7 | 2.2 |
| TPP-12942 | Animal 1 | | 2.7 | 1.7 | | Animal 1 | 2.2 | 1.3 | 2.3 |
| | Animal 2 | 2.3 | 2.3 | 2.6 | | Animal 2 | 3.3 | 1.9 | 2.0 |
| | Animal 3 | 2.2 | 1.8 | 2.1 | | Animal 3 | 3.0 | 2.1 | 2.5 |
| TPP-17078 | Animal 1 | 2.1 | 2.2 | 3.1 | | Animal 1 | 2.5 | 2.1 | 1.7 |
| | Animal 2 | 3.3 | 1.7 | 1.5 | | Animal 2 | 1.9 | 2.2 | 1.9 |
| | Animal 3 | 1.8 | 2.0 | 1.4 | | Animal 3 | 2.8 | 2.3 | 0.2 |
| TPP-17421 | Animal 1 | 2.7 | 3.1 | 3.3 | | Animal 1 | 1.9 | 2.7 | 2.0 |
| | Animal 2 | 3.3 | 3.4 | 2.1 | | Animal 2 | 2.1 | 2.9 | 2.2 |
| | Animal 3 | 3.3 | 2.4 | 2.8 | | Animal 3 | 2.4 | 1.7 | 2.3 |

TABLE 16.3
Biodistribution of LRRC15-TTCs in the human NSCLC xenograft model Calu-3 in
kidney or femur. LRRC15-TTCs are labeled based on the respective targeting
moiety (TPP). Tumors and organs were isolated at the respective timepoints.
Accumulated thorium-227 is given in % of injected dose per gram (ID/g).

| Kidneys | t in hours | 168 | 336 | 504 | Femur | t in hours | 168 | 336 | 504 |
|---|---|---|---|---|---|---|---|---|---|
| Isotype Control | Animal 1 | 4.0 | 2.9 | 1.9 | | Animal 1 | 3.0 | 4.1 | 5.1 |
| | Animal 2 | 3.4 | 2.8 | 1.4 | | Animal 2 | 2.8 | 5.2 | 6.0 |
| | Animal 3 | 2.9 | 3.1 | 1.9 | | Animal 3 | 3.4 | 4.7 | 6.5 |
| TPP-14389 | Animal 1 | 2.8 | 2.0 | 1.4 | | Animal 1 | 2.4 | 3.2 | 5.6 |
| | Animal 2 | 2.7 | 2.3 | 1.5 | | Animal 2 | 2.4 | 3.5 | 4.3 |
| | Animal 3 | 2.7 | 2.2 | 1.3 | | Animal 3 | 2.6 | 3.2 | 3.3 |
| TPP-13612 | Animal 1 | 2.2 | 1.7 | 1.2 | | Animal 1 | 2.5 | 3.6 | 3.8 |
| | Animal 2 | 2.4 | 2.3 | 1.2 | | Animal 2 | 2.3 | 2.8 | 5.7 |
| | Animal 3 | 2.1 | 2.3 | 1.4 | | Animal 3 | 2.2 | 3.7 | 5.5 |
| TPP-17074 | Animal 1 | 1.7 | 1.8 | 1.2 | | Animal 1 | 2.3 | 3.6 | 4.7 |
| | Animal 2 | 1.7 | 2.8 | 1.5 | | Animal 2 | 2.5 | 3.1 | 5.5 |
| | Animal 3 | 2.9 | 2.7 | 1.1 | | Animal 3 | 3.2 | 3.6 | 3.8 |
| TPP-12942 | Animal 1 | 2.5 | 1.7 | 0.9 | | Animal 1 | 2.5 | 3.0 | 4.0 |
| | Animal 2 | 2.9 | 1.6 | 1.3 | | Animal 2 | 2.6 | 3.3 | 4.6 |
| | Animal 3 | 3.0 | 1.5 | 1.2 | | Animal 3 | 2.7 | 3.1 | 4.5 |
| TPP-17078 | Animal 1 | 3.0 | 1.8 | 0.9 | | Animal 1 | 3.0 | 3.6 | 3.4 |
| | Animal 2 | 3.7 | 2.2 | 0.9 | | Animal 2 | 3.7 | 2.9 | 3.3 |
| | Animal 3 | 2.8 | 1.6 | 0.9 | | Animal 3 | 3.0 | 3.0 | 3.8 |
| TPP-17421 | Animal 1 | 2.8 | 2.4 | 0.7 | | Animal 1 | 2.7 | 3.0 | 4.4 |
| | Animal 2 | 2.4 | 1.6 | 1.0 | | Animal 2 | 2.9 | 2.8 | 3.5 |
| | Animal 3 | 2.4 | 1.6 | 1.1 | | Animal 3 | 2.4 | 2.3 | 4.3 |

### Example 13.2 Human Pancreatic Cancer Model BxPC-3

Efficacy of LRRC15-TTCs (TTCs with targeting moieties TPP-14389, TPP-12942, TPP-17078 and TPP-17421) was further tested in the human pancreatic cancer (PancCa) model BxPC-3. When administered two times at a dose of 750 kBq/kg at an interim of one-week, specific tumor growth inhibition of LRRC15-TTC was observed. The results are presented in FIG. 24.

### Example 13.3 Human HNSCC Model SCC-15

Efficacy of LRRC15-TTC (TTCs with targeting moiety TPP-17421) was further tested in the human HNSCC model SCC-15. LRRC15-TTC was administered two times at a dose of 250 kBq/kg at an interim of one week, however, the total antibody dose was varied between 0.14, 1.5 and 3.5 mg/kg. A radiolabeled isotype control was included for comparison. In parallel, tumor accumulation of LRRC15-TTC was studied and compared to a radiolabeled isotype control. The results are presented in FIG. 25 and corresponding Table 17.

LRRC15-TTC demonstrated specific tumor growth inhibition in comparison to vehicle and radiolabeled isotype control when administered two times at 250 kBq/kg at total antibody doses of 1.5 and 3 mg/kg. No tumor growth inhibition was observed at the same radioactive dose of 250 kBq/kg using a total antibody dose of 0.14 mg/kg.

Similarly, in the biodistribution study specific tumor accumulation was observed over the course of 336 h when LRRC15-TTC with targeting moiety TPP-17421 was administered at a fixed radioactivity dose of 250 kBq/kg using total antibody doses of 1.5 and 3 mg/kg (FIG. 26). The accumulated tumor activity reached 30-40% injected dose per gram (ID/g). At the total antibody dose of 0.14 mg/kg, tumor accumulation was around 10% ID/g, similar to the radiolabeled isotype control.

TABLE 17
Ratio of tumor volumes measured in the human HNSCC model SCC-15 for treatment
and control at study day 37 after administration of the first dose, with statistical
significance (one way annova) as indicated compared to vehicle.

| | Treatment/Control Ratio based on |
|---|---|
| Treatments | tumor volumes at study day 37 |
| Vehicle i.v. | 1.00 |
| Radiolabeled isotype control 3 mg/kg; 2 × 250 kBq/kg | 0.7; n.s. |
| LRRC15-TTC (TPP-17421) at 0.14 mg/kg; 2 × 250 kBq/kg | 0.7; n.s. |
| LRRC15-TTC (TPP-17421) at 1.5 mg/kg; 2 × 250 kBq/kg | 0.3; p < 0.05 |
| LRRC15-TTC (TPP-17421)at 3 mg/kg; 2 × 250 kBq/kg | 0.3; p < 0.05 |

### Example 13.4 Combination Treatment LRRC15-TTC with Anti-PD-L1 Antibody in Syngeneic Murine Breast Cancer Model

The efficacy of LRRC15-TTC (with targeting moiety TPP-17421) was further evaluated in the syngeneic murine breast cancer model 4T1 in immunocompetent mice. LRRC15-TTC was administered at a dose of 2×375 kBq/kg, total antibody dose of 0.14 mg/kg, given at an interim of two weeks. In an additional treatment group, LRRC15-TTC (with targeting moiety TPP-17421) was administered at the same treatment regimen as above, but in combination with an antibody binding to the immune checkpoint inhibitor PD-L1 (10 mg/kg; i.p.; dosed every third or fourth day). Respective radiolabeled isotype control groups were included for comparison as well as an anti-PD-L1 antibody monotherapy group. The results are presented in FIG. 27 and Table 18. LRRC15-TTC demonstrated statistically significant tumor growth inhibition at study day 12 compared to vehicle and radiolabeled isotype control. Combination with anti-PD-L1 antibody resulted in slightly decreased “treatment over control” (T/C) ratio but was not statistically significant to LRRC15-TTC monotherapy. Anti-PD-L1 monotherapy did not show statistically significant tumor growth inhibition in comparison to vehicle.

TABLE 18
Ratio of tumor volume measured in model 4T1 for treatment and control
at study day 12 after administration of the first dose, with statistical
significance (one way annova) as indicated compared to vehicle.

| | Treatment/Control Ratio based on |
|---|---|
| Treatments | tumor volumes at study day 12 |
| Vehicle | 1.00 |
| Isotype control, 2 × 375 kBq/kg i.v, 0.14 mg/kg | 0.82 |
| LRRC15-TTC (TPP-17421), 2 × 375 kBq/kg i.v, 0.14 mg/kg | 0.51 * # |
| anti-PD-L1 antibody, 10 mg/kg i.p. | 0.85 |
| anti-PD-L1 antibody, 10 mg/kg i.p./ | 0.61 * |
| isotype control (2 × 375 kBq/kg; i.v., 0.14 mg/kg) | |
| LRRC15-TTC (TPP-17421) 2 × 375 kBq/kg i.v, 0.14 mg/kg/ | 0.39 * # |
| anti-PD-L1 antibody, 10 mg/kg i.p | |

* p < 0.05 vs vehicle; # p < 0.05 vs isotype control.

### Example 14: LRRC15 Targeting Conjugates for Diagnosis and Imaging

To conduct in vivo positron emission tomography, the LRRC15-antibody-chelator conjugate based on TPP-14389 was radiolabeled with zirconium in vitro as described in example 10 for thorium.

Integrity of the radiolabeled product was analyzed by size-exclusion chromatography and was compared to non-radiolabeled LRRC15-antibody-chelator conjugate based on TPP-14389.

An increase in dimer content from 3 to 11%, 45 minutes after radiolabeling was observed which was stable for the following 24 hours, when the LRRC15-antibody-chelator conjugate based on TPP-14389 was radiolabeled with zirconium.

These results demonstrated feasibility of radiolabeling of the LRRC15-antibody-chelator conjugates with zirconium. The resulting conjugate can be used for PET imaging studies, e.g. for diagnosis and/or imaging in a human or non human subject.

Sequences

| | | Sequence | Seq | SEQ | |
|---|---|---|---|---|---|
| TPP ID | Sequence Name | Region | Type | ID | SEQ |
| TPP- | 060E-M016-G14- | VH | PRT | 1 | EVQLLESGGGLVQPGGSLRLSCAASGFTFSGYMMQ |
| 1633 | hIgG1 | | | | WVRQAPGKGLEWVSGIYPSGGYTNYADSVKGRFTIS |
| | | | | | RDNSKNTLYLQMNSLRAEDTATYYCAREKASDLSGSY |
| | | | | | SEALDYWGQGTLVTVSS |
| TPP- | 060E-M016-G14- | HCDR1 | PRT | 2 | GYMMQ |
| 1633 | hIgG1 | | | | |
| TPP- | 060E-M016-G14- | HCDR2 | PRT | 3 | GIYPSGGYTNYADSVKG |
| 1633 | hIgG1 | | | | |
| TPP- | 060E-M016-G14- | HCDR3 | PRT | 4 | EKASDLSGSYSEALDY |
| 1633 | hIgG1 | | | | |
| TPP- | 060E-M016-G14- | VL | PRT | 5 | DIQMTQSPDSLSASVGDRVTITCRASQDVGSWLAWY |
| 1633 | hIgG1 | | | | QQKPGKAPKLLIFAASSLESGIPSRFSGSGSGTDFTLTIS |
| | | | | | SLQPEDFATYYCQQANGFPLTFGGGTKVEIK |
| TPP- | 060E-M016-G14- | LCDR1 | PRT | 6 | RASQDVGSWLA |
| 1633 | hIgG1 | | | | |
| TPP- | 060E-M016-G14- | LCDR2 | PRT | 7 | AASSLES |
| 1633 | hIgG1 | | | | |
| TPP- | 060E-M016-G14- | LCDR3 | PRT | 8 | QQANGFPLT |
| 1633 | hIgG1 | | | | |
| TPP- | 060E-M016-G14- | Heavy | PRT | 9 | EVQLLESGGGLVQPGGSLRLSCAASGFTFSGYMMQ |
| 1633 | hIgG1 | Chain | | | WVRQAPGKGLEWVSGIYPSGGYTNYADSVKGRFTIS |
| | | | | | RDNSKNTLYLQMNSLRAEDTATYYCAREKASDLSGSY |
| | | | | | SEALDYWGQGTLVTVSSASTKGPSVFPLAPSSKSTSG |
| | | | | | GTAALGCLVKDYFPEPVTVSWNSGALTSGVHTFPAVL |
| | | | | | QSSGLYSLSSVVTVPSSSLGTQTYICNVNHKPSNTKVD |
| | | | | | KKVEPKSCDKTHTCPPCPAPELLGGPSVFLFPPKPKDT |
| | | | | | LMISRTPEVTCVVVDVSHEDPEVKFNWYVDGVEVHN |
| | | | | | AKTKPREEQYNSTYRVVSVLTVLHQDWLNGKEYKCK |
| | | | | | VSNKALPAPIEKTISKAKGQPREPQVYTLPPSRDELTK |
| | | | | | NQVSLTCLVKGFYPSDIAVEWESNGQPENNYKTTPP |
| | | | | | VLDSDGSFFLYSKLTVDKSRWQQGNVFSCSVMHEAL |
| | | | | | HNHYTQKSLSLSPG |
| TPP- | 060E-M016-G14- | Light | PRT | 10 | AQDIQMTQSPDSLSASVGDRVTITCRASQDVGSWLA |
| 1633 | hIgG1 | Chain | | | WYQQKPGKAPKLLIFAASSLESGIPSRFSGSGSGTDFT |
| | | | | | LTISSLQPEDFATYYCQQANGFPLTFGGGTKVEIKRTV |
| | | | | | AAPSVFIFPPSDEQLKSGTASVVCLLNNFYPREAKVQ |
| | | | | | WKVDNALQSGNSQESVTEQDSKDSTYSLSSTLTLSKA |
| | | | | | DYEKHKVYACEVTHQGLSSPVTKSFNRGEC |
| TPP- | huM25- | VH | PRT | 11 | EVQLVQSGAEVKKPGASVKVSCKASGYKFSSYWIEW |
| 12942 | hIgG1Kappa | | | | VKQAPGQGLEWIGEILPGSDTTNYNEKFKDRATFTSD |
| | | | | | TSINTAYMELSRLRSDDTAVYYCARDRGNYRAWFGY |
| | | | | | WGQGTLVTVSS |
| TPP- | huM25- | HCDR1 | PRT | 12 | SYWIE |
| 12942 | hIgG1Kappa | | | | |
| TPP- | huM25- | HCDR2 | PRT | 13 | EILPGSDTTNYNEKFKD |
| 12942 | hIgG1Kappa | | | | |
| TPP- | huM25- | HCDR3 | PRT | 14 | DRGNYRAWFGY |
| 12942 | hIgG1Kappa | | | | |
| TPP- | huM25- | VL | PRT | 15 | DIQMTQSPSSLSASVGDRVTITCRASQDISNYLNWYQ |
| 12942 | hIgG1Kappa | | | | QKPGGAVKFLIYYTSRLHSGVPSRFSGSGSGTDYTLTIS |
| | | | | | SLQPEDFATYFCQQGEALPWTFGGGTKVEIK |
| TPP- | huM25- | LCDR1 | PRT | 16 | RASQDISNYLN |
| 12942 | hIgG1Kappa | | | | |
| TPP- | huM25- | LCDR2 | PRT | 17 | YTSRLHS |
| 12942 | hIgG1Kappa | | | | |
| TPP- | huM25- | LCDR3 | PRT | 18 | QQGEALPWT |
| 12942 | hIgG1Kappa | | | | |
| TPP- | huM25- | Heavy | PRT | 19 | EVQLVQSGAEVKKPGASVKVSCKASGYKFSSYWIEW |
| 12942 | hIgG1Kappa | Chain | | | VKQAPGQGLEWIGEILPGSDTTNYNEKFKDRATFTSD |
| | | | | | TSINTAYMELSRLRSDDTAVYYCARDRGNYRAWFGY |
| | | | | | WGQGTLVTVSSASTKGPSVFPLAPSSKSTSGGTAALG |
| | | | | | CLVKDYFPEPVTVSWNSGALTSGVHTFPAVLQSSGLY |
| | | | | | SLSSVVTVPSSSLGTQTYICNVNHKPSNTKVDKKVEPK |
| | | | | | SCDKTHTCPPCPAPELLGGPSVFLFPPKPKDTLMISRT |
| | | | | | PEVTCVVVDVSHEDPEVKFNWYVDGVEVHNAKTKP |
| | | | | | REEQYNSTYRVVSVLTVLHQDWLNGKEYKCKVSNKA |
| | | | | | LPAPIEKTISKAKGQPREPQVYTLPPSRDELTKNQVSLT |
| | | | | | CLVKGFYPSDIAVEWESNGQPENNYKTTPPVLDSDGS |
| | | | | | FFLYSKLTVDKSRWQQGNVFSCSVMHEALHNHYTQK |
| | | | | | SLSLSPG |
| TPP- | huM25- | Light | PRT | 20 | DIQMTQSPSSLSASVGDRVTITCRASQDISNYLNWYQ |
| 12942 | hIgG1Kappa | Chain | | | QKPGGAVKFLIYYTSRLHSGVPSRFSGSGSGTDYTLTIS |
| | | | | | SLQPEDFATYFCQQGEALPWTFGGGTKVEIKRTVAAP |
| | | | | | SVFIFPPSDEQLKSGTASVVCLLNNFYPREAKVQWKV |
| | | | | | DNALQSGNSQESVTEQDSKDSTYSLSSTLTLSKADYEK |
| | | | | | HKVYACEVTHQGLSSPVTKSFNRGEC |
| TPP- | 13612-rec02- | VH | PRT | 21 | EVQLLESGGGLVQPGGSLRLSCAASGFTFSGYMMSW |
| 14389 | hIgG1Kappa | | | | VRQAPGKGLEWVSGIYPSPGYTYYADSVKGRFTISRD |
| | | | | | NSKNTLYLQMNSLRAEDTAVYYCAREKASDLFGSYSE |
| | | | | | ALDYWGQGTLVTVSS |
| TPP- | 13612-rec02- | HCDR1 | PRT | 22 | GYMMS |
| 14389 | hIgG1Kappa | | | | |
| TPP- | 13612-rec02- | HCDR2 | PRT | 23 | GIYPSPGYTYYADSVKG |
| 14389 | hIgG1Kappa | | | | |
| TPP- | 13612-rec02- | HCDR3 | PRT | 24 | EKASDLFGSYSEALDY |
| 14389 | hIgG1Kappa | | | | |
| TPP- | 13612-rec02- | VL | PRT | 25 | DIQMTQSPDSLSASVGDRVTITCRASQDVGSWLAWY |
| 14389 | hIgG1Kappa | | | | QQKPGKAPKLLIFAASSLESGIPSRFSGSGSGTDFTLTIS |
| | | | | | SLQPEDFATYYCQQANGFPLTFGGGTKVEIK |
| TPP- | 13612-rec02- | LCDR1 | PRT | 26 | RASQDVGSWLA |
| 14389 | hIgG1Kappa | | | | |
| TPP- | 13612-rec02- | LCDR2 | PRT | 27 | AASSLES |
| 14389 | hIgG1Kappa | | | | |
| TPP- | 13612-rec02- | LCDR3 | PRT | 28 | QQANGFPLT |
| 14389 | hIgG1Kappa | | | | |
| TPP- | 13612-rec02- | VH | DNA | 29 | GAGGTGCAGCTGCTGGAATCTGGCGGAGGATTGG |
| 14389 | hIgG1Kappa | | | | TTCAGCCTGGCGGCTCTCTGAGACTGTCTTGTGCCG |
| | | | | | CTTCTGGCTTCACCTTCTCCGGCTACATGATGTCCTG |
| | | | | | GGTCCGACAGGCTCCTGGCAAAGGACTGGAATGG |
| | | | | | GTGTCCGGCATCTATCCCAGTCCTGGCTACACCTAC |
| | | | | | TACGCCGACTCTGTGAAGGGCAGATTCACCATCAG |
| | | | | | CCGGGACAACTCCAAGAACACCCTGTACCTGCAGA |
| | | | | | TGAACTCCCTGAGAGCCGAGGACACCGCCGTGTAC |
| | | | | | TACTGTGCCAGAGAGAAGGCCTCTGACCTGTTCGG |
| | | | | | CTCTTACTCTGAGGCCCTGGATTATTGGGGCCAGG |
| | | | | | GCACACTGGTTACCGTGTCATCA |
| TPP- | 13612-rec02- | VL | DNA | 30 | GATATCCAGATGACCCAGTCTCCTGACTCTCTGTCC |
| 14389 | hIgG1Kappa | | | | GCCTCTGTGGGCGACAGAGTGACCATCACCTGTAG |
| | | | | | AGCCTCTCAGGACGTCGGCTCTTGGCTGGCTTGGT |
| | | | | | ATCAGCAGAAGCCTGGCAAGGCCCCTAAGCTGCTG |
| | | | | | ATCTTTGCCGCCTCCTCTCTGGAATCTGGCATCCCCT |
| | | | | | CTAGATTCTCCGGCTCTGGCTCTGGCACCGACTTTA |
| | | | | | CCCTGACAATCTCCAGCCTGCAGCCTGAGGACTTCG |
| | | | | | CCACCTACTACTGTCAGCAGGCCAACGGCTTCCCAC |
| | | | | | TGACATTTGGCGGCGGAACAAAGGTGGAAATCAA |
| | | | | | A |
| TPP- | 13612-rec02- | Heavy | PRT | 31 | EVQLLESGGGLVQPGGSLRLSCAASGFTFSGYMMSW |
| 14389 | hIgG1Kappa | Chain | | | VRQAPGKGLEWVSGIYPSPGYTYYADSVKGRFTISRD |
| | | | | | NSKNTLYLQMNSLRAEDTAVYYCAREKASDLFGSYSE |
| | | | | | ALDYWGQGTLVTVSSASTKGPSVFPLAPSSKSTSGGT |
| | | | | | AALGCLVKDYFPEPVTVSWNSGALTSGVHTFPAVLQS |
| | | | | | SGLYSLSSVVTVPSSSLGTQTYICNVNHKPSNTKVDKK |
| | | | | | VEPKSCDKTHTCPPCPAPELLGGPSVFLFPPKPKDTLM |
| | | | | | ISRTPEVTCVVVDVSHEDPEVKFNWYVDGVEVHNAK |
| | | | | | TKPREEQYNSTYRVVSVLTVLHQDWLNGKEYKCKVS |
| | | | | | NKALPAPIEKTISKAKGQPREPQVYTLPPSRDELTKNQ |
| | | | | | VSLTCLVKGFYPSDIAVEWESNGQPENNYKTTPPVLD |
| | | | | | SDGSFFLYSKLTVDKSRWQQGNVFSCSVMHEALHNH |
| | | | | | YTQKSLSLSPG |
| TPP- | 13612-rec02- | Light | PRT | 32 | DIQMTQSPDSLSASVGDRVTITCRASQDVGSWLAWY |
| 14389 | hIgG1Kappa | Chain | | | QQKPGKAPKLLIFAASSLESGIPSRFSGSGSGTDFTLTIS |
| | | | | | SLQPEDFATYYCQQANGFPLTFGGGTKVEIKRTVAAP |
| | | | | | SVFIFPPSDEQLKSGTASVVCLLNNFYPREAKVQWKV |
| | | | | | DNALQSGNSQESVTEQDSKDSTYSLSSTLTLSKADYEK |
| | | | | | HKVYACEVTHQGLSSPVTKSFNRGEC |
| TPP- | 13612-rec02- | Heavy | DNA | 33 | GAGGTGCAGCTGCTGGAATCTGGCGGAGGATTGG |
| 14389 | hIgG1Kappa | Chain | | | TTCAGCCTGGCGGCTCTCTGAGACTGTCTTGTGCCG |
| | | | | | CTTCTGGCTTCACCTTCTCCGGCTACATGATGTCCTG |
| | | | | | GGTCCGACAGGCTCCTGGCAAAGGACTGGAATGG |
| | | | | | GTGTCCGGCATCTATCCCAGTCCTGGCTACACCTAC |
| | | | | | TACGCCGACTCTGTGAAGGGCAGATTCACCATCAG |
| | | | | | CCGGGACAACTCCAAGAACACCCTGTACCTGCAGA |
| | | | | | TGAACTCCCTGAGAGCCGAGGACACCGCCGTGTAC |
| | | | | | TACTGTGCCAGAGAGAAGGCCTCTGACCTGTTCGG |
| | | | | | CTCTTACTCTGAGGCCCTGGATTATTGGGGCCAGG |
| | | | | | GCACACTGGTTACCGTGTCATCAGCCTCCACCAAG |
| | | | | | GGCCCCTCCGTGTTTCCTCTGGCCCCTTCCAGCAAG |
| | | | | | TCCACCTCTGGCGGAACAGCCGCTCTGGGCTGCCT |
| | | | | | CGTGAAGGACTACTTCCCCGAGCCTGTGACCGTGT |
| | | | | | CCTGGAACTCTGGCGCTCTGACATCCGGCGTGCAC |
| | | | | | ACCTTCCCTGCTGTGCTGCAGTCTAGCGGCCTGTAC |
| | | | | | TCCCTGTCCTCCGTCGTGACCGTGCCTTCCAGCTCTC |
| | | | | | TGGGCACCCAGACCTACATCTGCAACGTGAACCAC |
| | | | | | AAGCCCTCCAACACCAAGGTGGACAAGAAGGTGG |
| | | | | | AACCCAAGTCCTGCGACAAGACCCACACCTGTCCCC |
| | | | | | CTTGTCCTGCCCCTGAACTGCTGGGCGGACCTTCCG |
| | | | | | TGTTCCTGTTCCCCCCAAAGCCCAAGGACACCCTGA |
| | | | | | TGATCTCCCGGACCCCCGAAGTGACCTGCGTGGTG |
| | | | | | GTGGATGTGTCCCACGAGGACCCTGAAGTGAAGTT |
| | | | | | CAATTGGTACGTGGACGGCGTGGAAGTGCACAAC |
| | | | | | GCCAAGACCAAGCCTAGAGAGGAACAGTACAACTC |
| | | | | | CACCTACCGGGTGGTGTCCGTGCTGACCGTGCTGC |
| | | | | | ACCAGGATTGGCTGAACGGCAAAGAGTACAAGTG |
| | | | | | CAAGGTGTCCAACAAGGCCCTGCCTGCCCCCATCG |
| | | | | | AAAAGACCATCTCCAAGGCCAAGGGCCAGCCCCGG |
| | | | | | GAACCCCAGGTGTACACACTGCCCCCTAGCAGGGA |
| | | | | | CGAGCTGACCAAGAACCAGGTGTCCCTGACCTGTC |
| | | | | | TCGTGAAAGGCTTCTACCCCTCCGATATCGCCGTGG |
| | | | | | AATGGGAGTCCAACGGCCAGCCTGAGAACAACTAC |
| | | | | | AAGACCACCCCCCCTGTGCTGGACTCCGACGGCTC |
| | | | | | ATTCTTCCTGTACAGCAAGCTGACAGTGGACAAGT |
| | | | | | CCCGGTGGCAGCAGGGCAACGTGTTCTCCTGCTCC |
| | | | | | GTGATGCACGAGGCCCTGCACAACCACTACACCCA |
| | | | | | GAAGTCCCTGTCCCTGAGCCCTGGC |
| TPP- | 13612-rec02- | Light | DNA | 34 | GATATCCAGATGACCCAGTCTCCTGACTCTCTGTCC |
| 14389 | hIgG1Kappa | Chain | | | GCCTCTGTGGGCGACAGAGTGACCATCACCTGTAG |
| | | | | | AGCCTCTCAGGACGTCGGCTCTTGGCTGGCTTGGT |
| | | | | | ATCAGCAGAAGCCTGGCAAGGCCCCTAAGCTGCTG |
| | | | | | ATCTTTGCCGCCTCCTCTCTGGAATCTGGCATCCCCT |
| | | | | | CTAGATTCTCCGGCTCTGGCTCTGGCACCGACTTTA |
| | | | | | CCCTGACAATCTCCAGCCTGCAGCCTGAGGACTTCG |
| | | | | | CCACCTACTACTGTCAGCAGGCCAACGGCTTCCCAC |
| | | | | | TGACATTTGGCGGCGGAACAAAGGTGGAAATCAA |
| | | | | | ACGAACCGTGGCCGCTCCCTCCGTGTTCATCTTCCC |
| | | | | | ACCCTCCGACGAGCAGCTGAAGTCCGGCACCGCCA |
| | | | | | GCGTCGTGTGCCTGCTGAACAACTTCTACCCCCGCG |
| | | | | | AGGCCAAGGTGCAGTGGAAGGTGGACAACGCCCT |
| | | | | | GCAGTCCGGCAACTCCCAGGAATCCGTCACCGAGC |
| | | | | | AGGACTCCAAGGACAGCACCTACTCCCTGTCCTCCA |
| | | | | | CCCTGACCCTGTCCAAGGCCGACTACGAGAAGCAC |
| | | | | | AAGGTGTACGCCTGCGAAGTGACCCACCAGGGCCT |
| | | | | | GTCCAGCCCCGTGACCAAGTCCTTCAACCGGGGCG |
| | | | | | AGTGC |
| TPP- | 13612-rec05- | VH | PRT | 35 | EVQLLESGGGLVQPGGSLRLSCAASGFTFSGYMMSW |
| 14392 | hIgG1Kappa | | | | VRQAPGKGLEWVSGIYPSPGYTYYADSVKGRFTISRD |
| | | | | | NSKNTLYLQMNSLRAEDTAVYYCAREKASDLSGSYSE |
| | | | | | ALDYWGQGTLVTVSS |
| TPP- | 13612-rec05- | HCDR1 | PRT | 36 | GYMMS |
| 14392 | hIgG1Kappa | | | | |
| TPP- | 13612-rec05- | HCDR2 | PRT | 37 | GIYPSPGYTYYADSVKG |
| 14392 | hIgG1Kappa | | | | |
| TPP- | 13612-rec05- | HCDR3 | PRT | 38 | EKASDLSGSYSEALDY |
| 14392 | hIgG1Kappa | | | | |
| TPP- | 13612-rec05- | VL | PRT | 39 | DIQMTQSPDSLSASVGDRVTITCRASQDVGSWLAWY |
| 14392 | hIgG1Kappa | | | | QQKPGKAPKLLIFAASYLESGIPSRFSGSGSGTDFTLTIS |
| | | | | | SLQPEDFATYYCQQANGFPLTFGGGTKVEIK |
| TPP- | 13612-rec05- | LCDR1 | PRT | 40 | RASQDVGSWLA |
| 14392 | hIgG1Kappa | | | | |
| TPP- | 13612-rec05- | LCDR2 | PRT | 41 | AASYLES |
| 14392 | hIgG1Kappa | | | | |
| TPP- | 13612-rec05- | LCDR3 | PRT | 42 | QQANGFPLT |
| 14392 | hIgG1Kappa | | | | |
| TPP- | 13612-rec05- | VH | DNA | 43 | GAGGTGCAGCTGCTGGAATCTGGCGGAGGATTGG |
| 14392 | hIgG1Kappa | | | | TTCAGCCTGGCGGCTCTCTGAGACTGTCTTGTGCCG |
| | | | | | CTTCTGGCTTCACCTTCTCCGGCTACATGATGTCCTG |
| | | | | | GGTCCGACAGGCTCCTGGCAAAGGACTGGAATGG |
| | | | | | GTGTCCGGCATCTATCCCAGTCCTGGCTACACCTAC |
| | | | | | TACGCCGACTCTGTGAAGGGCAGATTCACCATCAG |
| | | | | | CCGGGACAACTCCAAGAACACCCTGTACCTGCAGA |
| | | | | | TGAACTCCCTGAGAGCCGAGGACACCGCCGTGTAC |
| | | | | | TACTGTGCCAGAGAGAAGGCCTCTGACCTGTCCGG |
| | | | | | CTCTTACTCTGAGGCCCTGGATTATTGGGGCCAGG |
| | | | | | GCACACTGGTTACCGTGTCATCA |
| TPP- | 13612-rec05- | VL | DNA | 44 | GATATCCAGATGACCCAGTCTCCTGACTCTCTGTCC |
| 14392 | hIgG1Kappa | | | | GCCTCTGTGGGCGACAGAGTGACCATCACCTGTAG |
| | | | | | AGCCTCTCAGGACGTCGGCTCTTGGCTGGCTTGGT |
| | | | | | ATCAGCAGAAGCCTGGCAAGGCCCCTAAGCTGCTG |
| | | | | | ATCTTCGCCGCCTCCTATCTGGAAAGCGGCATCCCT |
| | | | | | TCCAGATTCTCCGGCTCTGGCTCTGGCACCGACTTT |
| | | | | | ACCCTGACAATCTCCAGCCTGCAGCCTGAGGACTTC |
| | | | | | GCCACCTACTACTGTCAGCAGGCCAACGGCTTCCCA |
| | | | | | CTGACATTTGGCGGCGGAACAAAGGTGGAAATCA |
| | | | | | AA |
| TPP- | 13612-rec05- | Heavy | PRT | 45 | EVQLLESGGGLVQPGGSLRLSCAASGFTFSGYMMSW |
| 14392 | hIgG1Kappa | Chain | | | VRQAPGKGLEWVSGIYPSPGYTYYADSVKGRFTISRD |
| | | | | | NSKNTLYLQMNSLRAEDTAVYYCAREKASDLSGSYSE |
| | | | | | ALDYWGQGTLVTVSSASTKGPSVFPLAPSSKSTSGGT |
| | | | | | AALGCLVKDYFPEPVTVSWNSGALTSGVHTFPAVLQS |
| | | | | | SGLYSLSSVVTVPSSSLGTQTYICNVNHKPSNTKVDKK |
| | | | | | VEPKSCDKTHTCPPCPAPELLGGPSVFLFPPKPKDTLM |
| | | | | | ISRTPEVTCVVVDVSHEDPEVKFNWYVDGVEVHNAK |
| | | | | | TKPREEQYNSTYRVVSVLTVLHQDWLNGKEYKCKVS |
| | | | | | NKALPAPIEKTISKAKGQPREPQVYTLPPSRDELTKNQ |
| | | | | | VSLTCLVKGFYPSDIAVEWESNGQPENNYKTTPPVLD |
| | | | | | SDGSFFLYSKLTVDKSRWQQGNVFSCSVMHEALHNH |
| | | | | | YTQKSLSLSPG |
| TPP- | 13612-rec05- | Light | PRT | 46 | DIQMTQSPDSLSASVGDRVTITCRASQDVGSWLAWY |
| 14392 | hIgG1Kappa | Chain | | | QQKPGKAPKLLIFAASYLESGIPSRFSGSGSGTDFTLTIS |
| | | | | | SLQPEDFATYYCQQANGFPLTFGGGTKVEIKRTVAAP |
| | | | | | SVFIFPPSDEQLKSGTASVVCLLNNFYPREAKVQWKV |
| | | | | | DNALQSGNSQESVTEQDSKDSTYSLSSTLTLSKADYEK |
| | | | | | HKVYACEVTHQGLSSPVTKSFNRGEC |
| TPP- | 13612-rec05- | Heavy | DNA | 47 | GAGGTGCAGCTGCTGGAATCTGGCGGAGGATTGG |
| 14392 | hIgG1Kappa | Chain | | | TTCAGCCTGGCGGCTCTCTGAGACTGTCTTGTGCCG |
| | | | | | CTTCTGGCTTCACCTTCTCCGGCTACATGATGTCCTG |
| | | | | | GGTCCGACAGGCTCCTGGCAAAGGACTGGAATGG |
| | | | | | GTGTCCGGCATCTATCCCAGTCCTGGCTACACCTAC |
| | | | | | TACGCCGACTCTGTGAAGGGCAGATTCACCATCAG |
| | | | | | CCGGGACAACTCCAAGAACACCCTGTACCTGCAGA |
| | | | | | TGAACTCCCTGAGAGCCGAGGACACCGCCGTGTAC |
| | | | | | TACTGTGCCAGAGAGAAGGCCTCTGACCTGTCCGG |
| | | | | | CTCTTACTCTGAGGCCCTGGATTATTGGGGCCAGG |
| | | | | | GCACACTGGTTACCGTGTCATCAGCCTCCACCAAG |
| | | | | | GGCCCCTCCGTGTTTCCTCTGGCCCCTTCCAGCAAG |
| | | | | | TCCACCTCTGGCGGAACAGCCGCTCTGGGCTGCCT |
| | | | | | CGTGAAGGACTACTTCCCCGAGCCTGTGACCGTGT |
| | | | | | CCTGGAACTCTGGCGCTCTGACATCCGGCGTGCAC |
| | | | | | ACCTTCCCTGCTGTGCTGCAGTCTAGCGGCCTGTAC |
| | | | | | TCCCTGTCCTCCGTCGTGACCGTGCCTTCCAGCTCTC |
| | | | | | TGGGCACCCAGACCTACATCTGCAACGTGAACCAC |
| | | | | | AAGCCCTCCAACACCAAGGTGGACAAGAAGGTGG |
| | | | | | AACCCAAGTCCTGCGACAAGACCCACACCTGTCCCC |
| | | | | | CTTGTCCTGCCCCTGAACTGCTGGGCGGACCTTCCG |
| | | | | | TGTTCCTGTTCCCCCCAAAGCCCAAGGACACCCTGA |
| | | | | | TGATCTCCCGGACCCCCGAAGTGACCTGCGTGGTG |
| | | | | | GTGGATGTGTCCCACGAGGACCCTGAAGTGAAGTT |
| | | | | | CAATTGGTACGTGGACGGCGTGGAAGTGCACAAC |
| | | | | | GCCAAGACCAAGCCTAGAGAGGAACAGTACAACTC |
| | | | | | CACCTACCGGGTGGTGTCCGTGCTGACCGTGCTGC |
| | | | | | ACCAGGATTGGCTGAACGGCAAAGAGTACAAGTG |
| | | | | | CAAGGTGTCCAACAAGGCCCTGCCTGCCCCCATCG |
| | | | | | AAAAGACCATCTCCAAGGCCAAGGGCCAGCCCCGG |
| | | | | | GAACCCCAGGTGTACACACTGCCCCCTAGCAGGGA |
| | | | | | CGAGCTGACCAAGAACCAGGTGTCCCTGACCTGTC |
| | | | | | TCGTGAAAGGCTTCTACCCCTCCGATATCGCCGTGG |
| | | | | | AATGGGAGTCCAACGGCCAGCCTGAGAACAACTAC |
| | | | | | AAGACCACCCCCCCTGTGCTGGACTCCGACGGCTC |
| | | | | | ATTCTTCCTGTACAGCAAGCTGACAGTGGACAAGT |
| | | | | | CCCGGTGGCAGCAGGGCAACGTGTTCTCCTGCTCC |
| | | | | | GTGATGCACGAGGCCCTGCACAACCACTACACCCA |
| | | | | | GAAGTCCCTGTCCCTGAGCCCTGGC |
| TPP- | 13612-rec05- | Light | DNA | 48 | GATATCCAGATGACCCAGTCTCCTGACTCTCTGTCC |
| 14392 | hIgG1Kappa | Chain | | | GCCTCTGTGGGCGACAGAGTGACCATCACCTGTAG |
| | | | | | AGCCTCTCAGGACGTCGGCTCTTGGCTGGCTTGGT |
| | | | | | ATCAGCAGAAGCCTGGCAAGGCCCCTAAGCTGCTG |
| | | | | | ATCTTCGCCGCCTCCTATCTGGAAAGCGGCATCCCT |
| | | | | | TCCAGATTCTCCGGCTCTGGCTCTGGCACCGACTTT |
| | | | | | ACCCTGACAATCTCCAGCCTGCAGCCTGAGGACTTC |
| | | | | | GCCACCTACTACTGTCAGCAGGCCAACGGCTTCCCA |
| | | | | | CTGACATTTGGCGGCGGAACAAAGGTGGAAATCA |
| | | | | | AACGAACCGTGGCCGCTCCCTCCGTGTTCATCTTCC |
| | | | | | CACCCTCCGACGAGCAGCTGAAGTCCGGCACCGCC |
| | | | | | AGCGTCGTGTGCCTGCTGAACAACTTCTACCCCCGC |
| | | | | | GAGGCCAAGGTGCAGTGGAAGGTGGACAACGCCC |
| | | | | | TGCAGTCCGGCAACTCCCAGGAATCCGTCACCGAG |
| | | | | | CAGGACTCCAAGGACAGCACCTACTCCCTGTCCTCC |
| | | | | | ACCCTGACCCTGTCCAAGGCCGACTACGAGAAGCA |
| | | | | | CAAGGTGTACGCCTGCGAAGTGACCCACCAGGGCC |
| | | | | | TGTCCAGCCCCGTGACCAAGTCCTTCAACCGGGGC |
| | | | | | GAGTGC |
| TPP- | 438H-M113-N15- | VH | PRT | 49 | EVQLLESGGGLVQPGGSLRLSCAASGFTFSGYMMSW |
| 17073 | hIgG1 | | | | VRQAPGKGLEWVSGIYPSAGYTLYADSVKGRFTISRD |
| | | | | | NSKNTLYLQMNSLRAEDTAVYYCAREKAADLFGSYSE |
| | | | | | ALDYWGQGTLVTVSS |
| TPP- | 438H-M113-N15- | HCDR1 | PRT | 50 | GYMMS |
| 17073 | hIgG1 | | | | |
| TPP- | 438H-M113-N15- | HCDR2 | PRT | 51 | GIYPSAGYTLYADSVKG |
| 17073 | hIgG1 | | | | |
| TPP- | 438H-M113-N15- | HCDR3 | PRT | 52 | EKAADLFGSYSEALDY |
| 17073 | hIgG1 | | | | |
| TPP- | 438H-M113-N15- | VL | PRT | 53 | DIQMTQSPDSLSASVGDRVTITCRASQDVGSWLAWY |
| 17073 | hIgG1 | | | | QQKPGKAPKLLIFAASYLESGIPSRFSGSGSGTDFTLTIS |
| | | | | | SLQPEDFATYYCQQANGFPLTFGGGTKVEIK |
| TPP- | 438H-M113-N15- | LCDR1 | PRT | 54 | RASQDVGSWLA |
| 17073 | hIgG1 | | | | |
| TPP- | 438H-M113-N15- | LCDR2 | PRT | 55 | AASYLES |
| 17073 | hIgG1 | | | | |
| TPP- | 438H-M113-N15- | LCDR3 | PRT | 56 | QQANGFPLT |
| 17073 | hIgG1 | | | | |
| TPP- | 438H-M113-N15- | Heavy | PRT | 57 | EVQLLESGGGLVQPGGSLRLSCAASGFTFSGYMMSW |
| 17073 | hIgG1 | Chain | | | VRQAPGKGLEWVSGIYPSAGYTLYADSVKGRFTISRD |
| | | | | | NSKNTLYLQMNSLRAEDTAVYYCAREKAADLFGSYSE |
| | | | | | ALDYWGQGTLVTVSSASTKGPSVFPLAPSSKSTSGGT |
| | | | | | AALGCLVKDYFPEPVTVSWNSGALTSGVHTFPAVLQS |
| | | | | | SGLYSLSSVVTVPSSSLGTQTYICNVNHKPSNTKVDKK |
| | | | | | VEPKSCDKTHTCPPCPAPELLGGPSVFLFPPKPKDTLM |
| | | | | | ISRTPEVTCVVVDVSHEDPEVKFNWYVDGVEVHNAK |
| | | | | | TKPREEQYNSTYRVVSVLTVLHQDWLNGKEYKCKVS |
| | | | | | NKALPAPIEKTISKAKGQPREPQVYTLPPSRDELTKNQ |
| | | | | | VSLTCLVKGFYPSDIAVEWESNGQPENNYKTTPPVLD |
| | | | | | SDGSFFLYSKLTVDKSRWQQGNVFSCSVMHEALHNH |
| | | | | | YTQKSLSLSPG |
| TPP- | 438H-M113-N15- | Light | PRT | 58 | DIQMTQSPDSLSASVGDRVTITCRASQDVGSWLAWY |
| 17073 | hIgG1 | Chain | | | QQKPGKAPKLLIFAASYLESGIPSRFSGSGSGTDFTLTIS |
| | | | | | SLQPEDFATYYCQQANGFPLTFGGGTKVEIKRTVAAP |
| | | | | | SVFIFPPSDEQLKSGTASVVCLLNNFYPREAKVQWKV |
| | | | | | DNALQSGNSQESVTEQDSKDSTYSLSSTLTLSKADYEK |
| | | | | | HKVYACEVTHQGLSSPVTKSFNRGEC |
| TPP- | 438H-M161-K22- | VH | PRT | 59 | EVQLLESGGGLVQPGGSLRLSCAASGFTFSGYMMSW |
| 17074 | hIgG1 | | | | VRQAPGKGLEWVSGIYPSGGYALYADSVKGRFTISRD |
| | | | | | NSKNTLYLQMNSLRAEDTAVYYCAREKAADLFGSYSE |
| | | | | | ALDYWGQGTLVTVSS |
| TPP- | 438H-M161-K22- | HCDR1 | PRT | 60 | GYMMS |
| 17074 | hIgG1 | | | | |
| TPP- | 438H-M161-K22- | HCDR2 | PRT | 61 | GIYPSGGYALYADSVKG |
| 17074 | hIgG1 | | | | |
| TPP- | 438H-M161-K22- | HCDR3 | PRT | 62 | EKAADLFGSYSEALDY |
| 17074 | hIgG1 | | | | |
| TPP- | 438H-M161-K22- | VL | PRT | 63 | DIQMTQSPDSLSASVGDRVTITCRASQDVGSWLAWY |
| 17074 | hIgG1 | | | | QQKPGKAPKLLIFAASYLESGIPSRFSGSGSGTDFTLTIS |
| | | | | | SLQPEDFATYYCQQANGFPLTFGGGTKVEIK |
| TPP- | 438H-M161-K22- | LCDR1 | PRT | 64 | RASQDVGSWLA |
| 17074 | hIgG1 | | | | |
| TPP- | 438H-M161-K22- | LCDR2 | PRT | 65 | AASYLES |
| 17074 | hIgG1 | | | | |
| TPP- | 438H-M161-K22- | LCDR3 | PRT | 66 | QQANGFPLT |
| 17074 | hIgG1 | | | | |
| TPP- | 438H-M161-K22- | Heavy | PRT | 67 | EVQLLESGGGLVQPGGSLRLSCAASGFTFSGYMMSW |
| 17074 | hIgG1 | Chain | | | VRQAPGKGLEWVSGIYPSGGYALYADSVKGRFTISRD |
| | | | | | NSKNTLYLQMNSLRAEDTAVYYCAREKAADLFGSYSE |
| | | | | | ALDYWGQGTLVTVSSASTKGPSVFPLAPSSKSTSGGT |
| | | | | | AALGCLVKDYFPEPVTVSWNSGALTSGVHTFPAVLQS |
| | | | | | SGLYSLSSVVTVPSSSLGTQTYICNVNHKPSNTKVDKK |
| | | | | | VEPKSCDKTHTCPPCPAPELLGGPSVFLFPPKPKDTLM |
| | | | | | ISRTPEVTCVVVDVSHEDPEVKFNWYVDGVEVHNAK |
| | | | | | TKPREEQYNSTYRVVSVLTVLHQDWLNGKEYKCKVS |
| | | | | | NKALPAPIEKTISKAKGQPREPQVYTLPPSRDELTKNQ |
| | | | | | VSLTCLVKGFYPSDIAVEWESNGQPENNYKTTPPVLD |
| | | | | | SDGSFFLYSKLTVDKSRWQQGNVFSCSVMHEALHNH |
| | | | | | YTQKSLSLSPG |
| TPP- | 438H-M161-K22- | Light | PRT | 68 | DIQMTQSPDSLSASVGDRVTITCRASQDVGSWLAWY |
| 17074 | hIgG1 | Chain | | | QQKPGKAPKLLIFAASYLESGIPSRFSGSGSGTDFTLTIS |
| | | | | | SLQPEDFATYYCQQANGFPLTFGGGTKVEIKRTVAAP |
| | | | | | SVFIFPPSDEQLKSGTASVVCLLNNFYPREAKVQWKV |
| | | | | | DNALQSGNSQESVTEQDSKDSTYSLSSTLTLSKADYEK |
| | | | | | HKVYACEVTHQGLSSPVTKSFNRGEC |
| TPP- | 438H-M308-H05- | VH | PRT | 69 | EVQLVQSGAEVKKPGASVKVSCKASGYKFSSYWIEW |
| 17078 | hIgGkappa | | | | VKQAPGQGLEWIGEILPGSDTTNYNEKFKDRATFTSD |
| | | | | | TSINTAYMELSRLRSDDTAVYYCARDRGNYRAWFGY |
| | | | | | WGQGTLVTVSS |
| TPP- | 438H-M308-H05- | HCDR1 | PRT | 70 | SYWIE |
| 17078 | hIgGkappa | | | | |
| TPP- | 438H-M308-H05- | HCDR2 | PRT | 71 | EILPGSDTTNYNEKFKD |
| 17078 | hIgGkappa | | | | |
| TPP- | 438H-M308-H05- | HCDR3 | PRT | 72 | DRGNYRAWFGY |
| 17078 | hIgGkappa | | | | |
| TPP- | 438H-M308-H05- | VL | PRT | 73 | DIQMTQSPSSLSASVGDRVTITCRASQSISSYLNWYQ |
| 17078 | hIgGkappa | | | | QKPGGAPKFLIYYASSLQSGVPSRFSGSGSGTDYTLTIS |
| | | | | | SLQPEDFATYYCQQGFSLPWTFGGGTKVEIK |
| TPP- | 438H-M308-H05- | LCDR1 | PRT | 74 | RASQSISSYLN |
| 17078 | hIgGkappa | | | | |
| TPP- | 438H-M308-H05- | LCDR2 | PRT | 75 | YASSLQS |
| 17078 | hIgGkappa | | | | |
| TPP- | 438H-M308-H05- | LCDR3 | PRT | 76 | QQGFSLPWT |
| 17078 | hIgGkappa | | | | |
| TPP- | 438H-M308-H05- | VH | DNA | 77 | GAGGTGCAGCTGGTGCAGTCTGGCGCCGAAGTGA |
| 17078 | hIgGkappa | | | | AAAAGCCTGGCGCCTCTGTGAAGGTGTCCTGCAAG |
| | | | | | GCTTCCGGCTACAAGTTCTCCAGCTACTGGATCGAG |
| | | | | | TGGGTCAAGCAGGCTCCTGGACAGGGACTCGAGT |
| | | | | | GGATCGGAGAGATCCTGCCTGGCTCTGACACCACC |
| | | | | | AACTACAACGAGAAGTTCAAGGACCGGGCCACCTT |
| | | | | | CACCTCCGACACCTCTATCAACACCGCCTACATGGA |
| | | | | | ACTGTCCCGGCTGAGATCTGACGACACCGCCGTGT |
| | | | | | ACTACTGCGCCAGAGACAGAGGCAACTACAGAGCT |
| | | | | | TGGTTTGGCTACTGGGGCCAGGGCACACTGGTTAC |
| | | | | | AGTTAGCTCA |
| TPP- | 438H-M308-H05- | VL | DNA | 78 | GATATCCAGATGACCCAGTCTCCTTCCAGCCTGTCT |
| 17078 | hIgGkappa | | | | GCCTCTGTGGGCGACAGAGTGACCATCACCTGTCG |
| | | | | | GGCCTCTCAGTCCATCTCCTCCTACCTGAACTGGTA |
| | | | | | TCAGCAGAAGCCTGGCGGCGCTCCCAAGTTCCTGA |
| | | | | | TCTACTACGCTAGCTCCCTGCAGTCCGGCGTGCCCT |
| | | | | | CTAGATTTTCTGGCTCTGGATCCGGCACCGACTATA |
| | | | | | CCCTGACAATCTCCAGCCTGCAGCCTGAGGACTTCG |
| | | | | | CCACCTACTATTGCCAGCAGGGCTTCTCCCTGCCTT |
| | | | | | GGACATTTGGCGGCGGAACAAAGGTGGAAATCAA |
| | | | | | A |
| TPP- | 438H-M308-H05- | Heavy | PRT | 79 | EVQLVQSGAEVKKPGASVKVSCKASGYKFSSYWIEW |
| 17078 | hIgGkappa | Chain | | | VKQAPGQGLEWIGEILPGSDTTNYNEKFKDRATFTSD |
| | | | | | TSINTAYMELSRLRSDDTAVYYCARDRGNYRAWFGY |
| | | | | | WGQGTLVTVSSASTKGPSVFPLAPSSKSTSGGTAALG |
| | | | | | CLVKDYFPEPVTVSWNSGALTSGVHTFPAVLQSSGLY |
| | | | | | SLSSVVTVPSSSLGTQTYICNVNHKPSNTKVDKKVEPK |
| | | | | | SCDKTHTCPPCPAPELLGGPSVFLFPPKPKDTLMISRT |
| | | | | | PEVTCVVVDVSHEDPEVKFNWYVDGVEVHNAKTKP |
| | | | | | REEQYNSTYRVVSVLTVLHQDWLNGKEYKCKVSNKA |
| | | | | | LPAPIEKTISKAKGQPREPQVYTLPPSRDELTKNQVSLT |
| | | | | | CLVKGFYPSDIAVEWESNGQPENNYKTTPPVLDSDGS |
| | | | | | FFLYSKLTVDKSRWQQGNVFSCSVMHEALHNHYTQK |
| | | | | | SLSLSPG |
| TPP- | 438H-M308-H05- | Light | PRT | 80 | DIQMTQSPSSLSASVGDRVTITCRASQSISSYLNWYQ |
| 17078 | hIgGkappa | Chain | | | QKPGGAPKFLIYYASSLQSGVPSRFSGSGSGTDYTLTIS |
| | | | | | SLQPEDFATYYCQQGFSLPWTFGGGTKVEIKRTVAAP |
| | | | | | SVFIFPPSDEQLKSGTASVVCLLNNFYPREAKVQWKV |
| | | | | | DNALQSGNSQESVTEQDSKDSTYSLSSTLTLSKADYEK |
| | | | | | HKVYACEVTHQGLSSPVTKSFNRGEC |
| TPP- | 438H-M308-H05- | Heavy | DNA | 81 | GAGGTGCAGCTGGTGCAGTCTGGCGCCGAAGTGA |
| 17078 | hIgGkappa | Chain | | | AAAAGCCTGGCGCCTCTGTGAAGGTGTCCTGCAAG |
| | | | | | GCTTCCGGCTACAAGTTCTCCAGCTACTGGATCGAG |
| | | | | | TGGGTCAAGCAGGCTCCTGGACAGGGACTCGAGT |
| | | | | | GGATCGGAGAGATCCTGCCTGGCTCTGACACCACC |
| | | | | | AACTACAACGAGAAGTTCAAGGACCGGGCCACCTT |
| | | | | | CACCTCCGACACCTCTATCAACACCGCCTACATGGA |
| | | | | | ACTGTCCCGGCTGAGATCTGACGACACCGCCGTGT |
| | | | | | ACTACTGCGCCAGAGACAGAGGCAACTACAGAGCT |
| | | | | | TGGTTTGGCTACTGGGGCCAGGGCACACTGGTTAC |
| | | | | | AGTTAGCTCAGCCTCCACCAAGGGCCCCTCCGTGTT |
| | | | | | TCCTCTGGCCCCTTCCAGCAAGTCCACCTCTGGCGG |
| | | | | | AACAGCCGCTCTGGGCTGCCTCGTGAAGGACTACT |
| | | | | | TCCCCGAGCCTGTGACCGTGTCCTGGAACTCTGGC |
| | | | | | GCTCTGACATCCGGCGTGCACACCTTCCCTGCTGTG |
| | | | | | CTGCAGTCTAGCGGCCTGTACTCCCTGTCCTCCGTC |
| | | | | | GTGACCGTGCCTTCCAGCTCTCTGGGCACCCAGACC |
| | | | | | TACATCTGCAACGTGAACCACAAGCCCTCCAACACC |
| | | | | | AAGGTGGACAAGAAGGTGGAACCCAAGTCCTGCG |
| | | | | | ACAAGACCCACACCTGTCCCCCTTGTCCTGCCCCTG |
| | | | | | AACTGCTGGGCGGACCTTCCGTGTTCCTGTTCCCCC |
| | | | | | CAAAGCCCAAGGACACCCTGATGATCTCCCGGACC |
| | | | | | CCCGAAGTGACCTGCGTGGTGGTGGATGTGTCCCA |
| | | | | | CGAGGACCCTGAAGTGAAGTTCAATTGGTACGTGG |
| | | | | | ACGGCGTGGAAGTGCACAACGCCAAGACCAAGCCT |
| | | | | | AGAGAGGAACAGTACAACTCCACCTACCGGGTGGT |
| | | | | | GTCCGTGCTGACCGTGCTGCACCAGGATTGGCTGA |
| | | | | | ACGGCAAAGAGTACAAGTGCAAGGTGTCCAACAA |
| | | | | | GGCCCTGCCTGCCCCCATCGAAAAGACCATCTCCAA |
| | | | | | GGCCAAGGGCCAGCCCCGGGAACCCCAGGTGTAC |
| | | | | | ACACTGCCCCCTAGCAGGGACGAGCTGACCAAGAA |
| | | | | | CCAGGTGTCCCTGACCTGTCTCGTGAAAGGCTTCTA |
| | | | | | CCCCTCCGATATCGCCGTGGAATGGGAGTCCAACG |
| | | | | | GCCAGCCTGAGAACAACTACAAGACCACCCCCCCT |
| | | | | | GTGCTGGACTCCGACGGCTCATTCTTCCTGTACAGC |
| | | | | | AAGCTGACAGTGGACAAGTCCCGGTGGCAGCAGG |
| | | | | | GCAACGTGTTCTCCTGCTCCGTGATGCACGAGGCC |
| | | | | | CTGCACAACCACTACACCCAGAAGTCCCTGTCCCTG |
| | | | | | AGCCCTGGC |
| TPP- | 438H-M308-H05- | Light | DNA | 82 | GATATCCAGATGACCCAGTCTCCTTCCAGCCTGTCT |
| 17078 | hIgGkappa | Chain | | | GCCTCTGTGGGCGACAGAGTGACCATCACCTGTCG |
| | | | | | GGCCTCTCAGTCCATCTCCTCCTACCTGAACTGGTA |
| | | | | | TCAGCAGAAGCCTGGCGGCGCTCCCAAGTTCCTGA |
| | | | | | TCTACTACGCTAGCTCCCTGCAGTCCGGCGTGCCCT |
| | | | | | CTAGATTTTCTGGCTCTGGATCCGGCACCGACTATA |
| | | | | | CCCTGACAATCTCCAGCCTGCAGCCTGAGGACTTCG |
| | | | | | CCACCTACTATTGCCAGCAGGGCTTCTCCCTGCCTT |
| | | | | | GGACATTTGGCGGCGGAACAAAGGTGGAAATCAA |
| | | | | | ACGAACCGTGGCCGCTCCCTCCGTGTTCATCTTCCC |
| | | | | | ACCCTCCGACGAGCAGCTGAAGTCCGGCACCGCCA |
| | | | | | GCGTCGTGTGCCTGCTGAACAACTTCTACCCCCGCG |
| | | | | | AGGCCAAGGTGCAGTGGAAGGTGGACAACGCCCT |
| | | | | | GCAGTCCGGCAACTCCCAGGAATCCGTCACCGAGC |
| | | | | | AGGACTCCAAGGACAGCACCTACTCCCTGTCCTCCA |
| | | | | | CCCTGACCCTGTCCAAGGCCGACTACGAGAAGCAC |
| | | | | | AAGGTGTACGCCTGCGAAGTGACCCACCAGGGCCT |
| | | | | | GTCCAGCCCCGTGACCAAGTCCTTCAACCGGGGCG |
| | | | | | AGTGC |
| TPP- | 438H-M345-F05- | VH | PRT | 83 | EVQLVQSGAEVKKPGASVKVSCKASGYKFSSYWIEW |
| 17405 | hIgG1Kappa | | | | VKQAPGQGLEWIGEILPGSDTTNYNEKFKDRATFTSD |
| | | | | | TSINTAYMELSRLRSDDTAVYYCARDRGNYRAWFQY |
| | | | | | WGQGTLVTVSS |
| TPP- | 438H-M345-F05- | HCDR1 | PRT | 84 | SYWIE |
| 17405 | hIgG1Kappa | | | | |
| TPP- | 438H-M345-F05- | HCDR2 | PRT | 85 | EILPGSDTTNYNEKFKD |
| 17405 | hIgG1Kappa | | | | |
| TPP- | 438H-M345-F05- | HCDR3 | PRT | 86 | DRGNYRAWFQY |
| 17405 | hIgG1Kappa | | | | |
| TPP- | 438H-M345-F05- | VL | PRT | 87 | DIQMTQSPSSLSASVGDRVTITCRASQRISSYLNWYQ |
| 17405 | hIgG1Kappa | | | | QKPGGAPKFLIYYASSLQSGVPSRFSGSGSGTDYTLTIS |
| | | | | | SLQPEDFATYYCDQGLELPWTFGGGTKVEIK |
| TPP- | 438H-M345-F05- | LCDR1 | PRT | 88 | RASQRISSYLN |
| 17405 | hIgG1Kappa | | | | |
| TPP- | 438H-M345-F05- | LCDR2 | PRT | 89 | YASSLQS |
| 17405 | hIgG1Kappa | | | | |
| TPP- | 438H-M345-F05- | LCDR3 | PRT | 90 | DQGLELPWT |
| 17405 | hIgG1Kappa | | | | |
| TPP- | 438H-M345-F05- | Heavy | PRT | 91 | EVQLVQSGAEVKKPGASVKVSCKASGYKFSSYWIEW |
| 17405 | hIgG1Kappa | Chain | | | VKQAPGQGLEWIGEILPGSDTTNYNEKFKDRATFTSD |
| | | | | | TSINTAYMELSRLRSDDTAVYYCARDRGNYRAWFQY |
| | | | | | WGQGTLVTVSSASTKGPSVFPLAPSSKSTSGGTAALG |
| | | | | | CLVKDYFPEPVTVSWNSGALTSGVHTFPAVLQSSGLY |
| | | | | | SLSSVVTVPSSSLGTQTYICNVNHKPSNTKVDKKVEPK |
| | | | | | SCDKTHTCPPCPAPELLGGPSVFLFPPKPKDTLMISRT |
| | | | | | PEVTCVVVDVSHEDPEVKFNWYVDGVEVHNAKTKP |
| | | | | | REEQYNSTYRVVSVLTVLHQDWLNGKEYKCKVSNKA |
| | | | | | LPAPIEKTISKAKGQPREPQVYTLPPSRDELTKNQVSLT |
| | | | | | CLVKGFYPSDIAVEWESNGQPENNYKTTPPVLDSDGS |
| | | | | | FFLYSKLTVDKSRWQQGNVFSCSVMHEALHNHYTQK |
| | | | | | SLSLSPG |
| TPP- | 438H-M345-F05- | Light | PRT | 92 | DIQMTQSPSSLSASVGDRVTITCRASQRISSYLNWYQ |
| 17405 | hIgG1Kappa | Chain | | | QKPGGAPKFLIYYASSLQSGVPSRFSGSGSGTDYTLTIS |
| | | | | | SLQPEDFATYYCDQGLELPWTFGGGTKVEIKRTVAAP |
| | | | | | SVFIFPPSDEQLKSGTASVVCLLNNFYPREAKVQWKV |
| | | | | | DNALQSGNSQESVTEQDSKDSTYSLSSTLTLSKADYEK |
| | | | | | HKVYACEVTHQGLSSPVTKSFNRGEC |
| TPP- | 438H-M308- | VH | PRT | 93 | EVQLVQSGAEVKKPGASVKVSCKASGYKFSSYWIEW |
| 17418 | H05_B- | | | | VKQAPGQGLEWIGEILPGSDWTNYNEKFKDRATFTS |
| | hIgG1Kappa | | | | DTSINTAYMELSRLRSDDTAVYYCARDRGNYRAWFQ |
| | | | | | YWGQGTLVTVSS |
| TPP- | 438H-M308- | HCDR1 | PRT | 94 | SYWIE |
| 17418 | H05_B- | | | | |
| | hIgG1Kappa | | | | |
| TPP- | 438H-M308- | HCDR2 | PRT | 95 | EILPGSDWTNYNEKFKD |
| 17418 | H05_B- | | | | |
| | hIgG1Kappa | | | | |
| TPP- | 438H-M308- | HCDR3 | PRT | 96 | DRGNYRAWFQY |
| 17418 | H05_B- | | | | |
| | hIgG1Kappa | | | | |
| TPP- | 438H-M308- | VL | PRT | 97 | DIQMTQSPSSLSASVGDRVTITCRASQSISSYLNWYQ |
| 17418 | H05_B- | | | | QKPGGAPKFLIYYASSLQSGVPSRFSGSGSGTDYTLTIS |
| | hIgG1Kappa | | | | SLQPEDFATYYCQQGFSLPWTFGGGTKVEIK |
| TPP | 438H-M308- | LCDR1 | PRT | 98 | RASQSISSYLN |
| 17418 | H05_B- | | | | |
| | hIgG1Kappa | | | | |
| TPP- | 438H-M308- | LCDR2 | PRT | 99 | YASSLQS |
| 17418 | H05_B- | | | | |
| | hIgG1Kappa | | | | |
| TPP- | 438H-M308- | LCDR3 | PRT | 100 | QQGFSLPWT |
| 17418 | H05_B- | | | | |
| | hIgG1Kappa | | | | |
| TPP- | 438H-M308- | Heavy | PRT | 101 | EVQLVQSGAEVKKPGASVKVSCKASGYKFSSYWIEW |
| 17418 | H05_B- | Chain | | | VKQAPGQGLEWIGEILPGSDWTNYNEKFKDRATFTS |
| | hIgG1Kappa | | | | DTSINTAYMELSRLRSDDTAVYYCARDRGNYRAWFQ |
| | | | | | YWGQGTLVTVSSASTKGPSVFPLAPSSKSTSGGTAAL |
| | | | | | GCLVKDYFPEPVTVSWNSGALTSGVHTFPAVLQSSGL |
| | | | | | YSLSSVVTVPSSSLGTQTYICNVNHKPSNTKVDKKVEP |
| | | | | | KSCDKTHTCPPCPAPELLGGPSVFLFPPKPKDTLMISR |
| | | | | | TPEVTCVVVDVSHEDPEVKFNWYVDGVEVHNAKTK |
| | | | | | PREEQYNSTYRVVSVLTVLHQDWLNGKEYKCKVSNK |
| | | | | | ALPAPIEKTISKAKGQPREPQVYTLPPSRDELTKNQVS |
| | | | | | LTCLVKGFYPSDIAVEWESNGQPENNYKTTPPVLDSD |
| | | | | | GSFFLYSKLTVDKSRWQQGNVFSCSVMHEALHNHYT |
| | | | | | QKSLSLSPG |
| TPP- | 438H-M308- | Light | PRT | 102 | DIQMTQSPSSLSASVGDRVTITCRASQSISSYLNWYQ |
| 17418 | H05_B- | Chain | | | QKPGGAPKFLIYYASSLQSGVPSRFSGSGSGTDYTLTIS |
| | hIgG1Kappa | | | | SLQPEDFATYYCQQGFSLPWTFGGGTKVEIKRTVAAP |
| | | | | | SVFIFPPSDEQLKSGTASVVCLLNNFYPREAKVQWKV |
| | | | | | DNALQSGNSQESVTEQDSKDSTYSLSSTLTLSKADYEK |
| | | | | | HKVYACEVTHQGLSSPVTKSFNRGEC |
| TPP- | 438H-M307-H07- | VH | PRT | 103 | EVQLVQSGAEVKKPGASVKVSCKASGYKFSSYWIEW |
| 17419 | hIgG1Kappa | | | | VKQAPGQGLEWIGEILPGSDWTNYNEKFKDRATFTS |
| | | | | | DTSINTAYMELSRLRSDDTAVYYCARDRGNYRAWFQ |
| | | | | | YWGQGTLVTVSS |
| TPP- | 438H-M307-H07- | HCDR1 | PRT | 104 | SYWIE |
| 17419 | hIgG1Kappa | | | | |
| TPP- | 438H-M307-H07- | HCDR2 | PRT | 105 | EILPGSDWTNYNEKFKD |
| 17419 | hIgG1Kappa | | | | |
| TPP- | 438H-M307-H07- | HCDR3 | PRT | 106 | DRGNYRAWFQY |
| 17419 | hIgG1Kappa | | | | |
| TPP- | 438H-M307-H07- | VL | PRT | 107 | DIQMTQSPSSLSASVGDRVTITCRISQSISSYLNWYQQ |
| 17419 | hIgG1Kappa | | | | KPGGAPKFLIYYASSLQSGVPSRFSGSGSGTDYTLTISS |
| | | | | | LQPEDFATYYCQQGLRLPWTFGGGTKVEIK |
| TPP- | 438H-M307-H07- | LCDR1 | PRT | 108 | RISQSISSYLN |
| 17419 | hIgG1Kappa | | | | |
| TPP- | 438H-M307-H07- | LCDR2 | PRT | 109 | YASSLQS |
| 17419 | hIgG1Kappa | | | | |
| TPP- | 438H-M307-H07- | LCDR3 | PRT | 110 | QQGLRLPWT |
| 17419 | hIgG1Kappa | | | | |
| TPP- | 438H-M307-H07- | Heavy | PRT | 111 | EVQLVQSGAEVKKPGASVKVSCKASGYKFSSYWIEW |
| 17419 | hIgG1Kappa | Chain | | | VKQAPGQGLEWIGEILPGSDWTNYNEKFKDRATFTS |
| | | | | | DTSINTAYMELSRLRSDDTAVYYCARDRGNYRAWFQ |
| | | | | | YWGQGTLVTVSSASTKGPSVFPLAPSSKSTSGGTAAL |
| | | | | | GCLVKDYFPEPVTVSWNSGALTSGVHTFPAVLQSSGL |
| | | | | | YSLSSVVTVPSSSLGTQTYICNVNHKPSNTKVDKKVEP |
| | | | | | KSCDKTHTCPPCPAPELLGGPSVFLFPPKPKDTLMISR |
| | | | | | TPEVTCVVVDVSHEDPEVKFNWYVDGVEVHNAKTK |
| | | | | | PREEQYNSTYRVVSVLTVLHQDWLNGKEYKCKVSNK |
| | | | | | ALPAPIEKTISKAKGQPREPQVYTLPPSRDELTKNQVS |
| | | | | | LTCLVKGFYPSDIAVEWESNGQPENNYKTTPPVLDSD |
| | | | | | GSFFLYSKLTVDKSRWQQGNVFSCSVMHEALHNHYT |
| | | | | | QKSLSLSPG |
| TPP- | 438H-M307-H07- | Light | PRT | 112 | DIQMTQSPSSLSASVGDRVTITCRISQSISSYLNWYQQ |
| 17419 | hIgG1Kappa | Chain | | | KPGGAPKFLIYYASSLQSGVPSRFSGSGSGTDYTLTISS |
| | | | | | LQPEDFATYYCQQGLRLPWTFGGGTKVEIKRTVAAPS |
| | | | | | VFIFPPSDEQLKSGTASVVCLLNNFYPREAKVQWKVD |
| | | | | | NALQSGNSQESVTEQDSKDSTYSLSSTLTLSKADYEKH |
| | | | | | KVYACEVTHQGLSSPVTKSFNRGEC |
| TPP- | 438H-M306-C11- | VH | PRT | 113 | EVQLVQSGAEVKKPGASVKVSCKASGYKFSSYWIEW |
| 17421 | hIgG1Kappa | | | | VKQAPGQGLEWIGEILPGSDWTNYNEKFKDRATFTS |
| | | | | | DTSINTAYMELSRLRSDDTAVYYCARDRGNYRAWFQ |
| | | | | | YWGQGTLVTVSS |
| TPP- | 438H-M306-C11- | HCDR1 | PRT | 114 | SYWIE |
| 17421 | hIgG1Kappa | | | | |
| TPP- | 438H-M306-C11- | HCDR2 | PRT | 115 | EILPGSDWTNYNEKFKD |
| 17421 | hIgG1Kappa | | | | |
| TPP- | 438H-M306-C11- | HCDR3 | PRT | 116 | DRGNYRAWFQY |
| 17421 | hIgG1Kappa | | | | |
| TPP- | 438H-M306-C11- | VL | PRT | 117 | DIQMTQSPSSLSASVGDRVTITCRASQSISSYLNWYQ |
| 17421 | hIgG1Kappa | | | | QKPGGAPKFLIYYASSLQSGVPSRFSGSGSGTDYTLTIS |
| | | | | | SLQPEDFATYYCQQGLELPWTFGGGTKVEIK |
| TPP- | 438H-M306-C11- | LCDR1 | PRT | 118 | RASQSISSYLN |
| 17421 | hIgG1Kappa | | | | |
| TPP- | 438H-M306-C11- | LCDR2 | PRT | 119 | YASSLQS |
| 17421 | hIgG1Kappa | | | | |
| TPP- | 438H-M306-C11- | LCDR3 | PRT | 120 | QQGLELPWT |
| 17421 | hIgG1Kappa | | | | |
| TPP- | 438H-M306-C11- | VH | DNA | 121 | GAGGTGCAGCTGGTGCAGTCTGGCGCCGAAGTGA |
| 17421 | hIgG1Kappa | | | | AAAAGCCTGGCGCCTCTGTGAAGGTGTCCTGCAAG |
| | | | | | GCTTCCGGCTACAAGTTCTCCAGCTACTGGATCGAG |
| | | | | | TGGGTCAAGCAGGCTCCTGGACAGGGACTCGAGT |
| | | | | | GGATCGGAGAGATCCTGCCTGGCTCTGACTGGACC |
| | | | | | AACTACAACGAGAAGTTCAAGGACCGGGCCACCTT |
| | | | | | CACCTCCGACACCTCTATCAACACCGCCTACATGGA |
| | | | | | ACTGTCCCGGCTGAGATCTGACGACACCGCCGTGT |
| | | | | | ACTACTGCGCCAGAGACAGAGGCAACTACAGAGCC |
| | | | | | TGGTTTCAGTACTGGGGCCAGGGCACACTGGTCAC |
| | | | | | AGTTTCTTCA |
| TPP- | 438H-M306-C11- | VL | DNA | 122 | GATATCCAGATGACCCAGTCTCCTTCCAGCCTGTCT |
| 17421 | hIgG1Kappa | | | | GCCTCTGTGGGCGACAGAGTGACCATCACCTGTCG |
| | | | | | GGCCTCTCAGTCCATCTCCTCCTACCTGAACTGGTA |
| | | | | | TCAGCAGAAGCCTGGCGGCGCTCCCAAGTTCCTGA |
| | | | | | TCTACTACGCTAGCTCCCTGCAGTCCGGCGTGCCCT |
| | | | | | CTAGATTTTCTGGCTCTGGATCCGGCACCGACTATA |
| | | | | | CCCTGACAATCTCCAGCCTGCAGCCTGAGGACTTCG |
| | | | | | CCACCTACTACTGTCAGCAGGGACTCGAGCTGCCCT |
| | | | | | GGACATTTGGCGGAGGCACCAAGGTGGAAATCAA |
| | | | | | A |
| TPP- | 438H-M306-C11- | Heavy | PRT | 123 | EVQLVQSGAEVKKPGASVKVSCKASGYKFSSYWIEW |
| 17421 | hIgG1Kappa | Chain | | | VKQAPGQGLEWIGEILPGSDWTNYNEKFKDRATFTS |
| | | | | | DTSINTAYMELSRLRSDDTAVYYCARDRGNYRAWFQ |
| | | | | | YWGQGTLVTVSSASTKGPSVFPLAPSSKSTSGGTAAL |
| | | | | | GCLVKDYFPEPVTVSWNSGALTSGVHTFPAVLQSSGL |
| | | | | | YSLSSVVTVPSSSLGTQTYICNVNHKPSNTKVDKKVEP |
| | | | | | KSCDKTHTCPPCPAPELLGGPSVFLFPPKPKDTLMISR |
| | | | | | TPEVTCVVVDVSHEDPEVKFNWYVDGVEVHNAKTK |
| | | | | | PREEQYNSTYRVVSVLTVLHQDWLNGKEYKCKVSNK |
| | | | | | ALPAPIEKTISKAKGQPREPQVYTLPPSRDELTKNQVS |
| | | | | | LTCLVKGFYPSDIAVEWESNGQPENNYKTTPPVLDSD |
| | | | | | GSFFLYSKLTVDKSRWQQGNVFSCSVMHEALHNHYT |
| | | | | | QKSLSLSPG |
| TPP- | 438H-M306-C11- | Light | PRT | 124 | DIQMTQSPSSLSASVGDRVTITCRASQSISSYLNWYQ |
| 17421 | hIgG1Kappa | Chain | | | QKPGGAPKFLIYYASSLQSGVPSRFSGSGSGTDYTLTIS |
| | | | | | SLQPEDFATYYCQQGLELPWTFGGGTKVEIKRTVAAP |
| | | | | | SVFIFPPSDEQLKSGTASVVCLLNNFYPREAKVQWKV |
| | | | | | DNALQSGNSQESVTEQDSKDSTYSLSSTLTLSKADYEK |
| | | | | | HKVYACEVTHQGLSSPVTKSFNRGEC |
| TPP- | 438H-M306-C11- | Heavy | DNA | 125 | GAGGTGCAGCTGGTGCAGTCTGGCGCCGAAGTGA |
| 17421 | hIgG1Kappa | Chain | | | AAAAGCCTGGCGCCTCTGTGAAGGTGTCCTGCAAG |
| | | | | | GCTTCCGGCTACAAGTTCTCCAGCTACTGGATCGAG |
| | | | | | TGGGTCAAGCAGGCTCCTGGACAGGGACTCGAGT |
| | | | | | GGATCGGAGAGATCCTGCCTGGCTCTGACTGGACC |
| | | | | | AACTACAACGAGAAGTTCAAGGACCGGGCCACCTT |
| | | | | | CACCTCCGACACCTCTATCAACACCGCCTACATGGA |
| | | | | | ACTGTCCCGGCTGAGATCTGACGACACCGCCGTGT |
| | | | | | ACTACTGCGCCAGAGACAGAGGCAACTACAGAGCC |
| | | | | | TGGTTTCAGTACTGGGGCCAGGGCACACTGGTCAC |
| | | | | | AGTTTCTTCAGCCTCCACCAAGGGCCCCTCCGTGTT |
| | | | | | TCCTCTGGCCCCTTCCAGCAAGTCCACCTCTGGCGG |
| | | | | | AACAGCCGCTCTGGGCTGCCTCGTGAAGGACTACT |
| | | | | | TCCCCGAGCCTGTGACCGTGTCCTGGAACTCTGGC |
| | | | | | GCTCTGACATCCGGCGTGCACACCTTCCCTGCTGTG |
| | | | | | CTGCAGTCTAGCGGCCTGTACTCCCTGTCCTCCGTC |
| | | | | | GTGACCGTGCCTTCCAGCTCTCTGGGCACCCAGACC |
| | | | | | TACATCTGCAACGTGAACCACAAGCCCTCCAACACC |
| | | | | | AAGGTGGACAAGAAGGTGGAACCCAAGTCCTGCG |
| | | | | | ACAAGACCCACACCTGTCCCCCTTGTCCTGCCCCTG |
| | | | | | AACTGCTGGGCGGACCTTCCGTGTTCCTGTTCCCCC |
| | | | | | CAAAGCCCAAGGACACCCTGATGATCTCCCGGACC |
| | | | | | CCCGAAGTGACCTGCGTGGTGGTGGATGTGTCCCA |
| | | | | | CGAGGACCCTGAAGTGAAGTTCAATTGGTACGTGG |
| | | | | | ACGGCGTGGAAGTGCACAACGCCAAGACCAAGCCT |
| | | | | | AGAGAGGAACAGTACAACTCCACCTACCGGGTGGT |
| | | | | | GTCCGTGCTGACCGTGCTGCACCAGGATTGGCTGA |
| | | | | | ACGGCAAAGAGTACAAGTGCAAGGTGTCCAACAA |
| | | | | | GGCCCTGCCTGCCCCCATCGAAAAGACCATCTCCAA |
| | | | | | GGCCAAGGGCCAGCCCCGGGAACCCCAGGTGTAC |
| | | | | | ACACTGCCCCCTAGCAGGGACGAGCTGACCAAGAA |
| | | | | | CCAGGTGTCCCTGACCTGTCTCGTGAAAGGCTTCTA |
| | | | | | CCCCTCCGATATCGCCGTGGAATGGGAGTCCAACG |
| | | | | | GCCAGCCTGAGAACAACTACAAGACCACCCCCCCT |
| | | | | | GTGCTGGACTCCGACGGCTCATTCTTCCTGTACAGC |
| | | | | | AAGCTGACAGTGGACAAGTCCCGGTGGCAGCAGG |
| | | | | | GCAACGTGTTCTCCTGCTCCGTGATGCACGAGGCC |
| | | | | | CTGCACAACCACTACACCCAGAAGTCCCTGTCCCTG |
| | | | | | AGCCCTGGC |
| TPP- | 438H-M306-C11- | Light | DNA | 126 | GATATCCAGATGACCCAGTCTCCTTCCAGCCTGTCT |
| 17421 | hIgG1Kappa | Chain | | | GCCTCTGTGGGCGACAGAGTGACCATCACCTGTCG |
| | | | | | GGCCTCTCAGTCCATCTCCTCCTACCTGAACTGGTA |
| | | | | | TCAGCAGAAGCCTGGCGGCGCTCCCAAGTTCCTGA |
| | | | | | TCTACTACGCTAGCTCCCTGCAGTCCGGCGTGCCCT |
| | | | | | CTAGATTTTCTGGCTCTGGATCCGGCACCGACTATA |
| | | | | | CCCTGACAATCTCCAGCCTGCAGCCTGAGGACTTCG |
| | | | | | CCACCTACTACTGTCAGCAGGGACTCGAGCTGCCCT |
| | | | | | GGACATTTGGCGGAGGCACCAAGGTGGAAATCAA |
| | | | | | ACGAACCGTGGCCGCTCCCTCCGTGTTCATCTTCCC |
| | | | | | ACCCTCCGACGAGCAGCTGAAGTCCGGCACCGCCA |
| | | | | | GCGTCGTGTGCCTGCTGAACAACTTCTACCCCCGCG |
| | | | | | AGGCCAAGGTGCAGTGGAAGGTGGACAACGCCCT |
| | | | | | GCAGTCCGGCAACTCCCAGGAATCCGTCACCGAGC |
| | | | | | AGGACTCCAAGGACAGCACCTACTCCCTGTCCTCCA |
| | | | | | CCCTGACCCTGTCCAAGGCCGACTACGAGAAGCAC |
| | | | | | AAGGTGTACGCCTGCGAAGTGACCCACCAGGGCCT |
| | | | | | GTCCAGCCCCGTGACCAAGTCCTTCAACCGGGGCG |
| | | | | | AGTGC |
| TPP- | 438H-M313- | VH | PRT | 127 | EVQLVQSGAEVKKPGASVKVSCKASGYKFSSYWIEW |
| 17422 | J08_B- | | | | VKQAPGQGLEWIGEILPGSDTTNYNEKFKDRATFTSD |
| | hIgG1Kappa | | | | TSINTAYMELSRLRSDDTAVYYCARDRGNYRAWFGY |
| | | | | | WGQGTLVTVSS |
| TPP- | 438H-M313- | HCDR1 | PRT | 128 | SYWIE |
| 17422 | J08_B- | | | | |
| | hIgG1Kappa | | | | |
| TPP- | 438H-M313- | HCDR2 | PRT | 129 | EILPGSDTTNYNEKFKD |
| 17422 | J08_B- | | | | |
| | hIgG1Kappa | | | | |
| TPP- | 438H-M313- | HCDR3 | PRT | 130 | DRGNYRAWFGY |
| 17422 | J08_B- | | | | |
| | hIgG1Kappa | | | | |
| TPP- | 438H-M313- | VL | PRT | 131 | DIQMTQSPSSLSASVGDRVTITCRASQSISSYLNWYQ |
| 17422 | J08_B- | | | | QKPGGAPKFLIYYASSLQSGVPSRFSGSGSGTDYTLTIS |
| | hIgG1Kappa | | | | SLQPEDFATYYCQQGLSLPWTFGGGTKVEIK |
| TPP- | 438H-M313- | LCDR1 | PRT | 132 | RASQSISSYLN |
| 17422 | J08_B- | | | | |
| | hIgG1Kappa | | | | |
| TPP- | 438H-M313- | LCDR2 | PRT | 133 | YASSLQS |
| 17422 | J08_B- | | | | |
| | hIgG1Kappa | | | | |
| TPP- | 438H-M313- | LCDR3 | PRT | 134 | QQGLSLPWT |
| 17422 | J08_B- | | | | |
| | hIgG1Kappa | | | | |
| TPP- | 438H-M313- | Heavy | PRT | 135 | EVQLVQSGAEVKKPGASVKVSCKASGYKFSSYWIEW |
| 17422 | J08_B- | Chain | | | VKQAPGQGLEWIGEILPGSDTTNYNEKFKDRATFTSD |
| | hIgG1Kappa | | | | TSINTAYMELSRLRSDDTAVYYCARDRGNYRAWFGY |
| | | | | | WGQGTLVTVSSASTKGPSVFPLAPSSKSTSGGTAALG |
| | | | | | CLVKDYFPEPVTVSWNSGALTSGVHTFPAVLQSSGLY |
| | | | | | SLSSVVTVPSSSLGTQTYICNVNHKPSNTKVDKKVEPK |
| | | | | | SCDKTHTCPPCPAPELLGGPSVFLFPPKPKDTLMISRT |
| | | | | | PEVTCVVVDVSHEDPEVKFNWYVDGVEVHNAKTKP |
| | | | | | REEQYNSTYRVVSVLTVLHQDWLNGKEYKCKVSNKA |
| | | | | | LPAPIEKTISKAKGQPREPQVYTLPPSRDELTKNQVSLT |
| | | | | | CLVKGFYPSDIAVEWESNGQPENNYKTTPPVLDSDGS |
| | | | | | FFLYSKLTVDKSRWQQGNVFSCSVMHEALHNHYTQK |
| | | | | | SLSLSPG |
| TPP- | 438H-M313- | Light | PRT | 136 | DIQMTQSPSSLSASVGDRVTITCRASQSISSYLNWYQ |
| 17422 | J08_B- | Chain | | | QKPGGAPKFLIYYASSLQSGVPSRFSGSGSGTDYTLTIS |
| | hIgG1Kappa | | | | SLQPEDFATYYCQQGLSLPWTFGGGTKVEIKRTVAAP |
| | | | | | SVFIFPPSDEQLKSGTASVVCLLNNFYPREAKVQWKV |
| | | | | | DNALQSGNSQESVTEQDSKDSTYSLSSTLTLSKADYEK |
| | | | | | HKVYACEVTHQGLSSPVTKSFNRGEC |
| TPP- | hLRRC15 CT-His | Chain 1 | PRT | 137 | YHGCPSECTCSRASQVECTGARIVAVPTPLPWNAMS |
| 1545 | | | | | LQILNTHITELNESPFLNISALIALRIEKNELSRITPGAFR |
| | | | | | NLGSLRYLSLANNKLQVLPIGLFQGLDSLESLLLSSNQL |
| | | | | | LQIQPAHFSQCSNLKELQLHGNHLEYIPDGAFDHLVG |
| | | | | | LTKLNLGKNSLTHISPRVFQHLGNLQVLRLYENRLTDIP |
| | | | | | MGTFDGLVNLQELALQQNQIGLLSPGLFHNNHNLQ |
| | | | | | RLYLSNNHISQLPPSVFMQLPQLNRLTLFGNSLKELSP |
| | | | | | GIFGPMPNLRELWLYDNHISSLPDNVFSNLRQLQVLIL |
| | | | | | SRNQISFISPGAFNGLTELRELSLHTNALQDLDGNVFR |
| | | | | | MLANLQNISLQNNRLRQLPGNIFANVNGLMAIQLQ |
| | | | | | NNQLENLPLGIFDHLGKLCELRLYDNPWRCDSDILPLR |
| | | | | | NWLLLNQPRLGTDTVPVCFSPANVRGQSLIIINVNVA |
| | | | | | VPSVHVPEVPSYPETPWYPDTPSYPDTTSVSSTTELTS |
| | | | | | PVEDYTDLTTIQVTDDRSVWGMTQAQSGHHHHHH |
| TPP- | mLRRC15- | Chain 1 | PRT | 138 | YYGCPSECTCSRASQVECTGAQIVAMPSPLPWNAMS |
| 9045 | ECD_His6 | | | | LQILNTHITELPEDKFLNISALIALKMEKNELANIMPGA |
| | | | | | FRNLGSLRHLSLANNKLKNLPVRLFQDVNNLETLLLSN |
| | | | | | NQLVQIQPAQFSQFSNLKELQLYGNNLEYIPEGVFDH |
| | | | | | LVGLTKLNLGNNGFTHLSPRVFQHLGNLQVLRLYENR |
| | | | | | LSDIPMGTFDALGNLQELALQENQIGTLSPGLFHNNR |
| | | | | | NLQRLYLSNNHISHLPPGIFMQLPHLNKLTLFGNSLKE |
| | | | | | LSPGVFGPMPNLRELWLYNNHITSLPDNAFSHLNQL |
| | | | | | QVLILSHNQLSYISPGAFNGLTNLRELSLHTNALQDLD |
| | | | | | GNVFRSLANLRNVSLQNNRLRQLPGSIFANVNGLMTI |
| | | | | | QLQNNNLENLPLGIFDHLGNLCELRLYDNPWRCDSNI |
| | | | | | LPLHDWLILNRARLGTDTLPVCSSPASVRGQSLVIINV |
| | | | | | NFPGPSVQGPETPEVSSYPDTSSYPDSTSISSTTEITRST |
| | | | | | DDDYTDLNTIEPIDDRNTWGMTDAQSGAGHHHHH |
| | | | | | H |
| TPP- | macfasLRRC15- | Chain 1 | PRT | 139 | YYGCPSECTCSRASQVECTGARIVAVPTPLPWNAMSL |
| 9046 | ECD_His6 | | | | QILNTHITELSESPFLNISALIALRIEKNELSHIMPGAFR |
| | | | | | NLGSLRYLSLANNKLQVLPIGLFQGLDSLESLLLSSNQL |
| | | | | | VQIQPAHFSQCSNLKELQLHGNHLEYIPDGAFDHLVG |
| | | | | | LTKLNLGKNSLTHISPRVFQHLGNLQVLRLYENRLTDIP |
| | | | | | MGTFDGLVNLQELALQQNQIGLLSPGLFHNNHNLQ |
| | | | | | RLYLSNNHISQLPPSIFMQLPQLNRLTLFGNSLKELSPG |
| | | | | | IFGPMPNLRELWLYDNHITSLPDNVFSNLRQLQVLILS |
| | | | | | RNQISFISPGAFNGLTELRELSLHTNALQDLDGNVFR |
| | | | | | MLANLQNISLQNNRLRQLPGNIFANVNGLMTIQLQN |
| | | | | | NQLENLPLGIFDHLGKLCELRLYDNPWRCDSDILPLRN |
| | | | | | WLLLNQPRLGTDTVPVCFSPANVRGQSLIIINVNVAV |
| | | | | | PSVHVPEVPSYPETSWYPDTSSYPDTTSISSTTELTSPV |
| | | | | | EDYTDLTTIQVTDDRSVWGMTQAQSGAGHHHHHH |
| TPP- | Human germline | Chain 1 | PRT | 140 | QVQLVQSGAEVKKPGASVKVSCKASGYTFTGYYMH |
| 21468 | heavy chain (V- | | | | WVRQAPGQGLEWMGWINPNSGGTNYAQKFQGRV |
| | segment)-IGHV1- | | | | TMTRDTSISTAYMELSRLRSDDTAVYYCAR |
| | 2-02 | | | | |
| TPP- | Human germline | Chain 1 | PRT | 141 | DIQMTQSPSSLSASVGDRVTITCRASQGISNSLAWYQ |
| 21469 | light chain- | | | | QKPGKAPKLLLYAASRLESGVPSRFSGSGSGTDYTLTIS |
| | IGKV1-NL1-01- | | | | SLQPEDFATYYCQQYYSTPLTFGGGTKVEIK |
| | IGKJ4-01-02 | | | | |
| TPP- | Human germline | Chain 1 | PRT | 142 | YFDYWGQGTLVTVSS |
| 21470 | heavy chain (J- | | | | |
| | segment)-HV3- | | | | |
| | 23-J1 | | | | |
| TPP- | Human germline | Chain 1 | PRT | 143 | DIQMTQSPSSLSASVGDRVTITCRASQSISSYLNWYQ |
| 21479 | light chain- | | | | QKPGKAPKLLIYAASSLQSGVPSRFSGSGSGTDFTLTIS |
| | IGKV1-39-01- | | | | SLQPEDFATYYCQQSYSTPLTFGGGTKVEIK |
| | IGKJ4-01-02 | | | | |
| TPP- | Human germline | Chain 1 | PRT | 144 | EVQLLESGGGLVQPGGSLRLSCAASGFTFSSYAMSW |
| 21547 | heavy chain (V- | | | | VRQAPGKGLEWVSAISGSGGSTYYADSVKGRFTISRD |
| | segment)-IGHV3- | | | | NSKNTLYLQMNSLRAEDTAVYYCAK |
| | 23-01 | | | | |

1. A conjugate targeting LRRC15 comprising
a) at least one chelating group arranged for complexation of a radionuclide and
b) at least one targeting moiety binding to LRRC15, and
c) optionally a linker between the at least one chelating group and the at least one targeting moiety binding to LRRC15.

2. The conjugate targeting LRRC15 according to claim 1,
a) wherein the radionuclide is an α-particle-emitting radionuclide optionally thorium-227, or
b) wherein the radionuclide is a β-particle-emitting radionuclide optionally zirconium-89, and/or
c) wherein the conjugate targeting LRRC15 comprises a radionuclide according to a) or b).

3. The conjugate targeting LRRC15 according to claim 1, wherein the at least one chelating group arranged for complexation of a radionuclide comprises
a) hydroxypyridinone (HOPO),
b) 3-hydroxypyridin-2-one (3,2-HOPO),
c) 3-hydroxy-N-methyl-2-pyridinone (Me-3,2-HOPO),
d) 1,4,7,10-tetra-azacycloododecane-N,N′,N″,N′″-tetraacetic acid (DOTA), and/or
e) a structure according to formula I 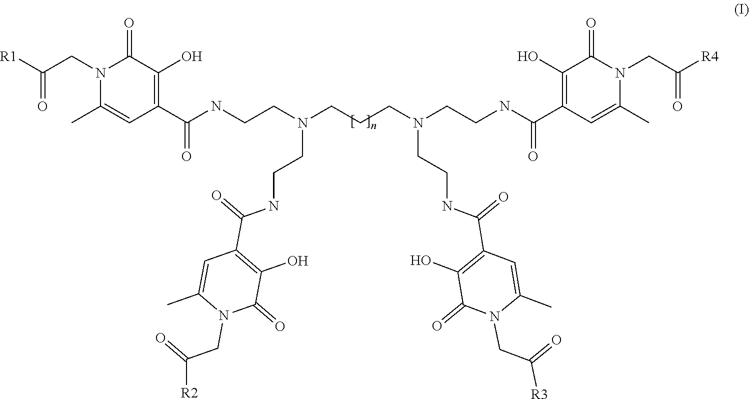
wherein:
n is 1, 2 or 3;
R1, R2, R3 and R4, independently represent OH or Q; and
Q represents a linkage to a targeting moiety binding LRRC15.

4. The conjugate targeting LRRC15 according to claim 1, wherein the LRRC15 is human, cynomolgus and/or murine LRRC15, optionally human and/or cynomolgus LRRC15.

5. The conjugate targeting LRRC15 according to claim 1, wherein the targeting moiety binding to LRRC15 is an antibody or antigen-binding fragment thereof, optionally an antibody or antigen-binding fragment.

6. The conjugate targeting LRRC15 according to claim 5, wherein the antibody or antigen-binding fragment is
a. an IgG1 antibody, optionally a human or humanized IgG1 antibody, and/or
b. an scFv, Fab, Fab′ fragment or a F(ab′)2 fragment.

7. An antibody or antigen-binding fragment thereof binding to human LRRC15 and comprising at least one, two, three, four, five and optionally six CDR sequences, wherein each of said CDR sequences has at least 90%, 95%, 98%, 99% or 100% sequence identity with one or more of
a) SEQ ID NO:2, SEQ ID NO:3, SEQ ID NO:4, SEQ ID NO:6, SEQ ID NO:7 and SEQ ID NO:8 (TPP-1633), or
b) SEQ ID NO:22, SEQ ID NO:23, SEQ ID NO:24, SEQ ID NO:26, SEQ ID NO:27 and SEQ ID NO:28 (TPP-14389), or
c) SEQ ID NO:36, SEQ ID NO:37, SEQ ID NO:38, SEQ ID NO:40, SEQ ID NO:41 and SEQ ID NO:42 (TPP-14392), or
d) SEQ ID NO:50, SEQ ID NO:51, SEQ ID NO:52, SEQ ID NO:54, SEQ ID NO:55, and SEQ ID NO:56 (TPP-17073), or
e) SEQ ID NO:60, SEQ ID NO:61, SEQ ID NO:62, SEQ ID NO:64, SEQ ID NO:65, and SEQ ID NO:66 (TPP-17074), or
f) SEQ ID NO:70, SEQ ID NO:71, SEQ ID NO:72, SEQ ID NO:74, SEQ ID NO:75, and SEQ ID NO:76 (TPP-17078), or
g) SEQ ID NO:84, SEQ ID NO:85, SEQ ID NO:86, SEQ ID NO:88, SEQ ID NO:89, and SEQ ID NO:90 (TPP-17405), or
h) SEQ ID NO:94, SEQ ID NO:95, SEQ ID NO:96, SEQ ID NO:98, SEQ ID NO:99, and SEQ ID NO:100 (TPP-17418), or
i) SEQ ID NO:104, SEQ ID NO:105, SEQ ID NO: 106, SEQ ID NO:108, SEQ ID NO:109, and SEQ ID NO:110 (TPP-17419), or
j) SEQ ID NO:114, SEQ ID NO:115, SEQ ID NO:116, SEQ ID NO:118, SEQ ID NO:119, and SEQ ID NO:120 (TPP-17421), or
k) SEQ ID NO:128, SEQ ID NO:129, SEQ ID NO: 130, SEQ ID NO:132, SEQ ID NO:133, and SEQ ID NO:134 (TPP-17422).

8. An antibody or antigen-binding fragment thereof binding to human LRRC15 and comprising
a) a variable heavy chain having at least 90%, 95%, 98%, 99% or 100% sequence identity with SEQ ID NO:1 and/or
a variable light chain having at least 90%, 95%, 98%, 99% or 100% sequence identity with SEQ ID NO:5 (TPP-1633), or
b) a variable heavy chain having at least 90%, 95%, 98%, 99% or 100% sequence identity with SEQ ID NO:21 and/or
a variable light chain having at least 90%, 95%, 98%, 99% or 100% sequence identity with SEQ ID NO:25 (TPP-14389), or
c) a variable heavy chain having at least 90%, 95%, 98%, 99% or 100% sequence identity with SEQ ID NO:35 and/or
a variable light chain having at least 90%, 95%, 98%, 99% or 100% sequence identity with SEQ ID NO:39 (TPP-14392), or
d) a variable heavy chain having at least 90%, 95%, 98%, 99% or 100% sequence identity with SEQ ID NO:49 and/or
a variable light chain having at least 90%, 95%, 98%, 99% or 100% sequence identity with SEQ ID NO:53 (TPP-17073), or
e) a variable heavy chain having at least 90%, 95%, 98%, 99% or 100% sequence identity with SEQ ID NO:59 and/or
a variable light chain having at least 90%, 95%, 98%, 99% or 100% sequence identity with SEQ ID NO:63 (TPP-17074), or
f) a variable heavy chain having at least 90%, 95%, 98%, 99% or 100% sequence identity with SEQ ID NO:69 and/or
a variable light chain having at least 90%, 95%, 98%, 99% or 100% sequence identity with SEQ ID NO:73 (TPP-17078), or
g) a variable heavy chain having at least 90%, 95%, 98%, 99% or 100% sequence identity with SEQ ID NO:83 and/or
a variable light chain having at least 90%, 95%, 98%, 99% or 100% sequence identity with SEQ ID NO:87 (TPP-17405), or
h) a variable heavy chain having at least 90%, 95%, 98%, 99% or 100% sequence identity with SEQ ID NO:93 and/or
a variable light chain having at least 90%, 95%, 98%, 99% or 100% sequence identity with SEQ ID NO:97 (TPP-17418), or
i) a variable heavy chain having at least 90%, 95%, 98%, 99% or 100% sequence identity with SEQ ID NO:103 and/or
a variable light chain having at least 90%, 95%, 98%, 99% or 100% sequence identity with SEQ ID NO:107 (TPP-17419), or
j) a variable heavy chain having at least 90%, 95%, 98%, 99% or 100% sequence identity with SEQ ID NO:113 and/or
a variable light chain having at least 90%, 95%, 98%, 99% or 100% sequence identity with SEQ ID NO:117 (TPP-17421), or
k) a variable heavy chain having at least 90%, 95%, 98%, 99% or 100% sequence identity with SEQ ID NO:127 and/or
a variable light chain having at least 90%, 95%, 98%, 99% or 100% sequence identity with SEQ ID NO:131 (TPP-17422).

9. An antibody or antigen-binding fragment thereof binding to human LRRC15 comprising
a) a heavy chain region having at least 90%, 95%, 98%, 99% or 100% sequence identity with SEQ ID NO:9 and/or
a light chain region having at least 90%, 95%, 98%, 99% or 100% sequence identity with SEQ ID NO:10 (TPP-1633), or
b) a heavy chain region having at least 90%, 95%, 98%, 99% or 100% sequence identity with SEQ ID NO:31 and/or
a light chain region having at least 90%, 95%, 98%, 99% or 100% sequence identity with SEQ ID NO:32 (TPP-14389), or
c) a heavy chain region having at least 90%, 95%, 98%, 99% or 100% sequence identity with SEQ ID NO:45 and/or
a light chain region having at least 90%, 95%, 98%, 99% or 100% sequence identity with SEQ ID NO:46 (TPP-14392), or
d) a heavy chain region having at least 90%, 95%, 98%, 99% or 100% sequence identity with SEQ ID NO:57 and/or
a light chain region having at least 90%, 95%, 98%, 99% or 100% sequence identity with SEQ ID NO:58 (TPP-17073), or
e) a heavy chain region having at least 90%, 95%, 98%, 99% or 100% sequence identity with SEQ ID NO:67 and/or
a light chain region having at least 90%, 95%, 98%, 99% or 100% sequence identity with SEQ ID NO:68 (TPP-17074), or
f) a heavy chain region having at least 90%, 95%, 98%, 99% or 100% sequence identity with SEQ ID NO:79 and/or
a light chain region having at least 90%, 95%, 98%, 99% or 100% sequence identity with SEQ ID NO:80 (TPP-17078), or
g) a heavy chain region having at least 90%, 95%, 98%, 99% or 100% sequence identity with SEQ ID NO:91 and/or
a light chain region having at least 90%, 95%, 98%, 99% or 100% sequence identity with SEQ ID NO:92 (TPP-17405), or
h) a heavy chain region having at least 90%, 95%, 98%, 99% or 100% sequence identity with SEQ ID NO:101 and/or
a light chain region having at least 90%, 95%, 98%, 99% or 100% sequence identity with SEQ ID NO:102 (TPP-17418), or
i) a heavy chain region having at least 90%, 95%, 98%, 99% or 100% sequence identity with SEQ ID NO:111 and/or
a light chain region having at least 90%, 95%, 98%, 99% or 100% sequence identity with SEQ ID NO:112 (TPP-17419), or
j) a heavy chain region having at least 90%, 95%, 98%, 99% or 100% sequence identity with SEQ ID NO:123 and/or
a light chain region having at least 90%, 95%, 98%, 99% or 100% sequence identity with SEQ ID NO:124 (TPP-17421), or
k) a heavy chain region having at least 90%, 95%, 98%, 99% or 100% sequence identity with SEQ ID NO:135 and/or
a light chain region having at least 90%, 95%, 98%, 99% or 100% sequence identity with SEQ ID NO:136 (TPP-17422).

10. The isolated antibody or antigen-binding fragment according to claim 7, wherein the antibody is an IgG antibody, optionally a human IgG1.

11. The isolated antibody or antigen-binding fragment according to claim 7, wherein said isolated antibody or antigen-binding fragment is an scFv, Fab, Fab′ fragment or a F(ab′)2 fragment.

12. The isolated antibody or antigen-binding fragment according to claim 7, wherein said isolated antibody or antigen-binding fragment is a bispecific antibody.

13. The conjugate targeting LRRC15 comprising an antibody or antigen-binding fragment according to claim 7.

14. The conjugate targeting LRRC15 according to claim 13, wherein the conjugate comprises
a. an α-particle-emitting radionuclide optionally ²¹¹At, ²¹²Pb, ²¹³Bi, ²²³Ra, ²²⁴Ra, ²²⁵Ac, or ²²⁷Th,
b. a β-particle-emitting radionuclide optionally ⁶⁷Cu, ⁸⁹Sr, ⁸⁹Zr, ⁹⁰Y ¹⁰⁵Rh, ¹³¹I, ¹⁴⁹Pm ¹⁶⁶Ho, ¹⁷⁷Lu, ¹⁸⁶Re, ¹⁸⁸Re, ¹⁹⁸Au,
c. a cytotoxic agent, optionally an auristatin, a maytansinoid, a kinesin-spindle protein inhibitor, a nicotinamide phosphoribosyltransferase inhibitor or a pyrrolobenzodiazepine derivative,
d. a detectable moiety,
e. a bispecific antibody, and/or
f. a chimeric antigen receptor.

15. A pharmaceutical composition comprising
a. a conjugate targeting LRRC15 according to claim 1,
b. an antibody or antigen-binding fragment binding to human LRRC15 or
c. a conjugate targeting LRRC15 comprising said b,
wherein the pharmaceutical composition optionally comprises one or more further therapeutically active compounds, optionally selected from an antibody or a small molecule targeting a checkpoint protein, optionally PD1, PD-L1 or CTLA-4.

16. The conjugate targeting LRRC15 according to claim 1 as a medicament.

17. The conjugate targeting LRRC15 according to claim 1 as a treatment of cancer or a disease characterized by LRRC15 expression.

18. The conjugate targeting LRRC15 according to claim 17, wherein the cancer is lung cancer, non-small cell lung carcinoma, head and neck cancer, head and neck squamous cell carcinoma, sarcoma, glioblastoma, melanoma, breast cancer, HER2 negative breast cancer, HER2 positive breast cancer, triple negative breast cancer, HR+ breast cancer, pancreatic cancer, pancreatic ductal adenocarcinoma or colorectal cancer.

19. The conjugate as a medicament according to claim 16, further comprising at least one further therapeutically active compound, optionally wherein said at least one further therapeutically active compound comprises an inhibitor of PD-L1 and/or PD-1, optionally an antibody specifically binding human PD-L1 or PD-1.

20. A conjugate targeting LRRC15 according to claim 1 as a diagnostic agent.

21. A polynucleotide encoding an antibody or antigen-binding fragment according claim 7.

22. A vector comprising a polynucleotide according to claim 22.

23. An isolated cell arranged for production of an isolated antibody or antigen-binding fragment according to claim 7.

24. A method for production of a conjugate according to claim 1 comprising coupling of the at least one chelating group arranged for complexation of a radionuclide to the at least one targeting moiety binding LRRC15, to obtain a tissue-targeting chelator complex.

25. The method according to claim 24, wherein the radionuclide is ²²⁷Th, and wherein said coupling is followed by contacting said tissue-targeting chelator complex with an aqueous solution comprising 4+ ions of the radionuclide.

26. A method for production of an antibody or antigen-binding fragment according to a claim 7, wherein the method comprises culturing an isolated cell and optionally purification of the antibody or antigen-binding fragment.

27. A kit of parts comprising an antibody or antigen-binding fragment according to claim 7 or a conjugate thereof with instructions for use.


SEQUENCE LISTING (144 sequences)

SEQ ID NO: 1 - protein, 125 aa, Artificial Sequence
  LRRC15 antibody
EVQLLESGGGLVQPGGSLRLSCAASGFTFSGYMMQWVRQAPGKGLEWVSGIYPSGGYTNYADSVKGRFTISRDNSKNTLYLQMNSLRAEDTATYYCAREKASDLSGSYSEALDYWGQGTLVTVSS

SEQ ID NO: 2 - protein, 5 aa, Artificial Sequence
  LRRC15 antibody
GYMMQ

SEQ ID NO: 3 - protein, 17 aa, Artificial Sequence
  LRRC15 antibody
GIYPSGGYTNYADSVKG

SEQ ID NO: 4 - protein, 16 aa, Artificial Sequence
  LRRC15 antibody
EKASDLSGSYSEALDY

SEQ ID NO: 5 - protein, 107 aa, Artificial Sequence
  LRRC15 antibody
DIQMTQSPDSLSASVGDRVTITCRASQDVGSWLAWYQQKPGKAPKLLIFAASSLESGIPSRFSGSGSGTDFTLTISSLQPEDFATYYCQQANGFPLTFGGGTKVEIK

SEQ ID NO: 6 - protein, 11 aa, Artificial Sequence
  LRRC15 antibody
RASQDVGSWLA

SEQ ID NO: 7 - protein, 7 aa, Artificial Sequence
  LRRC15 antibody
AASSLES

SEQ ID NO: 8 - protein, 9 aa, Artificial Sequence
  LRRC15 antibody
QQANGFPLT

SEQ ID NO: 9 - protein, 454 aa, Artificial Sequence
  LRRC15 antibody
EVQLLESGGGLVQPGGSLRLSCAASGFTFSGYMMQWVRQAPGKGLEWVSGIYPSGGYTNYADSVKGRFTISRDNSKNTLYLQMNSLRAEDTATYYCAREKASDLSGSYSEALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 216 aa, Artificial Sequence
  LRRC15 antibody
AQDIQMTQSPDSLSASVGDRVTITCRASQDVGSWLAWYQQKPGKAPKLLIFAASSLESGIPSRFSGSGSGTDFTLTISSLQPEDFATYYCQQANGFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 120 aa, Artificial Sequence
  LRRC15 antibody
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRGNYRAWFGYWGQGTLVTVSS

SEQ ID NO: 12 - protein, 5 aa, Artificial Sequence
  LRRC15 antibody
SYWIE

SEQ ID NO: 13 - protein, 17 aa, Artificial Sequence
  LRRC15 antibody
EILPGSDTTNYNEKFKD

SEQ ID NO: 14 - protein, 11 aa, Artificial Sequence
  LRRC15 antibody
DRGNYRAWFGY

SEQ ID NO: 15 - protein, 107 aa, Artificial Sequence
  LRRC15 antibody
DIQMTQSPSSLSASVGDRVTITCRASQDISNYLNWYQQKPGGAVKFLIYYTSRLHSGVPSRFSGSGSGTDYTLTISSLQPEDFATYFCQQGEALPWTFGGGTKVEIK

SEQ ID NO: 16 - protein, 11 aa, Artificial Sequence
  LRRC15 antibody
RASQDISNYLN

SEQ ID NO: 17 - protein, 7 aa, Artificial Sequence
  LRRC15 antibody
YTSRLHS

SEQ ID NO: 18 - protein, 9 aa, Artificial Sequence
  LRRC15 antibody
QQGEALPWT

SEQ ID NO: 19 - protein, 449 aa, Artificial Sequence
  LRRC15 antibody
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 214 aa, Artificial Sequence
  LRRC15 antibody
DIQMTQSPSSLSASVGDRVTITCRASQDISNYLNWYQQKPGGAVKFLIYYTSRLHSGVPSRFSGSGSGTDYTLTISSLQPEDFATYFCQQGEALPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 125 aa, Artificial Sequence
  LRRC15 antibody
EVQLLESGGGLVQPGGSLRLSCAASGFTFSGYMMSWVRQAPGKGLEWVSGIYPSPGYTYYADSVKGRFTISRDNSKNTLYLQMNSLRAEDTAVYYCAREKASDLFGSYSEALDYWGQGTLVTVSS

SEQ ID NO: 22 - protein, 5 aa, Artificial Sequence
  LRRC15 antibody
GYMMS

SEQ ID NO: 23 - protein, 17 aa, Artificial Sequence
  LRRC15 antibody
GIYPSPGYTYYADSVKG

SEQ ID NO: 24 - protein, 16 aa, Artificial Sequence
  LRRC15 antibody
EKASDLFGSYSEALDY

SEQ ID NO: 25 - protein, 107 aa, Artificial Sequence; identical to SEQ ID NO: 5
  LRRC15 antibody

SEQ ID NO: 26 - protein, 11 aa, Artificial Sequence
  LRRC15 antibody
RASQDVGSWLA

SEQ ID NO: 27 - protein, 7 aa, Artificial Sequence
  LRRC15 antibody
AASSLES

SEQ ID NO: 28 - protein, 9 aa, Artificial Sequence
  LRRC15 antibody
QQANGFPLT

SEQ ID NO: 29 - DNA, 375 bp, Artificial Sequence
  LRRC15 antibody
gaggtgcagctgctggaatctggcggaggattggttcagcctggcggctctctgagactgtcttgtgccgcttctggcttcaccttctccggctacatgatgtcctgggtccgacaggctcctggcaaaggactggaatgggtgtccggcatctatcccagtcctggctacacctactacgccgactctgtgaagggcagattcaccatcagccgggacaactccaagaacaccctgtacctgcagatgaactccctgagagccgaggacaccgccgtgtactactgtgccagagagaaggcctctgacctgttcggctcttactctgaggccctggattattggggccagggcacactggttaccgtgtcatca

SEQ ID NO: 30 - DNA, 321 bp, Artificial Sequence
  LRRC15 antibody
gatatccagatgacccagtctcctgactctctgtccgcctctgtgggcgacagagtgaccatcacctgtagagcctctcaggacgtcggctcttggctggcttggtatcagcagaagcctggcaaggcccctaagctgctgatctttgccgcctcctctctggaatctggcatcccctctagattctccggctctggctctggcaccgactttaccctgacaatctccagcctgcagcctgaggacttcgccacctactactgtcagcaggccaacggcttcccactgacatttggcggcggaacaaaggtggaaatcaaa

SEQ ID NO: 31 - protein, 454 aa, Artificial Sequence
  LRRC15 antibody
EVQLLESGGGLVQPGGSLRLSCAASGFTFSGYMMSWVRQAPGKGLEWVSGIYPSPGYTYYADSVKGRFTISRDNSKNTLYLQMNSLRAEDTAVYYCAREKASDLFGSYSEALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 32 - protein, 214 aa, Artificial Sequence
  LRRC15 antibody
DIQMTQSPDSLSASVGDRVTITCRASQDVGSWLAWYQQKPGKAPKLLIFAASSLESGIPSRFSGSGSGTDFTLTISSLQPEDFATYYCQQANGFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - DNA, 1362 bp, Artificial Sequence
  LRRC15 antibody
gaggtgcagctgctggaatctggcggaggattggttcagcctggcggctctctgagactgtcttgtgccgcttctggcttcaccttctccggctacatgatgtcctgggtccgacaggctcctggcaaaggactggaatgggtgtccggcatctatcccagtcctggctacacctactacgccgactctgtgaagggcagattcaccatcagccgggacaactccaagaacaccctgtacctgcagatgaactccctgagagccgaggacaccgccgtgtactactgtgccagagagaaggcctctgacctgttcggctcttactctgaggccctggattattggggccagggcacactggttaccgtgtcatcagcctccaccaagggcccctccgtgtttcctctggccccttccagcaagtccacctctggcggaacagccgctctgggctgcctcgtgaaggactacttccccgagcctgtgaccgtgtcctggaactctggcgctctgacatccggcgtgcacaccttccctgctgtgctgcagtctagcggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccctggc

SEQ ID NO: 34 - DNA, 642 bp, Artificial Sequence
  LRRC15 antibody
gatatccagatgacccagtctcctgactctctgtccgcctctgtgggcgacagagtgaccatcacctgtagagcctctcaggacgtcggctcttggctggcttggtatcagcagaagcctggcaaggcccctaagctgctgatctttgccgcctcctctctggaatctggcatcccctctagattctccggctctggctctggcaccgactttaccctgacaatctccagcctgcagcctgaggacttcgccacctactactgtcagcaggccaacggcttcccactgacatttggcggcggaacaaaggtggaaatcaaacgaaccgtggccgctccctccgtgttcatcttcccaccctccgacgagcagctgaagtccggcaccgccagcgtcgtgtgcctgctgaacaacttctacccccgcgaggccaaggtgcagtggaaggtggacaacgccctgcagtccggcaactcccaggaatccgtcaccgagcaggactccaaggacagcacctactccctgtcctccaccctgaccctgtccaaggccgactacgagaagcacaaggtgtacgcctgcgaagtgacccaccagggcctgtccagccccgtgaccaagtccttcaaccggggcgagtgc

SEQ ID NO: 35 - protein, 125 aa, Artificial Sequence
  LRRC15 antibody
EVQLLESGGGLVQPGGSLRLSCAASGFTFSGYMMSWVRQAPGKGLEWVSGIYPSPGYTYYADSVKGRFTISRDNSKNTLYLQMNSLRAEDTAVYYCAREKASDLSGSYSEALDYWGQGTLVTVSS

SEQ ID NO: 36 - protein, 5 aa, Artificial Sequence
  LRRC15 antibody
GYMMS

SEQ ID NO: 37 - protein, 17 aa, Artificial Sequence
  LRRC15 antibody
GIYPSPGYTYYADSVKG

SEQ ID NO: 38 - protein, 16 aa, Artificial Sequence
  LRRC15 antibody
EKASDLSGSYSEALDY

SEQ ID NO: 39 - protein, 107 aa, Artificial Sequence
  LRRC15 antibody
DIQMTQSPDSLSASVGDRVTITCRASQDVGSWLAWYQQKPGKAPKLLIFAASYLESGIPSRFSGSGSGTDFTLTISSLQPEDFATYYCQQANGFPLTFGGGTKVEIK

SEQ ID NO: 40 - protein, 11 aa, Artificial Sequence
  LRRC15 antibody
RASQDVGSWLA

SEQ ID NO: 41 - protein, 7 aa, Artificial Sequence
  LRRC15 antibody
AASYLES

SEQ ID NO: 42 - protein, 9 aa, Artificial Sequence
  LRRC15 antibody
QQANGFPLT

SEQ ID NO: 43 - DNA, 375 bp, Artificial Sequence
  LRRC15 antibody
gaggtgcagctgctggaatctggcggaggattggttcagcctggcggctctctgagactgtcttgtgccgcttctggcttcaccttctccggctacatgatgtcctgggtccgacaggctcctggcaaaggactggaatgggtgtccggcatctatcccagtcctggctacacctactacgccgactctgtgaagggcagattcaccatcagccgggacaactccaagaacaccctgtacctgcagatgaactccctgagagccgaggacaccgccgtgtactactgtgccagagagaaggcctctgacctgtccggctcttactctgaggccctggattattggggccagggcacactggttaccgtgtcatca

SEQ ID NO: 44 - DNA, 321 bp, Artificial Sequence
  LRRC15 antibody
gatatccagatgacccagtctcctgactctctgtccgcctctgtgggcgacagagtgaccatcacctgtagagcctctcaggacgtcggctcttggctggcttggtatcagcagaagcctggcaaggcccctaagctgctgatcttcgccgcctcctatctggaaagcggcatcccttccagattctccggctctggctctggcaccgactttaccctgacaatctccagcctgcagcctgaggacttcgccacctactactgtcagcaggccaacggcttcccactgacatttggcggcggaacaaaggtggaaatcaaa

SEQ ID NO: 45 - protein, 454 aa, Artificial Sequence
  LRRC15 antibody
EVQLLESGGGLVQPGGSLRLSCAASGFTFSGYMMSWVRQAPGKGLEWVSGIYPSPGYTYYADSVKGRFTISRDNSKNTLYLQMNSLRAEDTAVYYCAREKASDLSGSYSEALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 46 - protein, 214 aa, Artificial Sequence
  LRRC15 antibody
DIQMTQSPDSLSASVGDRVTITCRASQDVGSWLAWYQQKPGKAPKLLIFAASYLESGIPSRFSGSGSGTDFTLTISSLQPEDFATYYCQQANGFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - DNA, 1362 bp, Artificial Sequence
  LRRC15 antibody
gaggtgcagctgctggaatctggcggaggattggttcagcctggcggctctctgagactgtcttgtgccgcttctggcttcaccttctccggctacatgatgtcctgggtccgacaggctcctggcaaaggactggaatgggtgtccggcatctatcccagtcctggctacacctactacgccgactctgtgaagggcagattcaccatcagccgggacaactccaagaacaccctgtacctgcagatgaactccctgagagccgaggacaccgccgtgtactactgtgccagagagaaggcctctgacctgtccggctcttactctgaggccctggattattggggccagggcacactggttaccgtgtcatcagcctccaccaagggcccctccgtgtttcctctggccccttccagcaagtccacctctggcggaacagccgctctgggctgcctcgtgaaggactacttccccgagcctgtgaccgtgtcctggaactctggcgctctgacatccggcgtgcacaccttccctgctgtgctgcagtctagcggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccctggc

SEQ ID NO: 48 - DNA, 642 bp, Artificial Sequence
  LRRC15 antibody
gatatccagatgacccagtctcctgactctctgtccgcctctgtgggcgacagagtgaccatcacctgtagagcctctcaggacgtcggctcttggctggcttggtatcagcagaagcctggcaaggcccctaagctgctgatcttcgccgcctcctatctggaaagcggcatcccttccagattctccggctctggctctggcaccgactttaccctgacaatctccagcctgcagcctgaggacttcgccacctactactgtcagcaggccaacggcttcccactgacatttggcggcggaacaaaggtggaaatcaaacgaaccgtggccgctccctccgtgttcatcttcccaccctccgacgagcagctgaagtccggcaccgccagcgtcgtgtgcctgctgaacaacttctacccccgcgaggccaaggtgcagtggaaggtggacaacgccctgcagtccggcaactcccaggaatccgtcaccgagcaggactccaaggacagcacctactccctgtcctccaccctgaccctgtccaaggccgactacgagaagcacaaggtgtacgcctgcgaagtgacccaccagggcctgtccagccccgtgaccaagtccttcaaccggggcgagtgc

SEQ ID NO: 49 - protein, 125 aa, Artificial Sequence
  LRRC15 antibody
EVQLLESGGGLVQPGGSLRLSCAASGFTFSGYMMSWVRQAPGKGLEWVSGIYPSAGYTLYADSVKGRFTISRDNSKNTLYLQMNSLRAEDTAVYYCAREKAADLFGSYSEALDYWGQGTLVTVSS

SEQ ID NO: 50 - protein, 5 aa, Artificial Sequence
  LRRC15 antibody
GYMMS

SEQ ID NO: 51 - protein, 17 aa, Artificial Sequence
  LRRC15 antibody
GIYPSAGYTLYADSVKG

SEQ ID NO: 52 - protein, 16 aa, Artificial Sequence
  LRRC15 antibody
EKAADLFGSYSEALDY

SEQ ID NO: 53 - protein, 107 aa, Artificial Sequence; identical to SEQ ID NO: 39
  LRRC15 antibody

SEQ ID NO: 54 - protein, 11 aa, Artificial Sequence
  LRRC15 antibody
RASQDVGSWLA

SEQ ID NO: 55 - protein, 7 aa, Artificial Sequence
  LRRC15 antibody
AASYLES

SEQ ID NO: 56 - protein, 9 aa, Artificial Sequence
  LRRC15 antibody
QQANGFPLT

SEQ ID NO: 57 - protein, 454 aa, Artificial Sequence
  LRRC15 antibody
EVQLLESGGGLVQPGGSLRLSCAASGFTFSGYMMSWVRQAPGKGLEWVSGIYPSAGYTLYADSVKGRFTISRDNSKNTLYLQMNSLRAEDTAVYYCAREKAADLFGSYSEALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 58 - protein, 214 aa, Artificial Sequence; identical to SEQ ID NO: 46
  LRRC15 antibody

SEQ ID NO: 59 - protein, 125 aa, Artificial Sequence
  LRRC15 antibody
EVQLLESGGGLVQPGGSLRLSCAASGFTFSGYMMSWVRQAPGKGLEWVSGIYPSGGYALYADSVKGRFTISRDNSKNTLYLQMNSLRAEDTAVYYCAREKAADLFGSYSEALDYWGQGTLVTVSS

SEQ ID NO: 60 - protein, 5 aa, Artificial Sequence
  LRRC15 antibody
GYMMS

SEQ ID NO: 61 - protein, 17 aa, Artificial Sequence
  LRRC15 antibody
GIYPSGGYALYADSVKG

SEQ ID NO: 62 - protein, 16 aa, Artificial Sequence
  LRRC15 antibody
EKAADLFGSYSEALDY

SEQ ID NO: 63 - protein, 107 aa, Artificial Sequence; identical to SEQ ID NO: 39
  LRRC15 antibody

SEQ ID NO: 64 - protein, 11 aa, Artificial Sequence
  LRRC15 antibody
RASQDVGSWLA

SEQ ID NO: 65 - protein, 7 aa, Artificial Sequence
  LRRC15 antibody
AASYLES

SEQ ID NO: 66 - protein, 9 aa, Artificial Sequence
  LRRC15 antibody
QQANGFPLT

SEQ ID NO: 67 - protein, 454 aa, Artificial Sequence
  LRRC15 antibody
EVQLLESGGGLVQPGGSLRLSCAASGFTFSGYMMSWVRQAPGKGLEWVSGIYPSGGYALYADSVKGRFTISRDNSKNTLYLQMNSLRAEDTAVYYCAREKAADLFGSYSEALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 68 - protein, 214 aa, Artificial Sequence; identical to SEQ ID NO: 46
  LRRC15 antibody

SEQ ID NO: 69 - protein, 120 aa, Artificial Sequence; identical to SEQ ID NO: 11
  LRRC15 antibody

SEQ ID NO: 70 - protein, 5 aa, Artificial Sequence
  LRRC15 antibody
SYWIE

SEQ ID NO: 71 - protein, 17 aa, Artificial Sequence
  LRRC15 antibody
EILPGSDTTNYNEKFKD

SEQ ID NO: 72 - protein, 11 aa, Artificial Sequence
  LRRC15 antibody
DRGNYRAWFGY

SEQ ID NO: 73 - protein, 107 aa, Artificial Sequence
  LRRC15 antibody
DIQMTQSPSSLSASVGDRVTITCRASQSISSYLNWYQQKPGGAPKFLIYYASSLQSGVPSRFSGSGSGTDYTLTISSLQPEDFATYYCQQGFSLPWTFGGGTKVEIK

SEQ ID NO: 74 - protein, 11 aa, Artificial Sequence
  LRRC15 antibody
RASQSISSYLN

SEQ ID NO: 75 - protein, 7 aa, Artificial Sequence
  LRRC15 antibody
YASSLQS

SEQ ID NO: 76 - protein, 9 aa, Artificial Sequence
  LRRC15 antibody
QQGFSLPWT

SEQ ID NO: 77 - DNA, 360 bp, Artificial Sequence
  LRRC15 antibody
gaggtgcagctggtgcagtctggcgccgaagtgaaaaagcctggcgcctctgtgaaggtgtcctgcaaggcttccggctacaagttctccagctactggatcgagtgggtcaagcaggctcctggacagggactcgagtggatcggagagatcctgcctggctctgacaccaccaactacaacgagaagttcaaggaccgggccaccttcacctccgacacctctatcaacaccgcctacatggaactgtcccggctgagatctgacgacaccgccgtgtactactgcgccagagacagaggcaactacagagcttggtttggctactggggccagggcacactggttacagttagctca

SEQ ID NO: 78 - DNA, 321 bp, Artificial Sequence
  LRRC15 antibody
gatatccagatgacccagtctccttccagcctgtctgcctctgtgggcgacagagtgaccatcacctgtcgggcctctcagtccatctcctcctacctgaactggtatcagcagaagcctggcggcgctcccaagttcctgatctactacgctagctccctgcagtccggcgtgccctctagattttctggctctggatccggcaccgactataccctgacaatctccagcctgcagcctgaggacttcgccacctactattgccagcagggcttctccctgccttggacatttggcggcggaacaaaggtggaaatcaaa

SEQ ID NO: 79 - protein, 449 aa, Artificial Sequence; identical to SEQ ID NO: 19
  LRRC15 antibody

SEQ ID NO: 80 - protein, 214 aa, Artificial Sequence
  LRRC15 antibody
DIQMTQSPSSLSASVGDRVTITCRASQSISSYLNWYQQKPGGAPKFLIYYASSLQSGVPSRFSGSGSGTDYTLTISSLQPEDFATYYCQQGFSLPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - DNA, 1347 bp, Artificial Sequence
  LRRC15 antibody
gaggtgcagctggtgcagtctggcgccgaagtgaaaaagcctggcgcctctgtgaaggtgtcctgcaaggcttccggctacaagttctccagctactggatcgagtgggtcaagcaggctcctggacagggactcgagtggatcggagagatcctgcctggctctgacaccaccaactacaacgagaagttcaaggaccgggccaccttcacctccgacacctctatcaacaccgcctacatggaactgtcccggctgagatctgacgacaccgccgtgtactactgcgccagagacagaggcaactacagagcttggtttggctactggggccagggcacactggttacagttagctcagcctccaccaagggcccctccgtgtttcctctggccccttccagcaagtccacctctggcggaacagccgctctgggctgcctcgtgaaggactacttccccgagcctgtgaccgtgtcctggaactctggcgctctgacatccggcgtgcacaccttccctgctgtgctgcagtctagcggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccctggc

SEQ ID NO: 82 - DNA, 642 bp, Artificial Sequence
  LRRC15 antibody
gatatccagatgacccagtctccttccagcctgtctgcctctgtgggcgacagagtgaccatcacctgtcgggcctctcagtccatctcctcctacctgaactggtatcagcagaagcctggcggcgctcccaagttcctgatctactacgctagctccctgcagtccggcgtgccctctagattttctggctctggatccggcaccgactataccctgacaatctccagcctgcagcctgaggacttcgccacctactattgccagcagggcttctccctgccttggacatttggcggcggaacaaaggtggaaatcaaacgaaccgtggccgctccctccgtgttcatcttcccaccctccgacgagcagctgaagtccggcaccgccagcgtcgtgtgcctgctgaacaacttctacccccgcgaggccaaggtgcagtggaaggtggacaacgccctgcagtccggcaactcccaggaatccgtcaccgagcaggactccaaggacagcacctactccctgtcctccaccctgaccctgtccaaggccgactacgagaagcacaaggtgtacgcctgcgaagtgacccaccagggcctgtccagccccgtgaccaagtccttcaaccggggcgagtgc

SEQ ID NO: 83 - protein, 120 aa, Artificial Sequence
  LRRC15 antibody
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRGNYRAWFQYWGQGTLVTVSS

SEQ ID NO: 84 - protein, 5 aa, Artificial Sequence
  LRRC15 antibody
SYWIE

SEQ ID NO: 85 - protein, 17 aa, Artificial Sequence
  LRRC15 antibody
EILPGSDTTNYNEKFKD

SEQ ID NO: 86 - protein, 11 aa, Artificial Sequence
  LRRC15 antibody
DRGNYRAWFQY

SEQ ID NO: 87 - protein, 107 aa, Artificial Sequence
  LRRC15 antibody
DIQMTQSPSSLSASVGDRVTITCRASQRISSYLNWYQQKPGGAPKFLIYYASSLQSGVPSRFSGSGSGTDYTLTISSLQPEDFATYYCDQGLELPWTFGGGTKVEIK

SEQ ID NO: 88 - protein, 11 aa, Artificial Sequence
  LRRC15 antibody
RASQRISSYLN

SEQ ID NO: 89 - protein, 7 aa, Artificial Sequence
  LRRC15 antibody
YASSLQS

SEQ ID NO: 90 - protein, 9 aa, Artificial Sequence
  LRRC15 antibody
DQGLELPWT

SEQ ID NO: 91 - protein, 449 aa, Artificial Sequence
  LRRC15 antibody
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRGNYRAWFQYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 92 - protein, 214 aa, Artificial Sequence
  LRRC15 antibody
DIQMTQSPSSLSASVGDRVTITCRASQRISSYLNWYQQKPGGAPKFLIYYASSLQSGVPSRFSGSGSGTDYTLTISSLQPEDFATYYCDQGLELPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 120 aa, Artificial Sequence
  LRRC15 antibody
EVQLVQSGAEVKKPGASVKVSCKASGYKFSSYWIEWVKQAPGQGLEWIGEILPGSDWTNYNEKFKDRATFTSDTSINTAYMELSRLRSDDTAVYYCARDRGNYRAWFQYWGQGTLVTVSS

SEQ ID NO: 94 - protein, 5 aa, Artificial Sequence
  LRRC15 antibody
SYWIE

SEQ ID NO: 95 - protein, 17 aa, Artificial Sequence
  LRRC15 antibody
EILPGSDWTNYNEKFKD

SEQ ID NO: 96 - protein, 11 aa, Artificial Sequence
  LRRC15 antibody
DRGNYRAWFQY

SEQ ID NO: 97 - protein, 107 aa, Artificial Sequence; identical to SEQ ID NO: 73
  LRRC15 antibody

SEQ ID NO: 98 - protein, 11 aa, Artificial Sequence
  LRRC15 antibody
RASQSISSYLN

SEQ ID NO: 99 - protein, 7 aa, Artificial Sequence
  LRRC15 antibody
YASSLQS

SEQ ID NO: 100 - protein, 9 aa, Artificial Sequence
  LRRC15 antibody
QQGFSLPWT

SEQ ID NO: 101 - protein, 449 aa, Artificial Sequence
  LRRC15 antibody
EVQLVQSGAEVKKPGASVKVSCKASGYKFSSYWIEWVKQAPGQGLEWIGEILPGSDWTNYNEKFKDRATFTSDTSINTAYMELSRLRSDDTAVYYCARDRGNYRAWFQYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 102 - protein, 214 aa, Artificial Sequence; identical to SEQ ID NO: 80
  LRRC15 antibody

SEQ ID NO: 103 - protein, 120 aa, Artificial Sequence; identical to SEQ ID NO: 93
  LRRC15 antibody

SEQ ID NO: 104 - protein, 5 aa, Artificial Sequence
  LRRC15 antibody
SYWIE

SEQ ID NO: 105 - protein, 17 aa, Artificial Sequence
  LRRC15 antibody
EILPGSDWTNYNEKFKD

SEQ ID NO: 106 - protein, 11 aa, Artificial Sequence
  LRRC15 antibody
DRGNYRAWFQY

SEQ ID NO: 107 - protein, 107 aa, Artificial Sequence
  LRRC15 antibody
DIQMTQSPSSLSASVGDRVTITCRISQSISSYLNWYQQKPGGAPKFLIYYASSLQSGVPSRFSGSGSGTDYTLTISSLQPEDFATYYCQQGLRLPWTFGGGTKVEIK

SEQ ID NO: 108 - protein, 11 aa, Artificial Sequence
  LRRC15 antibody
RISQSISSYLN

SEQ ID NO: 109 - protein, 7 aa, Artificial Sequence
  LRRC15 antibody
YASSLQS

SEQ ID NO: 110 - protein, 9 aa, Artificial Sequence
  LRRC15 antibody
QQGLRLPWT

SEQ ID NO: 111 - protein, 449 aa, Artificial Sequence; identical to SEQ ID NO: 101
  LRRC15 antibody

SEQ ID NO: 112 - protein, 214 aa, Artificial Sequence
  LRRC15 antibody
DIQMTQSPSSLSASVGDRVTITCRISQSISSYLNWYQQKPGGAPKFLIYYASSLQSGVPSRFSGSGSGTDYTLTISSLQPEDFATYYCQQGLRLPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 113 - protein, 120 aa, Artificial Sequence; identical to SEQ ID NO: 93
  LRRC15 antibody

SEQ ID NO: 114 - protein, 5 aa, Artificial Sequence
  LRRC15 antibody
SYWIE

SEQ ID NO: 115 - protein, 17 aa, Artificial Sequence
  LRRC15 antibody
EILPGSDWTNYNEKFKD

SEQ ID NO: 116 - protein, 11 aa, Artificial Sequence
  LRRC15 antibody
DRGNYRAWFQY

SEQ ID NO: 117 - protein, 107 aa, Artificial Sequence
  LRRC15 antibody
DIQMTQSPSSLSASVGDRVTITCRASQSISSYLNWYQQKPGGAPKFLIYYASSLQSGVPSRFSGSGSGTDYTLTISSLQPEDFATYYCQQGLELPWTFGGGTKVEIK

SEQ ID NO: 118 - protein, 11 aa, Artificial Sequence
  LRRC15 antibody
RASQSISSYLN

SEQ ID NO: 119 - protein, 7 aa, Artificial Sequence
  LRRC15 antibody
YASSLQS

SEQ ID NO: 120 - protein, 9 aa, Artificial Sequence
  LRRC15 antibody
QQGLELPWT

SEQ ID NO: 121 - DNA, 360 bp, Artificial Sequence
  LRRC15 antibody
gaggtgcagctggtgcagtctggcgccgaagtgaaaaagcctggcgcctctgtgaaggtgtcctgcaaggcttccggctacaagttctccagctactggatcgagtgggtcaagcaggctcctggacagggactcgagtggatcggagagatcctgcctggctctgactggaccaactacaacgagaagttcaaggaccgggccaccttcacctccgacacctctatcaacaccgcctacatggaactgtcccggctgagatctgacgacaccgccgtgtactactgcgccagagacagaggcaactacagagcctggtttcagtactggggccagggcacactggtcacagtttcttca

SEQ ID NO: 122 - DNA, 321 bp, Artificial Sequence
  LRRC15 antibody
gatatccagatgacccagtctccttccagcctgtctgcctctgtgggcgacagagtgaccatcacctgtcgggcctctcagtccatctcctcctacctgaactggtatcagcagaagcctggcggcgctcccaagttcctgatctactacgctagctccctgcagtccggcgtgccctctagattttctggctctggatccggcaccgactataccctgacaatctccagcctgcagcctgaggacttcgccacctactactgtcagcagggactcgagctgccctggacatttggcggaggcaccaaggtggaaatcaaa

SEQ ID NO: 123 - protein, 449 aa, Artificial Sequence; identical to SEQ ID NO: 101
  LRRC15 antibody

SEQ ID NO: 124 - protein, 214 aa, Artificial Sequence
  LRRC15 antibody
DIQMTQSPSSLSASVGDRVTITCRASQSISSYLNWYQQKPGGAPKFLIYYASSLQSGVPSRFSGSGSGTDYTLTISSLQPEDFATYYCQQGLELPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 125 - DNA, 1347 bp, Artificial Sequence
  LRRC15 antibody
gaggtgcagctggtgcagtctggcgccgaagtgaaaaagcctggcgcctctgtgaaggtgtcctgcaaggcttccggctacaagttctccagctactggatcgagtgggtcaagcaggctcctggacagggactcgagtggatcggagagatcctgcctggctctgactggaccaactacaacgagaagttcaaggaccgggccaccttcacctccgacacctctatcaacaccgcctacatggaactgtcccggctgagatctgacgacaccgccgtgtactactgcgccagagacagaggcaactacagagcctggtttcagtactggggccagggcacactggtcacagtttcttcagcctccaccaagggcccctccgtgtttcctctggccccttccagcaagtccacctctggcggaacagccgctctgggctgcctcgtgaaggactacttccccgagcctgtgaccgtgtcctggaactctggcgctctgacatccggcgtgcacaccttccctgctgtgctgcagtctagcggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccctggc

SEQ ID NO: 126 - DNA, 642 bp, Artificial Sequence
  LRRC15 antibody
gatatccagatgacccagtctccttccagcctgtctgcctctgtgggcgacagagtgaccatcacctgtcgggcctctcagtccatctcctcctacctgaactggtatcagcagaagcctggcggcgctcccaagttcctgatctactacgctagctccctgcagtccggcgtgccctctagattttctggctctggatccggcaccgactataccctgacaatctccagcctgcagcctgaggacttcgccacctactactgtcagcagggactcgagctgccctggacatttggcggaggcaccaaggtggaaatcaaacgaaccgtggccgctccctccgtgttcatcttcccaccctccgacgagcagctgaagtccggcaccgccagcgtcgtgtgcctgctgaacaacttctacccccgcgaggccaaggtgcagtggaaggtggacaacgccctgcagtccggcaactcccaggaatccgtcaccgagcaggactccaaggacagcacctactccctgtcctccaccctgaccctgtccaaggccgactacgagaagcacaaggtgtacgcctgcgaagtgacccaccagggcctgtccagccccgtgaccaagtccttcaaccggggcgagtgc

SEQ ID NO: 127 - protein, 120 aa, Artificial Sequence; identical to SEQ ID NO: 11
  LRRC15 antibody

SEQ ID NO: 128 - protein, 5 aa, Artificial Sequence
  LRRC15 antibody
SYWIE

SEQ ID NO: 129 - protein, 17 aa, Artificial Sequence
  LRRC15 antibody
EILPGSDTTNYNEKFKD

SEQ ID NO: 130 - protein, 11 aa, Artificial Sequence
  LRRC15 antibody
DRGNYRAWFGY

SEQ ID NO: 131 - protein, 107 aa, Artificial Sequence
  LRRC15 antibody
DIQMTQSPSSLSASVGDRVTITCRASQSISSYLNWYQQKPGGAPKFLIYYASSLQSGVPSRFSGSGSGTDYTLTISSLQPEDFATYYCQQGLSLPWTFGGGTKVEIK

SEQ ID NO: 132 - protein, 11 aa, Artificial Sequence
  LRRC15 antibody
RASQSISSYLN

SEQ ID NO: 133 - protein, 7 aa, Artificial Sequence
  LRRC15 antibody
YASSLQS

SEQ ID NO: 134 - protein, 9 aa, Artificial Sequence
  LRRC15 antibody
QQGLSLPWT

SEQ ID NO: 135 - protein, 449 aa, Artificial Sequence; identical to SEQ ID NO: 19
  LRRC15 antibody

SEQ ID NO: 136 - protein, 214 aa, Artificial Sequence
  LRRC15 antibody
DIQMTQSPSSLSASVGDRVTITCRASQSISSYLNWYQQKPGGAPKFLIYYASSLQSGVPSRFSGSGSGTDYTLTISSLQPEDFATYYCQQGLSLPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - protein, 523 aa, Homo sapiens
YHGCPSECTCSRASQVECTGARIVAVPTPLPWNAMSLQILNTHITELNESPFLNISALIALRIEKNELSRITPGAFRNLGSLRYLSLANNKLQVLPIGLFQGLDSLESLLLSSNQLLQIQPAHFSQCSNLKELQLHGNHLEYIPDGAFDHLVGLTKLNLGKNSLTHISPRVFQHLGNLQVLRLYENRLTDIPMGTFDGLVNLQELALQQNQIGLLSPGLFHNNHNLQRLYLSNNHISQLPPSVFMQLPQLNRLTLFGNSLKELSPGIFGPMPNLRELWLYDNHISSLPDNVFSNLRQLQVLILSRNQISFISPGAFNGLTELRELSLHTNALQDLDGNVFRMLANLQNISLQNNRLRQLPGNIFANVNGLMAIQLQNNQLENLPLGIFDHLGKLCELRLYDNPWRCDSDILPLRNWLLLNQPRLGTDTVPVCFSPANVRGQSLIIINVNVAVPSVHVPEVPSYPETPWYPDTPSYPDTTSVSSTTELTSPVEDYTDLTTIQVTDDRSVWGMTQAQSGHHHHHH

SEQ ID NO: 138 - protein, 523 aa, Mus musculus (Mouse)
YYGCPSECTCSRASQVECTGAQIVAMPSPLPWNAMSLQILNTHITELPEDKFLNISALIALKMEKNELANIMPGAFRNLGSLRHLSLANNKLKNLPVRLFQDVNNLETLLLSNNQLVQIQPAQFSQFSNLKELQLYGNNLEYIPEGVFDHLVGLTKLNLGNNGFTHLSPRVFQHLGNLQVLRLYENRLSDIPMGTFDALGNLQELALQENQIGTLSPGLFHNNRNLQRLYLSNNHISHLPPGIFMQLPHLNKLTLFGNSLKELSPGVFGPMPNLRELWLYNNHITSLPDNAFSHLNQLQVLILSHNQLSYISPGAFNGLTNLRELSLHTNALQDLDGNVFRSLANLRNVSLQNNRLRQLPGSIFANVNGLMTIQLQNNNLENLPLGIFDHLGNLCELRLYDNPWRCDSNILPLHDWLILNRARLGTDTLPVCSSPASVRGQSLVIINVNFPGPSVQGPETPEVSSYPDTSSYPDSTSISSTTEITRSTDDDYTDLNTIEPIDDRNTWGMTDAQSGAGHHHHHH

SEQ ID NO: 139 - protein, 525 aa, Macaca fascicularis (Cynomolgus monkey)
YYGCPSECTCSRASQVECTGARIVAVPTPLPWNAMSLQILNTHITELSESPFLNISALIALRIEKNELSHIMPGAFRNLGSLRYLSLANNKLQVLPIGLFQGLDSLESLLLSSNQLVQIQPAHFSQCSNLKELQLHGNHLEYIPDGAFDHLVGLTKLNLGKNSLTHISPRVFQHLGNLQVLRLYENRLTDIPMGTFDGLVNLQELALQQNQIGLLSPGLFHNNHNLQRLYLSNNHISQLPPSIFMQLPQLNRLTLFGNSLKELSPGIFGPMPNLRELWLYDNHITSLPDNVFSNLRQLQVLILSRNQISFISPGAFNGLTELRELSLHTNALQDLDGNVFRMLANLQNISLQNNRLRQLPGNIFANVNGLMTIQLQNNQLENLPLGIFDHLGKLCELRLYDNPWRCDSDILPLRNWLLLNQPRLGTDTVPVCFSPANVRGQSLIIINVNVAVPSVHVPEVPSYPETSWYPDTSSYPDTTSISSTTELTSPVEDYTDLTTIQVTDDRSVWGMTQAQSGAGHHHHHH

SEQ ID NO: 140 - protein, 98 aa, Artificial Sequence
  Human germline heavy chain (V-segment) - IGHV1-2-02
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCAR

SEQ ID NO: 141 - protein, 107 aa, Artificial Sequence
  Human germline light chain - IGKV1-NL1-01-IGKJ4-01-02
DIQMTQSPSSLSASVGDRVTITCRASQGISNSLAWYQQKPGKAPKLLLYAASRLESGVPSRFSGSGSGTDYTLTISSLQPEDFATYYCQQYYSTPLTFGGGTKVEIK

SEQ ID NO: 142 - protein, 15 aa, Artificial Sequence
  Human germline heavy chain (J-segment) - HV3-23-J1
YFDYWGQGTLVTVSS

SEQ ID NO: 143 - protein, 107 aa, Artificial Sequence [sequence omitted]

SEQ ID NO: 144 - protein, 98 aa, Artificial Sequence
  Human germline heavy chain (V-segment) - IGHV3-23-01
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAK